(19) United States
(12) Patent Application Publication
(10) Pub. No.: US 2023/0211024 A1
(43) Pub. Date: Jul. 6, 2023

(54) METHODS OF IMAGING USING MULTIPLE IMAGING AGENTS

(71) Applicant: ImaginAb, Inc., Inglewood, CA (US)

(72) Inventors: Alessandro Mascioni, Los Angeles, CA (US); Ian Andrew Wilson, Venice, CA (US); Ivan Plavec, Sunnyvale, CA (US); William Huy Le, Allen, TX (US)

(21) Appl. No.: 17/782,132

(22) PCT Filed: Dec. 3, 2020
(86) PCT No.: PCT/US2020/063023

§ 371 Date: Jun. 2, 2022

Related U.S. Application Data
(60) Provisional application No. 62/944,183, filed on Dec. 5, 2019.

(51) Int. Cl. A61K 51/10; A61B 6/03

(52) U.S. Cl. CPC A61K 51/1042; A61B 6/037; A61K 51/1045; A61K 2039/505

(57) ABSTRACT

Methods of non-invasively imaging a subject using two or more antigen-binding constructs that selectively bind to immune cell markers are described.

### CROSS-REFERENCE TO RELATED APPLICATIONS

The present application is a U.S. national phase application under 35 U.S.C. § 371 of International Application No. PCT/US2020/063023, filed Dec. 3, 2020, which claims the benefit of U.S. Provisional Application No. 62/944,183, filed Dec. 5, 2019. The content of each of these related applications is hereby incorporated by reference in its entirety.

### REFERENCE TO SEQUENCE LISTING

The present application is being filed along with a Sequence Listing in electronic format. The Sequence Listing is provided as a file entitled IGNAB050WO_SeqList.TXT, which was created on Dec. 3, 2020, which is 294,103 bytes in size, and is replaced by a file entitled IGNAB050WO_Substitute_SeqList.TXT, which was created on Jan. 20, 2021 and is 295,570 bytes in size. The information in the electronic Sequence Listing is hereby incorporated by reference in its entirety.

### BACKGROUND

### Field

The technology generally relates to non-invasive imaging methods for diagnosis, prediction, prognosis, and treatment of a disease.

### Description of the Related Art

Clinical evaluation of a disease often focuses on the characterization of the diseased tissue or an etiologic agent of the disease. For example, in cancer, the TNM classification system stages a cancer based on the size of the tumor and its spread to surrounding tissue; spread of the cancer to nearby lymph nodes, and metastasis. However, these methods do not take into account the patient's own immune response to the disease or treatment, which may affect disease progression and treatment outcomes.

### SUMMARY

Provided herein are methods of imaging a subject, comprising: administering to a subject a first antigen-binding construct comprising a first radionuclide tracer, wherein the antigen-binding construct selectively binds a first target selected from CD3, CD4, and CD8; estimating a distribution and/or abundance of cells expressing the first target in one or more tissues of the subject using positron emission tomography (PET) or single photon emission computed tomography (SPECT) to measure a level of the first radionuclide tracer in the subject; administering to the subject a second antigen-binding construct comprising a second radionuclide tracer, wherein the antigen-binding construct selectively binds a second target selected from CD3, CD4, and CD8, and wherein the first and second targets are different; and estimating a distribution and/or abundance of cells expressing the second target in the one or more tissues of the subject using PET or SPECT to measure a level of the second radionuclide tracer in the subject. Optionally, the method includes administering to the subject a third antigen-binding construct comprising a third radionuclide tracer, wherein the antigen-binding construct selectively binds a third target selected from CD3, CD4, and CD8, and wherein the third target is different from the first and second targets; and estimating a distribution and/or abundance of cells expressing the third target in the one or more tissues of the subject using PET or SPECT to measure a level of the third radionuclide tracer in the subject. The distributions and/or abundances of the cells expressing the targets (e.g., cells expressing the first, second and/or third targets) obtained by methods of the present disclosure may provide an immune contexture of the one or more tissues of the subject. Optionally, the method includes determining a relative abundance among cells expressing any one of the targets compared to cells expressing another one of the targets in each of the one or more tissues.

In some embodiments, the method includes generating an image (e.g., an image representing the immune contexture of the one or more tissues of the subject) based on the distributions and/or abundances of the cells expressing the targets (e.g., cells expressing the first, second and/or third targets). Optionally, the image provides one or more of: an abundance of any two or more of CD3⁺, CD4⁺ and CD8⁺ cells; a relative abundance of any one of CD3⁺, CD4⁺ and CD8⁺ cells compared to another one of CD3⁺, CD4⁺ and CD8⁺ cells; and a ratio of any one of CD3⁺, CD4⁺ and CD8⁺ cells to another one of CD3⁺, CD4⁺ and CD8⁺ cells, in the one or more tissues of the subject.

Also provided herein are methods of treating a subject, comprising: administering to a subject having a disease a first antigen-binding construct comprising a first radionuclide tracer, wherein the antigen-binding construct selectively binds a first target selected from CD3, CD4, and CD8; imaging the subject by positron emission tomography (PET) or single photon emission computed tomography (SPECT) to acquire a distribution of cells expressing the first target in one or more tissues of the subject; administering to the subject a second antigen-binding construct comprising a second radionuclide tracer, wherein the antigen-binding construct selectively binds a second target selected from CD3, CD4, and CD8, and wherein the first and second targets are different; imaging the subject by PET or SPECT to acquire a distribution of cells expressing the second target in the one or more tissues; determining an immune contexture of the one or more tissues based on the distribution of cells expressing the first target and the distribution of cells expressing the second target in the one or more locations; and administering a treatment to the subject based on the immune contexture. Optionally, the method includes administering to the subject a third antigen-binding construct comprising a third radionuclide tracer, wherein the antigen-binding construct selectively binds a third target selected from CD3, CD4, and CD8, wherein the third target is different from the first and second targets; and imaging the subject by PET or SPECT to acquire a distribution of cells expressing the third target in the one or more locations. Optionally, the method includes generating an image based on the distributions of cells expressing the targets, wherein the image provides the immune contexture of the one or more tissues.

According to methods of the present disclosure, the immune contexture of the imaged tissue comprises an abundance of, or relative abundance among, one or more of cytotoxic T cells (CD8⁺), helper T cells (CD4⁺), CD4±/CD8⁺ double positive T cells, CD8+NK cells, memory T cells and regulatory T cells (Tregs) in the tissue. In some embodiments, the immune contexture of the imaged tissue comprises one or more of: a ratio of CD4⁺ cells to CD8⁺ cells; a ratio of CD3⁺ cells to CD8⁺ cells; a ratio of CD3⁺ cells to CD4⁺ cells; an abundance of CD8⁺ cells and an abundance of CD3⁺ cells; an abundance of CD4⁺ cells and an abundance of CD3⁺ cells; or an abundance of CD8⁺ cells and an abundance of CD4⁺ cells.

Also provided herein are methods of treating a subject, comprising: administering to a subject having a cancer a first antigen-binding construct comprising a first radionuclide tracer, wherein the antigen-binding construct selectively binds a first target selected from CD3, CD4, and CD8; imaging the subject by positron emission tomography (PET) or single photon emission computed tomography (SPECT) to acquire a distribution of cells expressing the first target in a tumor in the subject; administering to the subject a second antigen-binding construct comprising a second radionuclide tracer, wherein the antigen-binding construct selectively binds a second target selected from CD3, CD4, and CD8, wherein the first and second targets are different; imaging the subject by PET or SPECT to acquire a distribution of cells expressing the second target in the tumor; estimating a density of CD3⁺ cells, CD4⁺ cells and/or CD8⁺ cells in a core and/or invasive margin of the tumor based on the distributions of cells expressing the targets; and administering to the subject treatment for the cancer based on a determination that the core and/or invasive margin of the tumor is depleted for one or more of CD3⁺, CD4⁺, or CD8⁺ cells, and/or enriched for one or more of CD3⁺, CD4⁺, or CD8⁺ cells. Optionally, the method includes administering to the subject a third antigen-binding construct comprising a third radionuclide tracer, wherein the antigen-binding construct selectively binds a third target selected from CD3, CD4, and CD8, wherein the third target is different from the first and second targets; and imaging the subject by PET or SPECT to acquire a distribution of cells expressing the third target in the tumor.

Optionally, administration of the treatment for the cancer is based on a determination that the core and/or invasive margin of the tumor is: depleted for CD3⁺ cells and CD8⁺ cells; depleted for CD3⁺ cells and CD4⁺ cells; depleted for CD4⁺ cells and enriched for CD8⁺ cells; or depleted for CD8⁺ cells and enriched for CD4⁺ cells; or depleted for CD8⁺ cells and CD4⁺ cells. In some embodiments, the core and/or invasive margin of the tumor is determined to be depleted: for CD8⁺ cells when the estimated density is 150 cells/mm² or less; for CD4⁺ cells when the estimated density is 150 cells/mm² or less; or for CD3⁺ cells when the estimated density is 300 cells/mm² or less. In certain embodiments, the core and/or invasive margin of the tumor is determined to be enriched: for CD4⁺ cells when the estimated density is 150 cells/mm² or more; for CD8⁺ cells when the estimated density is 150 cells/mm² or more; or for CD3⁺ cells when the estimated density is 300 cells/mm² or more. Optionally, estimating the density of CD3⁺ cells, CD4⁺ cells and/or CD8⁺ cells comprises: generating an image based on the distributions of cells expressing the targets; and estimating the density of CD3⁺ cells, CD4⁺ cells and/or CD8⁺ cells in a core and/or invasive margin of the tumor based on the image.

Further provided herein are methods of treating a subject, comprising: administering to a subject having a cancer a first antigen-binding construct comprising a first radionuclide tracer, wherein the antigen-binding construct selectively binds a first target selected from CD3, CD4, and CD8; imaging the subject by positron emission tomography (PET) or single photon emission computed tomography (SPECT) to acquire a distribution of cells expressing the first target in a tumor in the subject; administering to the subject a second antigen-binding construct comprising a second radionuclide tracer, wherein the antigen-binding construct selectively binds a second target selected from CD3, CD4, and CD8, and wherein the first and second targets are different; imaging the subject by PET or SPECT to acquire a distribution of cells expressing the second target in the tumor; estimating a ratio of: CD4⁺ cells to CD8⁺ cells; and/or CD8⁺ cells to CD4⁺ cells; and/or CD4⁺ cells to CD3⁺ cells; and/or CD8⁺ cells to CD3⁺ cells, in the tumor based on the acquired distributions; and administering to the subject a treatment for the cancer based on a determination that the ratio of: CD4⁺ cells to CD8⁺ cells is below a threshold; and/or CD8⁺ cells to CD4⁺ cells is below a threshold; and/or CD4⁺ cells to CD3⁺ cells is at or below a threshold ratio; and/or CD8⁺ cells to CD3⁺ cells is below a threshold, in the tumor. Optionally, the method includes administering to the subject a third antigen-binding construct comprising a third radionuclide tracer, wherein the antigen-binding construct selectively binds a third target selected from CD3, CD4, and CD8, wherein the third target is different from the first and second targets; and imaging the subject by PET or SPECT to acquire a distribution of cells expressing the third target in the tumor. Optionally, estimating the ratio comprises: generating an image based on the distributions of cells expressing the targets; and estimating the ratio of: CD4⁺ cells to CD8⁺ cells; and/or CD3⁺ cells to CD8⁺ cells, in the tumor based on the image.

Also provided are methods for providing a prognosis for a cancer, comprising: administering to a subject having a cancer a first antigen-binding construct comprising a first radionuclide tracer, wherein the antigen-binding construct selectively binds a first target selected from CD3, CD4, and CD8; imaging the subject by positron emission tomography (PET) or single photon emission computed tomography (SPECT) to acquire a distribution of cells expressing the first target in a tumor in the subject; administering to the subject a second antigen-binding construct comprising a second radionuclide tracer, wherein the antigen-binding construct selectively binds a second target selected from CD3, CD4, and CD8, and wherein the first and second targets are different; imaging the subject by PET or SPECT to acquire a distribution of cells expressing the second target in the tumor; determining an abundance of, and/or a relative abundance among, CD3⁺, CD4⁺ and/or CD8⁺ cells in the tumor based on the distributions of cells expressing the targets; and providing a prognosis for the disease based on an evaluation of the abundance of, and/or the relative abundance among, CD3⁺, CD4⁺ and/or CD8⁺ cells in the tumor. Optionally, the method includes administering to the subject a third antigen-binding construct comprising a third radionuclide tracer, wherein the antigen-binding construct selectively binds a third target selected from CD3, CD4, and CD8, wherein the third target is different from the first and second targets; and imaging the subject by PET or SPECT to acquire a distribution of cells expressing the third target in the tumor. Optionally, determining the abundance of, or a relative abundance among, CD3⁺, CD4⁺ and/or CD8⁺ cells in the tumor comprises: generating an image based on the distributions of cells expressing the targets; and determining the abundance of, and/or a relative abundance among, CD3⁺, CD4⁺ and/or CD8⁺ cells in the tumor based on the image.

Also provided herein are methods for treating a subject, comprising: administering to a subject having a disease a first treatment for the disease; before administering the first treatment, monitoring, by positron emission tomography (PET) or single photon emission computed tomography (SPECT): a distribution of cells expressing a first target selected from CD3, CD4, and CD8 in one or more tissues of the subject; and a distribution of cells expressing a second target selected from CD3, CD4, and CD8 in the one or more tissues of the subject, wherein the first and second targets are different; after administering the first treatment, monitoring, by PET or SPECT: a distribution of cells expressing the first target in the one or more tissues of the subject; and a distribution of cells expressing the second target in the one or more tissues of the subject; and administering to the subject a second treatment for the disease based on comparisons of: the distributions of cells expressing the first target; and the distributions of cells expressing the second target. Optionally, the method includes, before administering the first treatment, monitoring, by PET or SPECT, a distribution of cells expressing a third target selected from CD3, CD4, and CD8 in the one or more locations of the subject, wherein the third target is different from the first and second targets; and after administering the first treatment, monitoring, by PET or SPECT, a distribution of cells expressing the third target in the one or more locations of the subject, wherein administration of the second treatment is further based on a comparison of the distributions of cells expressing the third target. In some embodiments, before administering the first treatment, monitoring the distribution of cells expressing the first target is performed within 1 hour to 2 weeks of monitoring the distribution of cells expressing the second target and/or monitoring the distribution of cells expressing the second target is performed within 1 hour to 2 weeks of monitoring the distribution of cells expressing the third target. In certain embodiments, after administering the first treatment, monitoring the distribution of cells expressing the first target is performed within 1 hour to 2 weeks of monitoring the distribution of cells expressing the second target and/or monitoring the distribution of cells expressing the second target is performed within 1 hour to 2 weeks of monitoring the distribution of cells expressing the third target. Optionally, monitoring the distributions comprise: administering to the subject a first antigen-binding construct comprising a first radionuclide tracer, wherein the antigen-binding construct selectively binds the first target; imaging the subject by PET or SPECT to acquire the distribution of cells expressing the first target in the one or more tissues of the subject; administering to the subject a second antigen-binding construct comprising a second radionuclide tracer, wherein the antigen-binding construct selectively binds the second target, and wherein the first and second targets are different; imaging the subject by PET or SPECT to acquire the distribution of cells expressing the second target in the one or more tissues; and/or administering to the subject a third antigen-binding construct comprising a third radionuclide tracer, wherein the antigen-binding construct selectively binds the third target; imaging the subject by PET or SPECT to acquire the distribution of cells expressing the third target in the one or more tissues.

According to certain methods of the present disclosure, administering the first antigen-binding construct and imaging to acquire the distribution of cells expressing the second target are performed within 1 hour to 2 weeks. In some embodiments, measuring the level of the first radionuclide tracer is done within 1 hour to 2 weeks of administering the first antigen-binding construct. In certain embodiments, measuring the level of the second radionuclide tracer is done within 1 hour to 2 weeks of administering the second antigen-binding construct. In some embodiments, measuring the level of the third radionuclide tracer is done within 1 hour to 2 weeks of administering the third antigen-binding construct.

Optionally, different antigen-binding constructs are administered on different days. In some embodiments, administering the first antigen-binding construct and administering the second antigen-binding construct are performed on different days. In some embodiments, measuring the level of the first radionuclide tracer is performed on the same day as administering the second antigen-binding construct. In certain embodiments, measuring the level of the second radionuclide tracer is performed on the same day as administering the third antigen-binding construct. In some embodiments, administering the first antigen-binding construct and measuring the level of the second radionuclide tracer are performed on the same day. In some embodiments, administering the second antigen-binding construct and measuring the level of the third radionuclide tracer are performed on the same day.

Optionally, methods of the present disclosure further comprise determining a relative abundance among cells expressing any one of the targets compared to cells expressing another one of the targets in each of the one or more tissues.

Optionally, the subject has received an earlier treatment for the disease before administering to the subject the first antigen-binding construct. In some embodiments, the treatment and the earlier treatment are different.

In some embodiments, a treatment received by the subject comprises one or more of immunotherapy, chemotherapy, hormone therapy, radiation therapy, vaccine therapy (including intratumoral vaccine therapy), oncolytic virus therapy, surgery, or cellular therapy. In some embodiments, a treatment comprises one or more of immunotherapy, chemotherapy, hormone therapy, radiation therapy, vaccine therapy, oncolytic virus therapy, surgery, or cellular therapy.

According to certain embodiments, radionuclide tracers are each selected from ¹⁸F, ⁸⁹Zr, ¹²³I, ⁶⁴Cu, ⁶⁸Ga and ⁹⁹mTc. Optionally, the first, second, and/or third radionuclide tracer is one of ¹⁸F, ⁶⁴Cu, and ⁶⁸Ga. Optionally, the second radionuclide tracer is ¹⁸F or ⁸⁹Zr. Optionally, the first, second and/or third radionuclide tracer is ¹²³I or ⁹⁹mTc. In some embodiments, the second radionuclide tracer is ¹²³I or ⁹⁹mTc, where the first and second radionuclide tracers are different.

According to certain embodiments, the one or more tissues imaged in the subject comprises one or more of a lung, liver, colon, intestine, stomach, heart, brain, kidney, spleen, pancreas, esophagus, lymph node, bone, bone marrow, prostate, cervix, ovary, breast, urethra, bladder, skin, neck, articulated joint or portions thereof. Optionally, the subject has a cancer. Optionally, the subject has a cancer of a lung, liver, colon, intestine, stomach, brain, kidney, spleen, pancreas, esophagus, lymph node, bone, bone marrow, prostate, cervix, ovary, breast, urethra, bladder, skin or neck. In some embodiments, the subject has melanoma, non-small-cell lung carcinoma (NSCLC), or renal cell cancer (RCC). In some embodiments, the one or more tissues imaged or monitored comprises a tumor. Optionally, methods of the present disclosure include identifying the one or more tissues as comprising cancerous tissue. In some embodiments, the one or more tissues are identified as comprising cancerous tissue using computed tomography (CT) scan, X-ray, FDG-PET, or magnetic resonance imaging (MRI).

Also provided herein is a method of imaging a subject, comprising: administering to a subject a first antigen-binding construct comprising a first detectable marker, wherein the antigen-binding construct selectively binds a first target selected from CD3, CD4, IFN-gamma, and CD8; estimating a distribution and/or abundance of cells expressing the first target in one or more tissues of the subject using non-invasive imaging to measure a level of the first detectable marker in the subject; administering to the subject a second antigen-binding construct comprising a second detectable marker, wherein the antigen-binding construct selectively binds a second target selected from CD3, CD4, IFN-gamma, and CD8, and wherein the first and second targets are different; estimating a distribution and/or abundance of cells expressing the second target in the one or more tissues of the subject using non-invasive imaging to measure a level of the second detectable marker in the subject; and generating an image based on the distributions and/or abundances of the cells expressing the targets, wherein the image provides an indication of the immune contexture of the one or more tissues. Optionally, administering the first antigen binding construct and administering the second antigen binding construct are performed on the same day. In some embodiments, using non-invasive imaging to measure the level of the first detectable marker and using non-invasive imaging to measure the level of the second detectable marker are performed on the same day. In some embodiments, the first detectable marker and the second detectable marker are different and are selected from a radionuclide, an optical dye, a fluorescent compound, a Cerenkov luminescence agent, a paramagnetic ion, an MRI contrast agent, an MRI enhancer agent and a nanoparticle. In some embodiments, the non-invasive imaging is selected from PET, SPECT, MRI, CT, gamma-ray imaging, optical imaging, and Cherenkov luminescence imaging (CLI).

According to certain embodiments, the antigen-binding construct is an antibody or fragment thereof. Optionally, the antigen-binding construct is a Fab′, F(ab′)2, Fab, Fv, rIgG (reduced IgG), a scFv fragment, a minibody, a diabody, a cys-diabody, or a nanobody. In some embodiments, the antigen-binding construct that binds CD8 comprises an amino acid sequence at least about 80% identical to any one of the amino acid sequences shown in FIGS. 7-36. In some embodiments, the antigen-binding construct that binds CD4 comprises an amino acid sequence at least about 80% identical to any one of the amino acid sequences shown in FIGS. 38-50. In some embodiments, the antigen-binding construct that binds CD3 comprises an amino acid sequence at least about 80% identical to any one of the amino acid sequences shown in FIGS. 52A-84I.

In some embodiments, the CD3 is human CD3, the CD4 is human CD4 and the CD8 is human CD8. Optionally, the human CD3 comprises the sequence set forth in SEQ ID NO: 186, the human CD4 comprises the sequence set forth in SEQ ID NO: 100, and the human CD8 comprises any one of the sequences set forth in SEQ ID NOs: 80-82.

Also provided herein is a method of imaging a subject, comprising: administering to a subject a first PET tracer that selectively binds a first target selected from CD3, CD4, and CD8; estimating a distribution and/or abundance of cells expressing the first target in one or more tissues of the subject using positron emission tomography (PET) or single photon emission computed tomography (SPECT) to measure a signal from the first PET tracer in the subject; administering to the subject a second PET tracer that selectively binds a second target selected from CD3, CD4, and CD8, and wherein the first and second targets are different; estimating a distribution and/or abundance of cells expressing the second target in the one or more tissues of the subject using PET or SPECT to measure a signal from the second PET tracer in the subject; and generating an image based on the distributions and/or abundances of the cells expressing the targets, wherein the image provides an indication of the immune contexture of the one or more tissues.

### BRIEF DESCRIPTION OF THE DRAWINGS

FIGS. 1A and 1B are flow charts showing methods of imaging a subject, according to some embodiments of the present disclosure.

FIG. 2 is a flow chart showing a method of treating a subject, according to some embodiments of the present disclosure.

FIG. 3 is a flow chart showing methods of diagnosing and/or treating a subject, according to some embodiments of the present disclosure.

FIG. 4 is a schematic diagram showing a timeline for non-invasive imaging of a subject using multiple antigen-binding constructs labeled with a radionuclide tracer, according to some embodiments of the present disclosure.

FIG. 5 is a schematic diagram showing a timeline for non-invasive imaging of a subject using multiple antigen-binding constructs labeled with a radionuclide tracer, according to some embodiments of the present disclosure.

FIG. 6 is a schematic diagram showing a timeline for non-invasive imaging of a subject using multiple antigen-binding constructs labeled with a radionuclide tracer, according to some embodiments of the present disclosure.

FIG. 7 illustrates an alignment of the OKT8 Variable Heavy (V_{H}) region against a human antibody (4D5v8) and a humanized V_{H} region of a CD8 binding construct (IAB-huCD8 construct). the CDR regions (Chothia) are indicated by the boxed region).

FIG. 8 illustrates an alignment of the OKT8 Variable Light (V_{L}) region against a humanized V_{L} region and the IAB-huCD8 construct. the CDR regions (Chothia) are indicated by the boxed region).

FIG. 9 illustrates a chimeric IAB-huCD8 minibody VL-VH sequence.

FIG. 10 illustrates a chimeric IAB-huCD8 minibody VH-VL sequence.

FIG. 11 illustrates a humanized IAB-huCD8 minibody VL-VH sequence.

FIG. 12 illustrates a humanized IAB-huCD8 minibody VH-VL sequence.

FIG. 13 illustrates a humanized IAB-huCD8 cys-diabody VL-5-VH sequence.

FIG. 14 illustrates a humanized IAB-huCD8 cys-diabody VH-5-VL sequence.

FIG. 15 illustrates a humanized IAB-huCD8 cys-diabody VL-8-VH sequence.

FIG. 16 illustrates a humanized IAB-huCD8 cys-diabody VH-8-VL sequence.

FIG. 17A depicts sequences for VL of a CD8 binding construct.

FIG. 17B depicts sequences for huVL of a CD8 binding construct.

FIG. 17C depicts sequences for VH of a CD8 binding construct.

FIG. 17D depicts sequences for huVH (version “a” from Version 1) of a CD8 binding construct.

FIG. 17E depicts sequences for huVH (version “b” from Version 1) of a CD8 binding construct.

FIG. 17F depicts sequences for huVH (version “c” from Version 2) of a CD8 binding construct.

FIG. 17G depicts sequences for huVH (version “c” from Version 2) of a CD8 binding construct.

FIG. 18 shows protein sequence information for a CD8 binding construct IAB22M γ2 EH1.

FIG. 19 shows protein sequence information for a CD8 binding construct IAB22M γ2 EH2 variant.

FIG. 20 shows protein sequence information for a CD8 binding construct IAB22M γ1 EH1.

FIG. 21 shows protein sequence information for a CD8 binding construct IAB22M γ2 NH1.

FIG. 22 shows protein sequence information for a CD8 binding construct IAB22M γ2 NH2.

FIG. 23 shows protein sequence information for a CD8 binding construct IAB22M γ1 EH3.

FIG. 24 shows protein sequence information for a CD8 binding construct IAB22M γ1 EH5.

FIG. 25 shows protein sequence information for a CD8 binding construct IAB22M γ3/γ1 EH6.

FIG. 26 shows protein sequence information for a CD8 binding construct IAB22M γ3/γ1 EH7.

FIG. 27 shows protein sequence information for a CD8 binding construct IAB22M γ3/γ1 EH8.

FIG. 28 shows protein sequence information for a CD8 binding construct IAB22M γ1 EH2.

FIG. 29 shows the DNA and translated protein sequence of a CD8 binding construct IAB22M γ1 EH3(M1). In boxes are shown the signal, CDR, linker and hinge sequences.

FIG. 30 shows the DNA and translated protein sequence of a CD8 binding construct IAB22M γ1 EH5(M1).

FIG. 31 shows the DNA and translated protein sequence of a CD8 binding construct IAB22M γ1 EH7(M1).

FIG. 32 shows the DNA and translated protein sequence of a CD8 binding construct IAB22M γ1 EH8(M1).

FIG. 33 shows the DNA and translated protein sequence of a CD8 binding construct IAB22M γ2 EH2(M1).

FIG. 34 shows the DNA and translated protein sequence of a CD8 binding construct IAB22M γ2 EH2(M1) with VH-K67R polymorphism.

FIG. 35 shows the protein sequence of a CD8 binding construct IAB22M VH domain.

FIG. 36 depicts a CD8 antigen binding minibody.

FIG. 37A provides an example of a CD8 alpha chain.

FIG. 37B shows the protein sequence of an embodiment of T-cell surface glycoprotein CD8 alpha chain from Homo sapiens.

FIG. 37C shows the protein sequence of an embodiment of T-cell surface glycoprotein CD8 beta chain from Homo sapiens.

FIG. 38 illustrates an anti-CD4 antigen binding construct VH and VL sequences. SEQ ID NO: 84 and 86 include the VH and VL sequences for IAB41-1.

FIG. 39 illustrates an amino acid sequence for a CD4 antigen binding minibody.

FIG. 40 illustrates an amino acid sequence for a CD4 antigen binding minibody.

FIG. 41 illustrates an amino acid sequence for a CD4 antigen binding minibody.

FIG. 42 illustrates an amino acid sequence for a CD4 antigen binding minibody.

FIG. 43 illustrates an amino acid sequence for a CD4 antigen binding cys-diabody.

FIG. 44 illustrates an amino acid sequence for a CD4 antigen binding cys-diabody.

FIG. 45 illustrates an amino acid sequence for a CD4 antigen binding cys-diabody.

FIG. 46 illustrates an amino acid sequence for a CD4 antigen binding cys-diabody.

FIG. 47 illustrates an amino acid sequence for a CD4 antigen binding cys-diabody.

FIG. 48 illustrates an amino acid sequence for a CD4 antigen binding cys-diabody.

FIG. 49 illustrates an amino acid sequence for a CD4 antigen binding cys-diabody.

FIG. 50 illustrates an amino acid sequence for a CD4 antigen binding cys-diabody.

FIG. 51 provides an amino acid sequence of human CD4.

FIGS. 52A and 52B depict sequences showing the humanization of OKT3 variable light (FIG. 52A) and heavy (FIG. 52B) regions. The shaded and bolded cysteine in HCDR3 indicates the cysteine that was modified to a serine for some of the present embodiments. In some embodiments, HCDR3 (YYDDHYCLDY) (SEQ ID NO: 222) can be swapped with YYDDHYSLDY (SEQ ID NO: 223) (HCDR3 is YYDDHY(C/S)LDY (SEQ ID NO: 224)). Mouse sequences were compared with human variable light and heavy germline genes in FIGS. 52A and 52B. The murine OKT3 variable amino acid sequences (muOKT3) aligned with the human acceptor variable sequences (Human) are shown. The humanized/CDR grafted sequences (murine OKT3 CDRs within the human framework) are shown below (huOKT3, referred to as huVL_vB (panel A) and huVH_vB (panel B)). The CDRs are boxed using Chothia definition and the asterisks indicate residues that differ between the murine and the human framework.

FIG. 53 depicts a minibody to CD3 (V_{L}V_{H} orientation, murine)

FIG. 54 depicts a minibody to CD3 (V_{L}V_{H} orientation—ABC1).

FIG. 55 depicts a minibody to CD3 (V_{L}V_{H} orientation, humanized).

FIG. 56 depicts a cys-diabody to CD3 (humanized).

FIG. 57 provides a CD3 antigen binding minibody.

FIG. 58 provides a CD3 antigen binding cys-diabody.

FIG. 59 provides a CD3 antigen binding cys-diabody.

FIG. 60 provides a CD3 antigen binding cys-diabody.

FIG. 61 provides CD3 antigen binding cys-diabody.

FIG. 62 provides a CD3 antigen binding minibody.

FIG. 63 provides a CD3 antigen binding cys-diabody.

FIG. 64 provides a CD3 antigen binding cys-diabody.

FIG. 65 provides a CD3 antigen binding cys-diabody.

FIG. 66 provides a CD3 antigen binding cys-diabody.

FIG. 67 provides a CD3 antigen binding minibody.

FIG. 68 provides a CD3 antigen binding cys-diabody.

FIG. 69 provides a CD3 antigen binding cys-diabody.

FIG. 70 provides a CD3 antigen binding cys-diabody.

FIG. 71 provides a CD3 antigen binding cys-diabody.

FIG. 72 provides a CD3 antigen binding minibody.

FIG. 73 provides a CD3 antigen binding minibody.

FIG. 74 provides a CD3 antigen binding cys-diabody.

FIG. 75 provides a CD3 antigen binding cys-diabody.

FIG. 76 provides a CD3 antigen binding cys-diabody.

FIG. 77 provides a CD3 antigen binding cys-diabody.

FIG. 78 provides a CD3 antigen binding minibody.

FIG. 79 provides a CD3 antigen binding minibody.

FIG. 80 provides a CD3 antigen binding cys-diabody.

FIG. 81 provides a CD3 antigen binding cys-diabody.

FIG. 82 provides a CD3 antigen binding cys-diabody.

FIG. 83 provides a CD3 antigen binding cys-diabody.

FIGS. 84A, 84B, 84C, 84D, 84E, 84F, 84G, 84H, 84I depict anti-CD3 variable light (V_{L}; 84A, 84B, 84C) and variable heavy (V_{H}; 84D, 84E, 84F, 84F, 84H, 84I) sequences. The DNA with the translated amino acid sequences is shown. The V_{H} residue at position 105 is underlined. CDRs are boxed using Chothia definition.

FIG. 85 depicts the sequence of human CD3 Epsilon (amino acid sequence). Residues shaded have been identified as the epitope for OKT3.

FIG. 86 shows protein sequence information of various hinge regions.

### DETAILED DESCRIPTION

Provided herein are methods of non-invasively imaging a subject to determine an immune contexture of a tissue in the subject. Where the subject has a disease, such as cancer, autoimmune disease or an infectious disease, the immune contexture of the tissue affected by the disease may provide diagnostic or prognostic information about the disease, and/or or prognostic information about the subject's response to therapy. The present disclosure provides methods for determining the immune contexture of a tissue using imaging agents (e.g., PET tracer) for non-invasive imaging, such as positron emission tomography (PET), computed tomography (CT), and single photon emission computed tomography (SPECT). The imaging agents can be radionuclide-labeled antigen-binding constructs that are specific for immune cell markers, and can be used to obtain, through non-invasive imaging of the subject, the distribution and/or abundance of the binding targets of the antigen-binding constructs, e.g., two or more populations of immune cells, in the tissue. Any suitable imaging agent, e.g., antigen-binding construct associated with, or conjugated to a detectable marker, for non-invasive imaging, as disclosed herein, can be used in the present methods. The obtained distribution and/or abundance of the binding targets of the antigen-binding constructs, e.g., the immune cells, may represent aspects of the immune contexture of the tissue. Thus, also disclosed herein are methods of determining an immune contexture of a tissue using non-invasive imaging of a subject having a disease affecting the tissue.

Immunoscores can be obtained from one or more of the immune parameters of biopsied tissue using sequential immunohistochemistry and staining techniques to determine the presence/absence of relevant immune cell markers. However, detection of the immune cell markers can involve using invasive procedures to obtain the biopsy samples.

Some embodiments herein provide an immunoscore using non-invasive procedures. In some embodiments, the present methods provide one or more of faster result and/or diagnosis than conventional approaches (e.g., analyzing a biopsy sample), the capacity for whole body imaging of multiple disease sites, and reduced risk associated with using biopsy, such as the risk that the biopsy sample will miss critical tissue areas relevant to diagnosis. In some embodiments, methods of the present disclosure include providing a prognosis and/or treatment recommendation to a subject having a disease, e.g., cancer, without taking a biopsy sample, e.g., of a tumor, from the subject.

“Immune contexture” as used herein has the customary and ordinary meaning as understood by one of ordinary skill in the art, in view of the present disclosure. The immune contexture of a tissue (e.g., a tumor) may include, without limitation, the type, function, activity, density and/or location of immune cells, or a suitable surrogate measure thereof, in or around the tissue. Without wishing to be bound by theory, the immune contexture of a tissue associated with a disease, (e.g., a tumor, an organ or an anatomical region) may be a prognostic marker for predicting the response of the disease to treatment. The immune contexture of a tissue may include abundance and/or distribution of immune cells in the tissue. The immune contexture may include one or more immune cell types, such as, but not limited to, cytotoxic T cells, helper T cells, memory T cells, regulatory T cells (Tregs), B cells, natural killer cells, dendritic cells (DC), myeloid derived suppressor cells (MDSC), macrophages and mast cells. Immune cell types may be associated with expression of one or more immune cell markers (e.g., cell-surface markers expressed by one or more immune cell types), such as, but not limited to, CD8, CD3, CD4, and CD45RO. In general terms, CD4 expression may serve as a marker for immune cells that have a helper function (e.g., antigen presentation by dendritic cells, T helper function by CD4⁺ T cells, and “microenvironment” function by macrophages). CD8 expression may serve as a marker for immune cells that have an effector, or cytotoxic, function (e.g., cell killing by CD8⁺ T cells and NK cells, phagocytosis by M1 macrophages). CD3 expression may serve as a marker for T-cells (including CD4⁺ and CD8⁺ T cells). In some embodiments, the immune contexture of a tissue can include abundance and/or distribution of a marker, e.g., a cytokine, produced by immune cells in the tissue. In some embodiments, interferon (IFN)-gamma in the tissue serves as a marker for immune cell activation, such as T cell activation. In some embodiments, IFN-gamma serves as a marker for T helper 1 cells and/or B cells presence and activation. Embodiments of the methods disclosed herein can allow immune contexture of a tissue associated with disease to be determined by non-invasive imaging of CD8⁺, CD4⁺ and CD3⁺ cells and/or IFN-gamma distribution in a subject.

In some cases, the immune contexture of a tissue may be represented by the pattern and/or level of expression of one or more immune markers in the tissue. In some cases, the immune contexture of a tissue may include the functional activity of immune cells in the tissue and/or a functional environment of the tissue. The functional environment of the tissue may include tumor metabolism, the presence of immune checkpoints or tumor immune suppression status. The functional activity of immune cells may include, without limitation, antitumor T-cell activity. The functional activity of immune cells in the tissue and/or a functional environment of the tissue may be associated with expression of one or more functional markers, such as, but not limited to, IFNγ, Granzyme B, PD-1, PD-L1, and TGFβ. Thus, in some cases, the immune contexture of a tissue may be represented by the pattern and/or level of expression of one or more functional markers in the tissue. In some embodiments, the immune contexture may include a tumor infiltrating lymphocyte (TIL) status. In some embodiments, the immune contexture may be represented by an Immunoscore, as described in, e.g., Jerome Galon, et al; “Towards the introduction of the ‘Immunoscore’ in the classification of malignant tumours”; J Pathol. 2014 January; 232(2): 199-209; or Frank Pages et al., “International validation of the consensus Immunoscore for the classification of colon cancer: a prognostic and accuracy study.”; Lancet. 2018 391:2128-39. In some embodiments, an immune contexture, e.g., immunoscore, determined by the present methods excludes biomarkers other than CD3, CD4, CD8 and IFN-gamma. In some embodiments, an immune contexture, e.g., immunoscore, determined by the present methods excludes one or more of the following biomarkers: three prime repair exonuclease 1 (TREX1), programmed death ligand 1 (PD-L1).

The present disclosure discloses methods that can provide non-invasive imaging for measurement of immune cells in tissues which are not amenable to biopsy. Examples of monitoring such tissues include assessment of joints of arthritic patients, cardiotoxicity induced by immunotherapy, recovery from stroke, brain injury or cardiac event, or transplant rejection, none of which are recommended for biopsy. Methods of the present disclosure may allow obtaining an immune contexture for such conditions based on non-invasive visualization of a patient's immune system. In some embodiments, the immune contexture may be represented by an immunoscore, as described herein. As used herein, “immunoscore” may apply to any disease or condition (e.g., cancer, autoimmune disorders, infectious disease, etc.) where the immune contexture of a tissue is relevant to diagnosis and/or treatment of the disease or condition. An immunoscore may be applied to any suitable cancer, including, without limitation, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, bone cancer, hepatic carcinoma, anal carcinoma, penile carcinoma, melanoma, multiple myeloma and B-cell lymphoma, brain, as well as head and neck cancer, and associated metastases. In some embodiments, an immunoscore is applied in the context of colorectal cancer.

### Definitions and Various Embodiments

Unless defined, the plain and ordinary meaning of terms as understood by one of ordinary skill in the art apply.

“Treating” or “treatment” of a condition may refer to preventing the condition, slowing the onset and/or rate of development of the condition, reducing the risk of developing the condition, preventing and/or delaying the development of symptoms associated with the condition, reducing or ending symptoms associated with the condition, generating a complete or partial regression of the condition, or some combination thereof. The term “prevent” does not require the absolute prohibition of the disorder or disease. Treatment includes altering the immune phenotype of the tumor or neoplasia in the subject (from desert to excluded to TIL positive) as well as the subsequent therapeutic application for the tumor for that particular phenotype. A tumor characterized as an immune desert (or “cold” tumors) may show no or very little immune cell infiltration into the tumor environment. A tumor that is immune-excluded may show immune cells aggregated at the tumor boundaries. A tumor may be TIL positive (“hot” or “inflamed”) where immune cells infiltrate into the tumor core.

A “therapeutically effective amount” or a “therapeutically effective dose” is an amount that produces a desired therapeutic effect in a subject, such as preventing, treating a target condition, delaying the onset of the disorder and/or symptoms, and/or alleviating symptoms associated with the condition. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and/or the route of administration. One skilled in the clinical and pharmacological arts is able to determine a therapeutically effective amount through routine experimentation, for example by monitoring a subject's response to administration of a compound and adjusting the dosage accordingly, given the present disclosure. For additional guidance, see Remington: The Science and Practice of Pharmacy 21.sup.st Edition, Univ. of Sciences in Philadelphia (USIP), Lippincott Williams & Wilkins, Philadelphia, Pa., 2005.

The term “antigen binding construct” includes all varieties of antibodies, including binding fragments thereof. Further included are constructs that include 1, 2, 3, 4, 5, and/or 6 CDRs. In some embodiments, these CDRs can be distributed between their appropriate framework regions in a traditional antibody. In some embodiments, the CDRs can be contained within a heavy and/or light chain variable region. In some embodiments, the CDRs can be within a heavy chain and/or a light chain. In some embodiments, the CDRs can be within a single peptide chain. In some embodiments, the CDRs can be within two or more peptides that are covalently linked together. In some embodiments, they can be covalently linked together by a disulfide bond. In some embodiments, they can be linked via a linking molecule or moiety. In some embodiments, the antigen binding proteins are non-covalent, such as a diabody and a monovalent scFv. Unless otherwise denoted herein, the antigen binding constructs described herein bind to the noted target molecule. The term also includes minibodies and cys-diabodies.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The term “neoplasia” encompasses the term tumor.

“Tumor” means solid tumor unless indicated otherwise; includes neoplasia and any aberrant cellular growth of human cells in a subject (but does not include infection by foreign organism).

“Surface of tumor” or “tumor surface” means the outer perimeter of the tumor mass which is in contact with normal (e.g. non-tumor and non-tumor induced) cells of the subject. It is sometimes referred to interchangeably as the “tumor margin”, or “invasive tumor margin” or “tumor border”. At a cellular level it may range from a few cells to a few hundred cells in thickness and may be unevenly integrated with surrounding normal cells.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, bone cancer, hepatic carcinoma, anal carcinoma, penile carcinoma, melanoma, multiple myeloma and B-cell lymphoma, brain, as well as head and neck cancer, and associated metastases. The term cancer includes adult and pediatric solid cancers. In some embodiments, the cancer can be a solid tumor.

The term “antibody” includes, but is not limited to, genetically engineered or otherwise modified forms of immunoglobulins, such as intrabodies, chimeric antibodies, fully human antibodies, humanized antibodies, antibody fragments, and heteroconjugate antibodies (e.g., bispecific antibodies, diabodies, triabodies, tetrabodies, etc.). The term “antibody” includes cys-diabodies and minibodies. Thus, each and every embodiment provided herein in regard to “antibodies” is also envisioned as cys-diabody and/or minibody embodiments, unless explicitly denoted otherwise. The term “antibody” includes a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. In some embodiments, a full length antibody can be composed of two identical pairs of polypeptide chains, each pair having one “light” and one “heavy” chain (, connected through a disulfide bond. The recognized immunoglobulin genes include the kappa, lambda, alpha, gamma, delta, epsilon, and mu constant region genes, as well as the myriad immunoglobulin variable region genes. For full length chains, the light chains are classified as either kappa or lambda. For full length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively. As used in this application, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (e.g., F(ab′)2) with the same binding specificity. In some embodiments, the antibody binds specifically to a desired target.

“Complementarity-determining domains” or “complementarity-determining regions (“CDRs”) interchangeably refer to the hypervariable regions of VL and VH. The CDRs are the target protein-binding site of the antibody chains that harbors specificity for such target protein. In some embodiments, there are three CDRs (CDR1-3, numbered sequentially from the N-terminus) in each VL and/or VH, constituting about 15-20% of the variable domains. The CDRs are structurally complementary to the epitope of the target protein and are thus directly responsible for the binding specificity. The remaining stretches of the VL or VH, the so-called framework regions (FRs), exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat (Wu, T. T., E. A. Kabat. 1970. An analysis of the sequences of the variable regions of Bence Jones proteins and myeloma light chains and their implications for antibody complementarity. J. Exp. Med. 132: 211-250; Kabat, E. A., Wu, T. T., Perry, H., Gottesman, K., and Foeller, C. (1991) Sequences of Proteins of Immunological Interest, 5th ed., NIH Publication No. 91-3242, Bethesda, Md.), Chothia (Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)), ImMunoGeneTics database (IMGT) (on the worldwide web at imgt.org/) Giudicelli, V., Duroux, P., Ginestoux, C., Folch, G., Jabado-Michaloud, J., Chaume, D. and Lefranc, M.-P. IMGT/LIGM-DB, the IMGT® comprehensive database of immunoglobulin and T cell receptor nucleotide sequences Nucl. Acids Res., 34, D781-D784 (2006), PMID: 16381979; Lefranc, M.-P., Pommié, C., Ruiz, M., Giudicelli, V., Foulquier, E., Truong, L., Thouvenin-Contet, V. and Lefranc, G., IMGT unique numbering for immunoglobulin and T cell receptor variable domains and Ig superfamily V-like domains Dev. Comp. Immunol., 27, 55-77 (2003). PMID: 12477501; Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008); AbM (Martin et al., Proc. Natl. Acad. Sci. USA, 86:9268-9272 (1989), North (North B., Lehmann A., Dunbrack R. L., A new clustering of antibody CDR loop conformations, J. Mol. Biol. (2011) 406(2): 228-256), AHo (Honegger A., Pluckthun, Yet another numbering scheme for immunoglobulin variable domains: an automatic modeling and analysis tool, J. Mol. Biol. (2001) 309, 657-670); the contact definition (MacCallum et al., J. Mol. Biol., 262:732-745 (1996)), and/or the automatic modeling and analysis tool Honegger A, Plückthun A. (world wide web at bioc dot uzh dot ch/antibody/Numbering/index dot html).

The term “binding specificity determinant” or “BSD” interchangeably refer to the minimum contiguous or non-contiguous amino acid sequence within a complementarity determining region necessary for determining the binding specificity of an antibody. A minimum binding specificity determinant can be within one or more CDR sequences. In some embodiments, the minimum binding specificity determinants reside within (i.e., are determined solely by) a portion or the full-length of the CDR3 sequences of the heavy and light chains of the antibody. In some embodiments, CDR3 of the heavy chain variable region is sufficient for the antigen binding construct specificity.

The terms “binds in a biased manner” and “binds in a non-biased manner” with respect to the surface of a tumor, refers to an image of a tumor where the detectable label is observed to bind either substantially to the tumor surface (with relatively reduced or absent binding in the interior of the tumor) (=“biased”) or where the detectable label is not significantly associated with the surface of the tumor (=“non-biased”) and for example may be dispersed evenly or unevenly throughout the interior of the tumor or absent from the tumor altogether. “Biased” includes binding selectively to any tumor margin without substantially perfusing the volume of the tumor.

The term “Region of interest” or “ROI” means, in or on an image of target distribution in a human subject, a sub-area of the image that is selected by a human operator, optionally assisted by an automated or semi-automated imaging processing method, which narrowly circumscribes a region of the image which identifies a tumor, or is expected to contain a tumor based on other diagnostic methods (e.g. FDG-PET, CT scan, MRI, biopsy, visual inspection, etc.).

The term “distribution”, in the context of monitoring, detecting, comparing, or observing a distribution of an antigen binding construct associated with radionuclide tracer which has been administered to a subject, means a visual image of the biodistribution of a detected label associated with an antigen binding construct in relation to a whole body or partial body scan of the subject, which image may be represented as a flat image (2-dimensional) or as computer assisted three-dimensional representation (including a hologram), and is in a format useful to the operator or clinician to observe distribution of the antigen binding construct at the individual tissue level, and the individual tumor level. In advanced forms of imaging, a “distribution” may not be a visual image of a whole or partial body scan but may instead be a report of a computerized assessment of the absence or presence of a tumor in the subject, and its TIL status. In some cases, “comparing a distribution of two or more cells expressing different markers requires aligning the scan of the subject in each distribution so that individual tissues and tumors can be compared.

An “antibody variable light chain” or an “antibody variable heavy chain” as used herein refers to a polypeptide comprising the VL or VH, respectively. The endogenous VL is encoded by the gene segments V (variable) and J (junctional), and the endogenous VH by V, D (diversity), and J. Each of VL or VH includes the CDRs as well as the framework regions. In this application, antibody variable light chains and/or antibody variable heavy chains may, from time to time, be collectively referred to as “antibody chains.” These terms encompass antibody chains containing mutations that do not disrupt the basic structure of VL or VH, as one skilled in the art will readily recognize. In some embodiments, full length heavy and/or light chains are contemplated. In some embodiments, only the variable region of the heavy and/or light chains are contemplated as being present.

Antibodies can exist as intact immunoglobulins or as a number of fragments produced by digestion with various peptidases. Thus, for example, pepsin digests an antibody below the disulfide linkages in the hinge region to produce F(ab)′2, a dimer of Fab′ which itself is a light chain (VL-CL) joined to VH-CH1 by a disulfide bond. The F(ab)′2 may be reduced under mild conditions to break the disulfide linkage in the hinge region, thereby converting the F(ab)′2 dimer into a Fab′ monomer. The Fab′ monomer is a Fab with part of the hinge region. (Paul, Fundamental Immunology 3d ed. (1993). While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “hinge” denotes at least a part of a hinge region for an antigen binding construct, such as an antibody or a minibody. A hinge region can include a combination of the upper hinge, core (or middle) hinge and lower hinge regions. In some embodiments, the hinge is defined according to any of the antibody hinge definitions. Native IgG1, IgG2, and IgG4 antibodies have hinge regions having of 12-15 amino acids. IgG3 has an extended hinge region, having 62 amino acids, including 21 prolines and 11 cysteines. The functional hinge region of naturally occurring antibodies, deduced from crystallographic studies, extends from amino acid residues 216-237 of the IgG1 H chain (EU numbering) and includes a small segment of the N terminus of the CH2 domain in the lower hinge, with the lower hinge being the N terminus of CH2 domain. The hinge can be divided into three regions; the “upper hinge,” the “core,” and the “lower hinge”.

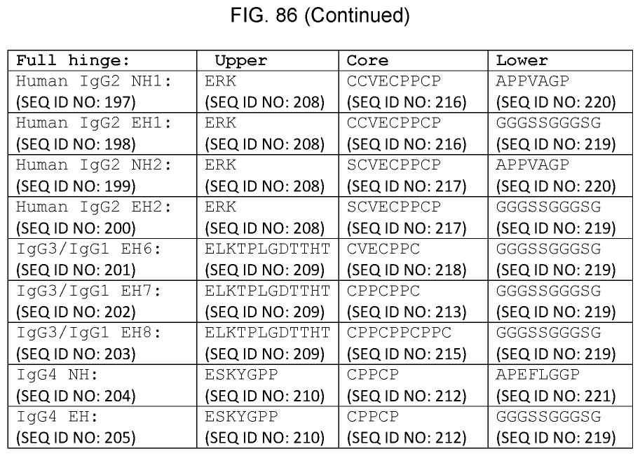

The term “upper hinge” denotes the first part of the hinge that starts at the end of the variable region of an antigen-binding construct, such as the end of the scFv. Examples of upper hinge regions can be found in FIG. 86. The upper hinge includes the amino acids from the end of the scFv up to, but not including, the first cysteine residue in the core hinge as shown in FIG. 86. As above, the term “effective upper hinge” denotes that enough of the sequence is present to allow the section to function as an upper hinge; the term encompasses functional variants and fragments of the designated hinge section.

The term “core hinge” denotes the second part of the hinge region that is C-terminal to the upper hinge. Examples of core hinge regions can be found in FIG. 86. The core hinge contains the inter-chain disulfide bridges and a high content of prolines. As above, the term “effective core hinge” denotes that enough of the sequence is present to allow the section to function as a core hinge; the term encompasses functional variants and fragments of the designated hinge section.

The term “lower hinge” denotes the third part of the hinge region that is C-terminal to the core hinge. Examples of lower hinge regions can be found in FIG. 86. In the context of a minibody or antibody fragment, the lower hinge connects to the C_{H}3 domain. As above, the term “effective lower hinge” denotes that enough of the sequence is present to allow the section to function as a lower hinge; the term encompasses functional variants and fragments of the designated hinge section. The term “lower hinge” as used herein can encompass various amino acid sequences including naturally occurring IgG lower hinge sequences and artificial extension sequences in place of one another or a combination thereof provided herein. In some embodiments, the various extensions can be considered to be a lower hinge region in its entirety or a replacement.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985; Advances in the production of human monoclonal antibodies Shixia Wang, Antibody Technology Journal 2011:1 1-4; J Cell Biochem. 2005 Oct. 1; 96(2):305-13; Recombinant polyclonal antibodies for cancer therapy; Sharon J, Liebman M A, Williams B R; and Drug Discov Today. 2006 July, 11(13-14):655-60, Recombinant polyclonal antibodies: the next generation of antibody therapeutics?, Haurum J S). Techniques for the production of single chain antibodies (U.S. Pat. No. 4,946,778) can be adapted to produce antibodies to polypeptides. Also, transgenic mice, or other organisms such as other mammals, may be used to express fully human monoclonal antibodies. Alternatively, phage display technology can be used to identify high affinity binders to selected antigens (see, e.g., McCafferty et al., supra; Marks et al., Biotechnology, 10:779-783, (1992)). B-cell cloning can be used to identify fully human antibodies directly from human subjects (Wardemann H., Busse E., Expression Cloning of Antibodies from Single Human B Cells, Methods Mol. Biol. (2019) 1956:105-125).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. In some embodiments, the terms “donor” and “acceptor” sequences can be employed. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies (as described in, e.g., U.S. Pat. No. 4,816,567) have substantially less than an intact human variable domain substituted by the corresponding sequence from a non-human species. In practice, humanized antibodies are typically human antibodies in which some complementarity determining region (“CDR”) residues and possibly some framework (“FR”) residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, and drug; or (b) the variable region, or a portion thereof, is altered, replaced or exchanged with a variable region having a different or altered antigen specificity.

Antibodies further include one or more immunoglobulin chains that are chemically conjugated to, or expressed as, fusion proteins with other proteins. In some embodiments, the antigen binding constructs can be a monovalent scFv constructs. In some embodiments, the antigen binding constructs can be a bispecific constructs. A bispecific or bifunctional antibody is an artificial hybrid antibody having two different heavy/light chain pairs and two different binding sites. Other antigen-binding fragments or antibody portions include bivalent scFv (diabody), bispecific scFv antibodies where the antibody molecule recognizes two different epitopes, single binding domains (sdAb or nanobodies), and minibodies.

The term “antibody fragment” includes, but is not limited to one or more antigen binding fragments of antibodies alone or in combination with other molecules, including, but not limited to Fab′, F(ab′)2, Fab, Fv, rIgG (reduced IgG), scFv fragments (monovalent, tri-valent, etc.), single domain fragments (nanobodies), peptibodies, minibodies, diabodies, and cys-diabodies. The term “scFv” refers to a single chain Fv (“fragment variable”) antibody in which the variable domains of the heavy chain and of the light chain of a traditional two chain antibody have been joined to form one chain.

A pharmaceutically acceptable carrier may be a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or some combination thereof. Each component of the carrier is “pharmaceutically acceptable” in that it is compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. The pharmaceutical compositions described herein may be administered by any suitable route of administration. A route of administration may refer to any administration pathway known in the art, including but not limited to aerosol, enteral, nasal, ophthalmic, oral, parenteral, rectal, transdermal (e.g., topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, intracranial, subarachnoid, subcapsular, subcutaneous, transmucosal, or transtracheal. In some embodiments, the antigen binding construct can be delivered intraoperatively as a local administration during an intervention or resection.

A minibody is an antibody format that has a smaller molecular weight than the full-length antibody while maintaining the bivalent binding property against an antigen. Because of its smaller size, the minibody has a faster clearance from the system and enhanced penetration when targeting tumor tissue. With the ability for strong and selective targeting combined with rapid clearance, the minibody is advantageous for diagnostic imaging and delivery of cytotoxic/radioactive payloads for which prolonged circulation times may result in adverse patient dosing or dosimetry.

The phrase “specifically bind” or “selectively bind,” when used in the context of describing the interaction between an antigen, e.g., a protein, to an antibody or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under designated immunoassay conditions, in some embodiments, the antibodies or binding agents with a particular binding specificity bind to a particular antigen at least two times the background and do not substantially bind in a significant amount to other antigens present in the sample. Specific binding to an antibody or binding agent under such conditions may require the antibody or agent to have been selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

The term “equilibrium dissociation constant (KD, M)” refers to the dissociation rate constant (kd, time⁻¹) divided by the association rate constant (ka, time⁻¹, M⁻¹). Equilibrium dissociation constants can be measured using any known method in the art. The antibodies provided herein can have an equilibrium dissociation constant of less than about 10⁻⁷ or 10⁻⁸ M, for example, less than about 10⁻⁹ M or 10⁻¹⁰ M, in some embodiments, less than about 10⁻¹¹ M, 10⁻¹² M, 10⁻¹³ M, 10⁻¹⁴ M or 10⁻¹⁵ M.

The term “isolated,” when applied to a nucleic acid or protein, denotes that the nucleic acid or protein is essentially free of other cellular components with which it is associated in the natural state. In some embodiments, it can be in either a dry or aqueous solution. Purity and homogeneity can be determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. In some embodiments, this can denote that the nucleic acid or protein is at least 85% pure, more preferably at least 95% pure, and most preferably at least 99% pure of molecules that are present under in vivo conditions.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the constructs provided herein.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” can be determined by comparing two optimally aligned sequences over a comparison window, wherein the portion of the polynucleotide sequence in the comparison window may comprise additions or deletions (i.e., gaps) as compared to the reference sequence (e.g., a polypeptide of the constructs provided herein), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

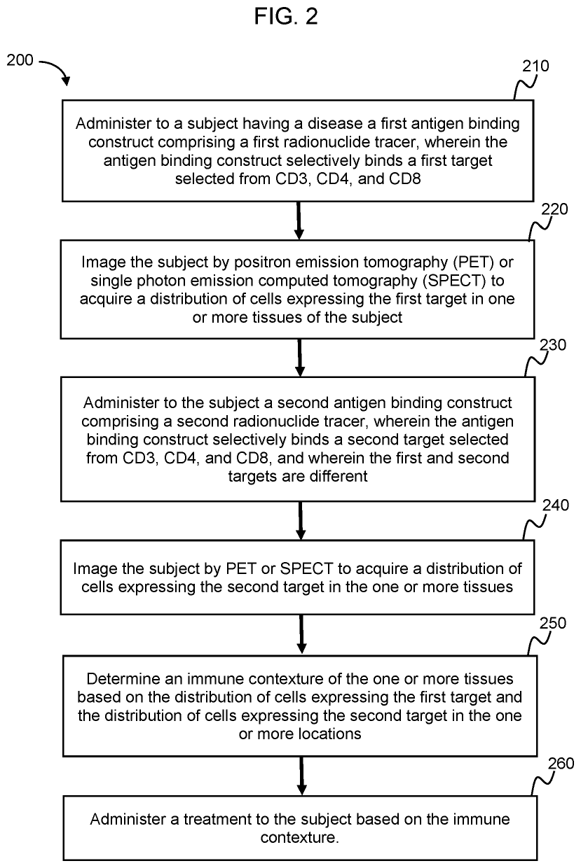

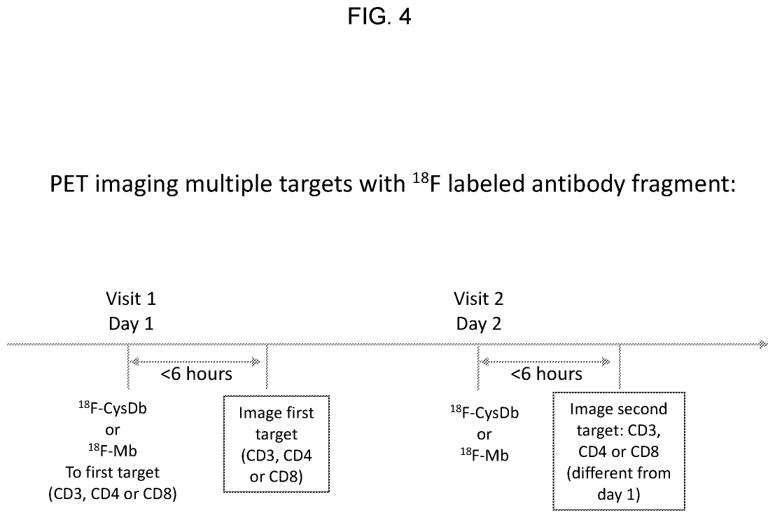

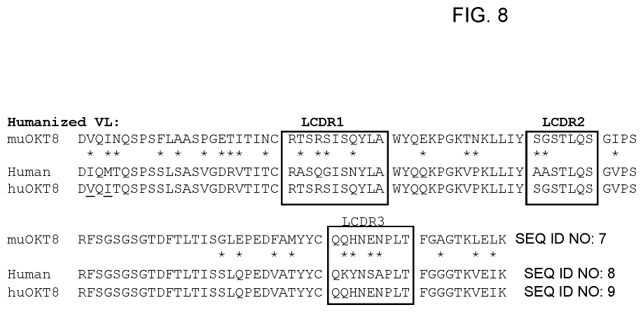

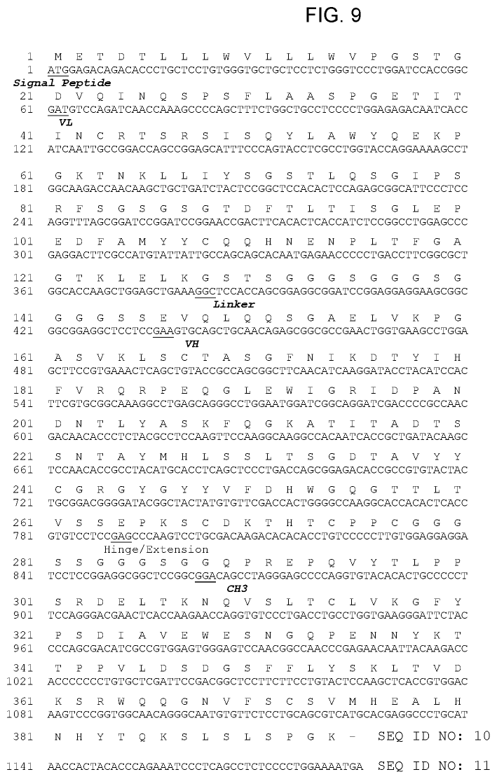

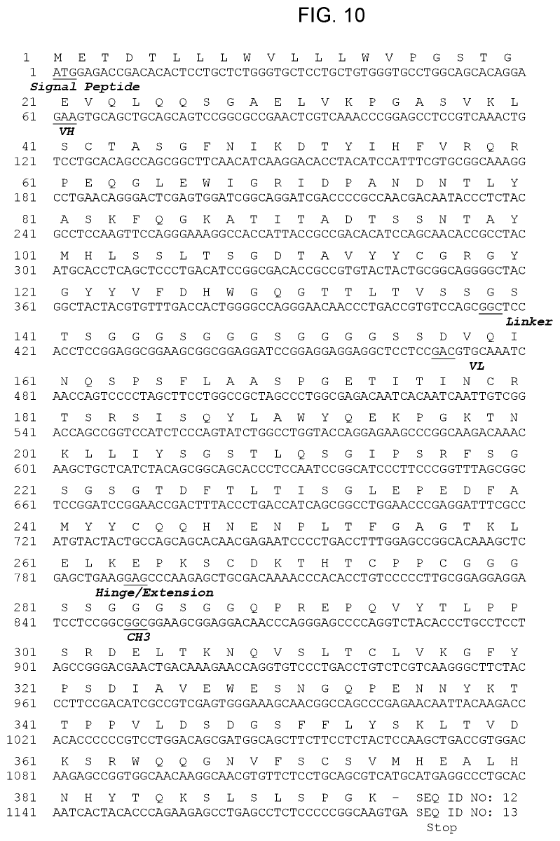

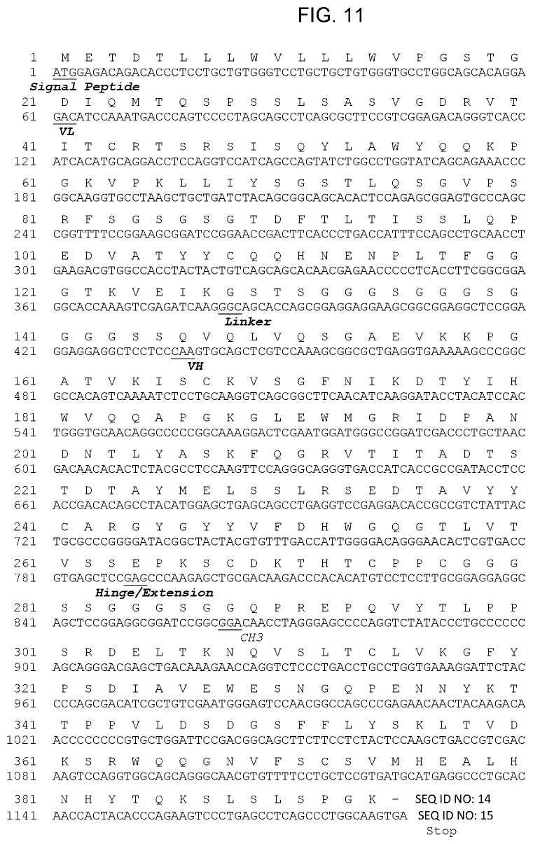

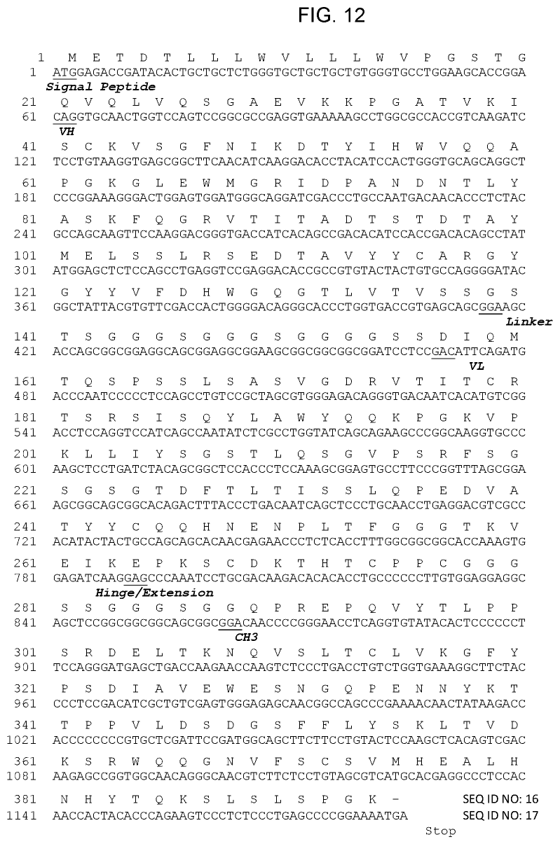

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Some embodiments provided herein provide polypeptides or polynucleotides that are substantially identical to the polypeptides or polynucleotides, respectively, exemplified herein (e.g., the variable regions exemplified in any one FIG. 2A, 2B, or 4-11, 12C-12I; the CDRs exemplified in any one of FIG. 2A, 2B, or 12C to 12I; the FRs exemplified in any one of FIG. 2A, 2B, or 12C-12I; and the nucleic acid sequences exemplified in any one of FIG. 12A-12I or 4-11). Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, in some embodiments, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, in some embodiments, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “subject,” “patient,” and “individual” interchangeably refer to an entity that is being examined and/or treated. This can include, for example, a mammal, for example, a human or a non-human primate mammal. The mammal can also be a laboratory mammal, e.g., mouse, rat, rabbit, hamster. In some embodiments, the mammal can be an agricultural mammal (e.g., equine, ovine, bovine, porcine, camelid) or domestic mammal (e.g., canine, feline).

The term “therapeutically acceptable amount” or “therapeutically effective dose” interchangeably refer to an amount sufficient to effect the desired result. In some embodiments, a therapeutically acceptable amount does not induce or cause undesirable side effects. A therapeutically acceptable amount can be determined by first administering a low dose, and then incrementally increasing that dose until the desired effect is achieved.

The term “co-administer” refers to the administration of two active agents in the blood of an individual or in a sample to be tested. Active agents that are co-administered can be concurrently or sequentially delivered.

“Label”, “detectable label” or “detectable marker” are used interchangeably herein and refer to a detectable compound or composition which is conjugated directly or indirectly associated with the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable.

The term “ImmunoPET” is a term used for positron emission tomography (PET) of radiolabeled antibodies and antibody fragments.

The term “cytotoxic agent” as used herein refers to a substance that inhibits or prevents a cellular function and/or causes cell death or destruction. The term is intended to include radioactive isotopes (e.g., At.sup.211, I.sup.131, I.sup.125, Y.sup.90, Re.sup.186, Re.sup.188, Sm.sup.153, Bi.sup.212, P.sup.32, Pb.sup.212 and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, toxins, growth inhibitory agents, drug moieties, and the various antitumor or anticancer agents disclosed below. Other cytotoxic agents are described below. A tumoricidal agent causes destruction of tumor cells.

A “toxin” is any substance capable of having a detrimental effect on the growth or proliferation of a cell.

A “chemotherapeutic agent” is a chemical compound useful in the treatment of cancer. Examples of chemotherapeutic agents include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ docetaxel (Rhone-Poulenc Rorer, Antony, France); chloranbucil; gemcitabine (GEMZAR™); 6-thioguanine; mercaptopurine; methotrexate; platinum analogs such as cisplatin and carboplatin; vinblastine (VELBAN™); platinum; etoposide (VP-16); ifosfamide; mitoxantrone; vincristine (ONCOVIN™); oxaliplatin; leucovovin; vinorelbine (NAVELBINE™); novantrone; edatrexate; daunomycin; aminopterin; ibandronate; topoisomerase inhibitor RFS 2000; difluoromethylornithine (DMFO); retinoids such as retinoic acid; capecitabine (XELODA™); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

A “cancer vaccine” means a vaccine that treats existing cancer or prevents development of a cancer. Cancer vaccine therapy includes intratumoral vaccine therapy such as that described in A Marabelle, L Tselikas, T de Baere, R Houot; “Intratumoral immunotherapy: using the tumor as the remedy” Annals of Oncology, Volume 28, Issue suppl 12, December 2017; and in Aurélien Marabelle, Holbrook Kohrt, Christophe Caux, and Ronald Levy; “Intratumoral Immunization: A New Paradigm for Cancer Therapy”; Clin Cancer Res. 2014 Apr. 1; 20(7): 1747-1756.

“Radiotherapy” means treatment using radiation or a radio-isotope with a therapeutic purpose. It includes radiation therapy intended to have abscopal effect as described in Yang Liu, Yinping Dong, Li Kong, Fang Shi, Hui Zhu & Jinming Yu; “Abscopal effect of radiotherapy combined with immune checkpoint inhibitors”; Journal of Hematology & Oncology volume 11, Article number: 104 (2018); and in Melek Tugce Yilmaz, Aysenur Elmali, and Gozde Yazici; “Abscopal Effect, From Myth to Reality: From Radiation Oncologists' Perspective”; Cureus. 2019 January; 11(1).

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX™ tamoxifen), EVISTA™ raloxifene, droloxifene, 4-hydroxytamoxifen, trioxifene, keoxifene, LY117018, onapristone, and FARESTON™ toremifene; anti-progesterones; estrogen receptor down-regulators (ERDs); agents that function to suppress or shut down the ovaries, for example, leutinizing hormone-releasing hormone (LHRH) agonists such as LUPRON™ and ELIGARD™ leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestanie, fadrozole, RIVISOR™ vorozole, FEMARA™ letrozole, and ARIMIDEX™ anastrozole. In addition, such definition of chemotherapeutic agents includes bisphosphonates such as clodronate (for example, BONEFOS™ or OSTAC™), DIDROCAL™ etidronate, NE-58095, ZOMETA™ zoledronic acid/zoledronate, FOSAMAX™ alendronate, AREDIA™ pamidronate, SKELID™ tiludronate, or ACTONEL™ risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE™ vaccine and gene therapy vaccines, for example, ALLOVECTIN™ vaccine, LEUVECTIN™ vaccine, and VAXID™ vaccine; LURTOTECAN™ topoisomerase 1 inhibitor; ABARELIX™ rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE™, Rhone-Poulenc Rorer), derived from the European yew, is a semisynthetic analogue of paclitaxel (TAXOL™, Bristol-Myers Squibb). Paclitaxel and docetaxel promote the assembly of microtubules from tubulin dimers and stabilize microtubules by preventing depolymerization, which results in the inhibition of mitosis in cells.

“Immunotherapy” (also called “Immunostimulation” and “IOT”) means the prevention or treatment of disease with a therapy (e.g. an agent or a course of treatment) that stimulates the host immune response to the disease. Many diseases are treatable with immunotherapy. Academic literature in recent years has often used immunotherapy to refer specifically immuno-oncology, which denotes cancer treatment which aims to reduce the immune avoidance characteristics of a tumor or neoplasia, thereby allowing natural or modified immune cells to identify and eliminate the cancerous tissue. “Immunotherapy” also may refer to an immunotherapy agent, or to a method of using such an agent, depending on context. Various immunotherapeutic agents are now available, and many more are in clinical and pre-clinical development. Well known immunotherapeutic agents include but are not limited to, checkpoint inhibitor (“CPI”) therapy (e.g. anti-PD-1 (Keytruda® pembrolizumab) or anti PD-L1 (Opdivo® nivolumab) binding agents), IL2 and fragments or prodrugs thereof (e.g. NKTR-214, a prodrug of PEG-conjugated IL2 (aldesleukin)), other CD122 (IL2RB interleukin 2 receptor subunit beta) binding ligands, GAd-NOUS-20 neoantigen vaccine (D'Alise et al 2017; which may enhance NKTR-214 activity), T-cell bi-specific agent therapy, therapy for reversal of T-cell exhaustion, inhibition of indoleamine 2,3-dioxygenase (IDO) (such as with epacadostat (INCB024360)), and CAR-T therapy.

The terms “Immune check point inhibitor” (sometimes referred to as “ICI”), or “checkpoint inhibitor” (sometimes “CPI”) or “immune checkpoint blockade inhibitor” and all similar terms, denote a subclass of immunotherapies. Examples include molecules that block certain proteins made by some types of immune system cells, such as T cells, and some cancer cells. These proteins help keep immune responses in check and can keep T cells from killing cancer cells. When these proteins are blocked, the immune system is free to be active and T cells are able to kill cancer cells. Some embodiments include anti-PD1 and anti-PD-L1 binding agents, anti-CTLA4 agents, and multi-specific agents including, but not limited to, anti-CTLA-4/B7-1/B7-2. Additional immunotherapies include checkpoint inhibitors such as ipilimumab (Yervoy), pembrolizumab (Keytruda), nivolumab (Opdivo), atezolizumab (Tecentriq), avelumab (Bavencio), and durvalumab (Imfinzi). IOTs also include tremelimumab and pidilizumab, Small molecule ICIs are also in development including BMS-1001, BMS-1116, CA-170, CA-327, Imiquimod, Resiquimod, 852A, VTX-2337, ADU-S100, MK-1454, Ibrutinib, 3AC, Idelalisib, IPI-549, Epacadostat, AT-38, CPI-444, Vipadenant, Preladenant, PBF, AZD4635, Galuniseritib, OTX015/MK-8628, CPI-0610 (c.f. Kerr and Chisolm (2019) The Journal of Immunology, 2019, 202: 11-19.)

IOTs also include other modalities which are not CPIs but which also activate the host immune system against the cancer, or render the tumor vulnerable to CPI therapy. Such alternative IOTs include but are not limited to: T-cell immunomodulators such as the cytokines IL-2, IL-7, IL-15, IL-21, IL-12, GM-CSF and IFNα (including THOR-707 of Synthorx Therapeutics; and NKTR-214 bempegaldesleukin of Nektar Therapeutics); Various other interferons and interleukins; TGβ1 inhibitors (such as SRK-181 in development by Scholar Rock); Oncolytic therapy (including oncolytic virus therapy); Adoptive cell therapy such as T cell-therapy (including CAR-T cell therapy); Cancer vaccines (both preventative and treatment based). Immunotherapy also includes strategies that increase the burden of neoantigens in tumour cells, including targeted therapies which cause a tumor cell to express or reveal tumor associated antigens. (c.f. Galon and Bruni (2019) Nature Reviews Drug Discovery v. 18, pages 197-218). Further IOTs include TLR9 ligands (Checkmate Pharmaceuticals), A2A/A2B dual antagonists (Arcus Biosciences) and vaccination peptides directed to endogenous enzymes such as IDO-1 and arginase (10 Biotech). IOTs include HS-110, HS-130 and PTX-35 (Heat Biologics).

Those skilled in the art recognize that immunotherapies may be used in combination with each other. Immunotherapies can also be used before, after, or in combination with other therapies for the disease, including in the case of cancer, radiation therapy, chemotherapy of all types (including the cytotoxic agents, chemotherapeutic agents, anti-hormonal agents, and growth inhibitory agents referred to above) and surgical resection.

“Tumor-infiltrating lymphocyte” or “TIL” refers to a lymphocyte which is found within the border of a tumor, e.g., a solid tumor.

“Tumor-infiltrating lymphocyte status” or “TIL” status or other similar term denotes the degree to which lymphocytes can penetrate into a tumor or neoplasia or tumor stroma. A TIL positive tumor may also be described as “T-cell inflamed”.

“PET” is a diagnostic technique that can be used to observe functions and metabolism of human organs and tissues at the molecular level. For PET, a positron radioactive drug (e.g., ¹⁸F-FDG) can be injected into a human body. If FDG is used, because the metabolism of fludeoxyglucose (FDG) is similar to glucose, the FDG will gather in cells that digest the glucose. The uptake of the radioactive drug by rapidly growing tumor tissues is different. A positron emitted by the decay of ¹⁸F and an electron in tissues will undergo an annihilation reaction to generate two gamma-photons with the same energy in opposite directions. A detector array surrounding the human body can detect the two photons using a coincidence measurement technique, and determine position information of the positron. A tomography image of positrons in the human body can then be constructed by processing the position information using an image reconstruction software. In some situations, Immuno-PET can be employed, where the label (e.g., ¹⁸F) is attached or associated with an antigen binding construct. In such embodiments, the distribution of the antigen binding construct can be monitored, which will depend upon the binding properties and distribution properties of the antigen binding construct. For example, if a CD8-directed minibody is used, then PET can be used to monitor the distribution of the CD8 molecules through the hosts' system. PET systems are known in the art and include, for example U.S. Pat. Pub. No. 20170357015, 20170153337, 20150196266, 20150087974, 20120318988, and 20090159804, the entireties of each of which are incorporated by reference herein for their description regarding PET and the use thereof.

Embodiments provided herein and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Methods

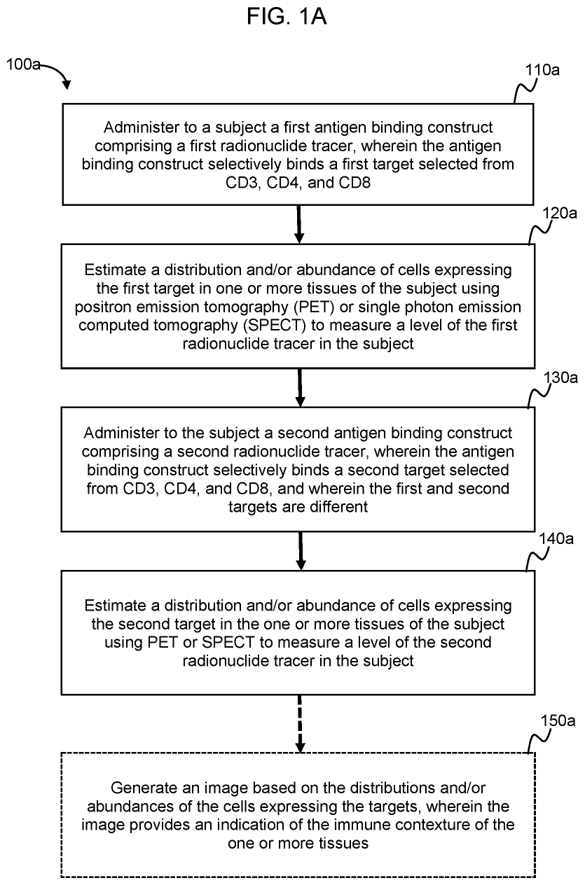

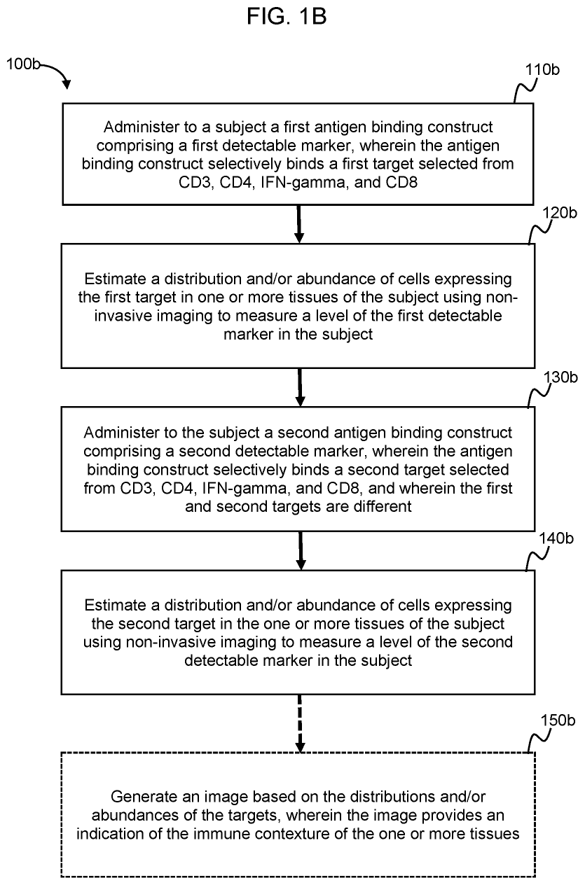

Provided herein are methods of imaging a subject using detectable markers, such as PET tracers (e.g., radionuclide-labeled antigen-binding constructs), that selectively bind immune cell markers for non-invasive imaging. With reference to FIGS. 1A and 1B, an implementation of a method 100a, 100b of imaging a subject is described. The method may include administering a first antigen binding construct comprising a first detectable marker 110b, such as a radionuclide tracer 110a (e.g., a PET tracer) to a subject. The antigen binding construct may selectively bind a first target, such as an immune cell marker. In some embodiments, the first target may be one of CD3, CD4, and CD8. In some embodiments, the antigen binding construct is an antibody, or antigen-binding fragment thereof, that binds selectively to the target. In some embodiments, the antigen binding construct is a minibody or a cys-diabody that binds selectively to the target, e.g., CD3, CD4, or CD8.

Then, the distribution or abundance of cells expressing the first target in one or more tissues of the subject is estimated using non-invasive imaging 120b, e.g., PET or SPECT 120a, to measure the signal from the detectable maker, e.g., the radionuclide signal, in the subject. In certain embodiments, the level of signal from the detectable marker, e.g., the radionuclide, measured using non-invasive imaging, e.g., PET or SPECT, at different locations across the subject's body serves as a proxy for the abundance of cells expressing the target at each location measured. Any suitable non-invasive imaging option may be used, as disclosed herein. In some embodiments, PET or SPECT may be performed using any suitable means. As described further herein, any suitable process may be used to convert the detected signal of the detectable marker, e.g., radionuclide signal, to an estimate of the abundance of cells expressing the target.

The method may further include administering a second antigen binding construct comprising a second detectable marker 130b, such as a radionuclide tracer (e.g., a PET tracer) 130a, to the subject. The second antigen binding construct may selectively bind a second target, such as an immune cell marker, where the second target is different from the first target. In some embodiments, the second target may be one of CD3, CD4, and CD8, where the second target is different from the first target. Any suitable combination of first and second targets may be used. In some embodiments, where one of the targets, e.g., the first target, is CD3, the other target, e.g., second target, may be CD4 or CD8. In certain embodiments, where one of the targets, e.g., the first target, is CD4, the other target, e.g., the second target, is CD8. In some embodiments, the antigen binding construct is an antibody, or antigen-binding fragment thereof, that binds selectively to the target. In some embodiments, the antigen binding construct is a minibody or a cys-diabody that binds selectively to the target, e.g., CD3, CD4 or CD8.

In some embodiments, the first target is one of CD3, CD4, IFN-gamma, and CD8. In some embodiments, the second target is one of CD3, CD4, IFN-gamma, and CD8, where the second target is different from the first target. Any suitable combination of first and second targets may be used. In some embodiments, where one of the targets, e.g., the first target, is CD3, the other target, e.g., the second target, may be CD4 or CD8 or IFN-gamma. In some embodiments, where one of the targets, e.g., the first target, is CD4, the other target, e.g., the second target, may be CD8 and/or IFN-gamma. In some embodiments, where one of the targets, e.g., the first target, is CD8, the other target, e.g., the second target, is IFN-gamma.

In some embodiments, the method includes administering a second antigen binding construct comprising a second radionuclide tracer (e.g., a PET tracer) to a subject, where the second radionuclide tracer is ⁸⁹Zr, and where a dose of about 0.5-1.5+/−20% mCi, e.g., about 1 mCi, of the radionuclide tracer is administered with about 0.2-10 mg of the antigen-binding construct.

Any suitable process may be used to estimate the distribution and/or abundance of cells expressing a target based on the level of the detectable marker, e.g., radionuclide tracer, measured by non-invasive imaging, e.g., PET or SPECT. In some embodiments, a signal intensity over a region of interest (ROI) may be used to estimate the distribution and/or abundance of cells expressing a target based on the level of the detectable marker, e.g., radionuclide tracer, measured by non-invasive imaging, e.g., PET or SPECT. In some embodiments, a signal intensity over an ROI normalized to a reference signal intensity may be used to estimate the distribution and/or abundance of cells expressing a target based on the level of the detectable marker, e.g., radionuclide tracer, measured by non-invasive imaging, e.g., PET or SPECT. In some embodiments, an average signal intensity over an ROI may be used to estimate the distribution and/or abundance of cells expressing a target based on the level of the detectable marker, e.g., radionuclide tracer, measured by non-invasive imaging, e.g., PET or SPECT. In some embodiments, an average signal intensity over an ROI may be used to estimate the distribution and/or abundance of cells expressing a target based on the level of the detectable marker, e.g., radionuclide tracer, measured by non-invasive imaging, e.g., PET or SPECT. In some embodiments, a standard uptake value (SUV) may be calculated to estimate the distribution and/or abundance of cells expressing a target based on the level of the detectable marker, e.g., radionuclide tracer, measured by non-invasive imaging, e.g., PET or SPECT. Suitable options are described in, e.g., International Application No. PCT/US2019/053642, filed Sep. 27, 2019, which is incorporated herein by reference.

The order in which the antigen binding constructs specific to the targets, e.g., CD3, CD4, IFN-gamma, or CD8, are administered may be any suitable order. In some embodiments, an antigen-binding construct specific to CD3 is administered first, and an antigen-binding construct specific to another target, e.g., CD4, CD8, or IFN-gamma, is administered second. In some embodiments, an antigen-binding construct specific to CD4 is administered first, and an antigen-binding constructs specific to another target, e.g., CD3, CD8, or IFN-gamma, is administered second. In some embodiments, an antigen binding constructs specific to CD3 is administered first, and an antigen-binding construct specific to CD4 is administered second. In some embodiments, an antigen-binding constructs specific to CD8 is administered first, and an antigen-binding construct specific to another target, e.g., CD4, CD3, or IFN-gamma, is administered second. In some embodiments, an antigen-binding construct specific to CD4 is administered first, and an antigen-binding construct specific to CD8 is administered second. In some embodiments, an antigen-binding construct specific to IFN-gamma is administered first, and an antigen-binding construct specific to another target, e.g., CD3, CD4, or CD8, is administered second. In some embodiments, different antigen-binding constructs are administered simultaneously, e.g., at the same time, or on the same day.

Then, the distribution or abundance of cells expressing the second target in one or more tissues of the subject is estimated using non-invasive imaging 140b, e.g., PET or SPECT 140a, to measure the second detectable marker, e.g., radionuclide signal, in the subject. The estimated distribution or abundance of cells expressing the first and second targets in a tissue may provide an immune contexture of the tissue.

In some embodiments, the method includes estimating a distribution or an abundance of cells expressing the target, e.g., CD3, CD4, CD8, or IFN-gamma based on the measured level of detectable marker, e.g., radionuclide tracer, associated with the antigen-binding construct that selectively binds to the target, e.g., CD3, CD4, CD8, or IFN-gamma respectively. In some embodiments, the distribution or an abundance of cells expressing one target selected from CD3, CD4, or CD8, or the abundance or distribution of IFN-gamma is estimated based on the measured levels of detectable markers, e.g., radionuclide tracers, associated with antigen-binding constructs that selectively binds to the other two targets. Without being bound to theory, the relationship between cells expressing CD3, CD4 and CD8 as measured by a non-invasive imaging process (e.g., PET or SPECT) of the present disclosure may be generally represented as: (abundance of CD3⁺ cells)<(abundance of CD4⁺ cells)+(abundance of CD8⁺ cells), within a fixed volume. The relationship between the estimated abundance and/or distribution of CD3⁺ cells, CD4⁺ cells, and CD8⁺ cells may depend on the sensitivity and/or resolution of the non-invasive imaging process. In some embodiments, the estimated abundance of CD3⁺ cells, CD4⁺ cells, and CD8⁺ cells depends on the sensitivity of the non-invasive imaging process, e.g., sensitivity of the PET camera. In some embodiments, the estimated distribution of CD3⁺ cells, CD4⁺ cells, and CD8⁺ cells depends on the resolution of the non-invasive imaging process, e.g., resolution of the PET camera. In general, the sum of the distribution and/or abundance of CD8⁺ cells and CD4⁺ cells estimated using a lower resolution and/or lower sensitivity imaging process can approximate the distribution and/or abundance of CD8⁺ cells. Further, the difference between the distribution and/or abundance of CD3⁺ cells and CD4⁺ cells estimated using a lower resolution and/or lower sensitivity imaging process can approximate the distribution and/or abundance of CD8⁺ cells. Similarly, the difference between the distribution and/or abundance of CD3⁺ cells and CD8⁺ cells estimated using the lower resolution and/or lower sensitivity imaging process can approximate the distribution and/or abundance of CD4⁺ cells. In some embodiments, the sum of the distribution of CD8⁺ cells and CD4⁺ cells estimated using a lower resolution imaging process, e.g., PET camera, can approximate the distribution of CD8⁺ cells. In some embodiments, the sum of the abundance of CD8⁺ cells and CD4⁺ cells estimated using a lower sensitivity imaging process, e.g., PET camera, can approximate the abundance of CD8⁺ cells. In some embodiments, the difference between the distribution of CD3⁺ cells and CD4⁺ cells estimated using a lower resolution imaging process, e.g., PET camera, can approximate the distribution of CD8⁺ cells. In some embodiments, the difference between the abundance of CD3⁺ cells and CD4⁺ cells estimated using a lower sensitivity imaging process, e.g., PET camera, can approximate the abundance of CD8⁺ cells. In some embodiments, the difference between the distribution of CD3⁺ cells and CD8⁺ cells estimated using the lower resolution imaging process, e.g., PET camera, can approximate the distribution of CD4⁺ cells. In some embodiments, the difference between the abundance of CD3⁺ cells and CD8⁺ cells estimated using the lower sensitivity imaging process, e.g., PET camera, can approximate abundance of CD4⁺ cells.

In some embodiments, the distribution or an abundance of cells expressing CD3 may be estimated by the sum of the estimated distribution or abundance of cells expressing CD4, based on non-invasive imaging, e.g., PET or SPECT, and the estimated distribution or abundance of cells expressing CD8, based on non-invasive imaging, e.g., PET or SPECT. In some embodiments, the distribution or an abundance of cells expressing CD4 may be estimated by the difference between the estimated distribution or abundance of cells expressing CD3, based on non-invasive imaging, e.g., PET or SPECT, and the estimated distribution or abundance of cells expressing CD8, based on non-invasive imaging, e.g., PET or SPECT. In some embodiments, the distribution or an abundance of cells expressing CD8 may be estimated by the difference between the estimated distribution or abundance of cells expressing CD3, based on non-invasive imaging, e.g., PET or SPECT, and the estimated distribution or abundance of cells expressing CD4, based on non-invasive imaging, e.g., PET or SPECT. The above may also be applied using IFN-gamma as an alternative or as an addition.

As the resolution and/or sensitivity of the imaging process used to measure the signal from the detectable marker, e.g., radionuclide signal, in the subject's body is increased, the sum of the estimated abundance of CD4⁺ cells and CD8⁺ cells may deviate from the abundance of CD3⁺ cells. Without being bound to theory, CD3 can be considered a specific marker for T cells. CD4 can be expressed on T cells as well as on monocytes/macrophages and dendritic cells. Similarly, CD8 can be expressed on T cells as well as NK cells and macrophages. Further, some T-cells may express both CD4 and CD8. Thus, where the resolution and/or sensitivity of a non-invasive imaging process (e.g., PET or SPECT) of the present disclosure is sufficiently high, the relationship between cells expressing CD3, CD4 and CD8 may be represented as: (abundance of CD3⁺ cells)<(abundance of CD4⁺ cells)+(abundance of CD8⁺ cells), within a fixed volume. In some embodiments, the immune contexture is determined by considering the relative abundances of cells expressing CD3, CD4 and CD8. The above may also be applied using IFN-gamma as an alternative or as an addition.

In some embodiments, the method also includes administering a third antigen-binding construct comprising a third detectable marker, such as a radionuclide tracer (e.g., a PET tracer), to a subject. The third antigen binding construct may selectively bind a third target (e.g., immune cell marker) selected from CD3, CD4, IFN-gamma, and CD8, that may be different from the first or second target. In some embodiments, the antigen binding construct is an antibody, or antigen-binding fragment thereof, that binds selectively to the target. Then, the distribution or abundance of cells expressing the third target in one or more tissues of the subject is estimated using non-invasive imaging, e.g., PET or SPECT, to measure the signal from the third detectable marker, e.g., third radionuclide signal, in the subject. The above may also be applied using IFN-gamma as an alternative or as an addition.

In some embodiments, the method includes administering a third antigen binding construct comprising a third radionuclide tracer (e.g., a PET tracer) to a subject, where the third radionuclide tracer is ⁸⁹Zr, and where a dose of about 0.5-1.5+/−20% mCi, e.g., about 1 mCi, of the radionuclide tracer is administered with between 0.2-10 mg of the antigen-binding construct.

In some embodiments, the method includes administering a fourth antigen binding construct to bind IFN-gamma comprising a fourth radionuclide tracer (e.g., a PET tracer), to the subject, where the fourth radionuclide tracer is ⁸⁹Zr, and where a dose of about 0.5-1.5+/−20% mCi, e.g., about 1 mCi, of the radionuclide tracer is administered.

The antigen binding constructs may be administered by any suitable route, including but not limited to aerosol, enteral, nasal, ophthalmic, oral, parenteral, rectal, transdermal (e.g., topical cream or ointment, patch), or vaginal. In some embodiments, the method includes administering an antigen binding construct transdermally, e.g., by using a topical cream or ointment or by means of a transdermal patch. In some embodiments, the method includes administering an antigen binding construct parenterally, e.g., by injection, including infraorbital, infusion, intraarterial, intracapsular, intracardiac, intradermal, intramuscular, intraperitoneal, intrapulmonary, intraspinal, intrasternal, intrathecal, intrauterine, intravenous, intracranial, subarachnoid, subcapsular, subcutaneous, transmucosal, or transtracheal injection. In some embodiments, the antigen binding construct can be delivered intraoperatively as a local administration during an intervention or resection.

In certain embodiments, the method includes determining a relative abundance among cells expressing any one of the targets compared to cells expressing another one of the targets in each of the one or more tissues. In regions of the subject's body, e.g., in a particular tissue of interest, the distribution of two or more cells expressing different targets, as determined by measuring the level of detectable markers, e.g., radionuclide tracers, associated with the antigen-binding constructs having binding specificity for the different targets, using any suitable process as described herein, may overlap. Then, the estimated abundance of the immune cell types in the tissue can be compared with each other to determine the immune contexture of that tissue. In some embodiments, the method includes determining an immune contexture that includes the relative abundance of CD3⁺ cells, CD4⁺ cells or CD8⁺ cells compared to another one of CD3⁺ cells, CD4⁺ cells or CD8⁺ cells in the tissue. The relative abundance of the cells compared to each other may be determined using any suitable method. In some embodiments, the relative abundance of the cells may be a difference between the estimate of a distribution and/or abundance of cells expressing a first target and the estimate of a distribution and/or abundance of cells expressing a second target. In some embodiments, the relative abundance of the cells may be a ratio of the estimate of a distribution and/or abundance of cells expressing a first target to the estimate of a distribution and/or abundance of cells expressing a second target. In some embodiments, an immune contexture, e.g., immunoscore, determined by the present methods includes more than the level of infiltration of immune cells expressing, or associated with, the targets, e.g., CD3, CD4, CD8 or IFN-gamma, of the antigen-binding constructs. Thus, in some embodiments, an immune contexture, e.g., immunoscore, determined by the present methods is based on a combination of two or more targets probed by non-invasive imaging options as disclosed herein, where the prognosis based on the immune contexture is more accurate and/or more discriminatory (e.g., better able to differentiate between patients based on prognosis) compared to a prognosis based on detecting the distribution of any one of the targets individually. In some embodiments, a prognosis for a subject based on the detected distribution or abundance of any one of the targets, e.g., CD3, CD4, CD8 or IFN-gamma, depends on the context of one or more of the other targets.

In some embodiments, the method includes determining an immune contexture that includes the abundance of CD4⁺ cells relative to CD3⁺ cells, or the abundance of CD8⁺ cells relative to CD3⁺ cells, or the abundance of CD4⁺ cells relative to CD8⁺ cells in the tissue. In some embodiments, the method includes determining an immune contexture that includes the abundance of each of CD4⁺, CD8⁺ and CD3⁺ cells relative to one another. In some embodiments, the method includes determining an immune contexture that includes the abundance of each of CD8⁺ and CD3⁺ cells relative to the abundance of CD4⁺ cells. In some embodiments, the method includes determining an immune contexture that includes the abundance of each of CD8⁺ and CD4⁺ cells relative to the abundance of CD3⁺ cells. In some embodiments, the method includes determining an immune contexture that includes the abundance of each of CD4⁺ and CD3⁺ cells relative to the abundance of CD8⁺ cells. In some embodiments, the method includes determining an immune contexture that includes the abundance of both CD8⁺ and CD4⁺ cells relative to the abundance of CD3⁺ cells.

In some embodiments, the method includes determining an immune contexture that includes the difference between the abundance of CD4⁺ cells and CD3⁺ cells, or the difference between the abundance of CD8⁺ cells and CD3⁺ cells, or the difference between the abundance of CD4⁺ cells and CD8⁺ cells in the tissue. In some embodiments, the method includes determining an immune contexture that includes the difference in abundance between each of CD4⁺, CD8⁺ and CD3⁺ cells and one another. In some embodiments, the method includes determining an immune contexture that includes the difference in abundance between CD8⁺ cells and CD4⁺ cells, and the difference in abundance between CD3⁺ cells and CD4⁺ cells. In some embodiments, the method includes determining an immune contexture that includes the difference in abundance between CD8⁺ cells and CD3⁺ cells, and the difference in abundance between CD4⁺ cells and CD3⁺ cells. In some embodiments, the method includes determining an immune contexture that includes the difference in abundance between CD4⁺ cells and CD8⁺ cells, and the difference in abundance between CD3⁺ cells and CD8⁺ cells. In some embodiments, the method includes determining an immune contexture that includes the difference between the sum of the abundance of CD8⁺ and CD4⁺ cells, and the abundance of CD3⁺ cells.

In certain embodiments, the method includes determining an immune contexture that includes the ratio of CD4⁺ cells to CD3⁺ cells, or the ratio of CD3⁺ cells to CD4⁺ cells, or the ratio of CD8⁺ cells to CD3⁺ cells, or the ratio of CD3⁺ cells to CD8⁺ cells, or the ratio of CD4⁺ cells to CD8⁺ cells, or the ratio of CD8⁺ cells to CD4⁺ cells in the tissue. In some embodiments, the method includes determining an immune contexture that includes the ratio of each of CD4⁺, CD8⁺ and CD3⁺ cells relative to one another. In some embodiments, the method includes determining an immune contexture that includes the ratio of each of CD8⁺ and CD3⁺ cells to CD4⁺ cells. In some embodiments, the method includes determining an immune contexture that includes the ratio of each of CD8⁺ and CD4⁺ cells to CD3⁺ cells. In some embodiments, the method includes determining an immune contexture that includes the ratio of each of CD4⁺ and CD3⁺ cells to CD8⁺ cells. In some embodiments, the method includes determining an immune contexture that includes the ratio of the sum of CD8⁺ and CD4⁺ cells to CD3⁺ cells.

In some embodiments, the distribution or intensity of IFN-gamma signal in the subject can be used to estimate whether immune cells, e.g., T-cells, identified based on CD4⁺, CD8⁺ and/or CD3⁺ status as disclosed herein, are active or dormant at the site in the subject's body. In general terms, the IFN-gamma signal provides an estimate for the activity of T cells. In some embodiments, the immune contexture includes a comparison of the distribution or intensity of IFN-gamma signal with the abundance and/or distribution of CD4⁺, CD8⁺ and CD3⁺ cells. In some embodiments, the method includes determining an immune contexture by weighting the abundance and/or distribution of CD4⁺, CD8⁺ and/or CD3⁺ cells, as estimated by the level of signal from the detectable markers specific to each, with the distribution or intensity of an IFN-gamma signal in the subject, such that a stronger IFN-gamma signal indicates greater activity of the T-cells.

In certain embodiments, an image can be generated 150a, 150b based on the distribution or abundance of the targets, e.g., the cells expressing the targets, where the image may provide an indication of the immune contexture of the tissue. The image may represent the distribution and/or abundance of cells expressing one or more of the targets probed by the antigen-binding construct administered to the subject, across one or more tissues, or across the entire body. Thus, the image may represent the distribution and/or abundance of cells expressing the first target probed by the first antigen-binding construct administered to the subject, across one or more tissues, or across the entire body. The image may further represent the distribution and/or abundance of cells expressing the second target probed by the second antigen-binding construct administered to the subject, across one or more tissues, or across the entire body. In certain embodiments, the image may represent the distribution and/or abundance of cells expressing the third target probed by the third antigen-binding construct administered to the subject, across one or more tissues, or across the entire body.

In some embodiments, the image provides an immune contexture that includes the abundance or distribution of CD3⁺ cells, CD4⁺ cells or CD8⁺ cells in the tissue. In some embodiments, the image represents an immune contexture that includes the relative abundance of CD3⁺ cells, CD4⁺ cells or CD8⁺ cells compared to another one of CD3⁺ cells, CD4⁺ cells or CD8⁺ cells in the tissue. Thus, in some embodiments, the image represents an immune contexture that includes the abundance of CD4⁺ cells relative to CD3⁺ cells, or the abundance of CD8⁺ cells relative to CD3⁺ cells, or the abundance of CD4⁺ cells relative to CD8⁺ cells in the tissue. In certain embodiments, the image provides an immune contexture that includes the ratio of CD4⁺ cells to CD3⁺ cells, or the ratio of CD3⁺ cells to CD4⁺ cells, or the ratio of CD8⁺ cells to CD3⁺ cells, or the ratio of CD3⁺ cells to CD8⁺ cells, or the ratio of CD4⁺ cells to CD8⁺ cells, or the ratio of CD8⁺ cells to CD4⁺ cells in the tissue. In certain embodiments, the image provides an immune contexture that includes the ratio of each of CD4⁺, CD8⁺ and CD3⁺ cells relative to one another. In some embodiments, the image provides an immune contexture that includes the abundance or distribution of immune cells associated with IFN-gamma expression in the tissue.

In some embodiments, the one or more tissues imaged is affected by a disease, e.g., cancer, autoimmune disease, or infectious disease. In some embodiments, the tissue includes a tumor. In certain embodiments, methods of the present disclosure includes identifying the one or more tissues as having a cancerous tissue (e.g., a tumor). Any suitable invasive or non-invasive means may be used to determine that an imaged tissue is cancerous or includes a tumor, including, without limitation, computed tomography (CT) scan, X-ray, FDG-PET, or magnetic resonance imaging (MRI) or biopsy. In some embodiments, a PET scan image is aligned with an MRI image to identify organs and tissues in the subject. In some embodiments, the PET or SPECT scan and MRI or CT scan may be conducted during the same scanning session using combined scanners.

Any suitable detectable marker for non-invasive in vivo imaging can be used in the present methods. As is well known in the art, at abundances or distributions that are low, the marker may be present but will be below a detectable level. Generally, the detectable marker is used at an amount sufficient to provide a detectable signal when specifically targeted. In some embodiments, the uptake and retention of the PET tracer is correlated with a number of cells present in the ROI. In some embodiments, the SUV for a PET tracer, which can be the level of detection of the marker, is correlated with a number of cells present in the ROI. The number of cells may be a relative level (relative to another cell type) or it may be an absolute number that correlates with (or is calibrated against) the results associated with IHC as described elsewhere herein. In some embodiments, when using ⁸⁹Zr-labeled CD8 minibody, ⁸⁹Zr-Df-IAB22M2C, the minimum detection level corresponds to about 400 cells/mm2 in a section which is 4 microns thick. In some embodiments, the lower cutoff limit for detection is an approximate cell density of 100,000 cells/mm³. In some embodiments, CD8⁺ T-cell densities from 400 to 12,000 cells per mm² is imaged with approximately linear increases in the detected SUV within this range. This corresponds to a range of 100,000 cells/mm³ to 3 million cells/mm³ that can be measured and detected for CD8⁺ T-cells. In some embodiments, 100,000 cells/mm³ or fewer cells are detected in methods of the present disclosure. Cell density calculations for any one of the agents employed in the method of the present disclosure (and each detectable marker employed) can be determined in a similar manner by those skilled in the art. In some embodiments, such cell density determinations provide a valuable tool for calculating the immunoscore and/or for making and instructing diagnosis, prognosis and/or treatment recommendations.

According to certain embodiments, the radionuclide tracers associated with the antigen-binding construct (e.g., a PET tracer) are each selected from ¹⁸F, ⁸⁹Zr, ⁶⁴Cu, ⁶⁸Ga, ¹²³I and ⁹⁹mTc. In some embodiments, the first, second and/or third radionuclide tracer, is selected from ¹⁸F, ⁶⁴Cu, and ⁶⁸Ga. In some embodiments, the first, second and/or third radionuclide tracer is each ⁸⁹Zr. In some embodiments, the first, second and/or third radionuclide tracer is ¹²³I. In some embodiments, the first, second and/or third radionuclide tracer is ⁹⁹mTc. In some embodiments, the first radionuclide tracer is ¹⁸F, ⁶⁴Cu, or ⁶⁸Ga and the second radionuclide tracer is ¹⁸F or ⁸⁹Zr. In some embodiments, each of the first, second and/or third radionuclide tracers is ¹²³I or ⁹⁹mTc. In some embodiments, the first radionuclide tracer is ¹²³I or ⁹⁹mTc and the second radionuclide tracer is ¹²³I or ⁹⁹mTc, where the first and second radionuclide tracers are different.

The order in which the first, second and third antigen-binding construct (e.g., PET tracer) is administered to the subject may be any suitable order. In some embodiments, administering the first antigen-binding construct is performed before administering the second antigen-binding construct. In some embodiments administering the first antigen-binding construct is performed after administering the second antigen-binding construct. In some embodiments, the first antigen-binding construct is administered first, the second antigen-binding construct is administered second, and the third antigen-binding construct is administered third. In some embodiments, the first antigen-binding construct is administered second, the second antigen-binding construct is administered first, and the third antigen-binding construct is administered third. In some embodiments, the first antigen-binding construct is administered third, the second antigen-binding construct is administered first, and the third antigen-binding construct is administered second. In some embodiments, the first antigen-binding construct is administered third, the second antigen-binding construct is administered second, and the third antigen-binding construct is administered first. In some embodiments, the first antigen-binding construct is administered first, the second antigen-binding construct is administered third, and the third antigen-binding construct is administered second.

In some embodiments, the order in which the first, second and third antigen-binding construct (e.g., PET tracer) is administered to the subject depends on the detectable marker, e.g., radionuclide tracer, associated with each antigen-binding construct. In some embodiments, the order in which the first, second and third antigen-binding construct (e.g., PET tracer) is administered to the subject depends on the radioactive half-life of the radionuclide tracer associated with each antigen-binding construct. In some embodiments, the antigen-binding construct administered first is labeled with ¹⁸F, ⁶⁴Cu, or ⁶⁸Ga. In some embodiments, the antigen-binding construct administered first is not labeled with ⁸⁹Zr. In some embodiments, the antigen-binding construct administered first is labeled with ¹⁸F, ⁶⁴Cu, or ⁶⁸Ga, and the antigen-binding construct administered second is labeled with ¹⁸F, ⁶⁴Cu, or ⁶⁸Ga. In some embodiments, the antigen-binding construct administered first is labeled with ¹⁸F, ⁶⁴Cu, or ⁶⁸Ga, and the antigen-binding construct administered second is labeled with ⁸⁹Zr.

The dose of the antigen-binding construct administered to the subject in any method of the present disclosure may include any suitable amount to measure the level of a detectable marker, e.g., radionuclide tracer, associated with the antigen-binding construct administered using PET or SPECT. In some embodiments, the dose includes an antigen-binding construct labeled with a radionuclide tracer that provides a radiation activity of about 0.5-3 mCi+/−20%. In some embodiments, the dose includes an antigen-binding construct labeled with a radionuclide tracer that provides a radiation activity of about 0.5-3 mCi+/−10%. In some embodiments, the dose includes an antigen-binding construct labeled with a radionuclide tracer that provides a radiation activity of about 0.5-3 mCi+/−5%. In some embodiments, the amount of radiation activity in the dose is between 0.5 and 3.6 mCi, for example 0.5, 0.6, 0.7, 0.8, 0.9, 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8, 2.9, 3.0, 3.1, 3.2, 3.3, 3.4, 3.5 and 3.6 mCi, including any amount defined by any two of the preceding values. In some embodiments, a dose of around 3 mCi allows for obtaining an initial image at 6, 7, 8, 9, 10, 12, 14, 16, 20, 25, 30, or 36 hours, or within a time interval defined by any two of the preceding times, plus a second image at 2, 3, 4, 5, 6, 7, 8, 9, 10, 11, 12, 13, or 14 days, or any interval defined by any two of the aforementioned number of days, without additional dose administration. In some embodiments, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more mCi of radiation is administered to the subject using 0.2, 0.5, 0.75, 1, 2, 3, 4, 5, 6, 7, 8, 9, or 10 or more mg of the minibody or antigen binding construct. In some embodiments, a dose of around 3 mCi allows for an initial image at 6-36 hours, plus a second image at 3-10 days, possibly 14 days, without additional dose administration. In some embodiments, particularly where a high efficiency PET scanner/detector is used, an adminstered dose of about 1.0 mCi is sufficient to generate a first image, and optionally, in the case of 89Zr, a second image can be generated at 3-10 days, possibly 14 days, without additional dose administration

In some embodiments, the method includes administering a dose of about 1 mCi of a radionuclide tracer, e.g., ⁸⁹Zr, associated with an antigen-binding construct. In some embodiments, the method includes imaging a subject after administering a dose of about 1 mCi of a radionuclide tracer, e.g., ⁸⁹Zr, associated with an antigen-binding construct; generating a first image after the administering, and imaging the subject 2, 3, 4, 5, 6, 7, 8, 9, 10, 11, 12, 13, or 14 days, or any interval defined by any two of the aforementioned number of days, after the initial imaging to generate a second image. In some embodiments, the subject may be imaged twice, or more times, to measure the signal from the same or different radionuclide tracer, after a single administration of the radionuclide-labeled antigen-binding construct(s). In some embodiments, the subject is imaged using a high efficiency PET scanner/detector, where the subject is imaged two or more times after a single administration of radionuclide-labeled antigen-binding construct, e.g., ⁸⁹Zr-labeled antigen-binding construct. In some embodiments, particularly where a high efficiency PET scanner/detector is used, an administered dose of about 1.0 mCi is sufficient to generate a first image, and optionally, in the case of 89Zr, a second image can be generated at 3-10 days, possibly 14 days, without additional dose administration.

In some embodiments, the dose includes an antigen-binding construct labeled with a detectable marker, e.g., radionuclide tracer, of between 0.2-10 mg of the antigen-binding construct. In some embodiments, the dose includes an antigen-binding construct labeled with a detectable marker, e.g., radionuclide tracer, of about 0.1, 0.2. 0.5, 1, 2.5, 5, 7.5, 10, 12.5, 15, 17.5, or 20 mg, or a value within a range defined by any two of the aforementioned values, of the antigen-binding construct.

The time between administering any of the antigen-binding constructs to the subject and measuring the level of the detectable marker, e.g., radionuclide tracer associated with the administered antigen-binding construct in the subject using non-invasive imaging, e.g., PET or SPECT, may be any suitable time interval for carrying out the non-invasive imaging, e.g., a PET or SPECT scan, on the subject to estimate the distribution and/or abundance of cells expressing the target to which the antigen-binding constructs selectively bind. In some cases, the time interval selected takes into account the radioactive half-life of the particular radionuclide tracer used to label the antigen-binding construct. In some embodiments, the time interval selected takes into account the in vivo half-life of the antigen-binding construct administered to the subject.

In some embodiments, measuring the level of the detectable marker, e.g., radionuclide tracer, in the subject is done within 1 or more hours, e.g., within 2 or more hours, within 3 or more hours, within 4 or more hours, within 5 or more hours, within 6 or more hours, within 8 or more hours, within 10 or more hours, within 12 or more hours, within 18 or more hours, within 24 or more hours, within 2 or more days, within 3 or more days, within 4 or more days, within 5 or more days, within 6 or more days, within 1 or more weeks, including within 2 or more weeks of administering the antigen-binding construct associated with the detectable marker, e.g., radionuclide tracer, to the subject. In some embodiments, measuring the level of the detectable marker, e.g., radionuclide tracer, in the subject is done within 2 or less weeks, e.g., within 1 or less weeks, within 6 or less days, within 5 or less days, within 4 or less days, within 3 or less days, within 2 or less days, within 24 or less hours, within 18 or less hours, within 12 or less hours, within 10 or less hours, within 8 or less hours, within 6 or less hours, within 4 or less hours, within 3 or less hours, including within 2 or less hours, of administering the antigen-binding construct associated with the detectable marker, e.g., radionuclide tracer, to the subject. In some embodiments, measuring the level of the detectable marker, e.g., radionuclide tracer, in the subject is done within 1 hour to 2 weeks, e.g., within 2 hours to 2 weeks, within 3 hours to 1 week, within 6 hours to 1 week, within 12 hours to 6 days, within 24 hours to 5 days, including within 2 days to 5 days of administering the antigen-binding construct associated with the detectable marker, e.g., radionuclide tracer, to the subject. In some embodiments, the detectable marker is a fast-decaying radionuclide tracer (e.g., ¹⁸F, ⁶⁴Cu, ⁶⁸Ga) and measuring the level of the detectable marker, in the subject is done within 1 or more hours, e.g., within 2 or more hours, within 3 or more hours, within 4 or more hours, within 5 or more hours, within 6 or more hours, within 8 or more hours, within 10 or more hours, within 12 or more hours, within 18 or more hours, within 24 or more hours, including within 2 or more days of administering the antigen-binding construct associated with the radionuclide tracer to the subject.

The time between administering any of the antigen-binding constructs to the subject and administering any other one of the antigen-binding constructs to the subject may be any suitable time interval for carrying out non-invasive imaging, e.g., a PET or SPECT scan, on the subject to estimate the distribution and/or abundance of cells expressing the target to which the antigen-binding constructs selectively bind. In some cases, the time interval selected takes into account the radioactive half-life of the radionuclide tracers used to label the different antigen-binding constructs. In some cases, the time interval selected takes into account the in vivo half-life of the different antigen-binding constructs administered to the subject. In some embodiments, the method includes performing a second scan or imaging to measure the level of the first detectable marker and administering the second antigen-binding construct to account for residual signal from the first detectable marker when measuring the level of the second detectable marker associated with the second antigen-binding construct. In some embodiments, the second scan or imaging for the first detectable marker is performed less than 1, 2, 3, 4, 5, 6, 7, 8, 9, 10, 11, 12 hours, or a period of time within the range defined by any two of the preceding values, before administering the second antigen-binding construct.

In some embodiments, administering an antigen-binding construct to the subject is done within 1 or more hours, e.g., within 2 or more hours, within 3 or more hours, within 4 or more hours, within 5 or more hours, within 6 or more hours, within 8 or more hours, within 10 or more hours, within 12 or more hours, within 18 or more hours, within 24 or more hours, within 2 or more days, within 3 or more days, within 4 or more days, within 5 or more days, within 6 or more days, within 1 or more weeks, including within 2 or more weeks of administering another antigen-binding construct (e.g., an antigen-binding construct that has a different binding target than the first antigen-binding construct) to the subject. In some embodiments, administering an antigen-binding construct to the subject is done within 2 or less weeks, e.g., within 1 or less weeks, within 6 or less days, within 5 or less days, within 4 or less days, within 3 or less days, within 2 or less days, within 24 or less hours, within 18 or less hours, within 12 or less hours, within 10 or less hours, within 8 or less hours, within 6 or less hours, within 4 or less hours, within 3 or less hours, including within 2 or less hours, of administering another antigen-binding construct (e.g., an antigen-binding construct that has a different binding target than the first antigen-binding construct) to the subject. In some embodiments, administering an antigen-binding construct to the subject is done within 1 hour to 2 weeks, e.g., within 2 hours to 2 weeks, within 3 hours to 1 week, within 6 hours to 1 week, within 12 hours to 6 days, within 24 hours to 5 days, including within 2 days to 5 days of administering another antigen-binding construct (e.g., an antigen-binding construct that has a different binding target than the first antigen-binding construct) to the subject.

In certain embodiments, different antigen-binding constructs (e.g., two or more antigen-binding constructs that have different binding targets) are administered on the same day. In some embodiments, administering a first antigen-binding construct and administering a second antigen-binding construct that is different from the first antigen-binding construct (e.g., different in the target specificity from the first antigen-binding construct) are performed on the same day. In certain embodiments, different antigen-binding constructs (e.g., two or more antigen-binding constructs that have different binding targets) are administered on different days. In some embodiments, administering a first antigen-binding construct and administering a second antigen-binding construct that is different from the first antigen-binding construct (e.g., has a different binding target than the first antigen-binding construct) are performed on different days.

The time between administering an antigen-binding construct to the subject and measuring a level of a detectable marker, e.g., radionuclide tracer, associated with a different antigen-binding construct (e.g., has a different binding target than the first antigen-binding construct) may be any suitable time interval for carrying out non-invasive imaging, e.g., a PET or SPECT scan, on the subject to estimate the distribution and/or abundance of cells expressing the target to which the antigen-binding constructs selectively bind. In some cases, the time interval selected takes into account the radioactive half-life of the radionuclide tracers used to label the different antigen-binding constructs. In some cases, the time interval selected takes into account the in vivo half-life of the different antigen-binding constructs administered to the subject.

In some embodiments, measuring the level of a detectable marker, e.g., radionuclide tracer, associated with an antigen-binding construct is performed within 1 or more hours, e.g., within 2 or more hours, within 3 or more hours, within 4 or more hours, within 5 or more hours, within 6 or more hours, within 8 or more hours, within 10 or more hours, within 12 or more hours, within 18 or more hours, within 24 or more hours, within 2 or more days, within 3 or more days, within 4 or more days, within 5 or more days, within 6 or more days, within 1 or more weeks, including within 2 or more weeks of administering a different antigen-binding construct (e.g., has a different binding target than the first antigen-binding construct associated with the label whose level is being measured). In some embodiments, measuring the level of a detectable marker, e.g., radionuclide tracer, associated with an antigen-binding construct is performed within 2 or less weeks, e.g., within 1 or less weeks, within 6 or less days, within 5 or less days, within 4 or less days, within 3 or less days, within 2 or less days, within 24 or less hours, within 18 or less hours, within 12 or less hours, within 10 or less hours, within 8 or less hours, within 6 or less hours, within 4 or less hours, within 3 or less hours, including within 2 or less hours, of administering a different antigen-binding construct (e.g., has a different binding target than the first antigen-binding construct associated with the label whose level is being measured). In some embodiments, measuring the level of a detectable marker, e.g., radionuclide tracer, associated with an antigen-binding construct is performed within 1 hour to 2 weeks, e.g., within 2 hours to 2 weeks, within 3 hours to 1 week, within 6 hours to 1 week, within 12 hours to 6 days, within 24 hours to 5 days, including within 2 days to 5 days of administering a different antigen-binding construct (e.g., has a different binding target than the first antigen-binding construct associated with the label whose level is being measured).

In some embodiments, measuring the level of a detectable marker, e.g., radionuclide tracer, associated with an antigen-binding construct is performed on the same day as administering a different antigen-binding construct (e.g. an antigen-binding construct having a binding target that is different from the binding target of the antigen-binding construct whose detectable marker, e.g., radionuclide tracer, is being measured). In some embodiments, measuring the level of a detectable marker, e.g., radionuclide tracer, associated with an antigen-binding construct is performed on a different day as administering a different antigen-binding construct (e.g. an antigen-binding construct having a binding target that is different from the binding target of the antigen-binding construct whose detectable marker, e.g., radionuclide tracer, is being measured).

With respect to FIG. 4, a schematic diagram showing a method of imaging a subject, according to some embodiments of the present disclosure, is described. On Day 1, a subject may be administered an antigen-binding construct (e.g., a minibody (“Mb”) or cys-diabody (“CysDb”)) labeled with a radionuclide tracer (e.g., ¹⁸F). The antigen-binding construct may selectively bind a target (e.g., CD3, CD4, or CD8). Within about 6 hours, e.g., within about 5 hours, within about 4 hours, within about 3 hours, within about 2 hours, including within about 1 hour of administering the antigen-binding construct, the subject may be imaged using PET scanning to measure the level of radioactivity in different parts of the subject's body. The measured level of radioactivity may be related to the concentration of the radionuclide tracer, and therefore to the concentration of the binding target of the antigen-binding construct associated with the radionuclide tracer. From the PET scan, the distribution and/or abundance of cells expressing the target may be estimated, using any suitable process. On Day 2, a second antigen-binding construct (e.g., a minibody (“Mb”) or cys-diabody (“CysDb”)) that is labeled with a second radionuclide tracer (e.g., ¹⁸F) and that selectively binds another target (e.g., CD3, CD4, or CD8) that is different from the target on Day 1 may be administered to the subject. Within about 6 hours, e.g., within about 5 hours, within about 4 hours, within about 3 hours, within about 2 hours, including within about 1 hour of administering the second antigen-binding construct, the subject may be imaged using PET scanning to measure the level of radioactivity in the subject's body. The measured level of radioactivity may be related to the concentration of the second radionuclide tracer, and therefore to the concentration of the binding target of the second antigen-binding construct associated with the second radionuclide tracer. From the PET scan, the distribution and/or abundance of cells expressing the second target may be estimated, using any suitable process. A comparison of the first and second scans can provide an immune contexture of the tissue and other parts of the subject's body. In some embodiments, the immune contexture may be represented in an image that combines the first and second scans. Day 1 and Day 2 may be 1 or more days, e.g., 2 or more days, 3 or more days, 4 or more days, including 5 or more days apart.

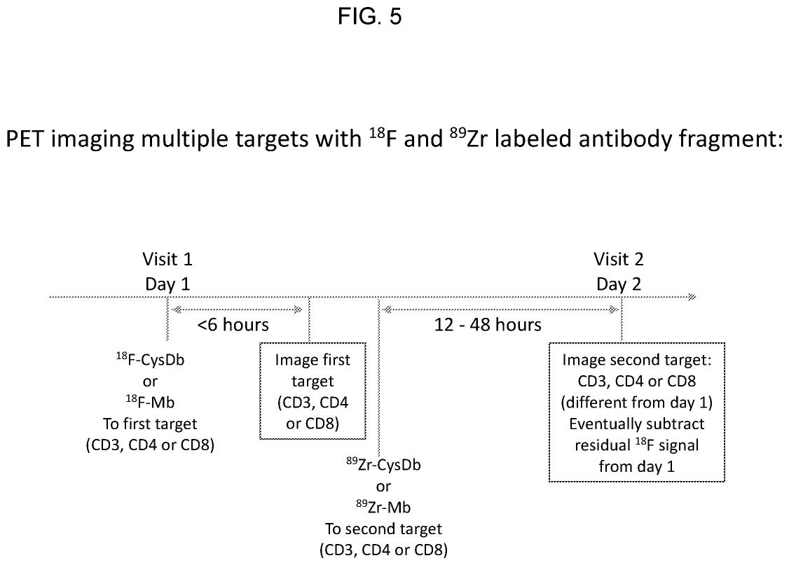

With respect to FIG. 5, a schematic diagram showing a method of imaging a subject, according to some embodiments of the present disclosure, is described. On Day 1, a subject may be administered an antigen-binding construct (e.g., a minibody (“Mb”) or cys-diabody (“CysDb”)) labeled with a radionuclide tracer (e.g., ¹⁸F, ^{M}Cu, or ⁶⁸Ga). The antigen-binding construct may selectively bind a target (e.g., CD3, CD4, or CD8). Within about 6 hours, e.g., within about 5 hours, within about 4 hours, within about 3 hours, within about 2 hours, including within about 1 hour of administering the antigen-binding construct, the subject may be imaged using PET scanning to measure the level of radioactivity in different parts of the subject's body. The level of radioactivity may be related to the concentration of the radionuclide tracer, and therefore to the concentration of the binding target of the antigen-binding construct associated with the radionuclide tracer. From the PET scan, the distribution and/or abundance of cells expressing the target may be estimated, using any suitable process. Still on Day 1, a second antigen-binding construct (e.g., a minibody (“Mb”) or cys-diabody (“CysDb”)) that is labeled with a second radionuclide tracer (e.g., ⁸⁹Zr) and that selectively binds another target (e.g., CD3, CD4, or CD8) that is different from the first target may be administered to the subject. On Day 2, which may be 12 to 48 hours after administering the second antigen-binding construct to the subject, the subject may be imaged using PET scanning to measure the level of radioactivity in the subject's body. The measured level of radioactivity can be related to the concentration of the second radionuclide tracer, and therefore to the concentration of the binding target of the second antigen-binding construct associated with the second radionuclide tracer. From the PET scan, the distribution and/or abundance of cells expressing the second target may be estimated, using any suitable process. A comparison of the first and second scans can provide an immune contexture of the tissue and other parts of the subject's body. In some embodiments, the immune contexture may be represented in an image that combines the first and second scans. Day 1 and Day 2 may be 1 or more days, e.g., 2 or more days, 3 or more days, 4 or more days, including 5 or more days apart.

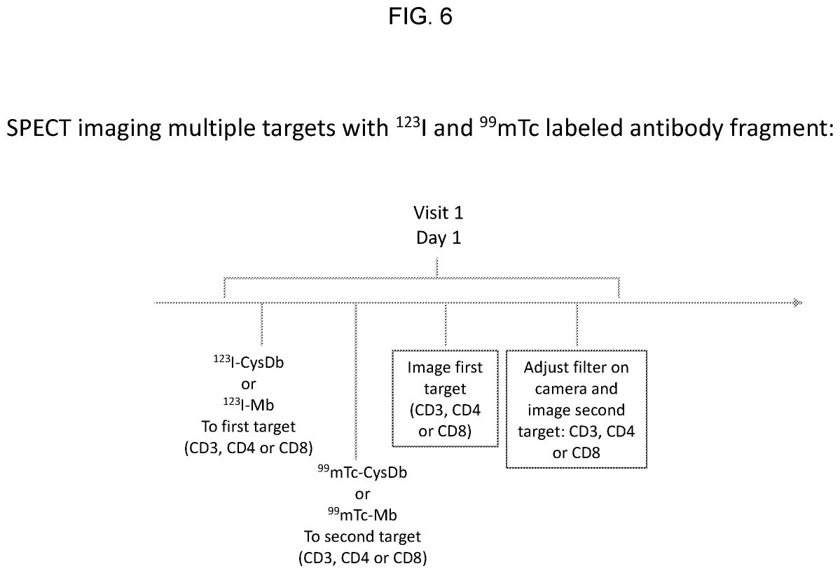

With respect to FIG. 6, a schematic diagram showing a method of imaging a subject is described, according to some embodiments of the present disclosure. A subject may be administered an antigen-binding construct (e.g., a minibody (“Mb”) or cys-diabody (“CysDb”)) labeled with a radionuclide tacer (e.g., ¹²³I). The antigen-binding construct may selectively bind a target (e.g., CD3, CD4, or CD8). On the same day, a second antigen-binding construct (e.g., a minibody (“Mb”) or cys-diabody (“CysDb”)) that is labeled with a second radionuclide tracer (e.g., ⁹⁹mTc) and that selectively binds another target (e.g., CD3, CD4, or CD8) that is different from the first target may be administered to the subject. Still on Day 1, the subject may be imaged using SPECT scanning to measure the level of radioactivity in different parts of the subject's body. The SPECT scanner energy window may be configured to preferentially detect radioactivity from the first radionuclide tracer (e.g., ¹²³I. This radioactivity may be related to the concentration of the first radionuclide tracer, and therefore to the concentration of the binding target of the first antigen-binding construct associated with the first radionuclide tracer. From the SPECT scan, the distribution and/or abundance of cells expressing the first target may be estimated, using any suitable process. After SPECT scanner energy window, the subject may be scanned to preferentially detect radioactivity from the first radionuclide tracer. The measured level of radioactivity may be related to the concentration of the second radionuclide tracer, and therefore to the concentration of the binding target of the second antigen-binding construct associated with the second radionuclide tracer. From the SPECT scan, the distribution and/or abundance of cells expressing the second target may be estimated, using any suitable process. A comparison of the first and second scans can provide an immune contexture of the tissue and other parts of the subject's body. In some embodiments, the immune contexture may be represented in an image that combines the first and second scans.

In some embodiments, the first and second antigen-binding constructs, each having a different radionuclide tracer detectable by PET and each selectively binding to different targets (e.g., CD3, CD4 or CD8), are co-administered, administered contemporaneously or in close sequence (e.g. during the same out-patient visit). The radionuclide tracers may be selected to be distinguishable by radioactive half-life. The subject can be imaged twice, e.g., using PET, first to generate a first image which identifies both targets, and second to identify only the second agent after the first radionuclide tracer has decayed and is no longer detectable or is not significant. The first imaging may take place within 1 hour to 3 days, e.g., within 1 hour to 2 days, within 1 hour to 1 day, within 1 to 18 hours, within 1 to 12 hours, within 1 to 10 hours, within 1 to 8 hours, including within 2 to 6 hours, after the co-administration of the antigen-binding constructs. The second imaging can take place after the first imaging, and after the first radionuclide tracer has decayed to a negligible level. This second imaging may be performed within 20 hours to 2 weeks, e.g., within 20 hours to 1 week, within 20 hours to 5 days, within 20 hours to 4 days, within 20 hours to 3 days, including within 1 to 2 days, after the co-administration of the antigen-binding constructs. In some embodiments, the second image is visually or algorithmically subtracted from the first image to provide distinct images of the two different targets. In certain embodiments, the first radionuclide tracer has a higher radio-emission intensity than the second radionuclide tracer, such that, during the window of PET scanning, the first image represents only the first target. A second imaging scan after the first radiolabel decays can generate a second image that represents the signal associated with the second target. In some embodiments, a third radionuclide tracer associated with an antigen-binding construct that selectively binds a third target that is different from the first and second targets can be co-administered/contemporaneously administered with the first and second antigen-binding constructs, and the signal from the third radionuclide tracer may be distinguished using a third imaging scan in a similar manner as described.

In some embodiments, methods of the present disclosure includes combining two or more, or three antigen-binding constructs, each of which selectively bind to a target selected from CD3, CD4 or CD8, where the antigen-binding constructs bind to different targets from each other, into a composition suitable for administering to a subject to be imaged by PET or SPECT, as described herein. The combination of radionuclide tracers associated with the antigen-binding constructs in the composition may be selected such that the signal from each radionuclide tracer measured by PET or SPECT is distinguishable, as described herein.

In some embodiments, methods of the present disclosure includes determining a functional activity of immune cells in the tissue. The functional activity of immune cells in the tissue may be determined using any suitable means. In some embodiments, the immune cell functional activity is measured using non-invasive imaging (e.g., PET or SPECT) of the subject administered with an imaging agent specific for IFNγ or Granzyme B. Any suitable imaging agent specific for IFNγ or Granzyme B may be used. Suitable imaging agents for IFNγ are described in, e.g., Gibson et al., Cancer Res. 2018 Oct. 1; 78(19):5706-5717. Suitable imaging agents for Granzyme B are described, e.g., in Larimer et al., Cancer Res. 2017 May 1; 77(9):2318-2327. The functional activity of immune cells in the tissue may be included as part of the immune contexture of the tissue. Imaging the subject for functional activity of immune cells in the tissue may be performed before, concurrent to, or after imaging the subject for the distribution and/or abundance of immune cell types in the tissue.

In some embodiments, methods of the present disclosure includes determining a functional environment of the tissue. The functional environment of the tissue may be determined using any suitable means. In some embodiments, the functional environment of the tissue is measured using non-invasive imaging (e.g., PET or SPECT) of the subject administered with an imaging agent specific for PD-1, PD-L1, or TGFβ, or an FDG-PET imaging agent. Any suitable imaging agent specific for PD-1, PD-L1, or TGFβ may be used. Suitable imaging agents for PD-1 and PD-L1 are described, e.g., in Niemeijer et al., Nat Commun. 2018 Nov. 7; 9(1):4664; Lv et al., J Nucl Med. 2019 Jun. 28. pii: jnumed.119.226712. doi: 10.2967/jnumed.119.226712. Suitable imaging agents for TGFβ are described, e.g., in den Hollander et al., J Nucl Med. 2015 September; 56(9):1310-4. Imaging the subject for the functional environment of the tissue may be performed before, concurrent to, or after imaging the subject for the distribution and/or abundance of immune cell types in the tissue.

The level of a detectable marker, e.g., radionuclide tracer, may be measured using non-invasive imaging, e.g., PET or SPECT, in any suitable tissue where the immune contexture of the tissue is sought. The tissue may be, without limitation, the lung, liver, colon, intestine, stomach, heart, brain, kidney, spleen, pancreas, esophagus, lymph node, bone, bone marrow, prostate, cervix, ovary, breast, urethra, bladder, skin, neck, articulated joint, or portions thereof. In some embodiments, the non-invasive imaging, e.g., PET or SPECT scan, is performed over substantially the subject's entire body. In some embodiments, the level of a detectable marker, e.g., radionuclide tracer, is measured over substantially the subject's entire body using non-invasive imaging, e.g., PET or SPECT.

The distribution of the targets, e.g., CD8⁺, CD4⁺ and CD3⁺ cells, and IFN-gamma, in the subject determined using the non-invasive imaging methods of the present disclosure can include a spatial distribution and/or temporal distribution of the cells. In some embodiments, the spatial distribution may be determined by scanning a subject to whom an antigen-binding construct has been administered, as described herein. In some embodiments, the temporal distribution of cells may be determined by comparing the spatial distribution or abundance of the targets, e.g., CD8⁺, CD4⁺ and CD3⁺ cells, and IFN-gamma, in the subject at a first time point using the non-invasive imaging methods, as described herein, with the spatial distribution or abundance of the corresponding targets, CD8⁺, CD4⁺ and CD3⁺ cells, and IFN-gamma, in the subject at a second time point using the non-invasive imaging methods, as described herein. In some embodiments, the temporal distribution of cells may be determined by imaging the subject at two or more time points after a single administration of a detectably-labeled, e.g., radionuclide-labeled, antigen-binding construct. The change (or lack of change) in the spatial distribution or abundance of the cells over time may contribute to the immune contexture (e.g., immunoscore). In some embodiments, the immune contexture determined using methods of the present disclosure includes the persistence of or a change over time in the distribution of targets, e.g., CD8⁺, CD4⁺ and CD3⁺ cells, and IFN-gamma, in the subject.

The temporal distribution of targets, e.g., CD8⁺, CD4⁺ and CD3⁺ cells, and IFN-gamma, in the subject may be monitored at any suitable time interval. In some embodiments, the time interval for monitoring the temporal distribution of targets, e.g., CD8⁺, CD4⁺ and CD3⁺ cells, in the subject is 1 day or more, e.g., 2 days or more, 3 days or more, 5 days or more, 1 week or more, 2 weeks or more, 3 weeks or more, 4 weeks or more, 2 months or more, 3 months or more, 6 months or more, including 1 year or more. In some embodiments, the time interval for monitoring the temporal distribution of targets, e.g., CD8⁺, CD4⁺ and CD3⁺ cells, in the subject is between 1 day and 1 year, e.g., between 1 day and 6 months, between 1 day and 3 months, between 1 day and 2 months, between 2 days and 4 weeks, between 2 days and 3 weeks, between 3 days and 2 weeks, including between 3 days and 1 week. The above may also be applied using IFN-gamma as an alternative or as an addition (using the same variations as noted for the other markers). In some embodiments, the time interval for monitoring the temporal distribution of targets, e.g., CD8⁺, CD4⁺ and CD3⁺ cells, and IFN-gamma, in the subject, is related to or is determined based on the clinical presentation of the patient. In some embodiments, the time interval for monitoring the temporal distribution of targets, e.g., CD8⁺, CD4⁺ and CD3⁺ cells, and IFN-gamma, in the subject is linked to the treatment cycle, e.g., imaging period after 1 or more cycles of treatment. In some embodiments, the target is IFN-gamma and the detectable marker is a fast-decaying radionuclide tracer (e.g., ¹⁸F, ⁶⁴Cu, ⁶⁸Ga), where measuring the level of the detectable marker in the subject is done within 0.5-1 hour, 1-2 hours, 2-3 hours, 3-4 hours, 4-5 hours, 5-6 hours, 6-8 hours, 8-12 hours, or 12-16 hours, of administering the antigen-binding construct associated with the radionuclide tracer to the subject, followed by imaging the subject 24 hours, 48 hours, 3 days, 1 week or more, or any time period in a range defined by any two of the preceding values, after administering the first antigen-binding construct for the same or a different target, as disclosed herein. In some embodiments, the second imaging is done using a second antigen-binding construct to CD3, CD4, CD8, or IFN-gamma, labeled with a detectable marker, e.g., a radionuclide tracer.

Also provided herein are methods of treating and/or diagnosing a subject using non-invasive imaging methods as described herein to obtain the immune contexture of a tissue in a subject in need of treatment. In certain embodiments, the immune contexture as determined using the imaging methods of the present disclosure may be provided to the subject or medical practitioner for making decisions about diagnosis, prognosis, and/or treatment of a disease the subject may have. With reference to FIG. 2, an implementation of a method 200 of treating a subject is described. The method may include administering 210 a first antigen binding construct comprising a first detectable marker, e.g., radionuclide tracer, to a subject having a disease. The antigen binding construct may selectively bind a first target, such as an immune cell marker. In some embodiments, the first target may be one of CD3, CD4, CD8, and/or IFN-gamma. In some embodiments, the antigen binding construct is an antibody, or antigen-binding fragment thereof, that binds selectively to the target. In some embodiments, the antigen binding construct is a minibody or a cys-diabody that binds selectively to the target.

Then, the subject is imaged 220 using non-invasive imaging, e.g., PET or SPECT, to acquire a distribution of cells expressing the first target in one or more tissues of the subject. The distribution of cells expressing a target selectively bound by an antigen-binding construct labeled with a radionuclide tracer (e.g., a PET tracer) may be acquired from the PET or SPECT imaging using any suitable process. As described above, the distribution or abundance of cells expressing the first target in one or more tissues of the subject can be estimated using PET or SPECT to measure the level of a radionuclide tracer (e.g., the level of the radioactive signal from the radionuclide tracer) in the subject.

The method may further include administering 230 a second antigen binding construct comprising a second detectable marker, e.g., radionuclide tracer, to a subject. The second antigen binding construct may selectively bind a second target, such as an immune cell marker, where the second target is different from the first target. In some embodiments, the second target may be one of CD3, CD4, and CD8, where the second target is different from the first target. In some embodiments, where the first target is CD3, the second target may be CD4 or CD8. In certain embodiments, where the first target is CD4, the second target may be CD8. In some embodiments, the antigen binding construct is an antibody, or antigen-binding fragment thereof, that binds selectively to the target. In some embodiments, the antigen binding construct is a minibody or a cys-diabody that binds selectively to the target. Then, the subject is imaged 240 using non-invasive imaging, e.g., PET or SPECT, to acquire a distribution of cells expressing the second target in one or more tissues of the subject. The above may also be applied using IFN-gamma as an alternative or as an addition (using the same variations as noted for the other markers).

In some embodiments, the method includes administering a third antigen-binding construct comprising a third detectable marker, such as a radionuclide tracer, (e.g., a PET tracer) to a subject. The third antigen binding construct may selectively bind a third target (e.g., immune cell marker) selected from CD3, CD4, and CD8, that may be different from the first or second target. In some embodiments, the antigen binding construct is an antibody, or antigen-binding fragment thereof, that binds selectively to the target. Then, the distribution or abundance of cells expressing the third target in one or more tissues of the subject is estimated using non-invasive imaging, e.g., PET or SPECT, to measure the third radionuclide signal in the subject. The above may also be applied using IFN-gamma as an alternative or as an addition (using the same variations as noted for the other markers).

In some embodiments, the method includes generating an image based on the distributions of cells expressing the targets, wherein the image can provide the immune contexture of the one or more tissues, as described above.

The distribution of cells expressing the first and second targets in a tissue may be used to determine 250 an immune contexture of the tissue. In some embodiments, the distribution of cells expressing the first, second and third targets in a tissue may be used to determine 250 an immune contexture of the tissue.

Based on the immune contexture, a treatment may be administered 260 to the subject. The treatment may be any suitable treatment for treating the disease based on the determined immune contexture. The treatment may be an immunotherapy, or it may be a chemotherapy, hormone therapy, radiation, vaccine (including intratumoral vaccine therapy), oncolytic virus therapy, surgery or cellular therapy.

Alternatively, or in addition to administering a treatment, a report may be generated, where the report provides the immune contexture determined based on the imaging methods described herein. In some embodiments, the report may include any additional clinically-relevant information about the subject, including results and/or analysis of other non-invasive tests, biopsies, biomarker tests, etc. In some embodiments, the report may include an immunoscore based on the determined immune contexture and/or other clinically relevant information. In some embodiments, the report may include a diagnosis and/or prognosis for the subject, based on the immune contexture, and optionally, any other relevant clinical information. In some embodiments, the report may include a recommended treatment for the subject, based on the immune contexture, and optionally, any other relevant clinical information.

The methods of the present disclosure may find use in treating or diagnosing any suitable disease or condition in which the immune contexture in the relevant tissue provides diagnostic/prognostic value, or is associated with treatment outcome. The subject can have a disease such as, without limitation, a cancer, autoimmune disease or infectious disease. The subject may have a condition such as a response or a reaction to a therapy which impacts the immune system such as an immunotherapy. Suitable immunotherapies include, without limitation, cell modifying therapies and adoptive cell therapies such as CAR-T, or other therapies such as chemotherapy, a cancer vaccine (including intratumoral vaccines) or radiotherapy (including radiotherapy intended to induce an abscopal effect). In some embodiments, the methods disclosed herein may be used to identify adverse events associated with immunotherapy such as arthritis (Smith and Bass (2019) Arthritis Care Res (Hoboken). March; 71(3):362-366) or cardiotoxicity (Asnani (2018) Curr Oncol Rep. Apr. 11; 20(6):44). The methods of the present disclosure may be used in clinical trials to determine if a patient is responding (positively or negatively) to a therapy or if a disease or condition is progressing. In some embodiments the subject is diagnosed with a cancer, autoimmune disease or infectious disease. The cancer may be a solid tumor, or a non-solid tumor. The autoimmune disease may include, without limitation, arthritis, transplant rejection, graft versus host disease, lupus, multiple sclerosis, type 1 diabetes, etc. Infectious diseases may include, without limitation, viral, bacterial, or fungal infections.

In some embodiments, the subject has cancer, or has been diagnosed with a cancer. In some embodiments, the subject has a cancer of a lung, liver, colon, intestine, stomach, brain, kidney, spleen, pancreas, esophagus, lymph node, bone, bone marrow, prostate, cervix, ovary, breast, urethra, bladder, skin or neck. In some embodiments, the subject has melanoma, non-small-cell lung carcinoma (NSCLC), or renal cell cancer (RCC). In some embodiments, the subject has a solid tumor.

In some embodiments, a method of treating or diagnosing a subject includes determining an immune contexture of a tumor by estimating a density of CD3⁺ cells, CD4⁺ cells and/or CD8⁺ cells in a core and/or invasive margin of the tumor based on the distributions of cells expressing the targets acquire by PET or SPECT. In some embodiments, the immune contexture that includes an estimated density of CD4⁺ cells and CD8⁺ cells in the core and/or invasive margin of the tumor is determined. In some embodiments, the immune contexture that includes an estimated density of CD3⁺ cells and CD8⁺ cells in the core and/or invasive margin of the tumor is determined. In some embodiments, the immune contexture that includes an estimated density of CD4⁺ cells and CD3⁺ cells in the core and/or invasive margin of the tumor is determined. The above may also be applied using IFN-gamma as an alternative or as an addition (using the same variations as noted for the other markers).

In some embodiments, the abundance or density of cells expressing CD3, CD4 or CD8 in a region of interest (ROI) in the subject's body is estimated based on the measured level of signal from the detectable marker, e.g., radioactivity from the radionuclide tracer, associated with the antigen-binding construct specific to CD3, CD4, IFN-gamma or CD8, respectively, in the ROI, as described herein. In some embodiments, the method includes determining whether a tissue (e.g., tumor, tissue, organ, or other anatomical region) in the subject is enriched or depleted for cells expressing CD3, CD4, IFN-gamma or CD8 based on the measured level of radioactivity from the detectable marker, e.g., radionuclide tracer, associated with the antigen-binding construct specific to CD3, CD4, IFN-gamma or CD8, respectively, in the tissue. In some embodiments, the method includes determining whether a tissue in the subject is enriched or depleted for cells expressing a target selected from CD3, CD4, IFN-gamma or CD8 based on the measured levels of detectable markers, e.g., radionuclide tracers, associated with antigen binding constructs that selectively binds to the other two targets in the tissue, using non-invasive imaging, e.g., PET or SPECT. In some embodiments, enrichment or depletion of cells expressing CD3 in a tissue is determined based on the sum of the estimated density or abundance of cells expressing CD4, using non-invasive imaging, e.g., PET or SPECT, and the estimated density or abundance of cells expressing CD8, using non-invasive imaging, e.g., PET or SPECT. In some embodiments, enrichment or depletion of cells expressing CD4 in a tissue is determined based on the difference between the estimated density or abundance of cells expressing CD3, using non-invasive imaging, e.g., PET or SPECT, and the estimated density or abundance of cells expressing CD8, using non-invasive imaging, e.g., PET or SPECT. In some embodiments, enrichment or depletion of cells expressing CD8 in a tissue is determined based on the difference between the estimated density or abundance of cells expressing CD3, using non-invasive imaging, e.g., PET or SPECT, and the estimated density or abundance of cells expressing CD4, using non-invasive imaging, e.g., PET or SPECT.

The immune contexture of the tumor, tissue, organ, or anatomical region, determined according to the present disclosure can indicate a likelihood that the subject will or will not benefit from a particular treatment for a disease or condition (e.g., the tumor). In some embodiments, the immune contexture provides a good prognosis (e.g., a longer disease-free survival, longer overall survival, or low chance of recurrence) when the core and/or invasive margin of the tumor is enriched for CD3⁺ cells and CD8⁺ cells; enriched for CD3⁺ cells and CD4⁺ cells; or enriched for CD4⁺ cells and CD8⁺ cells; depleted for CD8⁺ cells and enriched for CD4⁺ cells or depleted for CD4⁺ cells and enriched for CD8⁺ cells. In some embodiments, when the immune contexture indicates a good prognosis (e.g., a longer disease-free survival, longer overall survival, or low chance of recurrence) the subject may not receive a treatment. In some embodiments, the immune contexture provides a good prognosis (e.g., a longer disease-free survival, longer progression free survival, longer overall survival, improved quality of life, or low chance of recurrence) when the core and/or invasive margin of the tumor is enriched for IFN-gamma. In some embodiments, when the immune contexture indicates a good prognosis (e.g., a longer disease-free survival, longer overall survival, or low chance of recurrence) an adjuvant therapy may not be administered to the subject after an initial treatment of the subject for the cancer (e.g., surgical resection of the tumor).

In some embodiments, the core and/or invasive margin of the tumor is determined to be enriched for CD8⁺ cells when the estimated density is 50 cells/mm² or more, e.g., 100 cells/mm² or more, 150 cells/mm² or more, 200 cells/mm² or more, 250 cells/mm² or more, 300 cells/mm² or more, 350 cells/mm² or more, 400 cells/mm² or more, 500 cells/mm² or more, 750 cells/mm² or more, including 1000 cells/mm² or more. In some embodiments, the core and/or invasive margin of the tumor is determined to be depleted for CD8⁺ cells when the estimated density is 500 cells/mm² or less, e.g., 450 cells/mm² or less, 400 cells/mm² or less, 350 cells/mm² or less, 300 cells/mm² or less, 250 cells/mm² or less, 200 cells/mm² or less, 150 cells/mm² or less, 100 cells/mm² or less, including 50 cells/mm² or less. In certain embodiments, the core and/or invasive margin of the tumor is determined to be depleted for CD4⁺ cells when the estimated density is 500 cells/mm² or less, e.g., 450 cells/mm² or less, 400 cells/mm² or less, 350 cells/mm² or less, 300 cells/mm² or less, 250 cells/mm² or less, 200 cells/mm² or less, 150 cells/mm² or less, 100 cells/mm² or less, including 50 cells/mm² or less. In certain embodiments, the core and/or invasive margin of the tumor is determined to be enriched for CD4⁺ cells when the estimated density is 50 cells/mm² or more, e.g., 100 cells/mm² or more, 150 cells/mm² or more, 200 cells/mm² or more, 250 cells/mm² or more, 300 cells/mm² or more, 350 cells/mm² or more, 400 cells/mm² or more, 500 cells/mm² or more, 750 cells/mm² or more, including 1000 cells/mm² or more. In some embodiments, the core and/or invasive margin of the tumor is determined to be enriched for CD3⁺ cells when the estimated density is 50 cells/mm² or more, e.g., 100 cells/mm² or more, 150 cells/mm² or more, 200 cells/mm² or more, 250 cells/mm² or more, 300 cells/mm² or more, 350 cells/mm² or more, 400 cells/mm² or more, 500 cells/mm² or more, 750 cells/mm² or more, 1000 cells/mm² or more, including 2000 cells/mm² or more.

It is understood that density measurements herein listed in terms of two-dimensional (2D) area (e.g., cells/mm²) correspond to historical methods of analyzing tumor biopsy sections by immunohistochemistry (IHC). Imaging techniques contemplated in the present disclosure (e.g., PET and SPECT) may provide improved density assessment by measuring density in a three-dimensional (3D) volume. A density of cells as used herein may be represented based on a volume (e.g. cells/mm³) and a correlation with IHC results may be established so that historical data can be applied in the improved 3D analysis. For reference, the biopsy tissue samples used in 2D biopsy assessment for standard IHC techniques are typically between 4-50 microns thick, often 20-30 microns thick. An estimate of 3D density can be generated from the 2D density where the thickness of the IHC tissue sample is provided. Where density is reported in 2D terms (e.g., cells/mm²) herein to allow comparison to 2D IHC data, it is to be understood the density measurement can include a corresponding measurement of cell density in 3D.

In some embodiments, the immune contexture provides a good prognosis (e.g., a longer disease-free survival, longer overall survival, or low chance of recurrence) when the estimated ratio of: CD4⁺ cells to CD8⁺ cells is above a threshold; CD8⁺ cells to CD4⁺ cells is above a threshold; CD4⁺ cells to CD3⁺ cells is above a threshold and/or CD8⁺ cells to CD3⁺ cells is above a threshold, in the tumor. In some embodiments, the threshold ratio for CD4⁺ cells to CD8⁺ cells is about 0.5, 0.6, 0.7, 0.8, 0.9, 1, 1.1, 1.2, 1.3, 1.4, 1.5, 1.6, 1.7, 1.8, 1.9, 2, 2.5, 3, 4, 5, 10, 15, 20, 30, 40, 50, 100 or about 1,000. In some embodiments, the threshold ratio for CD8⁺ cells to CD4⁺ cells is about 0.5, 0.6, 0.7, 0.8, 0.9, 1, 1.1, 1.2, 1.3, 1.4, 1.5, 1.6, 1.7, 1.8, 1.9, 2, 2.5, 3, 4, 5, 10, 15, 20, 30, 40, 50, 100 or about 1,000. In some embodiments, the threshold ratio for CD4⁺ cells to CD3⁺ cells is about 0.0001, 0.001, 0.01, 0.1, 0.5, 0.6, 0.7, 0.8, 0.9, 1, 2, 5, 10, 20, 30, 40, 50, 60, 70, 80, 90, or about 100. In some embodiments, the threshold ratio for CD8⁺ cells to CD3⁺ cells is about 0.0001, 0.001, 0.01, 0.1, 0.5, 0.6, 0.7, 0.8, 0.9, 1, 2, 5, 10, 20, 30, 40, 50, 60, 70, 80, 90, or about 100.

In some embodiments, when the immune contexture indicates a poor prognosis (e.g., a shorter disease-free survival, shorter overall survival, or high chance of recurrence) the subject may receive a treatment. In some embodiments, when the immune contexture indicates a poor prognosis (e.g., a shorter disease-free survival, shorter overall survival, high chance of recurrence) the subject may receive adjuvant therapy after an initial treatment for the cancer (e.g., surgical resection of the tumor).

In some embodiments, the immune contexture provides a poor prognosis (e.g., a shorter disease-free survival, shorter overall survival, or higher chance of recurrence) when the core and/or invasive margin of the tumor is depleted for CD3⁺ cells and CD8⁺ cells; depleted for CD3⁺ cells and CD4⁺ cells; enriched for CD4⁺ cells and depleted for CD8⁺ cells; depleted for CD4⁺ cells and enriched for CD8⁺ cells; or depleted for CD8⁺ cells and CD4⁺ cells. In some embodiments, the core and/or invasive margin of the tumor is determined to be depleted for CD8⁺ cells when the estimated density is 500 cells/mm² or less, e.g., 450 cells/mm² or less, 400 cells/mm² or less, 350 cells/mm² or less, 300 cells/mm² or less, 250 cells/mm² or less, 200 cells/mm² or less, 150 cells/mm² or less, 100 cells/mm² or less, including 50 cells/mm² or less. In some embodiments, the core and/or invasive margin of the tumor is determined to be enriched for CD8⁺ cells when the estimated density is 50 cells/mm² or more, e.g., 100 cells/mm² or more, 150 cells/mm² or more, 200 cells/mm² or more, 250 cells/mm² or more, 300 cells/mm² or more, 350 cells/mm² or more, 400 cells/mm² or more, 500 cells/mm² or more, 750 cells/mm² or more, including 1000 cells/mm² or more. In certain embodiments, the core and/or invasive margin of the tumor is determined to be enriched for CD4⁺ cells when the estimated density is 50 cells/mm² or more, e.g., 100 cells/mm² or more, 150 cells/mm² or more, 200 cells/mm² or more, 250 cells/mm² or more, 300 cells/mm² or more, 350 cells/mm² or more, 400 cells/mm² or more, 500 cells/mm² or more, 750 cells/mm² or more, including 1000 cells/mm² or more. In certain embodiments, the core and/or invasive margin of the tumor is determined to be depleted for CD4⁺ cells when the estimated density is 500 cells/mm² or less, e.g., 450 cells/mm² or less, 400 cells/mm² or less, 350 cells/mm² or less, 300 cells/mm² or less, 250 cells/mm² or less, 200 cells/mm² or less, 150 cells/mm² or less, 100 cells/mm² or less, including 50 cells/mm² or less. In some embodiments, the core and/or invasive margin of the tumor is determined to be depleted for CD3⁺ cells when the estimated density is 1000 cells/mm² or less, e.g., 500 cells/mm² or less, 450 cells/mm² or less, 400 cells/mm² or less, 350 cells/mm² or less, 300 cells/mm² or less, 250 cells/mm² or less, 200 cells/mm² or less, 150 cells/mm² or less, 100 cells/mm² or less, including 50 cells/mm² or less.

In some embodiments, the immune contexture provides a poor prognosis (e.g., a shorter disease-free survival, shorter overall survival, or higher chance of recurrence) when the estimated ratio of: CD4⁺ cells to CD8⁺ cells is at or below a threshold ratio; CD8⁺ cells to CD4⁺ cells is at or below a threshold ratio; CD4⁺ cells to CD3⁺ cells is at or below a threshold ratio; and/or CD8⁺ cells to CD3⁺ cells is at or below a threshold ratio, in the tumor. In some embodiments, the threshold ratio for CD4⁺ cells to CD8⁺ cells is about 0.01, 0.1, 0.5, 0.6, 0.7, 0.8, 0.9, 1, 1.1, 1.2, 1.3, 1.4, 1.5, 1.6, 1.7, 1.8, 1.9, 2, 2.5, 3, 4, 5, 10, 15, 20, 30, 40, 50, 100 or about 1,000. In some embodiments, the threshold ratio for CD8⁺ cells to CD4⁺ cells is about 0.01, 0.1, 0.5, 0.6, 0.7, 0.8, 0.9, 1, 1.1, 1.2, 1.3, 1.4, 1.5, 1.6, 1.7, 1.8, 1.9, 2, 2.5, 3, 4, 5, 10, 15, 20, 30, 40, 50, 100 or about 1,000. In some embodiments, the threshold ratio for CD4⁺ cells to CD3⁺ cells is about 0.0001, 0.001, 0.01, 0.1, 0.5, 0.6, 0.7, 0.8, 0.9, 1, 2, 5, 10, 20, 30, 40, 50, 60, 70, 80, 90, or about 100. In some embodiments, the threshold ratio for CD8⁺ cells to CD3⁺ cells is about 0.0001, 0.001, 0.01, 0.1, 0.5, 0.6, 0.7, 0.8, 0.9, 1, 2, 5, 10, 20, 30, 40, 50, 60, 70, 80, 90, or about 100.

In some embodiments, the imaging methods of the present disclosure may be used to determine the effect of a treatment on the immune contexture of a tissue affected by the disease, and based on the response of the immune contexture to the treatment, determine whether to continue the treatment. In certain embodiments, a method of treating a subject includes monitoring, using non-invasive imaging, e.g., PET or SPECT, a distribution of cells expressing a target selected from CD3, CD4, IFN-gamma, and CD8 in one or more tissues of the subject and another distribution of cells expressing a different target selected from CD3, CD4, IFN-gamma, and CD8 in one or more tissues of the subject. In some embodiments, the distribution of cells expressing a third target that is different from the other two targets may be monitored using non-invasive imaging, e.g., PET or SPECT. The monitoring may be done using a suitable non-invasive imaging method as described herein. In some embodiments, the monitoring involves administering to the subject an antigen-binding construct comprising a detectable marker, e.g., radionuclide tracer, where the antigen-binding construct selectively binds the target; and imaging the subject by non-invasive imaging, e.g., PET or SPECT, to acquire the distribution of cells expressing the target in one or more tissues of the subject. The distribution of the different cells can provide the pre-treatment immune contexture of the tissue.

The subject may then be administered with a treatment for the disease, based on the one or more of the immune contexture of the tissue (e.g., tumor) and a change in the immune contexture in the tissue (e.g., tumor). Following the treatment, a post-treatment immune contexture of the tissue may be determined by monitoring, using non-invasive imaging, e.g., PET or SPECT, the distributions of cells expressing the different targets in the tissue. In some embodiments, the post-treatment monitoring involves administering to the subject an antigen-binding construct comprising a detectable marker, e.g., radionuclide tracer, where the antigen-binding construct selectively binds the target; and imaging the subject by non-invasive imaging, e.g., PET or SPECT to acquire the distribution of cells expressing the target in one or more tissues of the subject, as described herein. The distribution of the different cells can provide the post-treatment immune contexture of the tissue (e.g., tumor).

In some embodiments, the distribution of cells expressing the same targets may be monitored before and after the treatment to determine the change in the immune contexture based on the change in distribution of the cells expressing the same set of targets. In some embodiments, the cells monitored before treatment can be expressing a different set of targets than the set of targets expressed by cells monitored after the treatment.

In certain embodiments, a treatment administered to the subject based on the immune contexture determined according to methods of the present disclosure can include, without limitation, one or more of immunotherapy, chemotherapy, hormone therapy, radiation therapy, surgery, vaccine therapy (including intratumoral vaccine therapy), oncolytic virus therapy, or cellular therapy. A treatment received by the subject based on the immune contexture determined by methods of the present disclosure can include, without limitation, one or more of immunotherapy, chemotherapy, hormone therapy, radiation therapy, surgery, vaccine therapy (including intratumoral vaccine therapy), oncolytic virus therapy, or cellular therapy. In some embodiments, an adjuvant therapy administered to the subject based on the immune contexture determined according to methods of the present disclosure can include, without limitation, one or more of immunotherapy, chemotherapy, hormone therapy, radiation therapy, surgery, vaccine therapy (including intratumoral vaccine therapy), oncolytic virus therapy, or cellular therapy.

In some embodiments, methods of the present disclosure recommending against one or more therapies, recommending against continuation of a therapy, recommending one or more additional therapies, or recommending a change to a therapy, based on the immune contexture. In some embodiments, the recommendation provided in the present methods is extrapolated from recommendations developed based on conventional invasive techniques, such as biopsy and IHC.

In certain embodiments, methods of the present disclosure include testing the subject for one or more biomarkers in a bodily fluid, such as blood, urine, saliva, sweat vaginal fluid, semen, etc. In some embodiments, the biomarker is a blood biomarker. Suitable biomarkers include, without limitation, IL-6, C-reactive protein (CRP), VEGF, fibronectin, lactate dehydrogenase (LDH), soluble CD25, NY-ESO-1 antibody, IFN-γ, PD-L1, tumor-associated fibroblast (TAF) markers, FAP/CD8 (neutrophil/lymphocyte ratio), cancer associate fibrosis markers, tumor-associated macrophage markers (e.g., pan CD68; M1 CD86, CD169; M2 CD206, CD163) and chemokines. Suitable biomarkers may further be ascertained by, e.g., T-cell receptor sequencing from tumor and peripheral blood; by targeted gene expression of tumor; or in RNA extracted from the buffy coat fraction in patients' blood. Suitable biomarkers include, without limitation, measures of TCR clonality, TCR convergence, other assessments of clonal expansion, and variable gene polymorphism (e.g. TRBV polymorphism). Certain biomarkers can also include changes in frequency or ratio of CD8⁺ and CD4⁺ cells in the peripheral blood. Suitable biomarkers of interest for immunotherapy are set out in, e.g., Spencer et al. (2016) e493 asco.org/edbook (2016 ASCO (American Society of Clinical Oncology) EDUCATIONAL BOOK).

In some embodiments, methods of the present disclosure include analyzing a tissue biopsy (e.g., tumor biopsy). The biopsy may include any suitable assay to determine, e.g., mutational load, neoantigens load, T-cell receptor sequencing from a tumor sample, targeted gene expression in a tumor, presence or absence of checkpoints or checkpoint ligands, presence or absence of immune inhibitors, inflammatory markers, macrophage-secreted compounds, compounds secreted by myeloid-derived suppressor cells, etc. Suitable assays include, without limitation, high-throughput sequencing (e.g., sequencing of the tumor genome) or immunohistochemistry.

In some embodiments, methods of the present disclosure further include determining an immunoscore of the tissue (e.g., tumor) based on the tissue immune contexture determined as described herein. The non-invasive imaging methods of the present disclosure may substitute or may be used in addition to ways of generating an immunoscore using tissue biopsy and serum biomarkers. Generation of an immunoscore using tissue biopsy and serum biomarkers has been described in, e.g. Galon et al (2104) J Pathol 2014; 232: 199-209; and Blank et al. (2016) Science. Vol 352 Iss. 6286 at 358. According to some embodiments of the present disclosure an immunoscore may be determined non-invasively by measuring the density of one or more immune cells in the tissue of interest. In one embodiment, an immune contexture predictive of poor prognosis is given a lower immunoscore, and an immune contexture predictive of good prognosis is given a higher immunoscore. In another embodiment, a low immunoscore is given good prognosis; high immunoscore is given an unfavourable prognosis. (For the purpose of this disclosure, a high immunoscore will be treated as a favourable prognosis.) In some embodiments, the immunoscore further takes into account the functional activity of immune cells in the tissue, as described above. In some embodiments, the immunoscore further takes into account the functional environment of the tissue, as described above. In some embodiments, the immunoscore further takes into account the presence or absence of biomarkers in the subject's bodily fluid, as described above.

The present disclosure provides non-invasive imaging methods that can enable determination of the immune contexture of a region of interest (ROI) in a subject (e.g., determination of an immunoscore for the subject) to thereby diagnose, provide a prognosis for, recommend treatment options for and/or provide treatment to the subject. The diagnosis, prognosis, recommendation and/or treatment may be based on any suitable known relationship with an immune contexture of the tissue, organ or anatomical region affected by the disease or condition. In some embodiments, the immune contexture determined using methods of the present disclosure may be compared to an immune contexture based on measurements of CD8⁺, CD4⁺ and/or CD3⁺ cells, and/or IFN-gamma using invasive procedures, e.g., biopsy and immunohistochemistry (MC). Any suitable abundance measure of and/or ratio between any combination of CD8⁺, CD4⁺ and CD3⁺ cells, and/or IFN-gamma determined using invasive procedures, e.g., biopsy and IHC, maybe used to analyze the immune contexture determined using the non-invasive imaging methods of the present disclosure.

In general, and without being bound by theory, CD4 expression can represent helper function (antigen presentation by dendritic cells, T helper function by CD4 T cells and “microenvironment” function by macrophages) while CD8 expression can represent effector, or cytotoxic function (e.g., cell killing by CD8⁺ T cells and NK cells and phagocytosis by M1 macrophages). Thus measuring CD3 expression can identify the T cell count in an ROI and CD4±/CD8⁺ ratio can provide the immune status. In some cases, more CD8±/less CD4⁺ can provide a stronger likelihood of response to cancer therapy, depending on the cancer and the therapy. In some cases, low abundance of CD8⁺ cells can indicate good prognosis and/or response to treatment in autoimmune disease. In some cases, low abundance of CD4⁺ cells can indicate good prognosis and response to treatment in autoimmune disease. In some cases, high abundance of CD4⁺ cells can indicate good prognosis and/or response to treatment in autoimmune disease. In general, high CD3⁺ cells and high CD8⁺ cells/low or lower CD4⁺ cells in an immunoscore of a tumor is associated with favourable diagnosis and potential for response to treatment. CD3±/CD4⁺ and CD3±/CD8⁺ ratios can be particularly informative as they can provide guidance on the “immune status” e.g., the presence of a “high” or “low” effector function at the ROI. In some embodiments, the ratios of CD4⁺ and CD8⁺ cells are used to predict efficacy of PD-1 inhibitors or other IOTs. In certain embodiments, CD4⁺ signal (high and prolonged) is an indicator favourable towards PDL-1 and CTLA-4 IOT therapies. In some cases, the CD8⁺ signal is used to select therapy and show therapy induced tumor cell killing. In some embodiments, an increase in the CD4⁺ signal that is sustained and/or prolonged can be predictive of a cell-killing effect, but if the CD4⁺ signal drops, the patient can be advised to change therapy. In some embodiments, IFN-gamma⁺ provides a favorable prognosis for a cancer. In some embodiments, IFN-gamma⁺ provides a stronger likelihood of response to cancer therapy, depending on the cancer and the therapy. The present disclosure provides methods that can aid development of improved predictive immune contextures (e.g., distribution, abundance and/or ratio of CD8⁺, CD4⁺ and CD3⁺ cells, and/or IFN-gamma) for a wide variety of diseases, conditions and treatments.

It is recognized that certain features of the present disclosure, such as “generating an image” or “determin[ing] an immune contexture”, and the like, may involve the application of computerized methods such as radiomics. “Radiomics” as used herein may refer to computer-implemented processes for extracting a large number of features from radiographic medical images. Radiomics may allow identification of one or more features (e.g., radiomic features) that are associated with a disease that are otherwise not recognizable by visual inspection by a healthcare practitioner, such as a physician or an imaging technician. In certain embodiments, methods of the present disclosure may be used in conjunction with radiomics to enhance disease assessment and to identify unexpected disease conditions and correlations. In certain embodiments, methods of the present disclosure may be used in conjunction with radiomics to perform one or more aspects of the methods, to monitor/diagnose/provide prognosis for diseases and conditions other than solid tumors (e.g., to monitor/diagnose/provide prognosis for a non-solid tumor, infectious disease, autoimmune disease, etc.)

Sequential and Simultaneous Imaging of Immune Cell Markers

It will be apparent to those skilled in the art that the methods of the present disclosure, non-limiting examples of which are described in FIGS. 1-3, can be practiced by obtaining images sequentially in time (e.g., on different days), e.g., as shown in FIGS. 4 and 5, or, by obtaining images simultaneously (e.g., on the same day), e.g., as shown in FIG. 6. Either option—sequential or simultaneous—may be selected by the user performing a method of the present disclosure to obtain a diagnostic value of an immunoscore. In some embodiments, sequential imaging of immune cell markers allows the first detectable marker to decay and be eliminated from the body before the second detectable marker is administered to the patient. In some embodiments, sequential imaging permits the use of the same detectable marker for both targets (e.g., any two targets selected from CD3, CD4, CD8, IFNγ), for example, without limitation, use of ⁸⁹Zr, and both images can be generated using the same PET scanner. In some embodiments, a sufficient time period between scanning events is provided to allow the first marker to decay so that it does not interfere with imaging the second marker. In some embodiments, a second scan is taken of the first tracer, before administration of second tracer to allow subtraction of the first tracer image from the second scan from the combined tracer image of the first and second tracers. In some embodiments, imaging the first marker is performed during a different patient visit than imaging the second marker. In some embodiments, the immune contexture of a tissue disease site, and/or the marker signal shape and/or density does not fluctuate substantially between visits. In some embodiments, the immune contexture of a tissue disease site, and/or the marker signal shape and/or density fluctuates substantially between visits. In some embodiments, the analysis of the images from the first and second visits takes into account fluctuations in the immune contexture of a tissue disease site, and/or the marker signal shape and/or density as a function of time.

In some embodiments, simultaneous imaging reduces the number of patient visits and provides an assessment of immune cell markers at the same time point (e.g., for the same day). In some embodiments, in order to achieve simultaneous imaging, several parameters are coordinated, including, but not limited to, time of administration of the agents, the selection of the detectable marker, and other parameters now further described.

Time of administration for simultaneous imaging: In some embodiments, one parameter is the time interval for the imaging agent, or the tracer agent, to circulate, distribute, and bind to its target in the patient's body after administration. Each agent may have a different time requirement to achieve optimal target binding before it is finally cleared by normal elimination processes. In some embodiments, the CD8 marker binding imaging agent is IAB22M2C, and imaging occurs in an approximately 12-48 hour window, approximately 15-40 hour window, approximately 20-36 hour window, approximately 20-30 hour window, or around 24 hours after administration. In some embodiments, the CD8 marker binding imaging agent with a detectable marker can be detected as specifically binding CD8 cells outside of the preferred window, such as during the window 2-20 hours after administration or, on the other side, from 30 hours out to 7 days or longer (e.g. if labelled with ⁸⁹Zr and depending on the dose administered and detector sensitivity). Imaging agents for detecting CD4, CD3, IFNgamma or other markers may have the same time interval of 24 hours for optimal detection, or they may require shorter or longer time periods. In some embodiments, where simultaneous imaging is used, the first and second imaging agents are administered at time points in advance of scanning which are selected to allow sufficient or optimal target binding at the projected time of the scanning event. In some embodiments, different imaging agents may be administered at different times to provide for scanning or imaging at a scheduled time. In some embodiments, users may find a time window that is satisfactory to co-administer both imaging agents, thereby reducing patient visits and in hospital time. In some embodiments, the window for co-administration is 24 hours in advance of imaging. In some embodiments, the window for co-administration is 0.5-1 hour, 1-2 hours, 2-6 hours, 6-12 hours, 12-20 hours, 20-30 hours, or longer, in advance of scanning or imaging.

Selection of the detectable marker for simultaneous imaging: In some embodiments, one parameter for simultaneous imaging is the selection of detectable markers that can be distinguished by the scanner(s) employed. FIG. 6 provides a non-limiting example of using two detectable markers, 123-Iodine and 99m-Technicium, which may be distinguished by the energy of gamma radiation. 123-Iodine emits a maximum radiation at 159 keV. 99m-Technicium emits gamma radiation of 140 keV. By adjusting the filter/collimation on a gamma detector, a single scanner can effectively distinguish¹²³I from ⁹⁹mTc binding, and thereby distinguish individual components of the immune system to which these labels attach, simultaneously. As shown in Table 1 below, in some embodiments, imaging agents such as an ¹²³I-CD8-minibody and a ⁹⁹mTc-CD4-minibody could be separately imaged this way. The skilled artisan will appreciate that “simultaneous” in this context means during the same patient scan procedure, which may include two scans taken consecutively in the same gamma detector device with the different detector filters in place.

A wide variety of pairs or sets of detectable markers may be employed that can be detected simultaneously on common scanners. Common scanners may be selected from among PET, CT, MRI, SPECT, optical/luminescence imaging (including fluorescence imaging or Cerenkov imaging), heat mapping (including near-infrared), acoustic resonance, and photoacoustic resonance. Many health clinics employ clinical PET systems which are combinations of PET and computed tomography (CT) systems, integrating the strengths of both modalities. Another system uses the combination of PET and MRI (magnetic resonance imaging). The MRI modality provides an even higher resolution and soft tissue contrast than CT, allowing for functional imaging without causing any additional radiation burden to the patient. In some embodiments, the two modalities are employed separately to identify different aspects of the same tissue disease site, namely the presence (or absence) of two or more immune cell markers. The reader will appreciate that when two different devices are employed for detection of detectable markers, for example a PET scan and an optical dye scanner, “simultaneous” may include a period while the subject transitions between devices and is prepped for the second scanning procedure.

In some embodiments, MRI is used to detect MRI contrast agents, or enhancer agents which are detectable markers bound to a specific antigen binding construct. In some embodiments, the MRI contrast agent is a gadolinium (Gd) or manganese (Mn)-based contrast agent (e.g., a Gd chelate or a Mn chelate). In some embodiments, a CT scanner is used to detect markers which absorb X-ray transmission. In some embodiments, PET is used to identify PET detectable markers on a different antigen binding target. In some embodiments, imaging options suitable for the present methods include, without limitation, SPECT, optical/luminescence imaging (including fluorescence imaging or Cerenkov imaging), heat mapping (including near-infrared), acoustic resonance, and photoacoustic resonance.

In some embodiments, the combination of markers for use in the present methods is based on instrumentation and/or chemical compatibility. Those skilled in the art, will be able to identify and evaluate suitable marker combinations. Suitable, non-limiting combinations of targets, detectable markers and imaging options for use in the present methods are shown in Table 1.

TABLE 1
Simultaneous Imaging Table

| | | Time of | | |
|---|---|---|---|---|
| Immunoscore | Targeting agent with | Administration | | Simultaneous |
| components | detectable marker | prior to scanning | | Scanning device(s) |
| CD8/CD4 | 89Zr-CD8-minibody | 6-48 | hours | PET/Gamma |
| | 123I-CD4-minibody | | | |
| CD8/CD4 | 123I-CD8-minibody | 6-48 | hours | Gamma 159 keV/Gamma 140 keV |
| | 99mTc-CD4-minibody | | | |
| CD8/CD4/IFN | 89Zr-CD8-minibody | 6-48 | hours | PET/MRI/Optical |
| gamma | Gd-DTPA-CD4-cys-diabody | 1-12 | hours | imaging/Photoacoustic |
| | Mn-chelate-C4-cys diabody | | | |
| | ICG-IFNgamma-antigen- | 6-48 | hours | |
| | binding construct | | | |
| CD8/CD3 | 89Zr-CD8-minibody | 6-48 | hours | PET/MRI |
| | Gd-DTPA-CD3-minibody | | | |
| CD8/IFN | 89Zr-CD8-minibody | 6-48 | hours | PET/MRI |
| gamma | Fe-nano-IFNgamma-antigen- | | | |
| | binding construct | | | |
| CD8/PSMA | 89Zr-CD8-minibody | 6-48 | hours | PET/Optical imaging |
| | IR dye-PSMA-minibody | | | |
| CD8/CD4 | QuantumDot-CD8-minibody | 6-48 | hours | CT/Optical PET |
| | 89Zr-CD4-minibody | | | |
| CD4/CD3 | 18F or 68Ga-CD4-minibody | 6-48 | hours | PET/CT |
| | tantalum-CD3-cys-diabody | 1-12 | hours | |
| CD8/CD4 | 18F-CD8-nanobody | 1-4 | hours | PET/Gamma |
| | 123I-CD4-nanobody | | | |
| CD8/IFNgamma | 18F-CD8-binding ligand | 1-4 | hours | PET/Gamma |
| | (123I or 99mTc) -IFNgamma- | | | |
| | antigen-binding construct | | | |

In some embodiments, where the detectable marker is a radionuclide or other detectable marker which decay substantially during the period of administration prior to scanning, the administered amount of the detectable marker may be adjusted (e.g., increased or decreased) to provide a signal level of the detectable marker that is sufficient for imaging at the later time point of the scan. In some embodiments, an imaging agent, e.g., a radionuclide-labeled antigen-binding construct, such as a ⁸⁹Zr-CD8-minibody, is provided in a range of 0.5 to 3.6 millicurie to a human subject, which is suitable for detection in a time window of 20-30 hours post administration. In some embodiments, a detectable marker, e.g., a radionuclide, such as ¹⁸F, is administered at 8 millicurie. In some embodiments, the dose of the detectable marker, such as ¹⁸F, is adjusted, e.g., increased or decreased, depending on the distribution and circulation time required. In some embodiments, the dose of ¹⁸F is increased, as it has a half life of only 109.7 minutes.

Any suitable amount of each targeting agent can be administered. Those skilled in the art are familiar with multiple ascending dose trials that can be used in some embodiments to identify the optimal amount of agent to be administered. In some embodiments, a ⁸⁹Zr-CD8-minibody is administered at does in a range of between about 0.5 mg to about 10 mg of protein, and or a dose of about 1.5 mg. In some embodiments, the dose is 2.5 mg or lower, typically classified as a “microdose”.

As used herein “generating an image” or “determin[ing] an immune contexture” or “generating an immunoscore”, may refer to imaging and analysis by sequential scans or by simultaneous scans, as discussed herein. In some embodiment, an immunoscore analysis will be used based on the imaging data, to make or instruct a diagnosis, prognosis and/or treatment recommendation for the subject. The immunoscore analysis may include any suitable analysis, e.g., as described in WO2020/069433, and in Bruni et al. (The immune contexture and Immunoscore in cancer prognosis and therapeutic efficacy. Nat Rev Cancer 20, 662-680 (2020)), each of which is incorporated herein by reference.

### Additional Embodiments

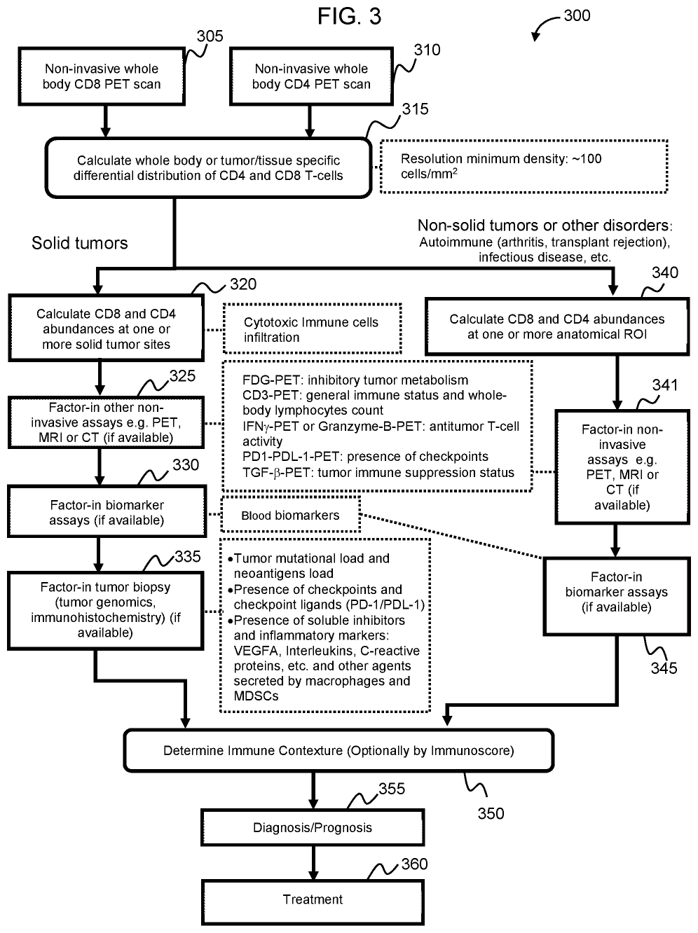

With reference to FIG. 3, a method 300 according to some embodiments of the present disclosure is shown. The method may include non-invasively imaging a subject using a whole-body PET scan 305, where the subject has been administered a radionuclide-labeled antigen-binding construct (e.g., a PET tracer) that selectively binds CD8. The subject may have a disease, such as a cancer, autoimmune disease or infectious disease. In some cases the subject has a solid tumor, or non-solid tumor. The method can further include non-invasively imaging the subject using a whole-body PET scan 310, where the subject has been administered a radionuclide-labeled antigen-binding construct that selectively binds CD4. The whole-body PET scan may measure the distribution of radioactivity in the subject's body that relates to the density of cells (e.g., immune cells, such as T cells) expressing CD4 or CD8. Based on the whole-body PET scans, a whole-body or tumor-/tissue-specific differential distribution of CD4 and CD8 T cells may be calculated 315. The PET scan may be capable of resolving cells at a minimum density of about 50 to 100 cells/mm².

Where the subject has a solid tumor, the method may include determining the extent of cytotoxic immune cell infiltration into the tumor environment. In some embodiments, the abundance of CD8⁺ and/or CD4⁺ cells at one or more tumor sites is calculated 320. In some embodiments, the spatial distribution of CD8⁺ and/or CD4⁺ cells in the tumor is determined. In some embodiments, the temporal distribution of CD8⁺ and/or CD4⁺ cells in the tumor is determined. In some embodiments, the overlap between CD8⁺ cells and CD4⁺ cells within the tumor is determined. In some embodiments, the ratio of the CD8 signal and CD4 signal at different sites in the tumor is compared. In some embodiments, the spatial distribution of CD8⁺ or CD4⁺ cells with respect to other cellular components of the tumor microenvironment is compared.

Based on the calculated differential distribution of CD4⁺ and CD8⁺ cells, and abundance of CD4⁺ and CD8⁺ cells in the tumor, an immune contexture (as represented by, e.g., an immunoscore) may be determined 350 for the tumor, tissue and/or the whole body of the subject. The immune contexture (e.g., immunoscore) can provide a prediction or prognosis for the subject's disease progression. In some embodiments, a high immunoscore indicates a favorable prognosis (e.g., lower chance of tumor recurrence after treatment) and a low immunoscore indicates a poor prognosis (e.g., higher chance of tumor recurrence after treatment). In another embodiment, this could be reversed with a low immunoscore being poor prognosis; high immunoscore being favourable. (For the purpose of this disclosure, a high immunoscore will be treated as a favourable prognosis.) Regardless, based on the immunoscore, the subject may be diagnosed 355, e.g., by a healthcare practitioner, such as a physician. In some embodiments, the subject may be recommended a course of treatment (e.g., selection of a particular therapy or treatment) based on the immunoscore. In some embodiments, the subject may be recommended no treatment (e.g., adjuvant therapy) after an initial treatment (e.g., surgical resection) based on the immunoscore (e.g., an immunoscore indicating good prognosis). In some embodiments, the subject may be recommended more frequent monitoring for tumor recurrence based on the immunoscore (e.g., an immunoscore indicating a poor prognosis).

In some embodiments, the subject may be given a treatment 360 based on an immunoscore and a subsequent diagnosis. The immunoscore and diagnosis can determine whether a subject should receive one or more of several treatments, in the case of cancer, including immunotherapy, chemotherapy, hormone therapy, radiation therapy, surgery, vaccine therapy, oncolytic virus therapy, or cellular therapy. In some embodiments, the subject may undergo surgery to remove the tumor. In some embodiments, a subject may be administered adjuvant therapy after the surgery when the subject's immunoscore for the tumor is found to be low. In some embodiments, a subject may be examined for tumor recurrence more frequently (e.g., monthly or yearly) after the surgery when the subject's immunoscore for the tumor is found to be low. In some embodiments, a subject may be not be administered any adjuvant therapy after the surgery when the subject's immunoscore for the tumor is found to be high. In some embodiments, a subject may be examined for tumor recurrence less frequently (e.g., once every five years or longer) after the surgery when the subject's immunoscore for the tumor is found to be high.

In some embodiments the immunoscore takes into account additional factors. Additional information about the immune status of the individual and/or the tumor that can contribute to the immunoscore include, without limitation, inhibitory tumor metabolism, general immune status, whole-body lymphocytes count, antitumor T-cell activity, presence of checkpoints, presence of inhibitory cytokine, presence of activating cytokines, presence of inhibitory chemokines, presence of activating chemokines, extent of tumor fibrosis, or tumor immune suppression status. In some embodiments, data from one or more non-invasive imaging assays can contribute to the immunoscore 325. Any suitable imaging assay for probing the immune status of the individual and/or the tumor may be used. Suitable non-invasive assays include, without limitation, FDG-PET, CD3-PET, IFNγ-PET, Granzyme B-PET, PD-1-PET, PD-L1-PET, TGFβ-PET.

In certain embodiments, the immunoscore can take into account one or more biomarker assay results 330. The biomarker may be a blood biomarker.

In some embodiments, the immunoscore can take into account results from a tumor biopsy 335. A tumor biopsy may be used to obtain information about tumor mutational load and neoantigens load, the presence of checkpoints and checkpoint ligands (PD-1/PDL-1), and/or the presence of soluble inhibitors and inflammatory markers, such as, but not limited to VEGFA, Interleukins, C-reactive proteins, etc. and other agents secreted by macrophages and myeloid-derived suppressor cells (MDSCs). A tumor biopsy can be tested using any suitable assay for determining these tumor characteristics relevant for diagnosis and/or prognosis. A biopsy may be tested using high through-put sequencing to carry out tumor genomics, or immunohistochemistry.

Where the subject has a non-solid tumor, an autoimmune disorder or an infectious disease, the immune contexture (as represented by, e.g., an immunoscore) may be based on the whole-body, or anatomical ROI, or tissue-specific differential distribution of CD4⁺ and CD8⁺ cells. As for the tumor describe above, the method may include calculating the abundance of CD8⁺ and/or CD4⁺ cells at one or more sites (e.g., tissues, anatomical ROI) is calculated 340. In some embodiments, the spatial and/or temporal distribution of CD8⁺ and/or CD4⁺ cells in the anatomical ROI is determined. The immune contexture (e.g., immunoscore) can further take into account the subject's general immune status, or optionally, a whole-body lymphocyte count through CD3-PET 341, and results of any optional blood biomarker tests 345.

Boxes 325 and 341 indicate that other non-invasive assays such as PET scans targeting other biological markers, magnetic resonance imaging (MRI), and/or computed tomography (CT) are suitable for use in combination with the methods of the present disclosure to determine immune contexture of an anatomical ROI 350. In some cases, MRI may be used to confirm immune contexture, and MRI may itself be correlated with diagnosis, prognosis and treatment recommendations based on validation established by methods of the present disclosure.

Also provided herein is a method of imaging a subject, comprising: administering to a subject a first antigen-binding construct comprising a first detectable marker, wherein the antigen-binding construct selectively binds a first target selected from CD3, CD4, IFN-gamma, and CD8; estimating a distribution and/or abundance of cells expressing the first target in one or more tissues of the subject using non-invasive imaging to measure a level of the first detectable marker in the subject; administering to the subject a second antigen-binding construct comprising a second detectable marker, wherein the antigen-binding construct selectively binds a second target selected from CD3, CD4, IFN-gamma, and CD8, and wherein the first and second targets are different; estimating a distribution and/or abundance of cells expressing the second target in the one or more tissues of the subject using non-invasive imaging to measure a level of the second detectable marker in the subject; and generating an image based on the distributions and/or abundances of the cells expressing the targets, wherein the image provides an indication of the immune contexture of the one or more tissues. The first antigen binding construct and the second antigen binding construct can be administered at any suitable time relative to each other. In some embodiments, administering the first antigen binding construct and administering the second antigen binding construct are performed within about 1, 2, 3, 4, 5, 6, 8, 10, 12, 16, 20, 24, 36, 48, 60, 72, 96, or 120 hours or more, or a time interval within a range between any two of the preceding values, of each other. In some embodiments, administering the first antigen binding construct and administering the second antigen binding construct are performed on the same day. In some embodiments, administering the first antigen binding construct and administering the second antigen binding construct are performed on different days, e.g., during separate patient visits, such as 1, 2, 3, 4, 5, 6, 7, 8, 9, 10, 11, 12, 13, 14 or more days, apart from each other. Imaging the subject to measure the level of the first detectable marker and the second detectable marker can be done at any suitable time relative to each other. In some embodiments, using non-invasive imaging to measure the level of the first detectable marker and using non-invasive imaging to measure the level of the second detectable marker are performed within about 1, 2, 3, 4, 5, 6, 8, 10, 12, 16, 20, 24, 36, 48, 60, 72, 96, or 120 hours or more, or a time interval within a range between any two of the preceding values, of each other. In some embodiments, using non-invasive imaging to measure the level of the first detectable marker and using non-invasive imaging to measure the level of the second detectable marker are performed on the same day. In some embodiments, using non-invasive imaging to measure the level of the first detectable marker and using non-invasive imaging to measure the level of the second detectable marker are performed on different days, e.g., during separate patient visits, such as 1, 2, 3, 4, 5, 6, 7, 8, 9, 10, 11, 12, 13, 14 or more days, apart from each other.

In some embodiments, the method includes administering to a subject a third antigen-binding construct comprising a third detectable marker, wherein the antigen-binding construct selectively binds a third target selected from CD3, CD4, IFN-gamma, and CD8, where the third target is different from the first and second targets; and estimating a distribution and/or abundance of cells expressing the third target in one or more tissues of the subject using non-invasive imaging to measure a level of the third detectable marker in the subject.

In some embodiments, the first detectable marker and the second detectable marker are different and are selected from a radionuclide, an optical dye, a fluorescent compound, a Cerenkov luminescence agent, a paramagnetic ion, an MRI contrast agent, an MRI enhancer agent and a nanoparticle, as disclosed herein. In some embodiments, the non-invasive imaging is selected from PET, SPECT, MRI, CT, gamma-ray imaging, optical imaging, and Cherenkov luminescence imaging (CLI), as disclosed herein.

Detectable Markers, Pet Tracers, Antigen-Binding Constructs and Targets

The term “PET tracer” denotes any molecule that can associate or selectively bind to a target (e.g., CD3, CD4 or CD8) and associate a marker or label with the target. This includes aspects such as antigen binding constructs, antibodies, minibodies, diabodies, cys-diabodies, nanobodies, etc. Further included within the scope of a PET tracer are small peptides and small molecules that selectively bind to a target, and to which a PET marker or PET detectable label can be associated (e.g., linked or covalently bonded to). In some embodiments, the PET tracer is less than 200kDA, 170 kDa, 150 kDa, 120 kDa, 105 kDa, 100 kDa, 80 kDa, 50 kDa, 30 kDa, 10 kDa, 5 kDa, or 2 kDa. Where an antigen-binding construct is referenced in the present disclosure, a suitable PET tracer is also contemplated.

An example of CD8 PET tracers include CD8 specific capture agents, such as those disclosed in WO2017/176769, the entirety of which is incorporated herein by reference with respect to such CD8-specific capture agents. In some embodiments, any of the methods provided herein can employ a CD8 capture agent (or just the “ligands”) as provided in WO2017/176769, including the capture agent of any of the following:

(1) HGRGH (SEQ ID NO:225)-Linker-wplrf (SEQ ID NO:226), targeted against Epitope 2C (AAEGLDTQR (SEQ ID NO:227)) and Epitope IN (SQFRVSPLD (SEQ ID NO:228)).

(2) HGRGH (SEQ ID NO:225)-Linker-AKYRG (SEQ ID NO:229), targeted against Epitope 2C (AAEGLDTQR (SEQ ID NO:227)) and Epitope IN (SQFRVSPLD (SEQ ID NO:228)).

(3) Ghtwp (SEQ ID NO:245)-Linker-hGrGh (SEQ ID NO:246), targeted against Epitope 2N (FLLYLSQNKP (SEQ ID NO:230)) and Epitope 2C (AAEGLDTQR (SEQ ID NO:227)).

(4) PWTHG (SEQ ID NO:231)-Linker-AKYRG (SEQ ID NO:229), targeted against Epitope 2N (FLLYLSQNKP (SEQ ID NO:230)) and Epitope IN (SQFRVSPLD (SEQ ID NO:228)).

In some embodiments, the molecule that binds to CD8 consists or comprises one or more of:

1) A sequence that is 80-100% identical to at least one of: a. HGSYG (SEQ ID NO:232); b. KRLGA (SEQ ID NO233); c. AKYRG (SEQ ID NO:229); d. hallw (SEQ ID NO:234); e. lrGyw (SEQ ID NO:235); f. vashf (SEQ ID NO:236); g. nGnvh (SEQ ID NO:237); h. wplrf (SEQ ID NO:226); i. rwfnv (SEQ ID NO:238); j. havwh (SEQ ID NO:239); k. wvplw (SEQ ID NO:240); 1. Ffrly (SEQ ID NO:241); and m. wyyGf (SEQ ID NO:242); or

2) A sequence 80-100% identical to an amino acid sequence selected from the group consisting of: a AGDSW (SEQ ID NO:243); b. HVRHG (SEQ ID NO:244); c. HGRGH (SEQ ID NO:225); d. THPTT (SEQ ID NO:247); e. FAGYH (SEQ ID NO:248); f. WTEHG (SEQ ID NO:249); g. PWTHG (SEQ ID NO:231); h. TNDFD (SEQ ID NO:250); i. LFPFD (SEQ ID NO:251); j. slrfG (SEQ ID NO:252); k. yfrGs (SEQ ID NO:253); 1. wnwvG (SEQ ID NO:254); m. vawlG (SEQ ID NO:255); n. fhvhG (SEQ ID NO:256); o. wvsnv (SEQ ID NO:257); p. wsvnv (SEQ ID NO:258); q. InshG (SEQ ID NO:259); r. yGGvr (SEQ ID NO:260); s. nsvhG (SEQ ID NO:261); t. ttvhG (SEQ ID NO:262); u. fdvGh (SEQ ID NO:263); v. rhGwk (SEQ ID NO:264); w. Ghtwp (SEQ ID NO:245); and x. hGrGh (SEQ ID NO:265).

Antigen-binding constructs suitable for use in methods of the present disclosure include any suitable antibody, or antigen-binding fragments thereof, that selectively bind to a target (e.g., immune cell marker). Suitable antigen-binding constructs include, without limitation, an antibody, Fab′, F(ab′)2, Fab, Fv, rIgG (reduced IgG), a scFv fragment, a minibody, a diabody, a cys-diabody, or a nanobody. The target to which an antigen-binding construct binds may be any suitable immune cell marker (e.g., cell-surface marker) for identifying immune cell types that contribute to the immune contexture of a tissue.

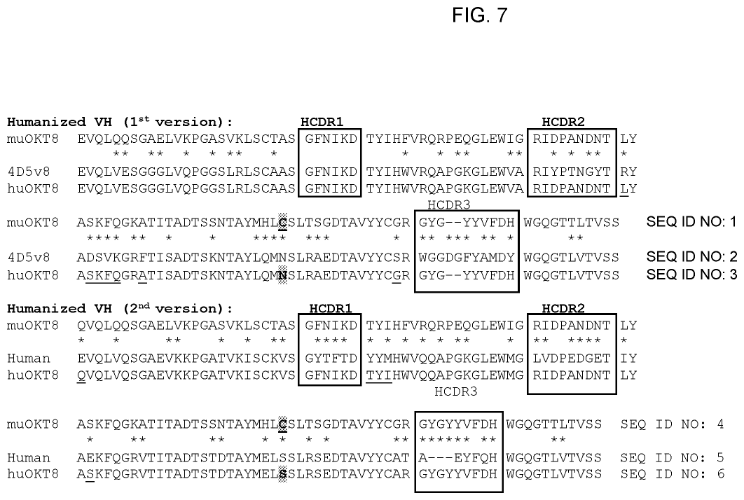

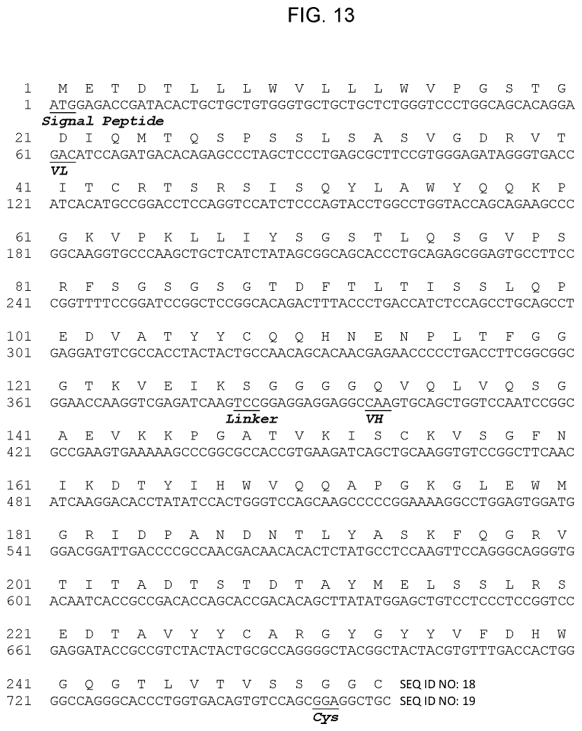

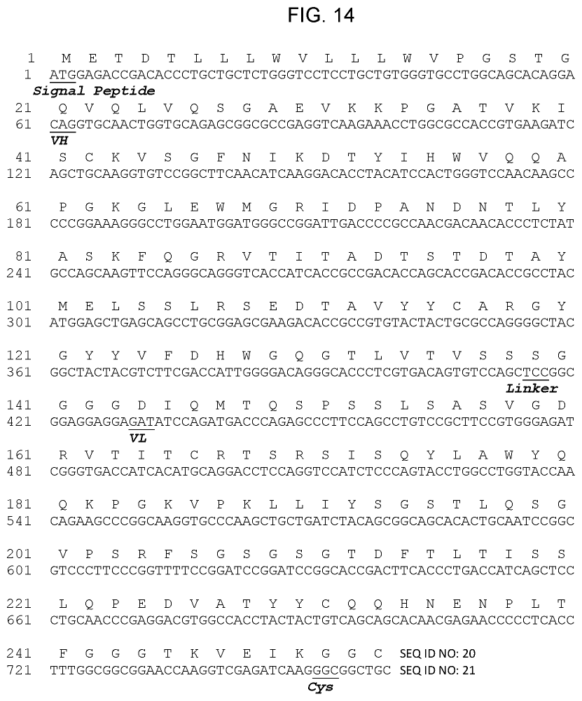

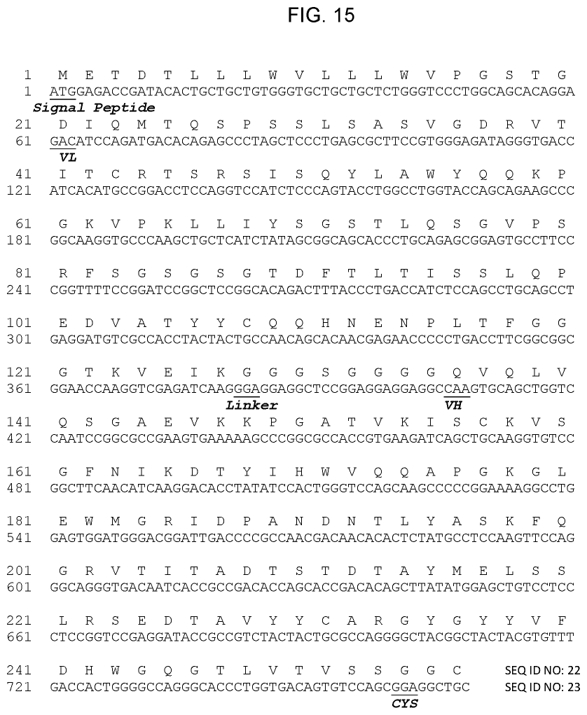

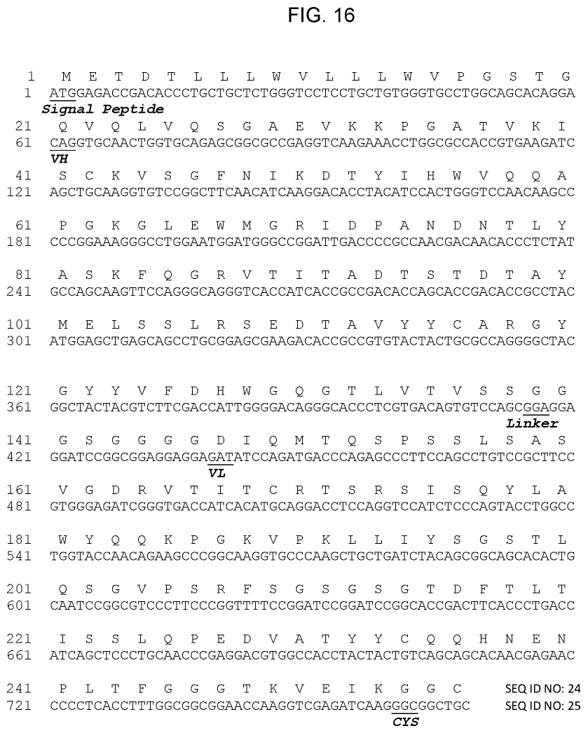

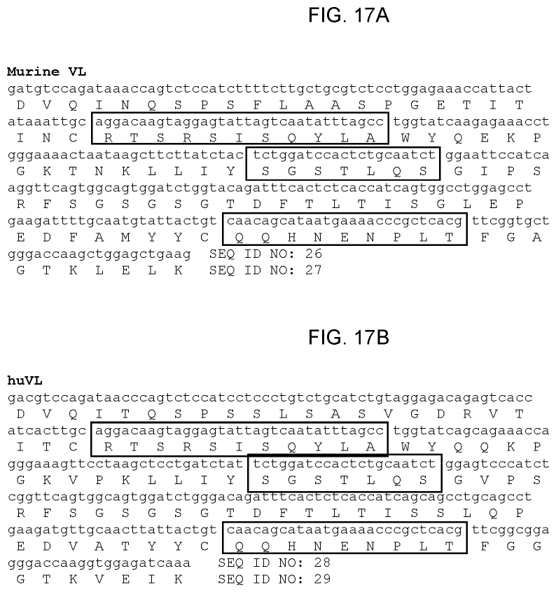

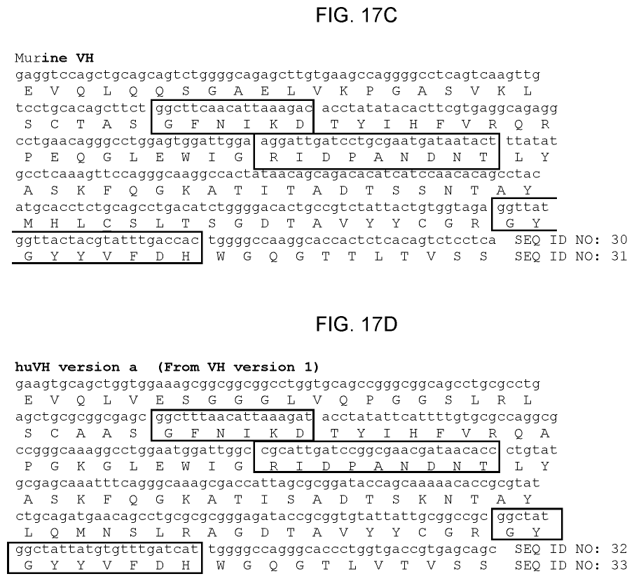

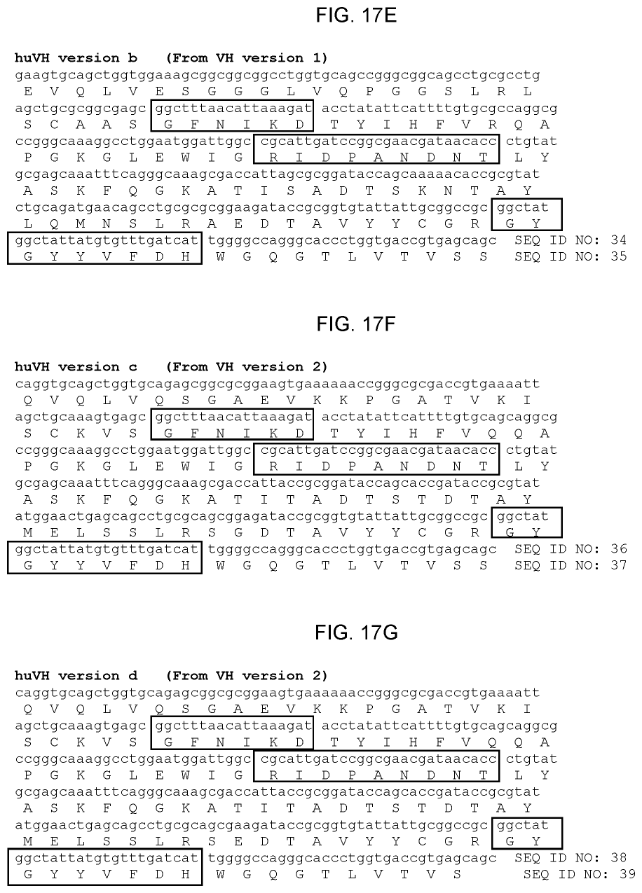

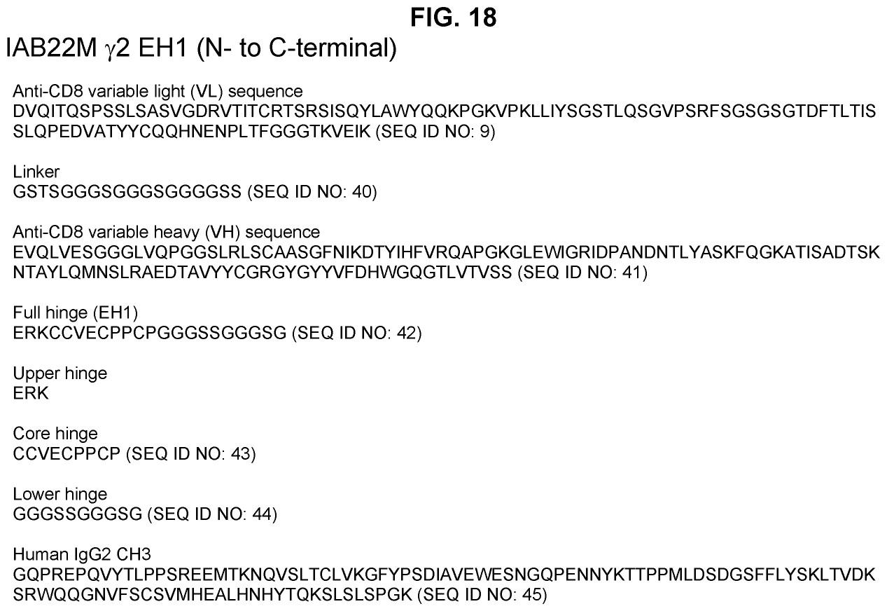

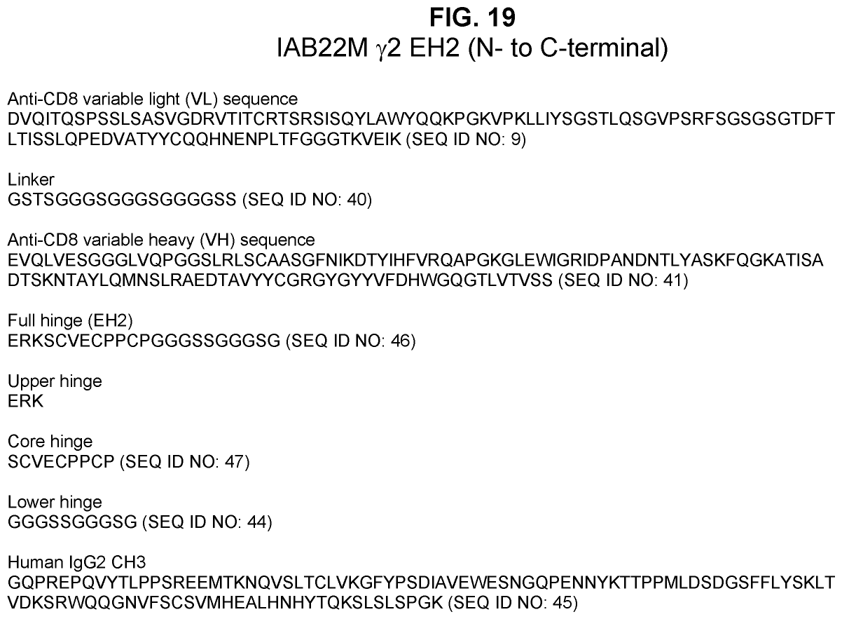

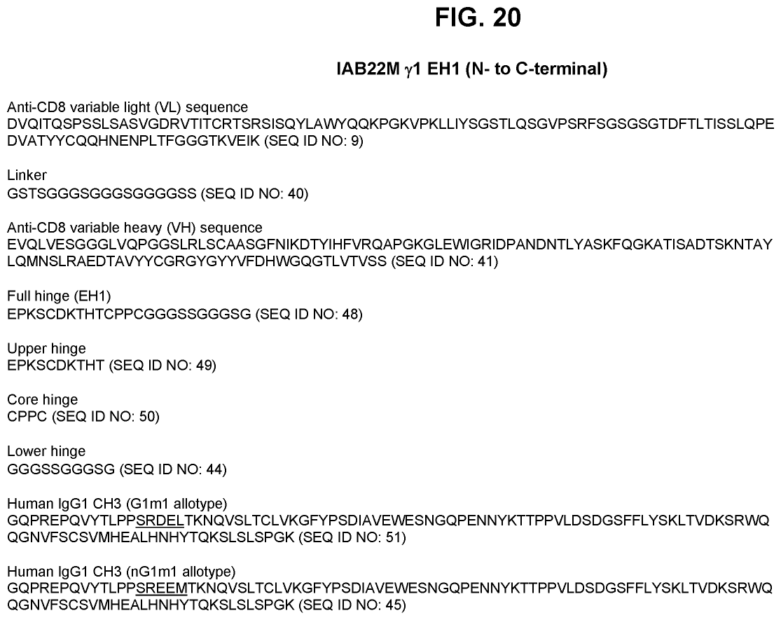

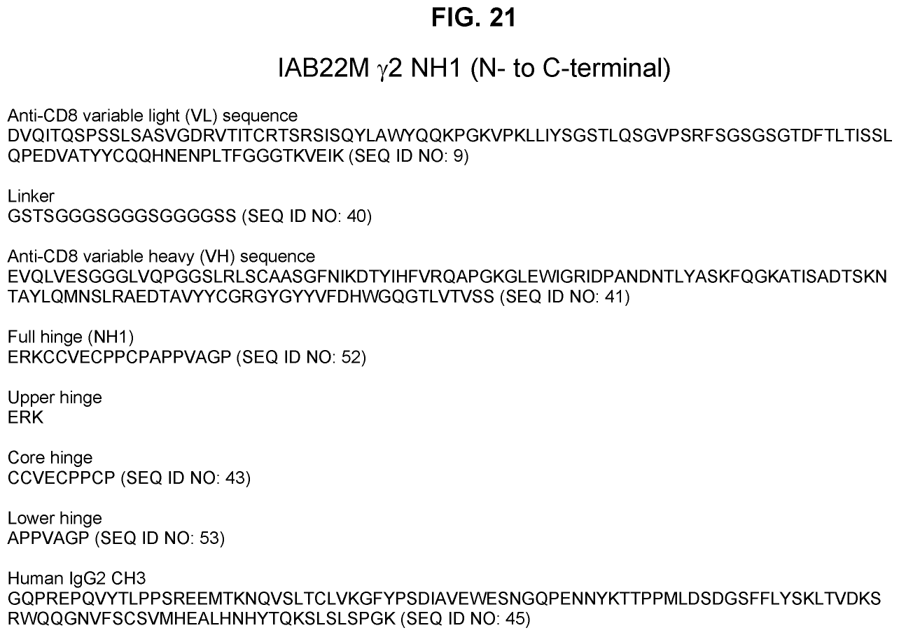

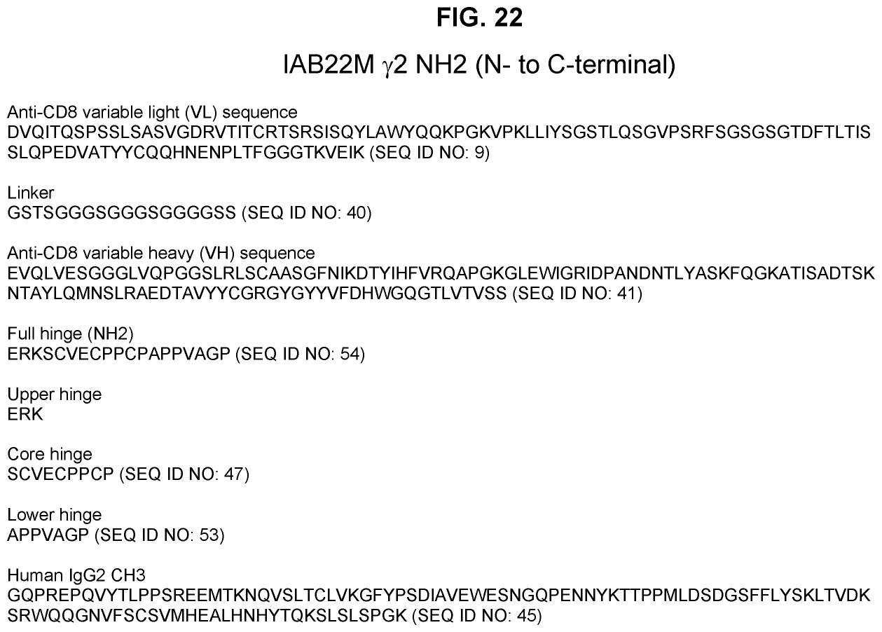

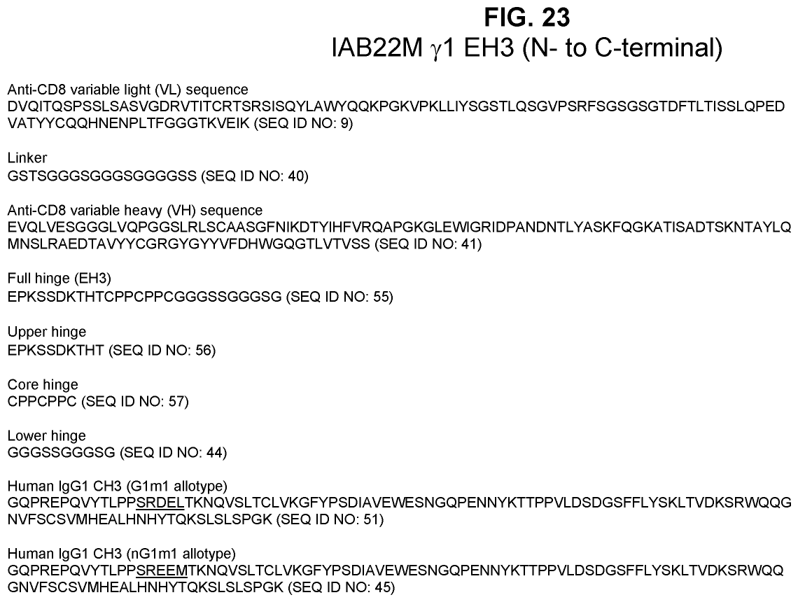

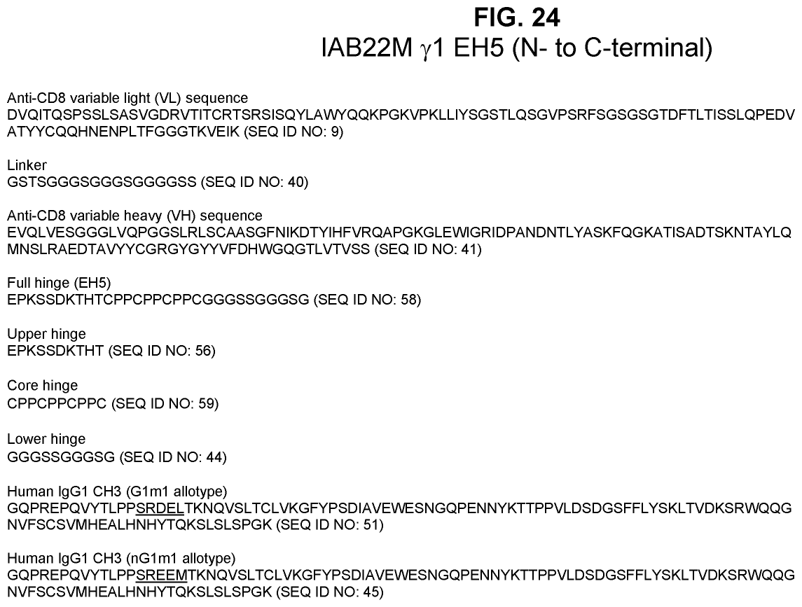

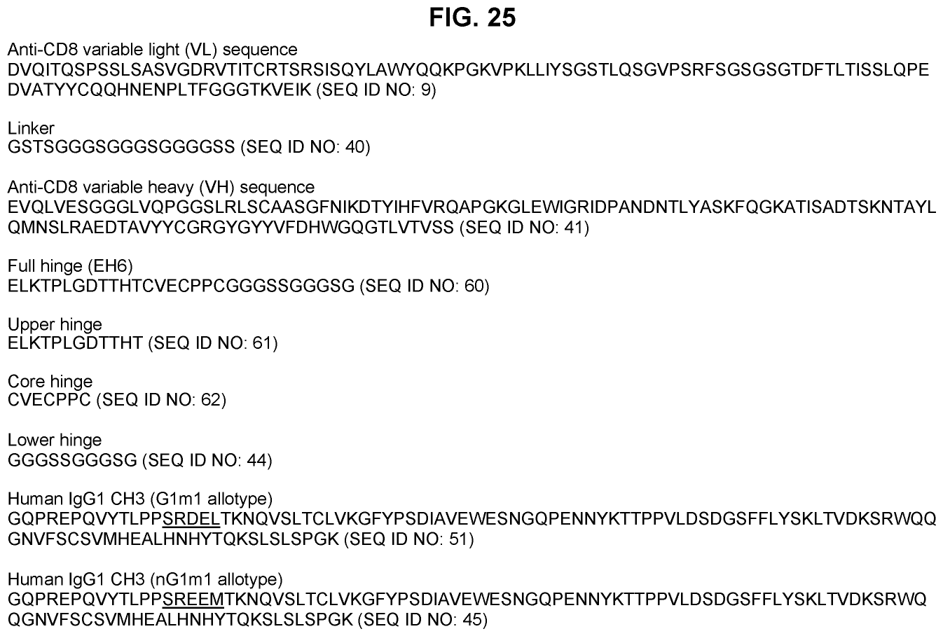

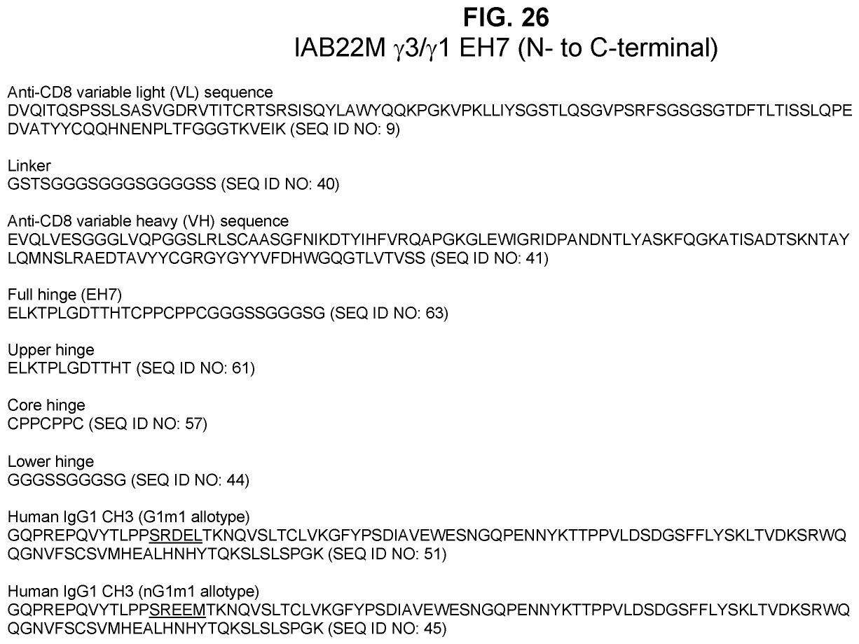

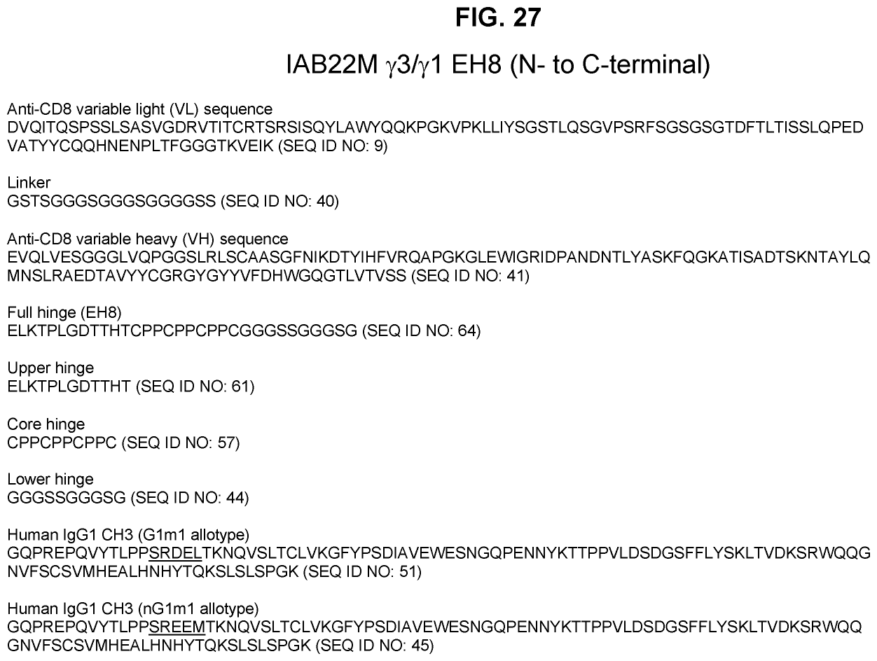

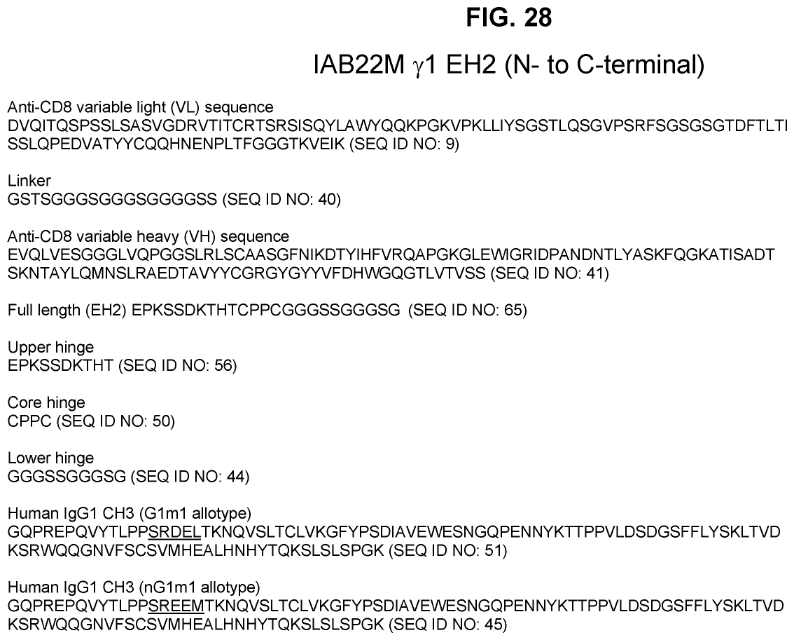

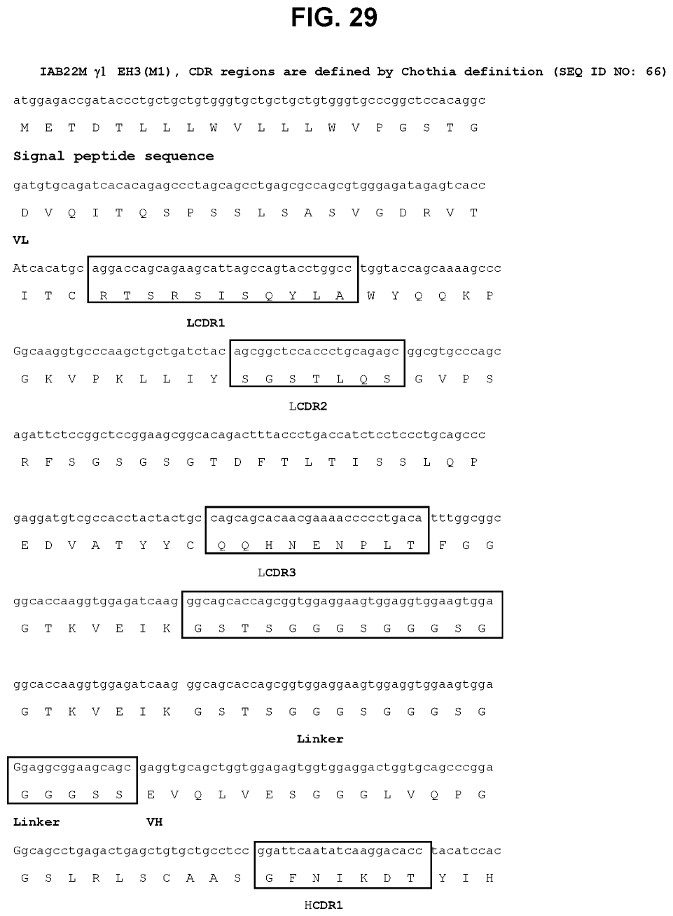

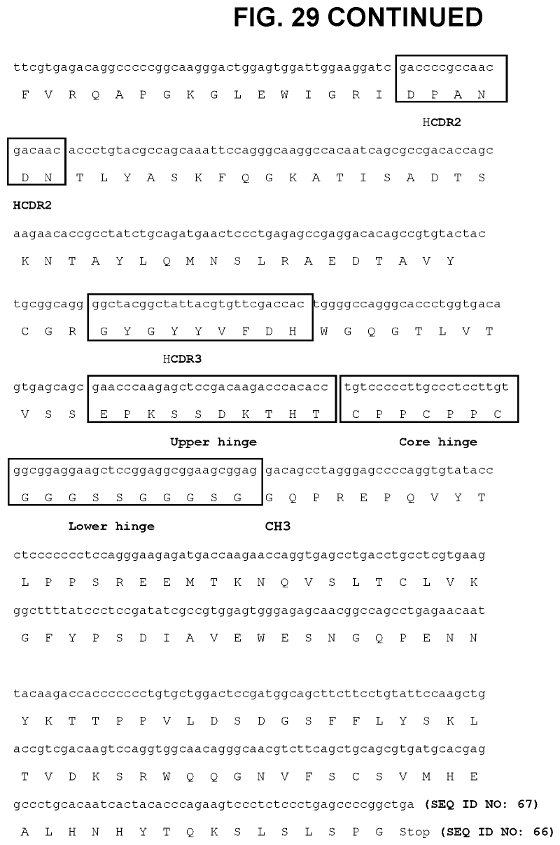

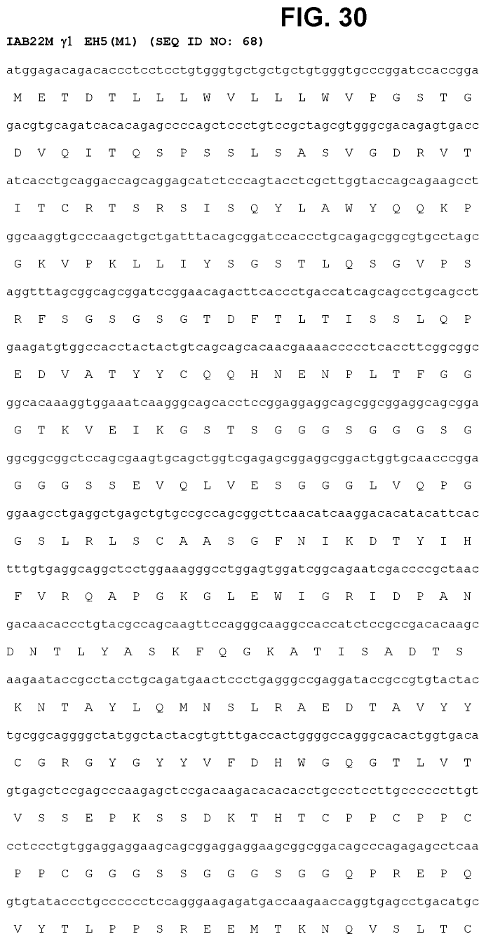

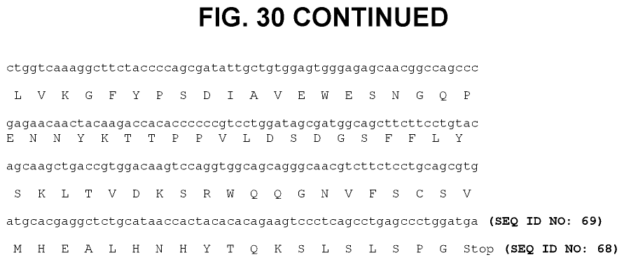

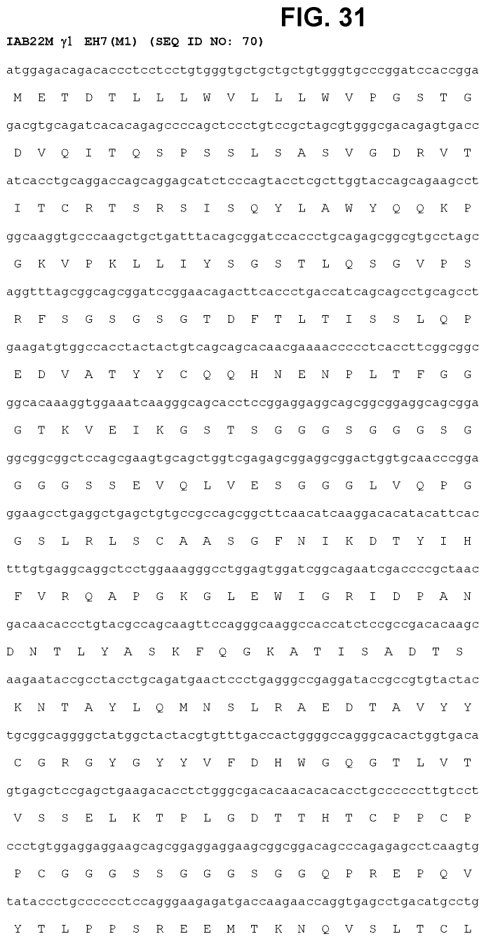

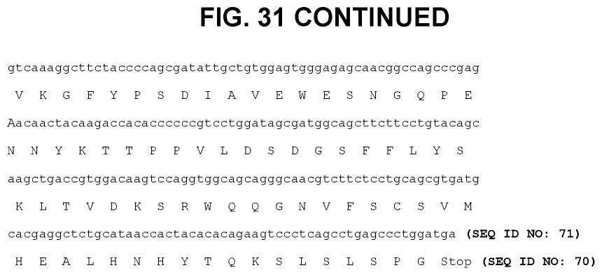

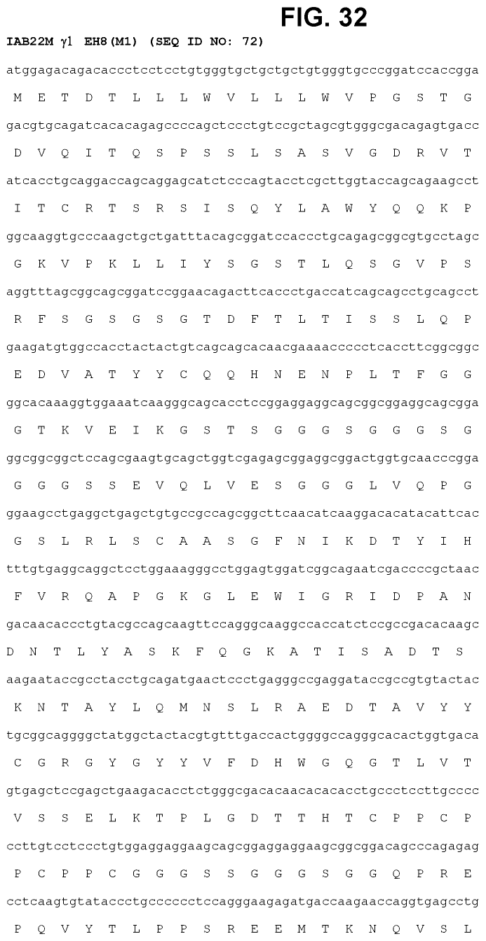

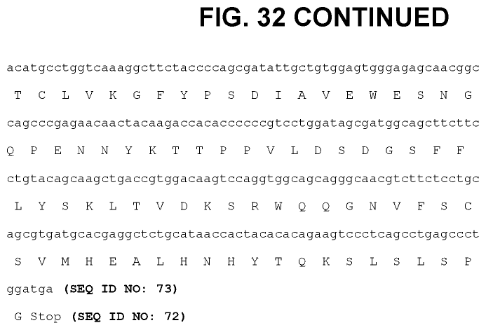

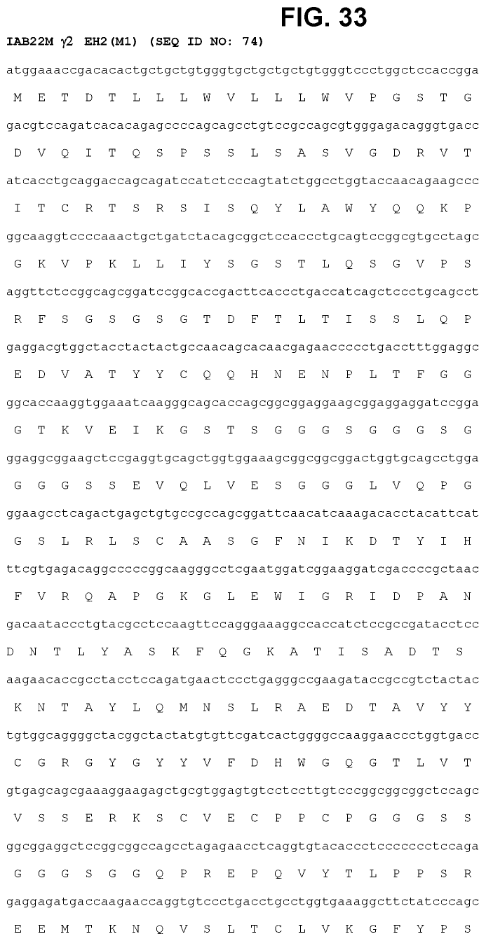

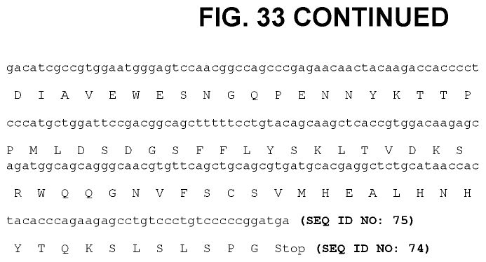

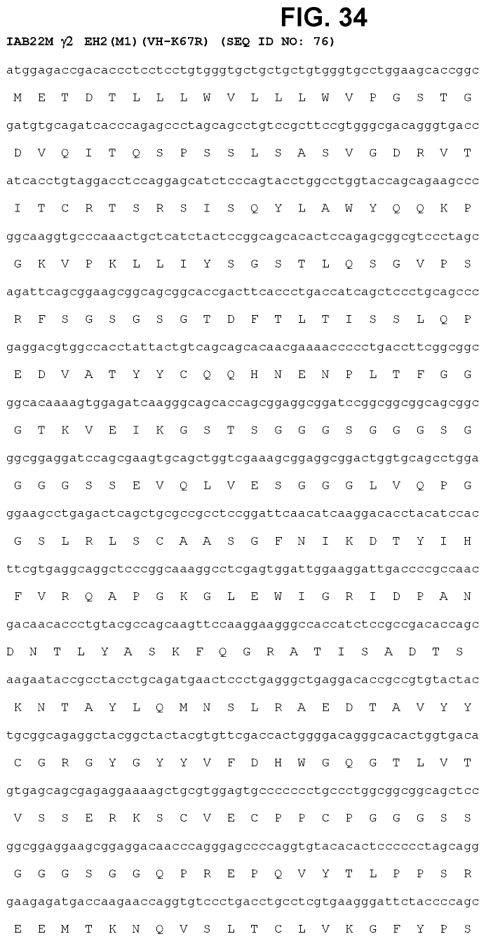

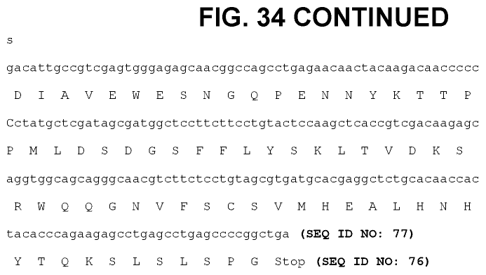

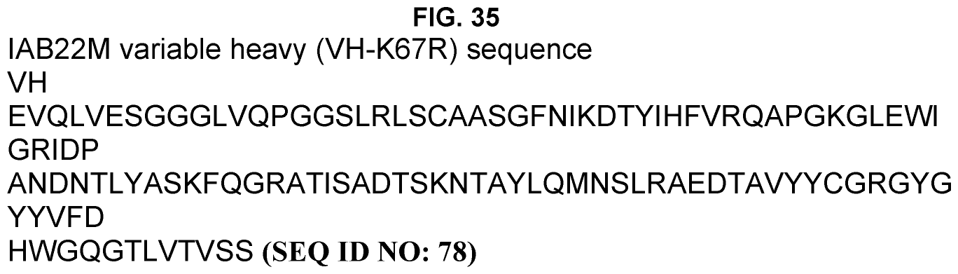

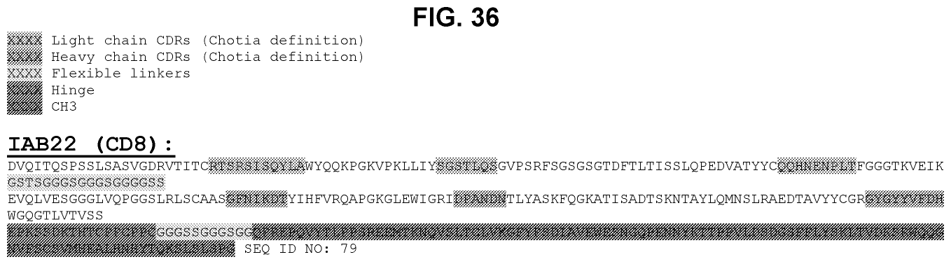

Suitable antigen-binding constructs that selectively bind CD8 are described, e.g., in International Application No. PCT/US2019/053642, filed Sep. 27, 2019, and U.S. Patent Publication No. 20170029507, which are incorporated herein by reference. In some embodiments, a CD8 antigen-binding construct suitable for use in methods of the present disclosure include any of the amino acid sequences described in FIGS. 7-36. In some embodiments, a CD8 antigen-binding construct suitable for use in methods of the present disclosure include an amino acid sequence at least about 80%, 85%, 86%, 87%, 88%, 89%, 90%, 91%, 92%, 93, 94, 95%, 96%, 97%, 98%, or 99% identical to any of the amino acid sequences described in FIGS. 7-36. In certain embodiments, a CD8 antigen-binding construct suitable for use in methods of the present disclosure include any antigen-binding fragment or a portion thereof, such as any one of or all the CDRs, heavy chain variable (V_{H}) region, light chain variable (V_{L}) region, heavy and light chain variable regions, hinge region, etc., of the amino acid sequences depicted in FIGS. 7-36. In some embodiments, the CD8 antigen-binding construct comprises a V_{H} region having an amino acid sequence at least about 80%, 85%, 86%, 87%, 88%, 89%, 90%, 91%, 92%, 93, 94, 95%, 96%, 97%, 98%, 99%, or 100% identical to the V_{H} region of any one of SEQ ID NOs: 1-6, 10, 12, 14, 16, 18, 20, 22, 24, 31, 33, 35, 37, 39, 41, 66, 68, 70, 72, 74, 76, 78, 79. In some embodiments, the CD8 antigen-binding construct comprises a V_{L} region having an amino acid sequence at least about 80%, 85%, 86%, 87%, 88%, 89%, 90%, 91%, 92%, 93, 94, 95%, 96%, 97%, 98%, 99%, or 100% identical to the V_{L} region of any one of SEQ ID NOs: 7-9, 10, 12, 14, 16, 18, 20, 22, 24, 27, 29, 66, 68, 70, 72, 74, 76, 79.

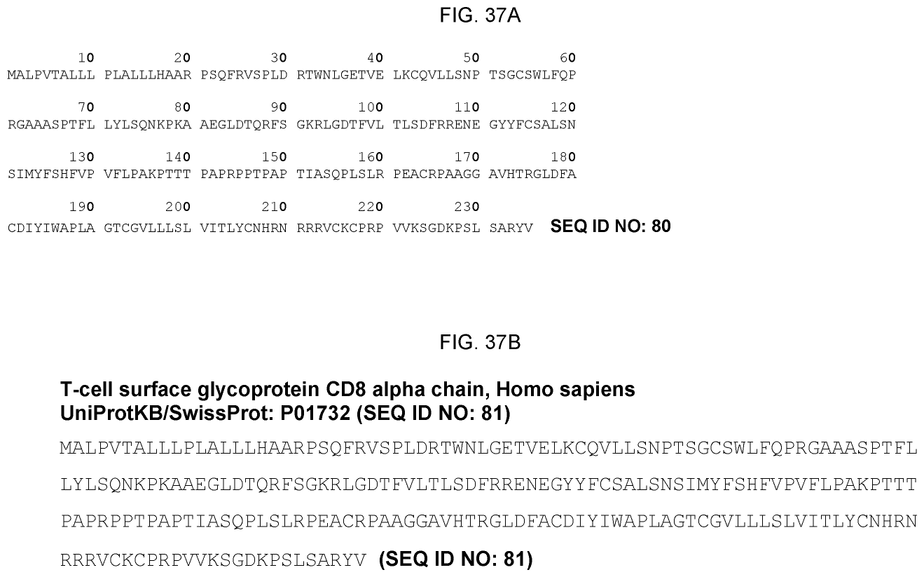

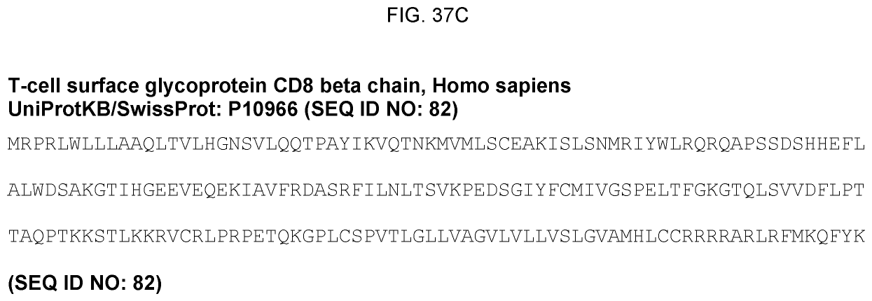

In some embodiments, a CD8 antigen-binding construct suitable for use in the present disclosure selectively binds to human CD8. In some embodiments, a CD8 antigen-binding construct suitable for used in the present disclosure selectively binds to a CD8 having any one of the amino acid sequences of shown in FIGS. 37A-37C.

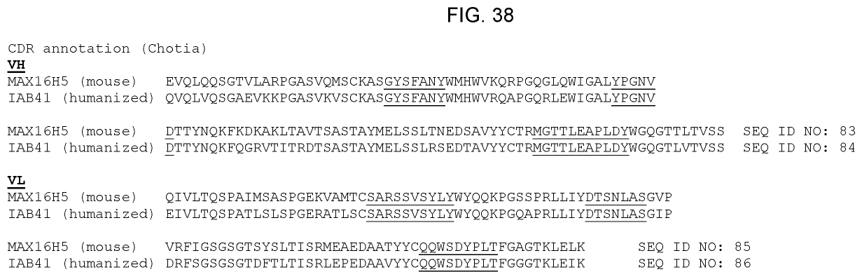

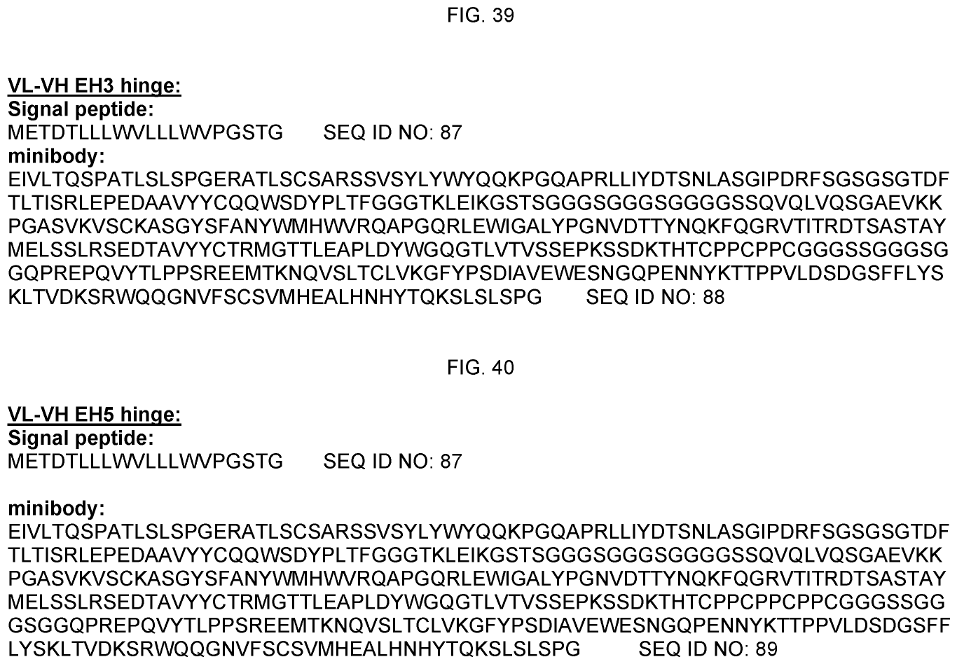

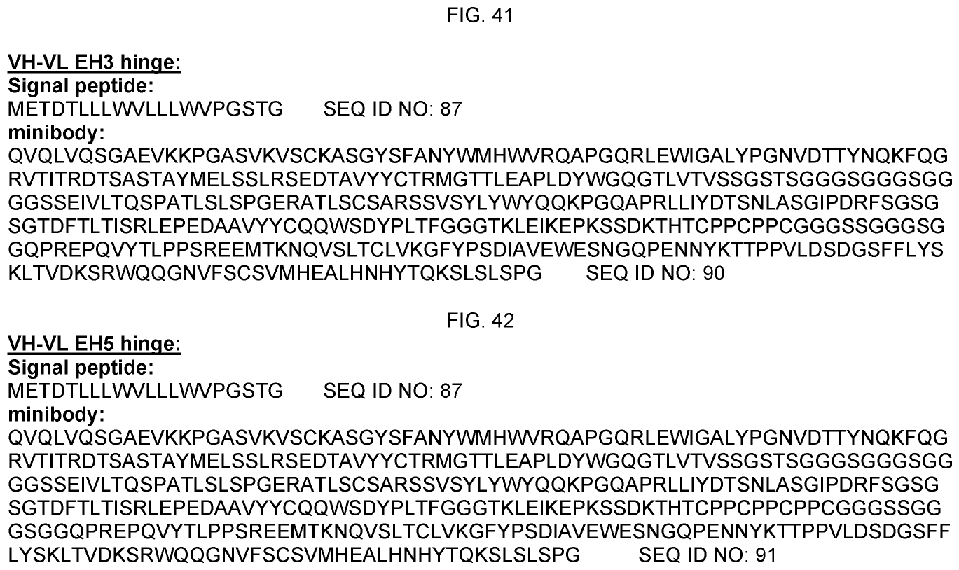

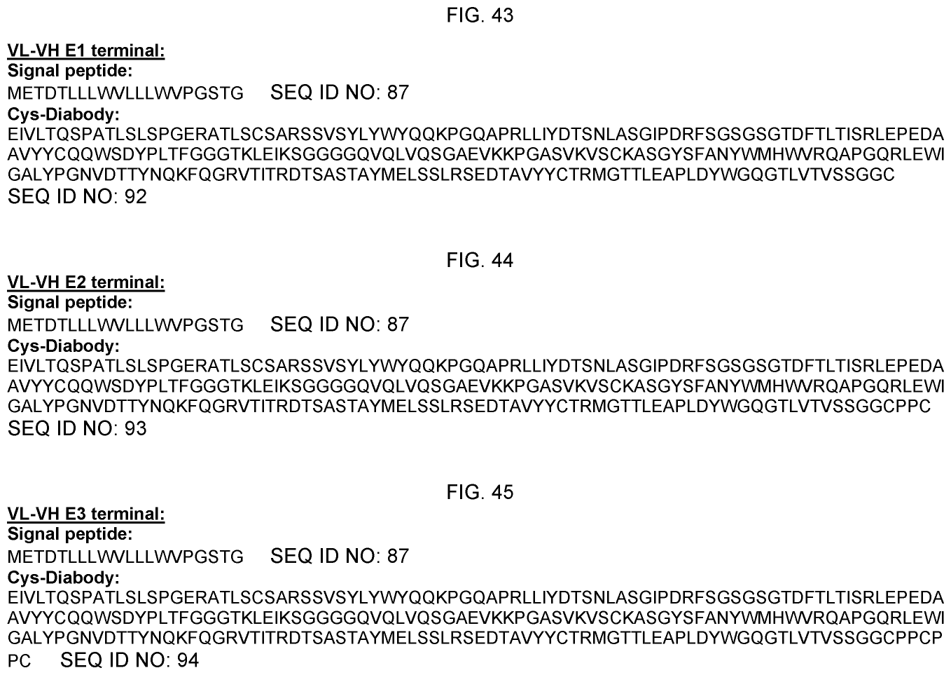

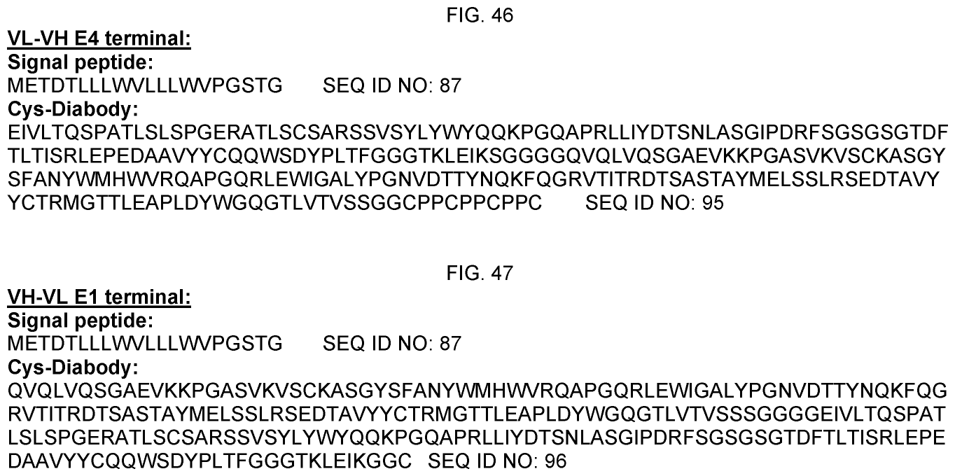

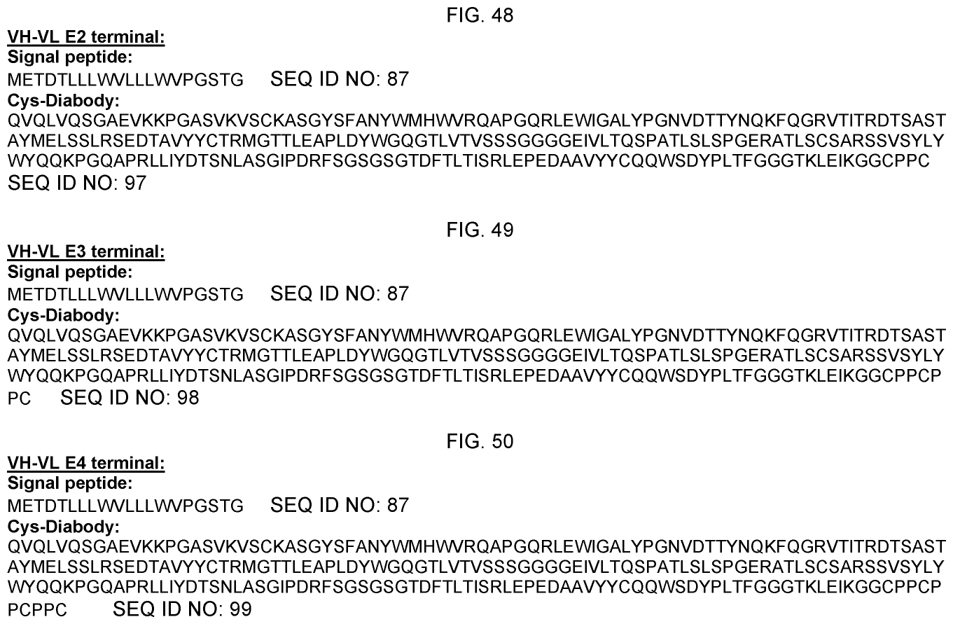

Suitable antigen-binding constructs that selectively bind CD4 are described, e.g., in International Application No. PCT/US2019/035550, filed Jun. 5, 2019, which is incorporated herein by reference. In some embodiments, a CD4 antigen-binding construct suitable for use in methods of the present disclosure include any of the amino acid sequences described in FIGS. 38-50. In some embodiments, a CD4 antigen-binding construct suitable for use in methods of the present disclosure include an amino acid sequence at least about 80%, 85%, 86%, 87%, 88%, 89%, 90%, 91%, 92%, 93, 94, 95%, 96%, 97%, 98%, or 99% identical to any of the amino acid sequences described in FIGS. 38-50. In certain embodiments, a CD4 antigen-binding construct suitable for use in methods of the present disclosure include any antigen-binding fragment or a portion thereof, such as any one of or all the CDRs, heavy chain variable region, light chain variable region, heavy and light chain variable regions, hinge region, etc., of the amino acid sequences depicted in FIGS. 38-50. In some embodiments, the CD4 antigen-binding construct comprises a V_{H} region having an amino acid sequence at least about 80%, 85%, 86%, 87%, 88%, 89%, 90%, 91%, 92%, 93, 94, 95%, 96%, 97%, 98%, 99%, or 100% identical to the V_{H} region of any one of SEQ ID NOs: 83, 84, 88-99. In some embodiments, the CD4 antigen-binding construct comprises a V_{L} region having an amino acid sequence at least about 80%, 85%, 86%, 87%, 88%, 89%, 90%, 91%, 92%, 93, 94, 95%, 96%, 97%, 98%, 99%, or 100% identical to the V_{L} region of any one of SEQ ID NOs: 85, 86, 88-99.

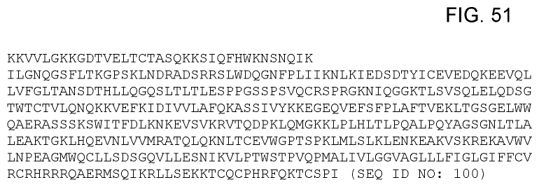

In some embodiments, a CD4 antigen-binding construct suitable for use in the present disclosure selectively binds to human CD4. In some embodiments, a CD4 antigen-binding construct suitable for used in the present disclosure selectively binds to a CD4 having an amino acid sequence of FIG. 51.

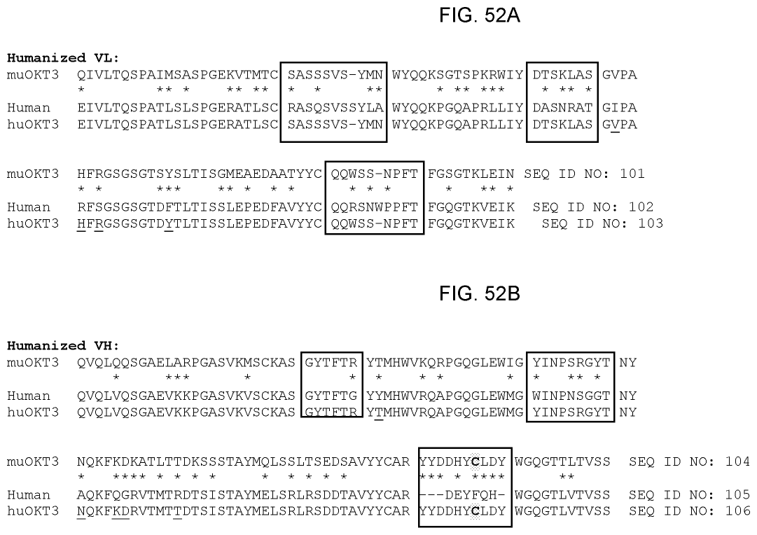

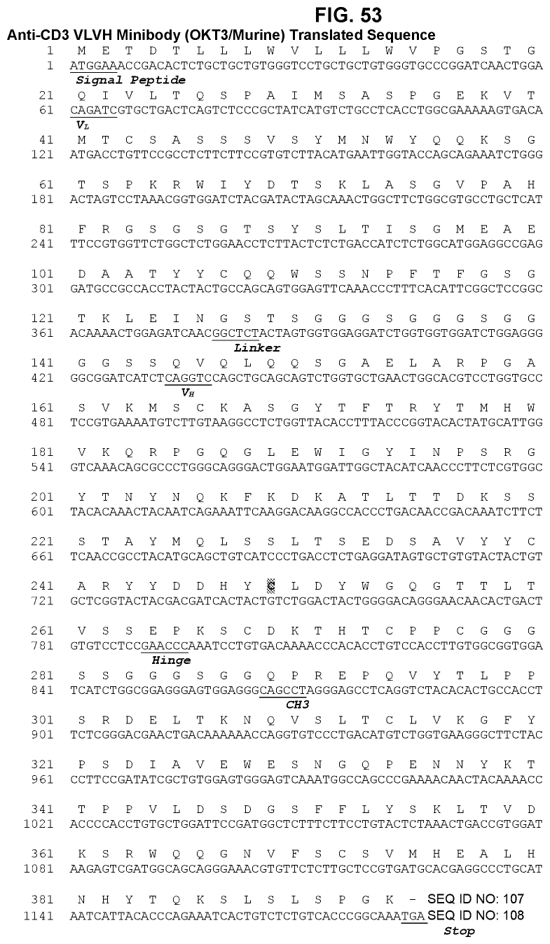

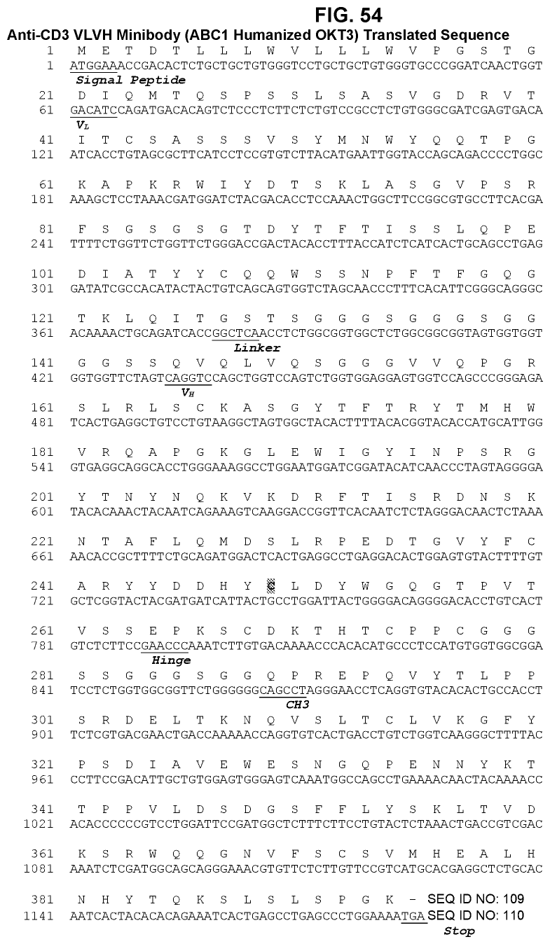

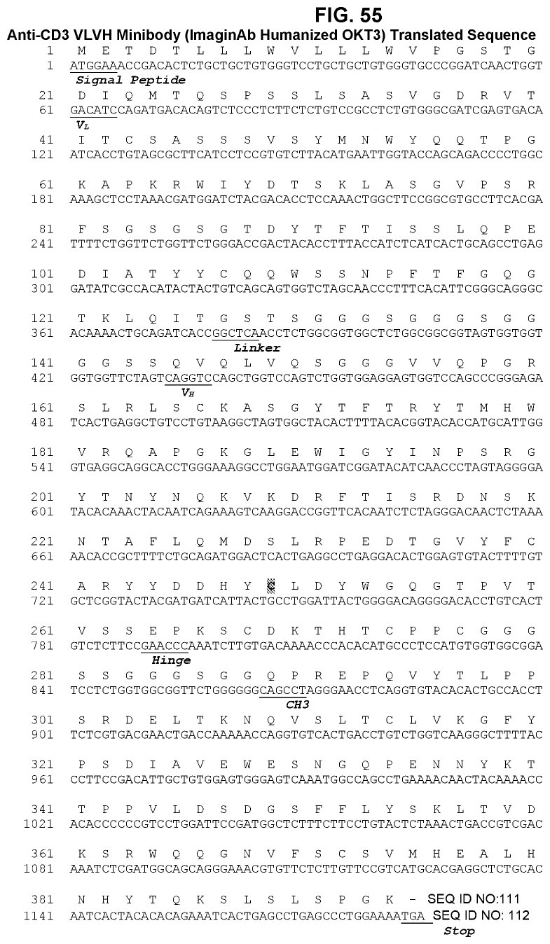

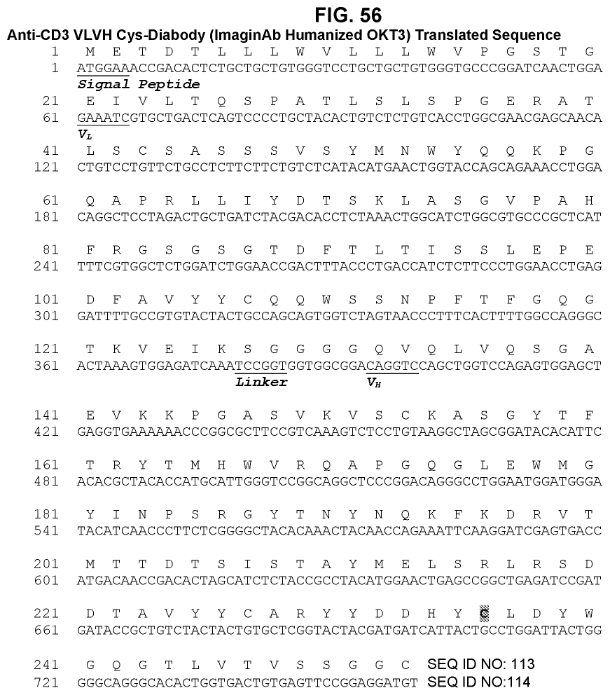

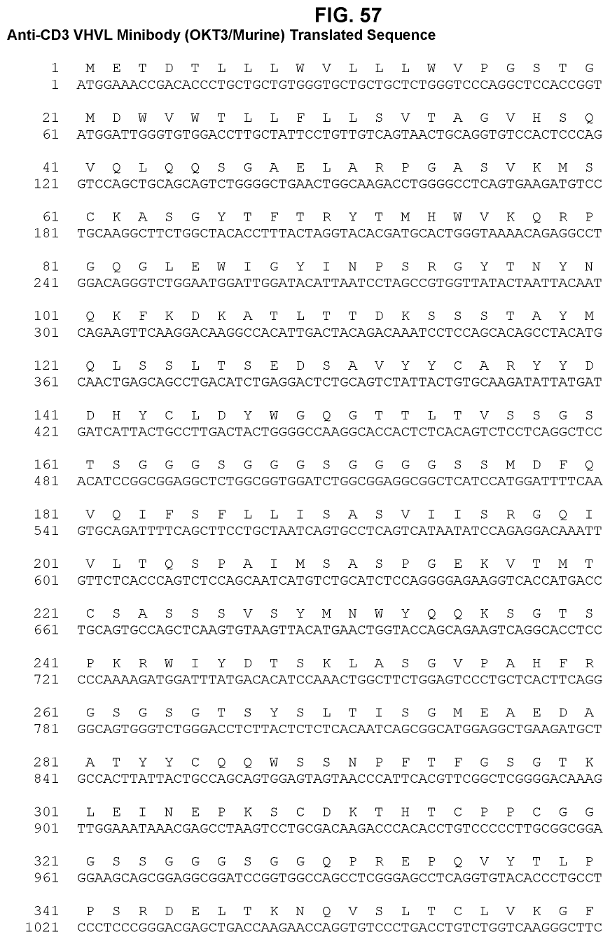

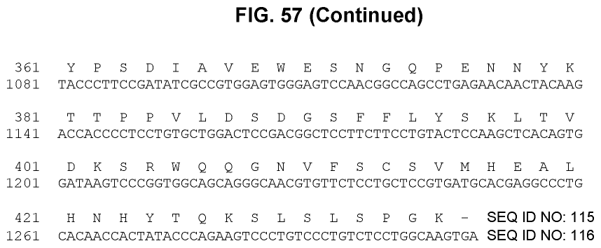

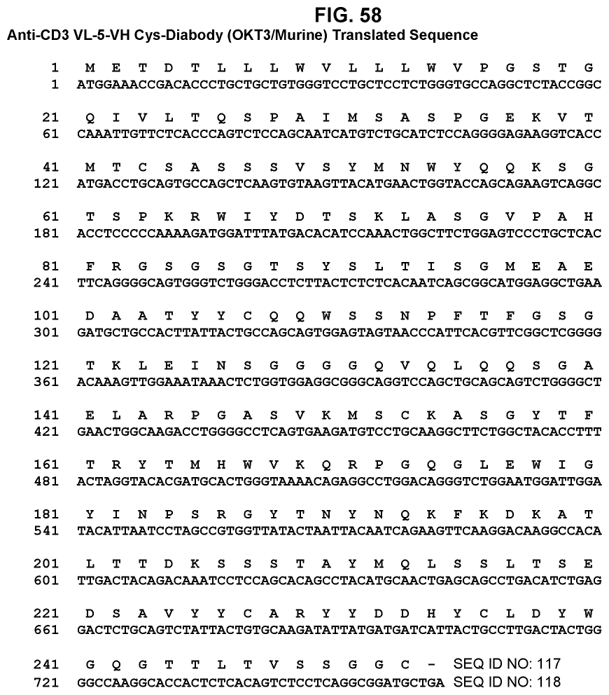

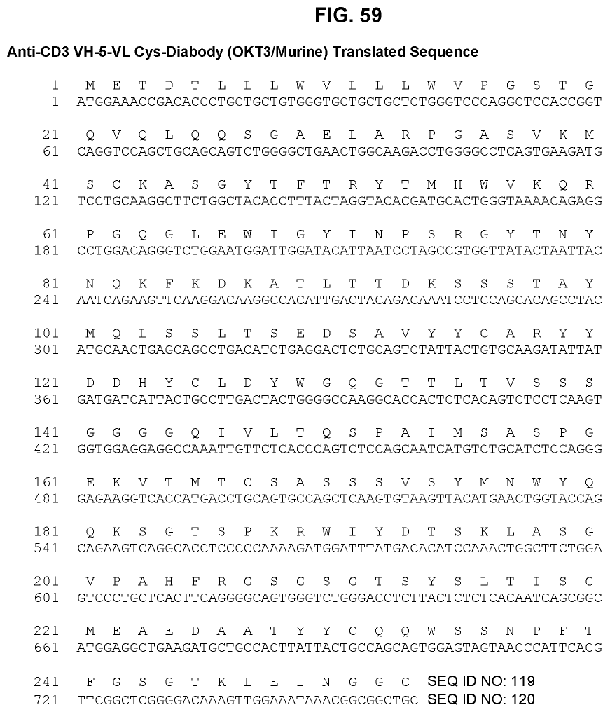

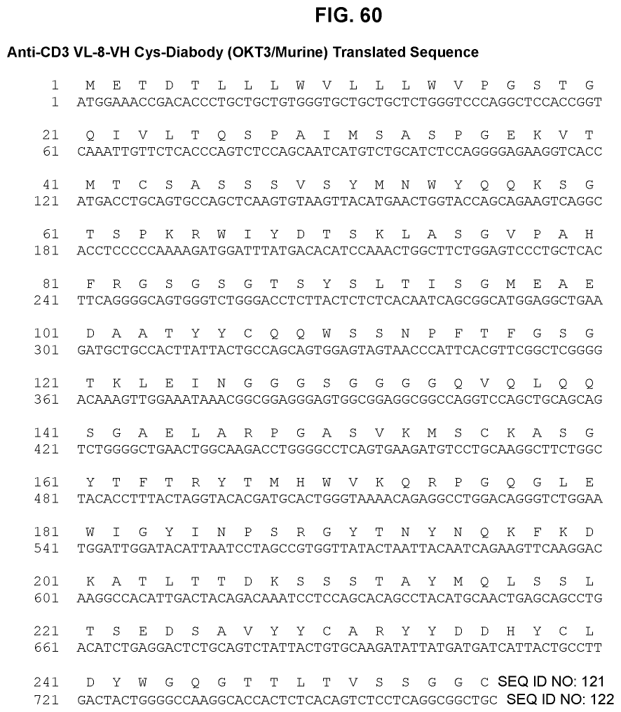

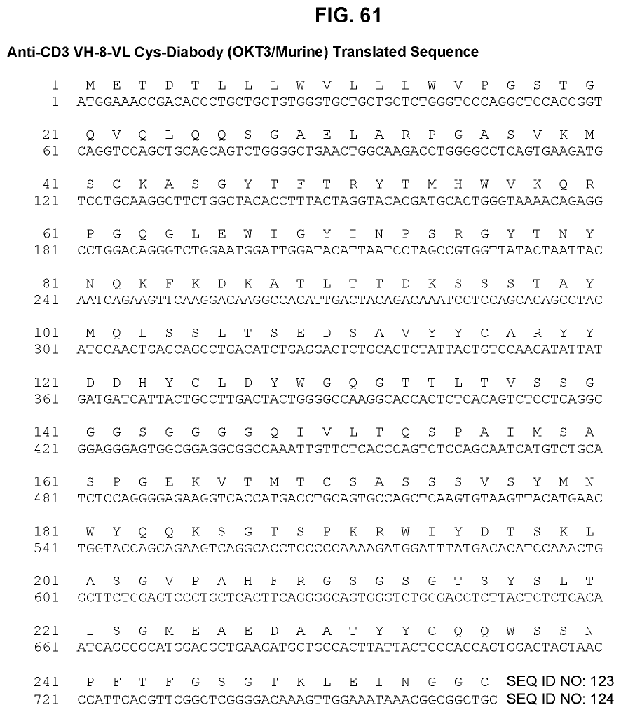

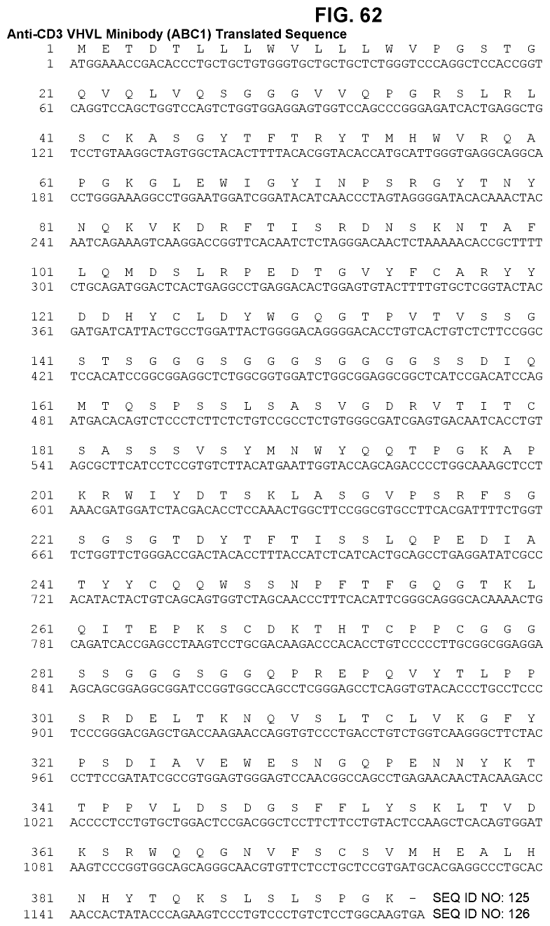

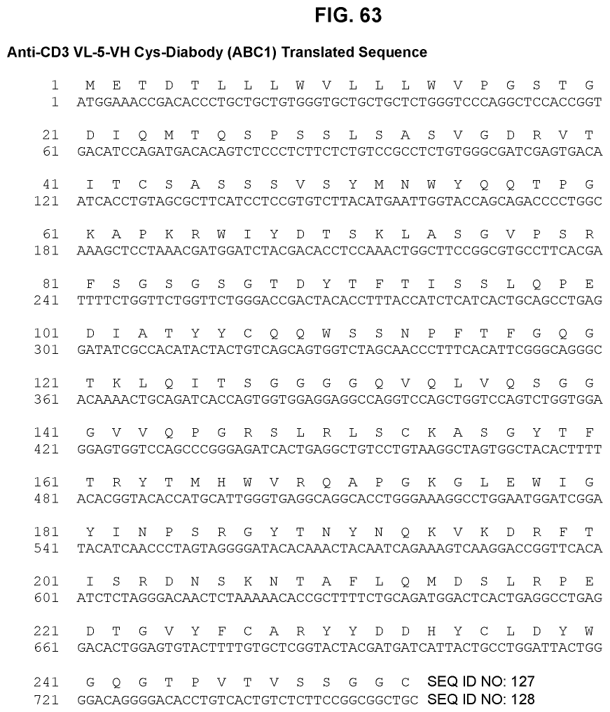

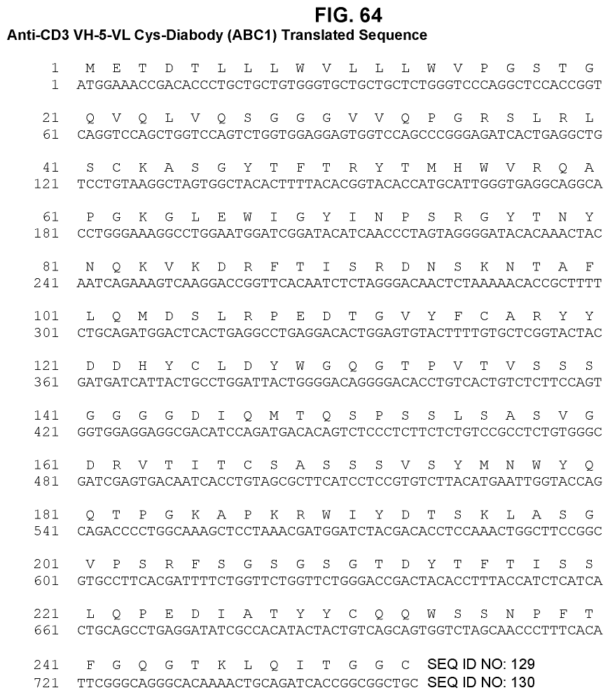

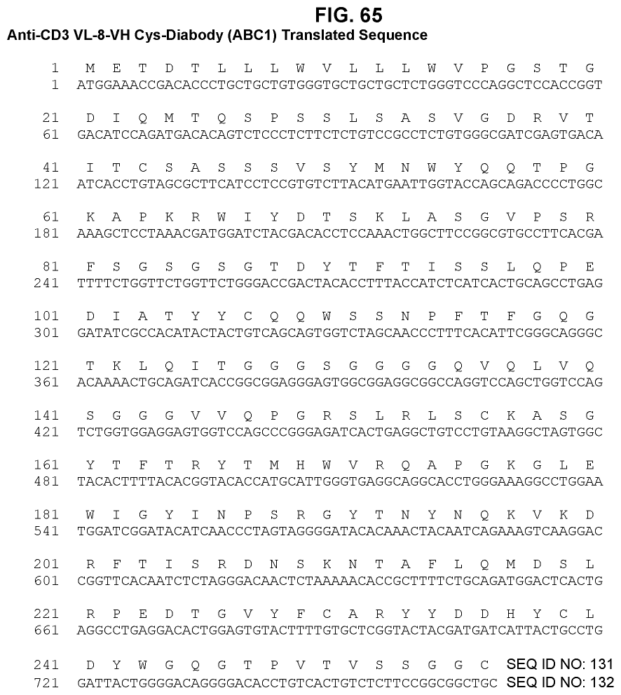

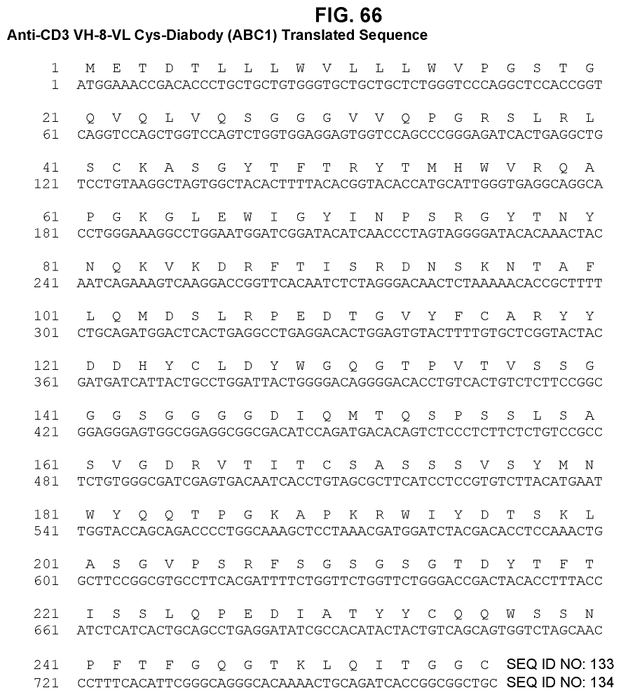

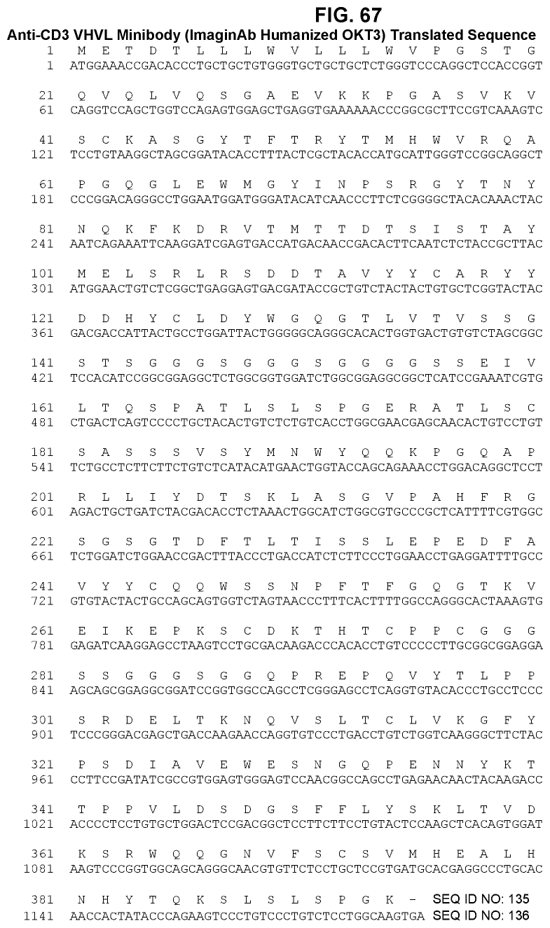

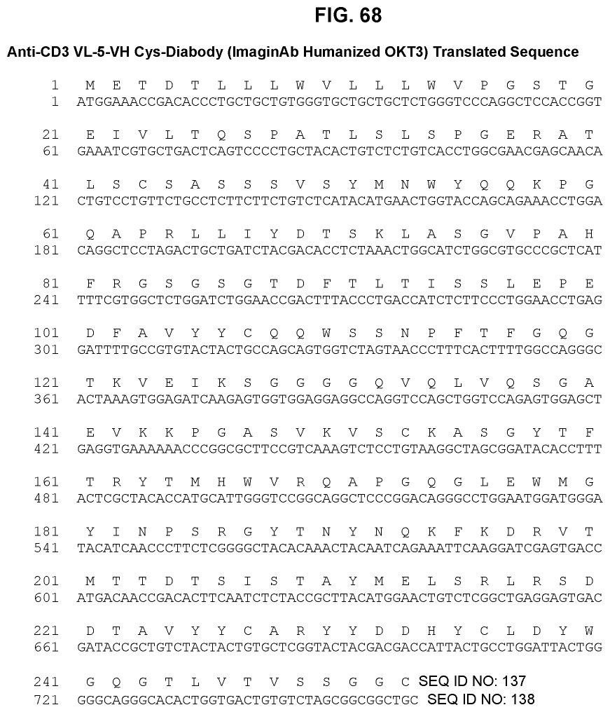

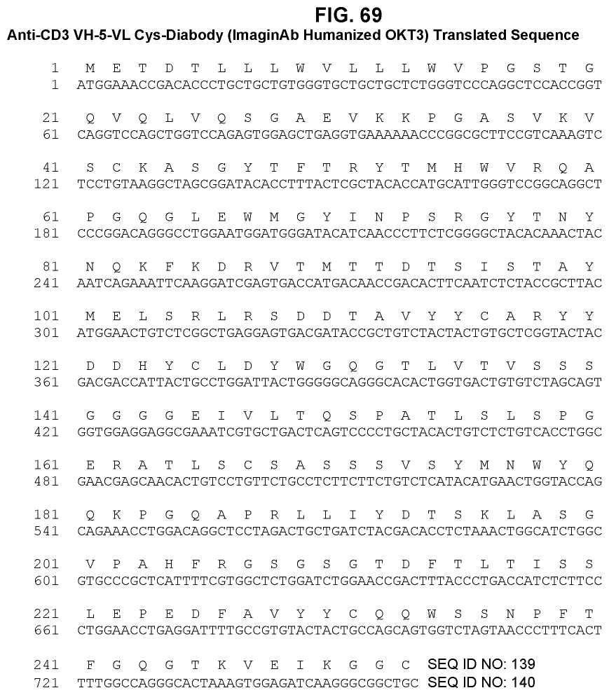

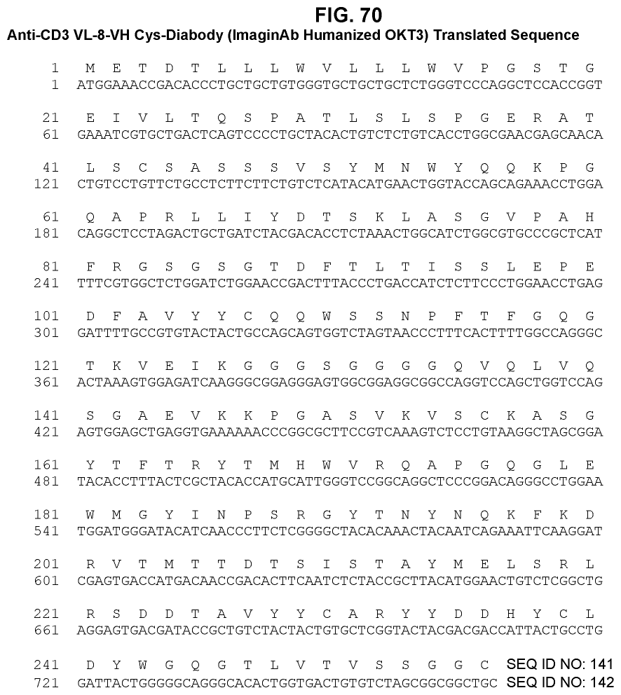

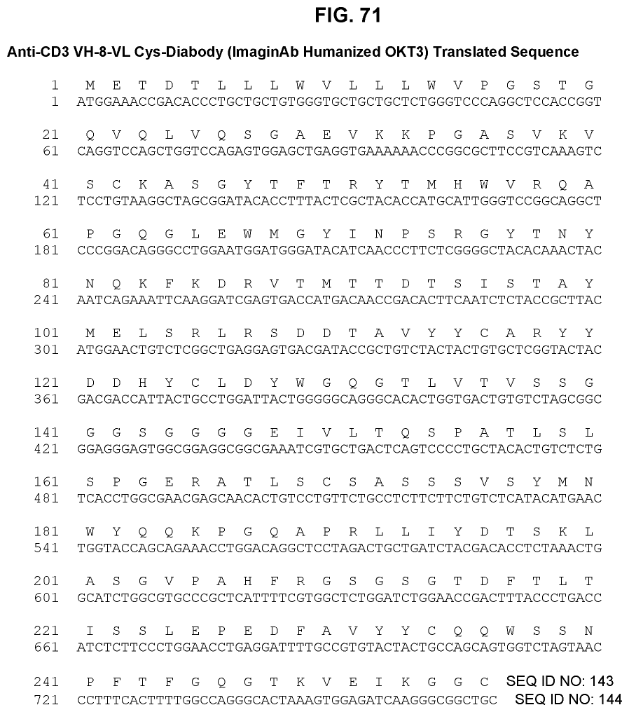

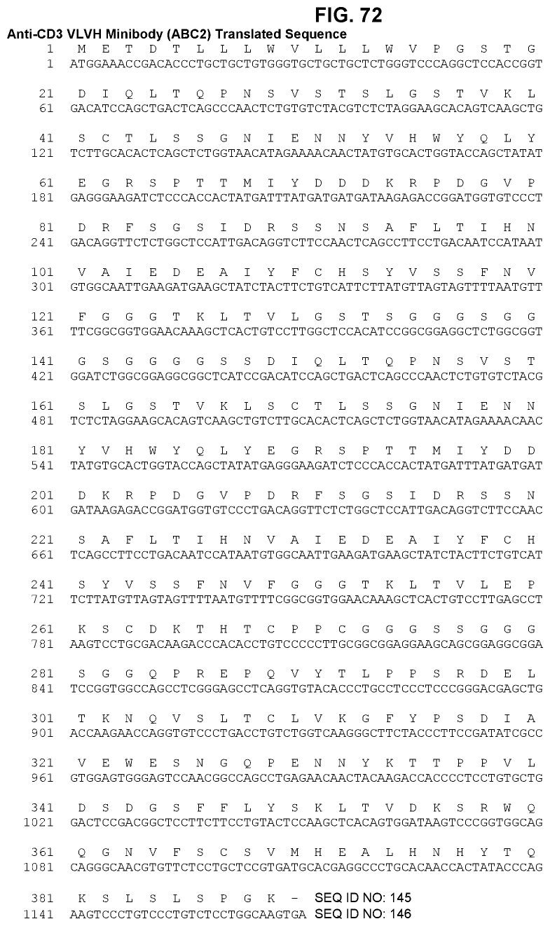

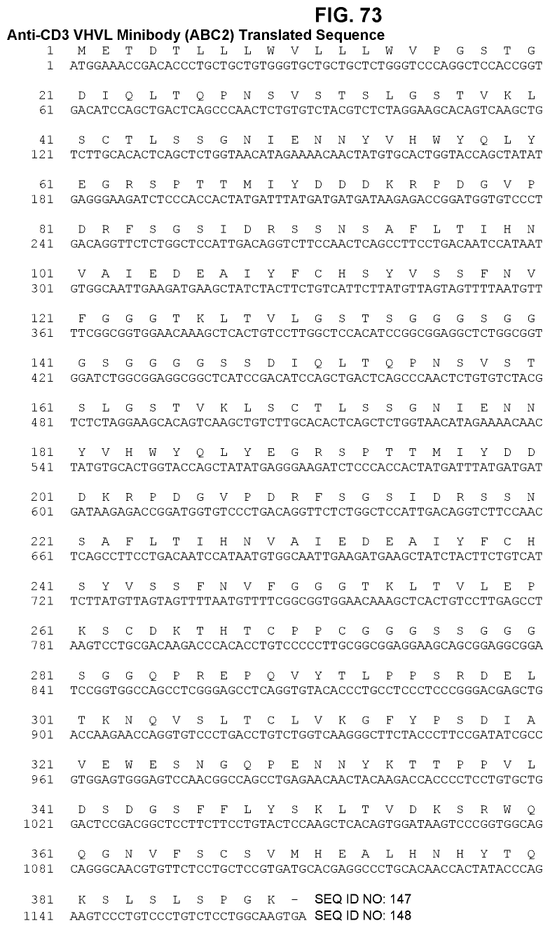

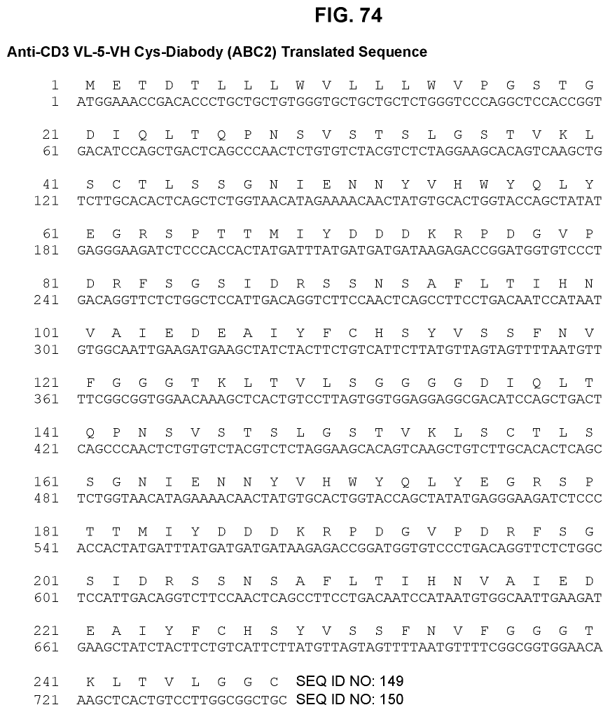

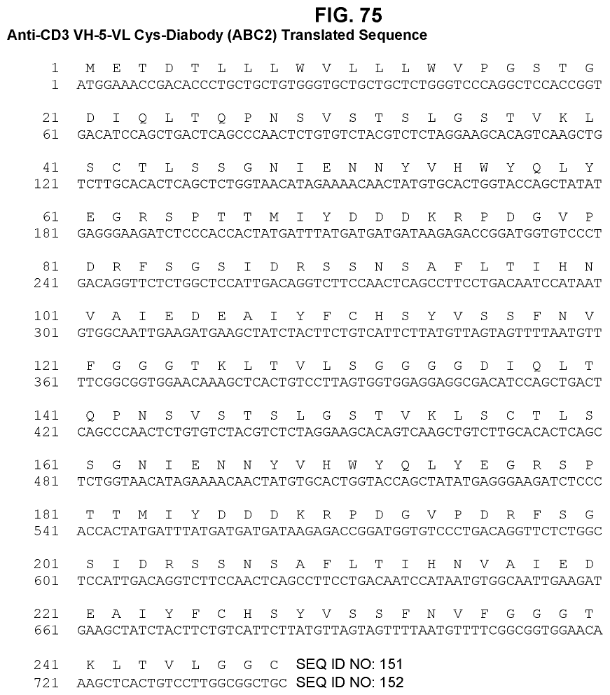

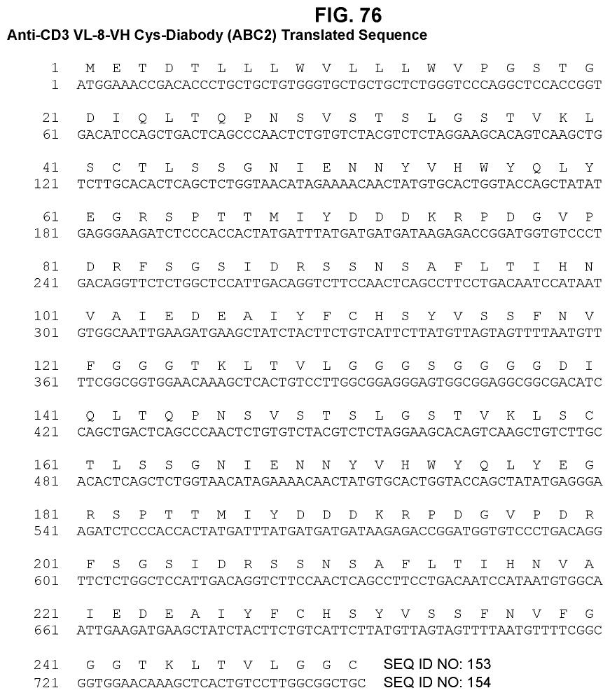

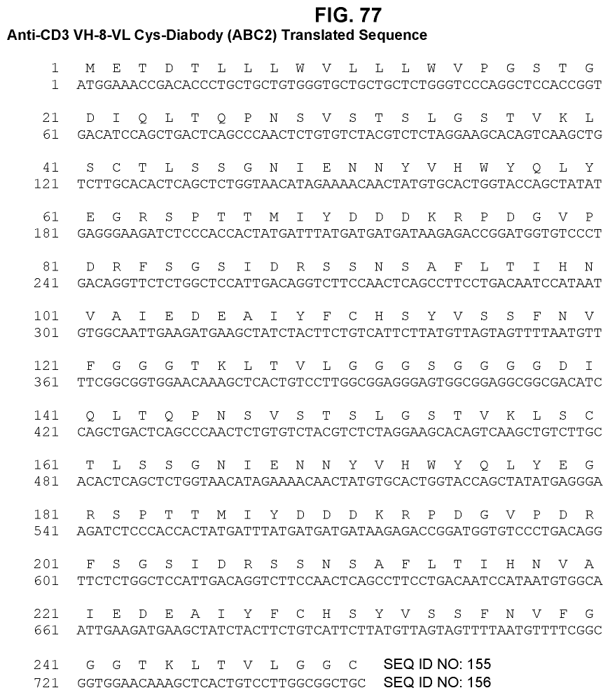

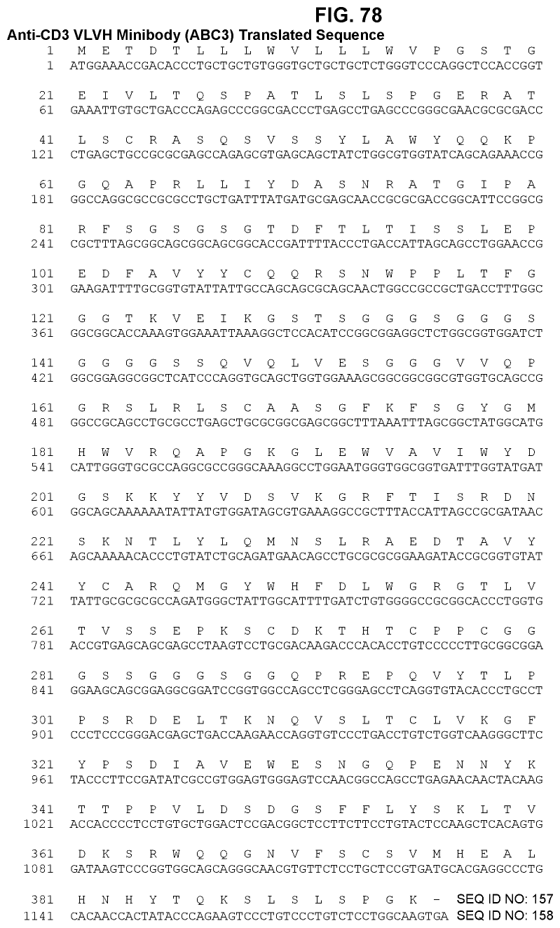

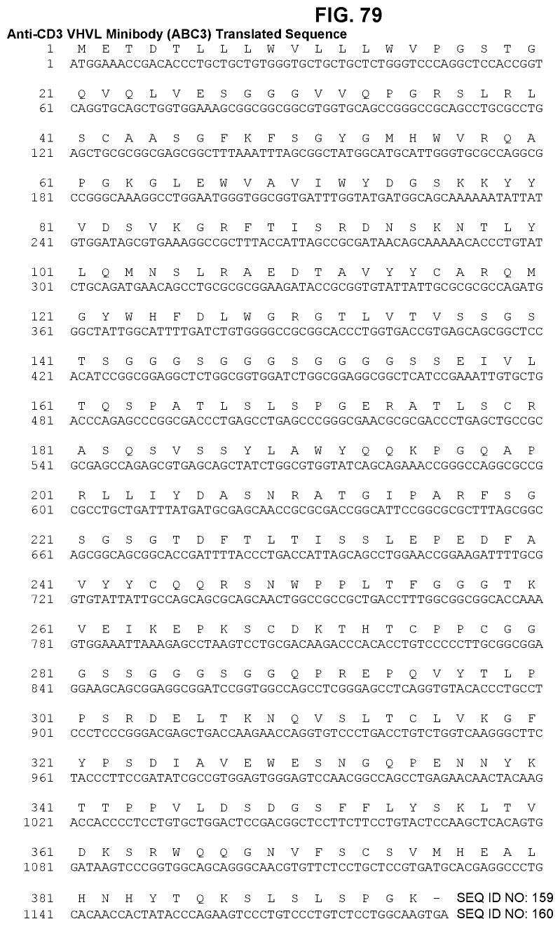

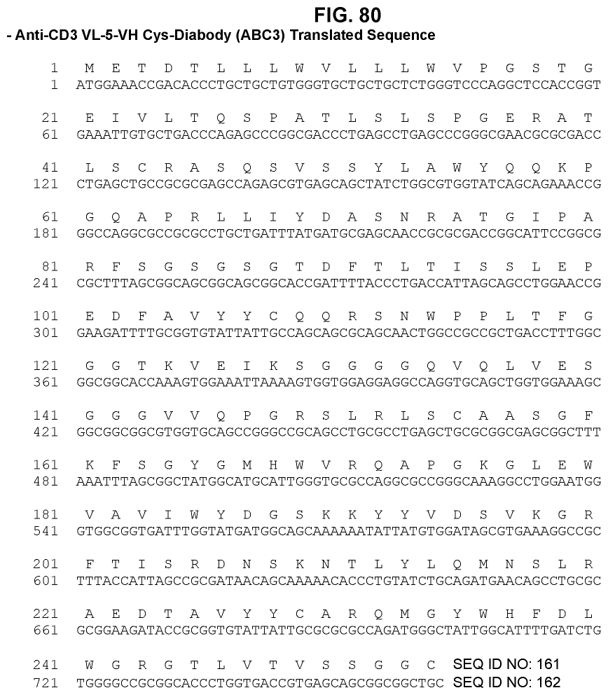

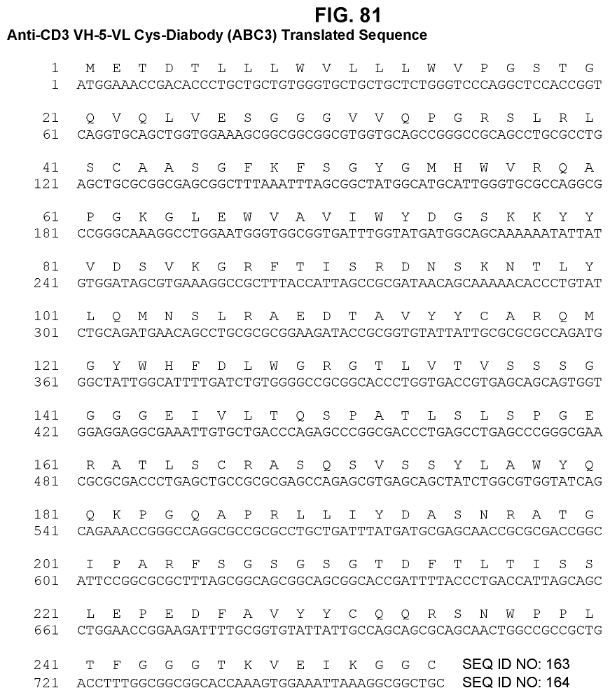

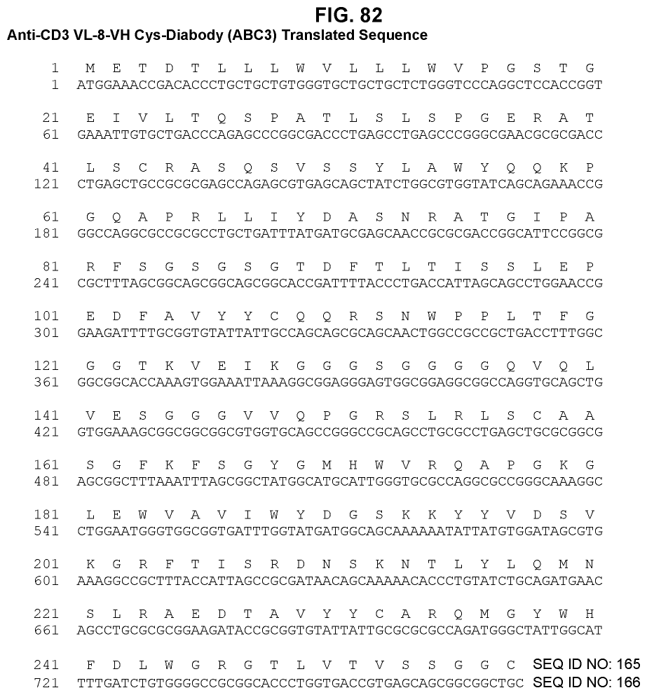

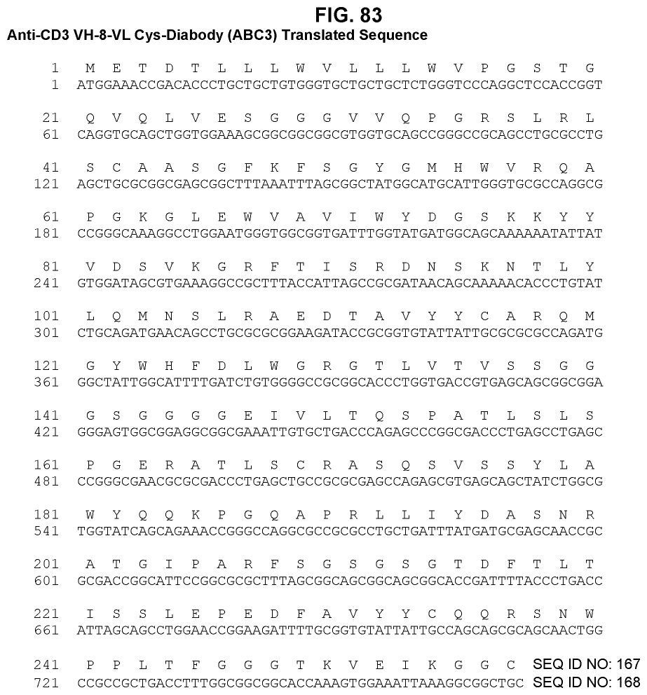

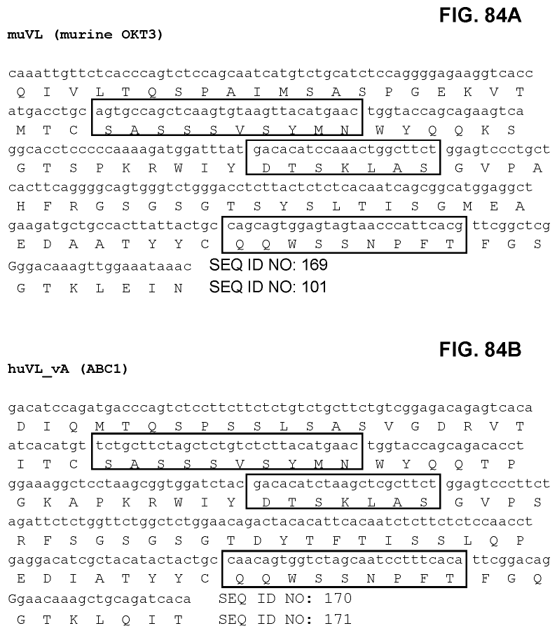

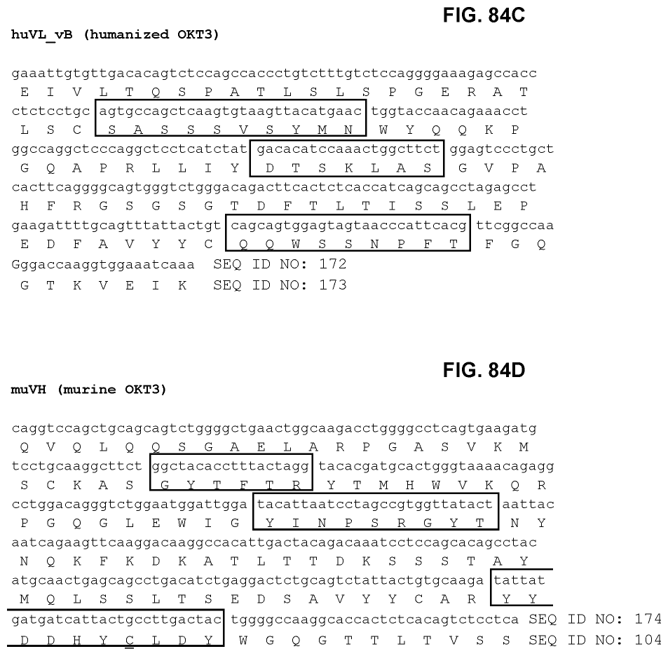

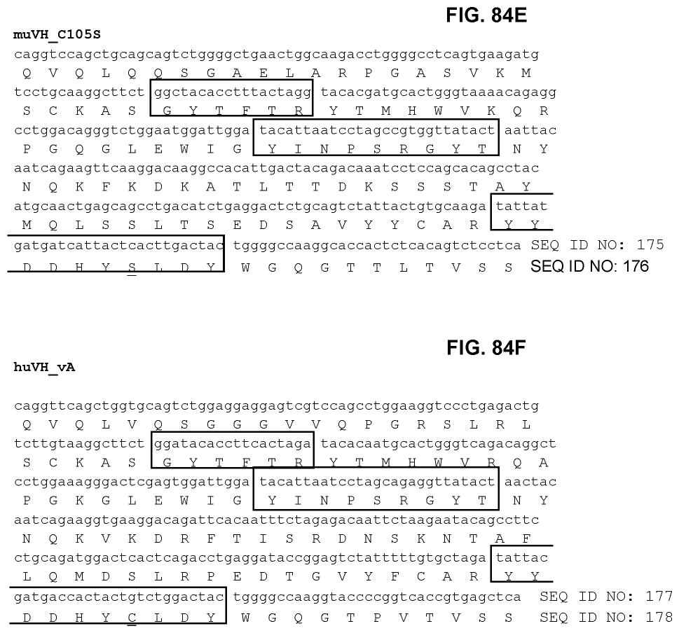

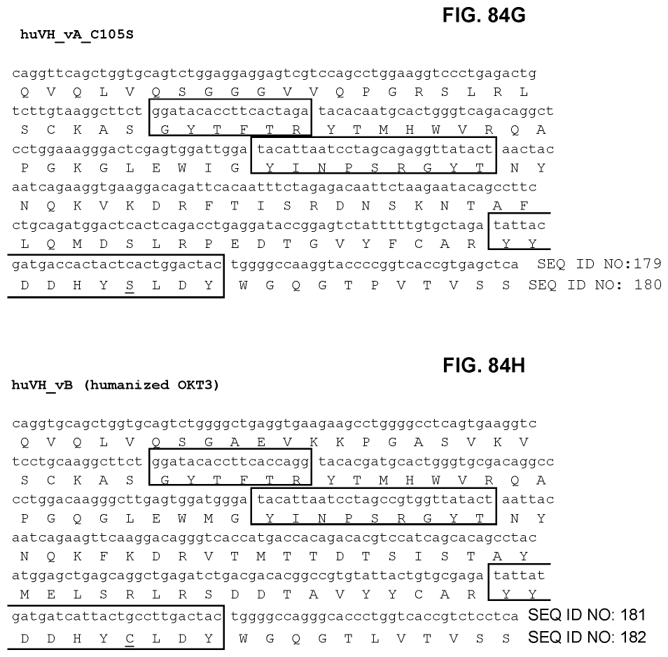

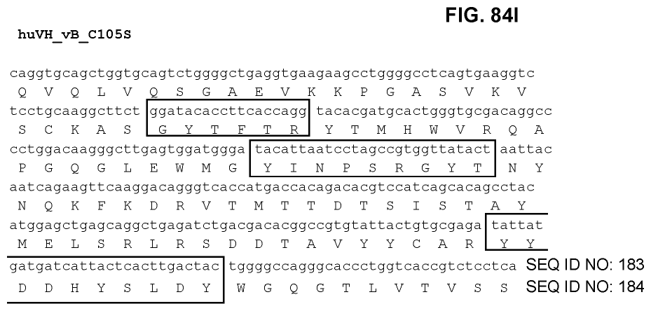

Suitable antigen-binding constructs that selectively bind CD3 are described, e.g., in PCT Publication No. WO 2013/188693, which is incorporated herein by reference. In some embodiments, a CD3 antigen-binding construct suitable for use in methods of the present disclosure include any of the amino acid sequences described in FIGS. 52A-84I. In some embodiments, a CD3 antigen-binding construct suitable for use in methods of the present disclosure include an amino acid sequence at least about 80%, 85%, 86%, 87%, 88%, 89%, 90%, 91%, 92%, 93, 94, 95%, 96%, 97%, 98%, or 99% identical to any of the amino acid sequences described in FIGS. 52A-84I. In certain embodiments, a CD3 antigen-binding construct suitable for use in methods of the present disclosure include any antigen-binding fragment or a portion thereof, such as any one of or all the CDRs, heavy chain variable region, light chain variable region, heavy and light chain variable regions, hinge region, etc., of the amino acid sequences depicted in FIGS. 52A-84I. In some embodiments, the CD3 antigen-binding construct comprises a V_{H} region having an amino acid sequence at least about 80%, 85%, 86%, 87%, 88%, 89%, 90%, 91%, 92%, 93, 94, 95%, 96%, 97%, 98%, 99%, or 100% identical to the V_{H} region of any one of SEQ ID NOs:104-106, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 176, 178, 180, 182, 184. In some embodiments, the CD3 antigen-binding construct comprises a V_{L} region having an amino acid sequence at least about 80%, 85%, 86%, 87%, 88%, 89%, 90%, 91%, 92%, 93, 94, 95%, 96%, 97%, 98%, 99%, or 100% identical to the V_{L} region of any one of SEQ ID NOs: 101-103, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 171, 173.

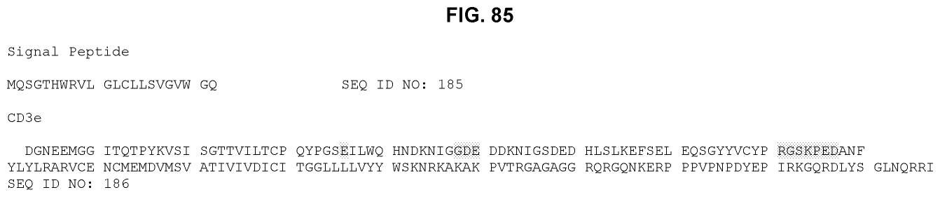

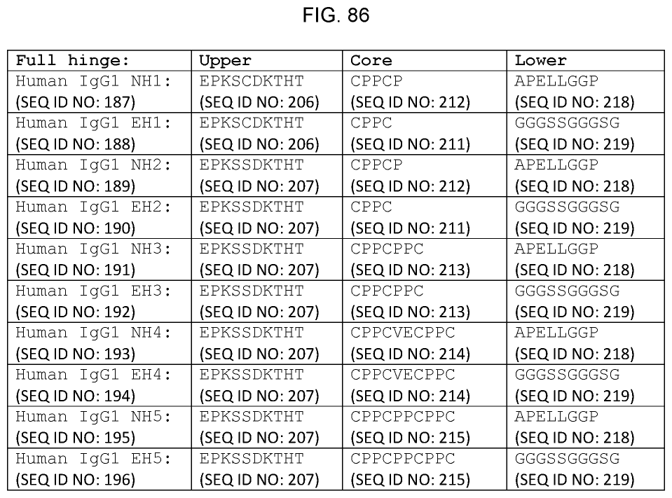

In some embodiments, a CD3 antigen-binding construct suitable for use in the present disclosure selectively binds to human CD3. In some embodiments, a CD3 antigen-binding construct suitable for used in the present disclosure selectively binds to a CD3 having an amino acid sequence of shown in FIG. 85 (SEQ ID NO: 186). It is noted that CD3 comprises chains (CD3γ, CD3δ, and CD3ε) and that antigen-binding constructs directed to any of them can be used for imaging according to the methods of the present disclosure. In general, a CD3 PET tracer used in the methods described herein may avoid non-specific activation of T cells to minimize the risk of undesirable activation in diagnostic imaging.

In some embodiments, an antigen-binding construct, e.g., an antibody or antigen-binding fragment thereof, minibody, etc., may include a hinge region. The hinge region may have one or more hinge sequences (e.g., one or more of any of the upper, core and lower hinge sequences, one or more of any combination of the upper and core hinge sequences, or one or more of any combination of the upper, core and lower hinge sequences) shown in FIG. 86. In some embodiments, the hinge sequence is located between a variable region and Ig domain.

In some embodiments, an antigen-binding construct is associated with (e.g., is conjugated to) a detectable marker. As used herein, a “detectable marker” includes an atom, molecule, or compound that is useful in diagnosing, detecting or visualizing a location and/or quantity of a target molecule, cell, tissue, organ and the like by non-invasive imaging techniques. Detectable markers that can be used in accordance with the embodiments herein include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions). In addition, some nanoparticles, for example quantum dots and metal nanoparticles (described below) can be suitable for use as a detection agent. In some embodiments, the detectable marker is IndoCyanine Green (ICG).

An antigen-binding construct may be associated or labeled with a radionuclide tracer by any suitable means. In some embodiments, an antigen binding construct is conjugated to the radionuclide tracer. Any suitable radionuclide tracer for non-invasive in vivo imaging may be used. Suitable radionuclide tracers include, without limitation, positron emitters, beta emitters and gamma emitters. Exemplary paramagnetic ion substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu. Methods for site-specific tagging of proteins and oligonucleotides with paramagnetic molecules are described at Su and Otting, J Biomol NMR. 2010 January; 46(1):101-12. doi: 10.1007/s10858-009-9331-1. In certain embodiments, preferred paramagnetic tags include nitroxide radicals and metal chelators. Exemplary radionuclide tracers that can be used in accordance with the embodiments herein include, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Te, ⁹⁴Tc, ⁹⁹mTc, ⁹⁹Mo, ¹⁰⁵Pd, ¹⁰⁵Rb, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lᵤ, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac.

In some embodiments, the radionuclide tracer can be reacted with a reagent having a long tail with one or more chelating groups attached to the long tail for binding these ions. The long tail can be a polymer such as a polylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. In some embodiments, the metal chelator is deferoxamine (“DF”). In some embodiments, the metal chelator is DOTA. In some embodiments, the metal chelator is PCTA. In some embodiments, the metal chelator is DTPA. In some embodiments, the metal chelator is NODAGA. In some embodiments, any of these (or others) can be used to carry modifications as isothiocyanate, NHS-esters, CHX-A″-DTPA, HBED, NOTA, DO2P, cyclam, TETA, TE2P, SBAD, NOTAM, DOTAM, PCTA, NO2A, or maleimide to allow conjugation to the protein.

The chelator can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

Exemplary X-ray contrast agents that can be used as detectable markers in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, Tantalum oxide, thallous chloride, or combinations thereof.

Suitable MRI contrast agents for MRI contrast enhancement may be gadolinium-based. Gadolinium (III) is generally regarded as safe when administered as a chelated compound, such as Gd-DTPA. Examples of MRI contrast enhancement using a gadolinium chelated antibodies include Shahbazi-Gahrouei, et al (2002) Australasian Physics & Engineering Sciences in Medicine volume 25:31. Two types of iron oxide MRI contrast agents include superparamagnetic iron oxide (SPIO) and ultrasmall superparamagnetic iron oxide (USPIO). SPIO and USPIO contrast agents have been used successfully for liver tumor enhancement.

Bioluminescent and fluorescent compounds or molecules and dyes that can be used as detectable markers in accordance with the embodiments of the disclosure include, but are not limited to, fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP), phycoerythrin, and the like), autoquenched fluorescent compounds that are activated by tumor-associated proteases, enzymes (e.g., luciferase, horseradish peroxidase, alkaline phosphatase, and the like), nanoparticles, biotin, digoxigenin or combination thereof.

Enzymes that can be used as detectable markers in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the antigen binding construct is conjugated to a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light or x-rays rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles which may be detected by fluorescence or scattering of an electromagnetic beam. Such nanoscale particles, when conjugated to an antigen binding construct, can be used as imaging agents for the in vivo detection of T-cells as described herein.

In some embodiments, the detectable marker will be suitable for Cerenkov (or Cherenkov) imaging. A Cerenkov luminescence agent, as used herein, is a radionuclide which induces Cerenkov radiation in a biological tissue which radiation may be detected by Cherenkov luminescence imaging (CLI). Cerenkov radiation can be observed from a range of positron-, β-, and α-emitting radionuclides using standard optical imaging devices. Visible light emissions from Cerenkov (or Cherenkov) luminescence has been observed in biological settings from a range of radionuclides including the positron emitters ¹⁸F, ^{M}Cu, ⁸⁹Zr, and ¹²⁴I; β-emitter ¹³¹I; and α-particle emitter ²²⁵Ac. Use of Cerenkov luminescence imaging (CLI) of tumors in vivo has been described, inter alia, at Ruggiero, A; Holland, J. P.; Lewis, J. S.; Grimm, J (2010). “Cerenkov luminescence imaging of medical isotopes”. Journal of Nuclear Medicine. 51 (7): 1123-1130.

Single photon emission computed tomography (SPECT) employs a detectable marker that emits gamma radiation. The radionuclides typically used as SPECT as detectable markers are iodine-123, technetium-99m, xenon-133, thallium-201, and fluorine-18. Others are also possible. Those skilled in the art are familiar with techniques for attaching a SPECT detectable marker to an antigen-binding construct that selectively binds a target. Such constructs can be used in the methods provided herein to determine the immune contexture of a tissue.

Some detectable markers may be “multimodal imaging agents” which allow detection of the marker by two different means e.g. by PET and separately by MRI. Diverse multimodal imaging agents are in development, see for example Truillet et al (2015) Contrast Media Mol. Imaging 2015, 10 309-319. Such agents may be used in the methods provided herein for labelling an antigen-binding construct as long as the resulting image can distinguish it from a second detectable marker attached to a second antigen-binding construct being used to establish the immune contexture being of the tissue.

Kits

Also provided herein are kits that include a first and second antigen-binding constructs, each labeled with a detectable marker, e.g., a radionuclide tracer, where the first antigen-binding construct binds selectively to a first target selected from CD3, CD4, IFN-gamma, or CD8, and wherein the second antigen-binding construct binds selectively to a second target selected from CD3, CD4, IFN-gamma, or CD8, where the first and second targets are different. In some embodiments, the kit may include a third antigen-binding construct labeled with a detectable marker, e.g., radionuclide tracer, where the third antigen-binding construct binds selectively to a third target selected from CD3, CD4, IFN-gamma, or CD8, where the third target is different from the first and second targets. The kits of the present disclosure find use in performing the methods of imaging, treating, diagnosing, recommending a treatment or providing a prognosis for a subject having a disease (e.g., a cancer), as disclosed herein. The detectable marker, e.g., radionuclide tracer, associated with each antigen-binding construct may be any suitable detectable marker, e.g., radionuclide tracer, as described herein for imaging the subject. In some embodiments, the kit includes any other suitable imaging agent for performing, without limitation, FDG-PET, CD3-PET, IFNγ-PET, Granzyme B-PET, PD-1-PET, PD-L1-PET, TGFβ-PET, as described herein. The components of the kit may be disposed in one or more containers. In some embodiments the kit includes instructions for administering the labeled antigen-binding constructs to a subject and imaging the subject using non-invasive imaging, e.g., PET or SPECT scan, as described herein.

Compositions

Also provided herein are compositions for use in the present methods. The composition can include a first and second antigen-binding constructs, each labeled with a detectable marker, e.g., a radionuclide tracer, where the first antigen-binding construct binds selectively to a first target selected from CD3, CD4, IFN-gamma, or CD8, and wherein the second antigen-binding construct binds selectively to a second target selected from CD3, CD4, IFN-gamma, or CD8, where the first and second targets are different. In some embodiments, the composition may include a third antigen-binding construct labeled with a detectable marker, e.g., a radionuclide tracer, where the third antigen-binding construct binds selectively to a third target selected from CD3, CD4, IFN-gamma, or CD8, where the third target is different from the first and second targets. The antigen-binding construct may be any suitable antigen-binding construct that selectively binds a desired target, as described herein. The detectable marker, e.g., radionuclide tracer, associated with each antigen-binding construct may be any suitable detectable marker, e.g., radionuclide tracer, for measuring the distribution and/or abundance of the target or cells expressing the target using non-invasive imaging, e.g., PET or SPECT, as described herein. In some embodiments, the combination of radionuclide tracers in the composition is selected based on the radioactive half-life of each radionuclide tracer such that after administering the composition, which may be a pharmaceutically acceptable composition, to the subject, the subject may be imaged at appropriate time points to measure a combined signal from all the radionuclide tracers at an earlier time point, and then one or more individual signals from a radionuclide tracer having a longer radioactive half-life at a later time point, as described herein. Alternatively, the combination of radionuclide tracers in the composition is selected based on the radio-emission intensity of each radionuclide tracer such that after administering the composition, (e.g. pharmaceutically acceptable composition), to the subject, the subject may be imaged at different windows to distinguish signal from the different targets, as described herein. The composition may include the antigen-binding constructs in any suitable amounts to deliver and target the detectable markers, e.g., radionuclide tracers to the corresponding targets and tissues for non-invasive imaging, e.g., PET or SPECT imaging, as described herein.

In some embodiments, the composition includes at least: 0.5-3.0±20% mCi of a radionuclide-labeled antigen binding construct, for each of the different antigen binding constructs, 20 mM Histidine, 5% sucrose, 51-62 mM Sodium Chloride, 141-194 mM Arginine, and 2-20 mM Glutamic acid. In some embodiments, the amount of radiation is between 0.5 and 3.6 mCi, for example 0.5, 0.6, 0.7, 0.8, 0.9, 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8, 2.9, 3.0, 3.1, 3.2, 3.3, 3.4, 3.5 and 3.6 mCi, including any amount defined by any two of the preceding values, for each of the different radionuclide tracers. In some embodiments, the composition includes about 1 mCi of a radionuclide tracer, e.g., ⁸⁹Zr, associated with at least one of the antigen-binding constructs. In some embodiments, the composition includes an antigen-binding construct labeled with a radionuclide tracer of about 0.1, 0.2. 0.5, 1, 2.5, 5, 7.5, 10, 12.5, 15, 17.5, or 20 mg, or a value within a range defined by any two of the aforementioned values, of the antigen-binding construct, for each of the different antigen binding constructs. In some embodiments, 10, 15, 20, 25, or 30 mM of histidine can be present, including any amount defined by any two of the preceding values, can be employed. In some embodiments, 2, 3, 4, 5, 6, 7, 8, 9 or 10% sucrose or an alternative to sucrose, including any amount defined by any two of the preceding values, can be employed. In some embodiments, the amount of sodium chloride can be 40, 45, 50, 55, 60, 65, or 70 mM, including any amount defined by any two of the preceding values, can be employed. In some embodiments, the amount of arginine can be 120, 125, 130, 135, 140, 145, 150, 155, or 160 mM, including any amount defined by any two of the preceding values, can be employed. In some embodiments, the amount of glutamic acid can be 1, 2, 5, 10, 20, 25, or 30 mM, including any amount defined by any two of the preceding values, can be employed.

Some embodiments of the present disclosure are provided by the following numbered options.

Option 1. A method of treating a subject, comprising:
administering to a subject having a disease a first antigen-binding construct comprising a first radionuclide tracer, wherein the antigen-binding construct selectively binds a first target selected from CD3, CD4, and CD8;
imaging the subject by positron emission tomography (PET) or single photon emission computed tomography (SPECT) to acquire a distribution of cells expressing the first target in one or more tissues of the subject;
administering to the subject a second antigen-binding construct comprising a second radionuclide tracer, wherein the antigen-binding construct selectively binds a second target selected from CD3, CD4, and CD8, and wherein the first and second targets are different;
imaging the subject by PET or SPECT to acquire a distribution of cells expressing the second target in the one or more tissues;
determining an immune contexture of the one or more tissues based on the distribution of cells expressing the first target and the distribution of cells expressing the second target in the one or more locations; and
administering a treatment to the subject based on the immune contexture.

Option 2. The method of option 1, further comprising:
administering to the subject a third antigen-binding construct comprising a third radionuclide tracer, wherein the antigen-binding construct selectively binds a third target selected from CD3, CD4, and CD8, wherein the third target is different from the first and second targets; and
imaging the subject by PET or SPECT to acquire a distribution of cells expressing the third target in the one or more locations.

Option 3. The method of option 1 or 2, further comprising generating an image based on the distributions of cells expressing the targets, wherein the image provides the immune contexture of the one or more tissues.

Option 4. The method of any one of options 1 to 3, further comprising determining a relative abundance among cells expressing any one of the targets compared to cells expressing another one of the targets in each of the one or more tissues.

Option 5. The method of any one of options 1 to 4, wherein the immune contexture comprises an abundance of, or relative abundance among, one or more of cytotoxic T cells (CD8⁺), helper T cells (CD4⁺), CD4±/CD8⁺ double positive T cells, memory T cells and regulatory T cells (Tregs).

Option 6. The method of any one of options 1 to 5, wherein the immune contexture comprises one or more of:
a ratio of CD4⁺ cells to CD8⁺ cells;
a ratio of CD3⁺ cells to CD8⁺ cells;
a ratio of CD3⁺ cells to CD4⁺ cells;
an abundance of CD8⁺ cells and an abundance of CD3⁺ cells; or
an abundance of CD4⁺ cells and an abundance of CD3⁺ cells; or
an abundance of CD8⁺ cells and an abundance of CD4⁺ cells.

Option 7. The method of any one of options 1 to 6, wherein the one or more tissues comprises a tumor.

Option 8. The method of any one of options 1 to 7, wherein the one or more tissues comprises one or more of a lung, liver, colon, intestine, stomach, brain, kidney, spleen, pancreas, esophagus, lymph node, bone, bone marrow, prostate, cervix, ovary, breast, urethra, bladder, skin, neck, articulated joint, or portions thereof.

Option 9. The method of any one of options 1 to 8, wherein the disease comprises a cancer.

Option 10. The method of any one of options 1 to 9, wherein the disease comprises a cancer of a lung, liver, colon, intestine, stomach, brain, kidney, spleen, pancreas, esophagus, lymph node, bone, bone marrow, prostate, cervix, ovary, breast, urethra, bladder, skin or neck.

Option 11. The method of option 10, wherein the subject has melanoma, non small-cell lung carcinoma (NSCLC), or renal cell cancer (RCC).

Option 12. The method of any one of options 1 to 11, further comprising identifying the one or more tissues as comprising cancerous tissue.

Option 13. The method of option 12, wherein the one or more tissues are identified as comprising cancerous tissue using computed tomography (CT) scan, X-ray, FDG-PET, or magnetic resonance imaging (MRI).

Option 14. The method of any one of options 1 to 13, the treatment comprises one or more of immunotherapy, chemotherapy, hormone therapy, radiation therapy, surgery, vaccine therapy, oncolytic virus therapy, or cellular therapy.

Option 15. The method of any one of options 1 to 14, wherein the subject has received an earlier treatment for the disease before administering to the subject the first antigen-binding construct.

Option 16. The method of option 15, wherein the earlier treatment comprises one or more of immunotherapy, chemotherapy, hormone therapy, radiation therapy, surgery, or cellular therapy.

Option 17. The method of option 15 or 16, wherein the treatment and the earlier treatment are different.

Option 18. A method of treating a subject, comprising:
administering to a subject having a cancer a first antigen-binding construct comprising a first radionuclide tracer, wherein the antigen-binding construct selectively binds a first target selected from CD3, CD4, and CD8;
imaging the subject by positron emission tomography (PET) or single photon emission computed tomography (SPECT) to acquire a distribution of cells expressing the first target in a tumor in the subject;
administering to the subject a second antigen-binding construct comprising a second radionuclide tracer, wherein the antigen-binding construct selectively binds a second target selected from CD3, CD4, and CD8, wherein the first and second targets are different;
imaging the subject by PET or SPECT to acquire a distribution of cells expressing the second target in the tumor;
estimating a density of CD3⁺ cells, CD4⁺ cells and/or CD8⁺ cells in a core and/or invasive margin of the tumor based on the distributions of cells expressing the targets; and
administering to the subject a treatment for the cancer based on a determination that the core and/or invasive margin of the tumor is depleted for one or more of CD3⁺, CD4⁺, and CD8⁺ cells, and/or enriched for one or more of CD3⁺, CD4⁺ or CD8⁺ cells.

Option 19. The method of option 18, further comprising:
administering to the subject a third antigen-binding construct comprising a third radionuclide tracer, wherein the antigen-binding construct selectively binds a third target selected from CD3, CD4, and CD8, wherein the third target is different from the first and second targets; and
imaging the subject by PET or SPECT to acquire a distribution of cells expressing the third target in the tumor.

Option 20. The method of option 18 or 19, wherein administration of the treatment for the cancer is based on a determination that the core and/or invasive margin of the tumor is:
depleted for CD3⁺ cells and CD8⁺ cells;
depleted for CD3⁺ cells and CD4⁺ cells;
depleted for CD4⁺ cells and enriched for CD8⁺ cells;
depleted for CD8⁺ cells and enriched for CD4⁺ cells; or
depleted for CD8⁺ cells and CD4⁺ cells.

Option 21. The method of any one of options 18 to 19, wherein the core and/or invasive margin of the tumor is determined to be depleted:
for CD8⁺ cells when the estimated density of CD8⁺ cells is 118 cells/mm² or less;
for CD4⁺ cells when the estimated density of CD4⁺ cells is 118 cells/mm² or less; or
for CD3⁺ cells when the estimated density of CD3⁺ cells is 300 cells/mm² or less.

Option 22. The method of any one of options 18 to 21, wherein the core and/or invasive margin of the tumor is determined to be enriched:
for CD4⁺ cells when the estimated density of CD4⁺ cells is 118 cells/mm² or more;
for CD8⁺ cells when the estimated density of CD8⁺ cells is 118 cells/mm² or more; or
for CD3⁺ cells when the estimated density of CD3⁺ cells is 300 cells/mm² or more.

Option 23. The method of any one of options 18 to 22, wherein estimating the density of CD3⁺ cells, CD4⁺ cells and/or CD8⁺ cells comprises:
generating an image based on the distributions of cells expressing the targets; and
estimating the density of CD3⁺ cells, CD4⁺ cells and/or CD8⁺ cells in a core and/or invasive margin of the tumor based on the image.

Option 24. A method of treating a subject, comprising:
administering to a subject having a cancer a first antigen-binding construct comprising a first radionuclide tracer, wherein the antigen-binding construct selectively binds a first target selected from CD3, CD4, and CD8;
imaging the subject by positron emission tomography (PET) or single photon emission computed tomography (SPECT) to acquire a distribution of cells expressing the first target in a tumor in the subject;
administering to the subject a second antigen-binding construct comprising a second radionuclide tracer, wherein the antigen-binding construct selectively binds a second target selected from CD3, CD4, and CD8, and wherein the first and second targets are different;
imaging the subject by PET or SPECT to acquire a distribution of cells expressing the second target in the tumor;
estimating a ratio of:
   CD4⁺ cells to CD8⁺ cells; and/or
   CD8⁺ cells to CD4⁺ cells; and/or
   CD3⁺ cells to CD4⁺ cells; and/or
   CD3⁺ cells to CD8⁺ cells,
   in the tumor based on the acquired distributions; and
administering to the subject treatment for the cancer based on a determination that the ratio of:
   CD4⁺ cells to CD8⁺ cells is below a threshold ratio; and/or
   CD8⁺ cells to CD4⁺ cells is below a threshold ratio; and/or
   CD4⁺ cells to CD3⁺ cells is below a threshold ratio; and/or
   CD8⁺ cells to CD3⁺ cells is below a threshold ratio,
   in the tumor.

Option 25. The method of option 24, further comprising:
administering to the subject a third antigen-binding construct comprising a third radionuclide tracer, wherein the antigen-binding construct selectively binds a third target selected from CD3, CD4, and CD8, wherein the third target is different from the first and second targets; and
imaging the subject by PET or SPECT to acquire a distribution of cells expressing the third target in the tumor.

Option 26. The method of option 24 or 25, wherein the treatment is administered based on a determination that the ratio of CD4⁺ cells to CD8⁺ cells is at or below a threshold ratio.

Option 27. The method of option 24 or 25, wherein the treatment is administered based on a determination that the ratio of CD8⁺ cells to CD4⁺ cells is at or below a threshold ratio.

Option 28. The method of option 24 or 25, wherein the treatment is administered based on a determination that the ratio of CD8⁺ cells to CD3⁺ cells is at or below a threshold ratio.

Option 29. The method of option 24 or 25, wherein the treatment is administered based on a determination that the ratio of CD4⁺ cells to CD3⁺ cells is at or below a threshold ratio.

Option 30. The method of any one of options 24-29, wherein estimating the ratio comprises:
generating an image based on the distributions of cells expressing the targets; and
estimating the ratio of:
   CD4⁺ cells to CD8⁺ cells; and/or
   CD3⁺ cells to CD4⁺ cells; and/or
   CD3⁺ cells to CD8⁺ cells,
   in the tumor based on the image.

Option 31. The method of any one of options 18 to 30, wherein the treatment comprises one or more of immunotherapy, chemotherapy, hormone therapy, radiation therapy, surgery, vaccine therapy, oncolytic virus therapy, or cellular therapy.

Option 32. A method of treating a subject, comprising:
administering to a subject having a disease a first treatment for the disease;
before administering the first treatment, monitoring, by positron emission tomography (PET) or single photon emission computed tomography (SPECT):
   a distribution of cells expressing a first target selected from CD3, CD4, and CD8 in one or more tissues of the subject; and
   a distribution of cells expressing a second target selected from CD3, CD4, and CD8 in the one or more tissues of the subject, wherein the first and second targets are different;
after administering the first treatment, monitoring, by PET or SPECT:
   a distribution of cells expressing the first target in the one or more tissues of the subject; and
   a distribution of cells expressing the second target in the one or more tissues of the subject; and
administering to the subject a second treatment for the disease based on comparisons of:
   the distributions of cells expressing the first target; and
   the distributions of cells expressing the second target.

Option 33. The method of option 32, further comprising:
before administering the first treatment, monitoring, by PET or SPECT, a distribution of cells expressing a third target selected from CD3, CD4, and CD8 in the one or more locations of the subject, wherein the third target is different from the first and second targets; and
after administering the first treatment, monitoring, by PET or SPECT, a distribution of cells expressing the third target in the one or more locations of the subject,
wherein administration of the second treatment is further based on a comparison of the distributions of cells expressing the third target.

Option 34. The method of option 32 or 33, wherein, before administering the first treatment, monitoring the distribution of cells expressing the first target is performed within 1 hour to 2 weeks of monitoring the distribution of cells expressing the second target and/or monitoring the distribution of cells expressing the second target is performed within 1 hour to 2 weeks of monitoring the distribution of cells expressing the third target.

Option 35. The method of any one of options 32 to 34, wherein, after administering the first treatment, monitoring the distribution of cells expressing the first target is performed within 1 hour to 2 weeks of monitoring the distribution of cells expressing the second target and/or monitoring the distribution of cells expressing the second target is performed within 1 hour to 2 weeks of monitoring the distribution of cells expressing the third target.

Option 36. The method of any one of options 32 to 35, wherein the disease is a cancer.

Option 37. The method of any one of options 32 to 36, wherein the subject has received a third treatment for the disease before monitoring the distributions of cells before administering the first treatment.

Option 38. The method of option 37, wherein the first, second and third treatment each comprises one or more of immunotherapy, chemotherapy, hormone therapy, radiation therapy, surgery, vaccine therapy, oncolytic virus therapy, or cellular therapy.

Option 39. The method of any one of options 32 to 38, further comprising identifying the one or more tissues as comprising cancerous tissue.

Option 40. The method of option 39, wherein the one or more tissues are identified as comprising cancerous tissue using computed tomography (CT) scan, X-ray, FDG-PET, or magnetic resonance imaging (MRI).

Option 41. The method of any one of options 32 to 40, wherein the one or more tissues in the subject comprises one or more of a lung, liver, colon, intestine, stomach, brain, kidney, spleen, pancreas, esophagus, lymph node, bone, bone marrow, prostate, cervix, ovary, breast, urethra, bladder, skin, neck, articulated joint, or portions thereof.

Option 42. The method of any one of options 32 to 41, wherein monitoring the distributions comprise:
administering to the subject a first antigen-binding construct comprising a first radionuclide tracer, wherein the antigen-binding construct selectively binds the first target;
imaging the subject by PET or SPECT to acquire the distribution of cells expressing the first target in the one or more tissues of the subject;
administering to the subject a second antigen-binding construct comprising a second radionuclide tracer, wherein the antigen-binding construct selectively binds the second target, and wherein the first and second targets are different;
imaging the subject by PET or SPECT to acquire the distribution of cells expressing the second target in the one or more tissues; and/or
administering to the subject a third antigen-binding construct comprising a third radionuclide tracer, wherein the antigen-binding construct selectively binds the third target;
imaging the subject by PET or SPECT to acquire the distribution of cells expressing the third target in the one or more tissues.

Option 43. The method of option 42, wherein administering the first antigen-binding construct and imaging to acquire the distribution of cells expressing the second target are performed within 1 hour to 2 weeks.

Option 44. The method of option 42 or 43, wherein measuring the level of the first radionuclide tracer is done within 1 hour to 2 weeks of administering the first antigen-binding construct.

Option 45. The method of any one of options 42 to 44, wherein measuring the level of the second radionuclide tracer is done within 1 hour to 2 weeks of administering the second antigen-binding construct.

Option 46. The method of any one of options 42 to 45, wherein measuring the level of the third radionuclide tracer is done within 1 hour to 2 weeks of administering the third antigen-binding construct.

Option 47. The method of any one of options 42 to 46, wherein different antigen-binding constructs are administered on different days.

Option 48. The method of any one of options 42 to 46, wherein administering the first antigen-binding construct and administering the second antigen-binding construct are performed on different days.

Option 49. The method of any one of options 42 to 47, wherein measuring the level of the first radionuclide tracer is performed on the same day as administering the second antigen-binding construct.

Option 50. The method of any one of options 42 to 48, wherein measuring the level of the second radionuclide tracer is performed on the same day as administering the third antigen-binding construct.

Option 51. The method of any one of options 42 to 46, wherein administering the first antigen-binding construct and measuring the level of the second radionuclide tracer are performed on the same day.

Option 52. The method of any one of options 42 to 46, wherein administering the second antigen-binding construct and measuring the level of the third radionuclide tracer are performed on the same day.

Option 53. The method of any one of options 42 to 52, wherein the radionuclide tracers are each selected from ¹⁸F, ⁶⁴Cu, ⁶⁸Ga, ⁸⁹Zr, ¹²³I and ⁹⁹mTc.

Option 54. The method of any one of options 42 to 53, wherein the first radionuclide tracer is ¹⁸F, ⁶⁴Cu, or ⁶⁸Ga.

Option 55. The method of any one of options 42 to 54, wherein the second radionuclide tracer is ¹⁸F or ⁸⁹Zr.

Option 56. The method of any one of options 42 to 53, wherein the first radionuclide tracer is ¹²³I or ⁹⁹mTc.

Option 57. The method of option 56, wherein the second radionuclide tracer is ¹²³I or ⁹⁹mTc, wherein the first and second radionuclide tracers are different.

Option 58. The method of any one of options 42 to 57, wherein the antigen-binding construct is an antibody or antigen-binding fragment thereof.

Option 59. The method of option 58, wherein the antigen-binding construct is a Fab′, F(ab′)2, Fab, Fv, rIgG (reduced IgG), a scFv fragment, a minibody, a diabody, a cys-diabody, or a nanobody.

Option 60. The method of any one of options 18 to 59, wherein the cancer is melanoma, neck cancer, breast cancer, bladder cancer, ovarian cancer, esophageal cancer, colorectal cancer, renal cell carcinoma, prostate cancer, lung cancer, pancreatic cancer, cervical cancer, liver cancer, or lymphoma, squamous cell cervical carcinoma or nasopharyngeal carcinoma, or bone cancer.

Option 61. The method of any one of options 18 to 60, wherein the subject has melanoma, non small-cell lung carcinoma (NSCLC), or renal cell cancer (RCC).

Option 62. A method of imaging a subject, comprising:
administering to a subject a first PET tracer that selectively binds a first target selected from CD3, CD4, and CD8;
estimating a distribution and/or abundance of cells expressing the first target in one or more tissues of the subject using positron emission tomography (PET) or single photon emission computed tomography (SPECT) to measure a signal from the first PET tracer in the subject;
administering to the subject a second PET tracer that selectively binds a second target selected from CD3, CD4, and CD8, and wherein the first and second targets are different;
estimating a distribution and/or abundance of cells expressing the second target in the one or more tissues of the subject using PET or SPECT to measure a signal from the second PET tracer in the subject; and
generating an image based on the distributions and/or abundances of the cells expressing the targets, wherein the image provides an indication of the immune contexture of the one or more tissues.

Option 63. The method of any one of the preceding options, wherein:
the first target is CD3, and the second target is CD4;
the first target is CD3 and the second target is CD8; or
the first target is CD4 and the second target is CD8.

Option 64. The method of any one of the preceding options, wherein the CD3 is human CD3, the CD4 is human CD4 and the CD8 is human CD8.

Option 65. The method of option 64, wherein the human CD3 comprises the sequence set forth in SEQ ID NO: 186, the human CD4 comprises the sequence set forth in SEQ ID NO: 100, and the human CD8 comprises any one of the sequences set forth in SEQ ID NOs: 80-82.

Singular Terms

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments.

### INCORPORATION BY REFERENCE

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls.

### EQUIVALENTS

The foregoing description details certain embodiments. It will be appreciated, however, that no matter how detailed the foregoing may appear in text, the invention may be practiced in many ways and the invention should be construed in accordance with the appended claims and any equivalents thereof.

1. A method of imaging a subject, comprising:
administering to a subject a first antigen-binding construct comprising a first radionuclide tracer, wherein the antigen-binding construct selectively binds a first target selected from CD3, CD4, IFN-gamma, and CD8;
estimating a distribution and/or abundance of the first target and/or of cells expressing the first target in one or more tissues of the subject using positron emission tomography (PET) or single photon emission computed tomography (SPECT) to measure a level of the first radionuclide tracer in the subject;
administering to the subject a second antigen-binding construct comprising a second radionuclide tracer, wherein the antigen-binding construct selectively binds a second target selected from CD3, CD4, IFN-gamma, and CD8, and wherein the first and second targets are different;
estimating a distribution and/or abundance of the second target and/or of cells expressing the second target in the one or more tissues of the subject using PET or SPECT to measure a level of the second radionuclide tracer in the subject; and
generating an image based on the distributions and/or abundances of the targets and/or the cells expressing the targets, wherein the image provides an indication of the immune contexture of the one or more tissues.

2. The method of claim 1, further comprising:
administering to the subject a third antigen-binding construct comprising a third radionuclide tracer, wherein the antigen-binding construct selectively binds a third target selected from CD3, CD4, IFN-gamma, and CD8, and wherein the third target is different from the first and second targets;
estimating a distribution and/or abundance of the third target and/or of cells expressing the third target in the one or more tissues of the subject using PET or SPECT to measure a level of the third radionuclide tracer in the subject.

3. The method of claim 1, further comprising determining a relative abundance among cells expressing any one of the targets compared to cells expressing another one of the targets in each of the one or more tissues.

4. The method of claim 1, further comprising identifying one or more areas of overlap between:
the distribution and/or abundance of the first target and/or of cells expressing the first target and the distribution and/or abundance the second target and/or of cells expressing the second target;
the distribution and/or abundance of the first target and/or of cells expressing the first target and the distribution and/or abundance of cells expressing the third target; or
the distribution and/or abundance of the third target and/or of cells expressing the second target and the distribution and/or abundance of cells expressing the third target.

5. The method of claim 1, wherein the image provides one or more of:
an abundance of any two or more of CD3⁺, CD4⁺ and CD8⁺ cells;
a relative abundance of any one of CD3⁺, CD4⁺ and CD8⁺ cells compared to another one of CD3⁺, CD4⁺ and CD8⁺ cells; and
a ratio of any one of CD3⁺, CD4⁺ and CD8⁺ cells to another one of CD3⁺, CD4⁺ and CD8⁺ cells,
in the one or more tissues of the subject.

6. The method of claim 1, wherein the radionuclide tracers are each selected from ¹⁸F, ⁶⁴Cu, ⁶⁸Ga, ⁸⁹Zr, ¹²³I and ⁹⁹mTc.

7. (canceled)

8. The method of claim 1, wherein the second radionuclide tracer is ¹⁸F or ⁸⁹Zr.

9. (canceled)

10. (canceled)

11. The method of claim 1, wherein measuring the level of the radionuclide tracer is done within 1 hour to 2 weeks of administering the antigen-binding construct comprising the radionuclide tracer.

12. The method of claim 1, wherein measuring the level of the first radionuclide tracer is done within 1 hour to 2 weeks of administering the first antigen-binding construct.

13. (canceled)

14. (canceled)

15. The method of claim 1, wherein different antigen-binding constructs are administered on different days.

16. (canceled)

17. The method of claim 1, wherein measuring the level of the first radionuclide tracer is performed on the same day as administering the second antigen-binding construct.

18. The method of claim 1, wherein measuring the level of the second radionuclide tracer is performed on the same day as administering the third antigen-binding construct.

19.-24. (canceled)

25. The method of claim 1, wherein the subject has a cancer.

26. (canceled)

27. The method of claim 25, claim 26, wherein the one or more tissues are identified as comprising cancerous tissue using computed tomography (CT) scan, X-ray, FDG-PET, or magnetic resonance imaging (MRI).

28. (canceled)

29. The method of claim 1, wherein the subject has a disease or condition selected from a solid tumor, a non-solid tumor, an autoimmune disease, an infectious disease (including without limitation, viral, bacterial, or fungal infections), or is recovering from stroke or cardiac event, or is being assessed for a response to therapy for the disease or condition or as part of a clinical trial of a therapeutic agent for treating the disease or condition.

30. (canceled)

31. The method of claim 1, wherein the immune contexture of the one or more tissues is reported as an immunoscore, or is used to generate an immunoscore with other diagnostic results.

32. The method of claim 31 wherein the immunoscore comprises assessment of one or more biomarkers selected from IL-6, C-reactive protein (CRP), VEGF, fibronectin, lactate dehydrogenase (LDH), soluble CD25, NY-ESO-1 antibody, IFN-γ, PD-L1, tumor-associated fibroblast (TAF) markers, FAP/CD8 (neutrophil/lymphocyte ratio), cancer associate fibrosis markers, tumor-associated macrophage markers and chemokines.

33. A method for providing a prognosis for a cancer, comprising:
administering to a subject having a cancer a first antigen-binding construct comprising a first radionuclide tracer, wherein the antigen-binding construct selectively binds a first target selected from CD3, CD4, and CD8;
imaging the subject by positron emission tomography (PET) or single photon emission computed tomography (SPECT) to acquire a distribution of cells expressing the first target in a tumor in the subject;
administering to the subject a second antigen-binding construct comprising a second radionuclide tracer, wherein the antigen-binding construct selectively binds a second target selected from CD3, CD4, and CD8, and wherein the first and second targets are different;
imaging the subject by PET or SPECT to acquire a distribution of cells expressing the second target in the tumor;
determining an abundance of, and/or a relative abundance among, CD3⁺, CD4⁺ and/or CD8⁺ cells in the tumor based on the distributions of cells expressing the targets; and
providing a prognosis for the disease based on an evaluation of the abundance of, and/or the relative abundance among, CD3⁺, CD4⁺ and/or CD8⁺ cells in the tumor.

34. (canceled)

35. The method of claim 33 or 31, wherein determining the abundance of, or a relative abundance among, CD3⁺, CD4±_and/or CD8⁺ cells in the tumor comprises:
generating an image based on the distributions of cells expressing the targets; and
determining the abundance of, and/or a relative abundance among, CD3⁺, CD4⁺ and/or CD8⁺ cells in the tumor based on the image.

36. The method of claim 33, wherein the subject has received a treatment for the cancer before administering to the subject the first antigen-binding construct.

37. (canceled)

38. The method of claim 33, further comprising scanning the subject using computed tomography (CT) scan, X-ray, FDG-PET, or magnetic resonance imaging (MRI) to identify the tumor in the subject.

39.-55. (canceled)

56. The method of claim 1, further comprising:
administering to the subject an antigen-binding construct that binds to IFN-gamma and that comprises a radionuclide tracer; and
estimating a distribution and/or abundance of IFN-gamma in the one or more tissues of the subject using PET or SPECT to measure a level of the radionuclide tracer in the subject.

57. A method of imaging a subject, comprising:
administering to a subject a first antigen-binding construct comprising a first detectable marker, wherein the antigen-binding construct selectively binds a first target selected from CD3, CD4, IFN-gamma, and CD8;
estimating a distribution and/or abundance of cells expressing the first target in one or more tissues of the subject using non-invasive imaging to measure a level of the first detectable marker in the subject;
administering to the subject a second antigen-binding construct comprising a second detectable marker, wherein the antigen-binding construct selectively binds a second target selected from CD3, CD4, IFN-gamma, and CD8, and wherein the first and second targets are different;
estimating a distribution and/or abundance of cells expressing the second target in the one or more tissues of the subject using non-invasive imaging to measure a level of the second detectable marker in the subject; and
generating an image based on the distributions and/or abundances of the cells expressing the targets, wherein the image provides an indication of the immune contexture of the one or more tissues.

58.-61. (canceled)

62. The method of claim 1, wherein the antigen-binding construct is an antibody or antigen-binding fragment thereof.

63. (canceled)

64. The method of claim 1, wherein the antigen-binding construct that binds CD8 comprises an amino acid sequence at least about 80% identical to any one of the amino acid sequences shown in FIGS. 7-36.

65. The method of claim 1, wherein the antigen-binding construct that binds CD4 comprises an amino acid sequence at least about 80% identical to any one of the amino acid sequences shown in FIGS. 38-50.

66. The method of claim 1, wherein the antigen-binding construct that binds CD3 comprises an amino acid sequence at least about 80% identical to any one of the amino acid sequences shown in FIGS. 52A-84I.

67. (canceled)

68. (canceled)


SEQUENCE LISTING (265 sequences)

SEQ ID NO: 1 - protein, 118 aa, Murine
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 2 - protein, 120 aa, Homo sapiens [sequence omitted]

SEQ ID NO: 3 - protein, 118 aa, Artificial Sequence
  Antigen binding construct or fragment thereof
EVQLVESGGGLVQPGGSLRLSCAASGFNIKDTYIHWVRQAPGKGLEWVARIDPANDNTLYASKFQGRATISADTSKNTAYLQMNSLRAEDTAVYYCGRGYGYYVFDHWGQGTLVTVSS

SEQ ID NO: 4 - protein, 118 aa, Murine
QVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 5 - protein, 115 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATAEYFQHWGQGTLVTVSS

SEQ ID NO: 6 - protein, 118 aa, Artificial Sequence
  Antigen binding construct or fragment thereof
QVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSS

SEQ ID NO: 7 - protein, 107 aa, Murine
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELK

SEQ ID NO: 8 - protein, 107 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 9 - protein, 107 aa, Artificial Sequence
  Antigen binding construct or fragment thereof
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 10 - protein, 394 aa, Artificial Sequence
  Antigen binding construct or fragment thereof
METDTLLLWVLLLWVPGSTGDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKGSTSGGGSGGGSGGGGSSEVQLQQSGAELVKPGASVKLSCTASGFNIKDTYIHFVRQRPEQGLEWIGRIDPANDNTLYASKFQGKATITADTSSNTAYMHLSSLTSGDTAVYYCGRGYGYYVFDHWGQGTTLTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1185 bp, Artificial Sequence
  Antigen binding construct or fragment thereof
atggagacagacaccctgctcctgtgggtgctgctcctctgggtccctggatccaccggcgatgtccagatcaaccaaagccccagctttctggctgcctcccctggagagacaatcaccatcaattgccggaccagccggagcatttcccagtacctcgcctggtaccaggaaaagcctggcaagaccaacaagctgctgatctactccggctccacactccagagcggcattccctccaggtttagcggatccggatccggaaccgacttcacactcaccatctccggcctggagcccgaggacttcgccatgtattattgccagcagcacaatgagaaccccctgaccttcggcgctggcaccaagctggagctgaaaggctccaccagcggaggcggatccggaggaggaagcggcggcggaggctcctccgaagtgcagctgcaacagagcggcgccgaactggtgaagcctggagcttccgtgaaactcagctgtaccgccagcggcttcaacatcaaggatacctacatccacttcgtgcggcaaaggcctgagcagggcctggaatggatcggcaggatcgaccccgccaacgacaacaccctctacgcctccaagttccaaggcaaggccacaatcaccgctgatacaagctccaacaccgcctacatgcacctcagctccctgaccagcggagacaccgccgtgtactactgcggacggggatacggctactatgtgttcgaccactggggccaaggcaccacactcaccgtgtcctccgagcccaagtcctgcgacaagacacacacctgtcccccttgtggaggaggatcctccggaggcggctccggcggacagcctagggagccccaggtgtacacactgcccccttccagggacgaactcaccaagaaccaggtgtccctgacctgcctggtgaagggattctaccccagcgacatcgccgtggagtgggagtccaacggccaacccgagaacaattacaagaccaccccccctgtgctcgattccgacggctccttcttcctgtactccaagctcaccgtggacaagtcccggtggcaacagggcaatgtgttctcctgcagcgtcatgcacgaggccctgcataaccactacacccagaaatccctcagcctctcccctggaaaatga

SEQ ID NO: 12 - protein, 394 aa, Artificial Sequence
  Antigen binding construct or fragment thereof
METDTLLLWVLLLWVPGSTGEVQLQQSGAELVKPGASVKLSCTASGFNIKDTYIHFVRQRPEQGLEWIGRIDPANDNTLYASKFQGKATITADTSSNTAYMHLSSLTSGDTAVYYCGRGYGYYVFDHWGQGTTLTVSSGSTSGGGSGGGSGGGGSSDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1185 bp, Artificial Sequence
  Antigen binding construct or fragment thereof
atggagaccgacacactcctgctctgggtgctcctgctgtgggtgcctggcagcacaggagaagtgcagctgcagcagtccggcgccgaactcgtcaaacccggagcctccgtcaaactgtcctgcacagccagcggcttcaacatcaaggacacctacatccatttcgtgcggcaaaggcctgaacagggactcgagtggatcggcaggatcgaccccgccaacgacaataccctctacgcctccaagttccagggaaaggccaccattaccgccgacacatccagcaacaccgcctacatgcacctcagctccctgacatccggcgacaccgccgtgtactactgcggcaggggctacggctactacgtgtttgaccactggggccagggaacaaccctgaccgtgtccagcggctccacctccggaggcggaagcggcggaggatccggaggaggaggctcctccgacgtgcaaatcaaccagtcccctagcttcctggccgctagccctggcgagacaatcacaatcaattgtcggaccagccggtccatctcccagtatctggcctggtaccaggagaagcccggcaagacaaacaagctgctcatctacagcggcagcaccctccaatccggcatcccttcccggtttagcggctccggatccggaaccgactttaccctgaccatcagcggcctggaacccgaggatttcgccatgtactactgccagcagcacaacgagaatcccctgacctttggagccggcacaaagctcgagctgaaggagcccaagagctgcgacaaaacccacacctgtcccccttgcggaggaggatcctccggcggcggaagcggaggacaacccagggagccccaggtctacaccctgcctcctagccgggacgaactgacaaagaaccaggtgtccctgacctgtctcgtcaagggcttctacccttccgacatcgccgtcgagtgggaaagcaacggccagcccgagaacaattacaagaccacaccccccgtcctggacagcgatggcagcttcttcctctactccaagctgaccgtggacaagagccggtggcaacaaggcaacgtgttctcctgcagcgtcatgcatgaggccctgcacaatcactacacccagaagagcctgagcctctcccccggcaagtga

SEQ ID NO: 14 - protein, 394 aa, Artificial Sequence
  Antigen binding construct or fragment thereof
METDTLLLWVLLLWVPGSTGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1185 bp, Artificial Sequence
  Antigen binding construct or fragment thereof
atggagacagacaccctcctgctgtgggtcctgctgctgtgggtgcctggcagcacaggagacatccaaatgacccagtcccctagcagcctcagcgcttccgtcggagacagggtcaccatcacatgcaggacctccaggtccatcagccagtatctggcctggtatcagcagaaacccggcaaggtgcctaagctgctgatctacagcggcagcacactccagagcggagtgcccagccggttttccggaagcggatccggaaccgacttcaccctgaccatttccagcctgcaacctgaagacgtggccacctactactgtcagcagcacaacgagaaccccctcaccttcggcggaggcaccaaagtcgagatcaagggcagcaccagcggaggaggaagcggcggaggctccggaggaggaggctcctcccaagtgcagctcgtccaaagcggcgctgaggtgaaaaagcccggcgccacagtcaaaatctcctgcaaggtcagcggcttcaacatcaaggatacctacatccactgggtgcaacaggcccccggcaaaggactcgaatggatgggccggatcgaccctgctaacgacaacacactctacgcctccaagttccagggcagggtgaccatcaccgccgatacctccaccgacacagcctacatggagctgagcagcctgaggtccgaggacaccgccgtctattactgcgcccggggatacggctactacgtgtttgaccattggggacagggaacactcgtgaccgtgagctccgagcccaagagctgcgacaagacccacacatgtcctccttgcggaggaggcagctccggaggcggatccggcggacaacctagggagccccaggtctataccctgccccccagcagggacgagctgacaaagaaccaggtctccctgacctgcctggtgaaaggattctaccccagcgacatcgctgtcgaatgggagtccaacggccagcccgagaacaactacaagacaaccccccccgtgctggattccgacggcagcttcttcctctactccaagctgaccgtcgacaagtccaggtggcagcagggcaacgtgttttcctgctccgtgatgcatgaggccctgcacaaccactacacccagaagtccctgagcctcagccctggcaagtga

SEQ ID NO: 16 - protein, 394 aa, Artificial Sequence
  Antigen binding construct or fragment thereof
METDTLLLWVLLLWVPGSTGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGSTSGGGSGGGSGGGGSSDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1185 bp, Artificial Sequence
  Antigen binding construct or fragment thereof
atggagaccgatacactgctgctctgggtgctgctgctgtgggtgcctggaagcaccggacaggtgcaactggtccagtccggcgccgaggtgaaaaagcctggcgccaccgtcaagatctcctgtaaggtgagcggcttcaacatcaaggacacctacatccactgggtgcagcaggctcccggaaagggactggagtggatgggcaggatcgaccctgccaatgacaacaccctctacgccagcaagttccaaggacgggtgaccatcacagccgacacatccaccgacacagcctatatggagctctccagcctgaggtccgaggacaccgccgtgtactactgtgccaggggatacggctattacgtgttcgaccactggggacagggcaccctggtgaccgtgagcagcggaagcaccagcggcggaggcagcggaggcggaagcggcggcggcggatcctccgacattcagatgacccaatccccctccagcctgtccgctagcgtgggagacagggtgacaatcacatgtcggacctccaggtccatcagccaatatctcgcctggtatcagcagaagcccggcaaggtgcccaagctcctgatctacagcggctccaccctccaaagcggagtgccttcccggtttagcggaagcggcagcggcacagactttaccctgacaatcagctccctgcaacctgaggacgtcgccacatactactgccagcagcacaacgagaaccctctcacctttggcggcggcaccaaagtggagatcaaggagcccaaatcctgcgacaagacacacacctgccccccttgtggaggaggcagctccggcggcggcagcggcggacaaccccgggaacctcaggtgtatacactccccccttccagggatgagctgaccaagaaccaagtctccctgacctgtctggtgaaaggcttctacccctccgacatcgctgtcgagtgggagagcaacggccagcccgaaaacaactataagaccaccccccccgtgctcgattccgatggcagcttcttcctgtactccaagctcacagtcgacaagagccggtggcaacagggcaacgtcttctcctgtagcgtcatgcacgaggccctccacaaccactacacccagaagtccctctccctgagccccggaaaatga

SEQ ID NO: 18 - protein, 253 aa, Artificial Sequence
  Antigen binding construct or fragment thereof
METDTLLLWVLLLWVPGSTGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKSGGGGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGGC

SEQ ID NO: 19 - DNA, 759 bp, Artificial Sequence
  Antigen binding construct or fragment thereof
atggagaccgatacactgctgctgtgggtgctgctgctctgggtccctggcagcacaggagacatccagatgacacagagccctagctccctgagcgcttccgtgggagatagggtgaccatcacatgccggacctccaggtccatctcccagtacctggcctggtaccagcagaagcccggcaaggtgcccaagctgctcatctatagcggcagcaccctgcagagcggagtgccttcccggttttccggatccggctccggcacagactttaccctgaccatctccagcctgcagcctgaggatgtcgccacctactactgccaacagcacaacgagaaccccctgaccttcggcggcggaaccaaggtcgagatcaagtccggaggaggaggccaagtgcagctggtccaatccggcgccgaagtgaaaaagcccggcgccaccgtgaagatcagctgcaaggtgtccggcttcaacatcaaggacacctatatccactgggtccagcaagcccccggaaaaggcctggagtggatgggacggattgaccccgccaacgacaacacactctatgcctccaagttccagggcagggtgacaatcaccgccgacaccagcaccgacacagcttatatggagctgtcctccctccggtccgaggataccgccgtctactactgcgccaggggctacggctactacgtgtttgaccactggggccagggcaccctggtgacagtgtccagcggaggctgc

SEQ ID NO: 20 - protein, 394 aa, Artificial Sequence; identical to SEQ ID NO: 14
  Antigen binding construct or fragment thereof

SEQ ID NO: 21 - DNA, 759 bp, Artificial Sequence
  Antigen binding construct or fragment thereof
atggagaccgacaccctgctgctctgggtcctcctgctgtgggtgcctggcagcacaggacaggtgcaactggtgcagagcggcgccgaggtcaagaaacctggcgccaccgtgaagatcagctgcaaggtgtccggcttcaacatcaaggacacctacatccactgggtccaacaagcccccggaaagggcctggaatggatgggccggattgaccccgccaacgacaacaccctctatgccagcaagttccagggcagggtcaccatcaccgccgacaccagcaccgacaccgcctacatggagctgagcagcctgcggagcgaagacaccgccgtgtactactgcgccaggggctacggctactacgtcttcgaccattggggacagggcaccctcgtgacagtgtccagctccggcggaggaggagatatccagatgacccagagcccttccagcctgtccgcttccgtgggagatcgggtgaccatcacatgcaggacctccaggtccatctcccagtacctggcctggtaccaacagaagcccggcaaggtgcccaagctgctgatctacagcggcagcacactgcaatccggcgtcccttcccggttttccggatccggatccggcaccgacttcaccctgaccatcagctccctgcaacccgaggacgtggccacctactactgtcagcagcacaacgagaaccccctcacctttggcggcggaaccaaggtcgagatcaagggcggctgc

SEQ ID NO: 22 - protein, 256 aa, Artificial Sequence
  Antigen binding construct or fragment thereof
METDTLLLWVLLLWVPGSTGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGGGSGGGGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGGC

SEQ ID NO: 23 - DNA, 768 bp, Artificial Sequence
  Antigen binding construct or fragment thereof
atggagaccgatacactgctgctgtgggtgctgctgctctgggtccctggcagcacaggagacatccagatgacacagagccctagctccctgagcgcttccgtgggagatagggtgaccatcacatgccggacctccaggtccatctcccagtacctggcctggtaccagcagaagcccggcaaggtgcccaagctgctcatctatagcggcagcaccctgcagagcggagtgccttcccggttttccggatccggctccggcacagactttaccctgaccatctccagcctgcagcctgaggatgtcgccacctactactgccaacagcacaacgagaaccccctgaccttcggcggcggaaccaaggtcgagatcaagggaggaggctccggaggaggaggccaagtgcagctggtccaatccggcgccgaagtgaaaaagcccggcgccaccgtgaagatcagctgcaaggtgtccggcttcaacatcaaggacacctatatccactgggtccagcaagcccccggaaaaggcctggagtggatgggacggattgaccccgccaacgacaacacactctatgcctccaagttccagggcagggtgacaatcaccgccgacaccagcaccgacacagcttatatggagctgtcctccctccggtccgaggataccgccgtctactactgcgccaggggctacggctactacgtgtttgaccactggggccagggcaccctggtgacagtgtccagcggaggctgc

SEQ ID NO: 24 - protein, 256 aa, Artificial Sequence
  Antigen binding construct or fragment thereof
METDTLLLWVLLLWVPGSTGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGGGSGGGGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGGC

SEQ ID NO: 25 - DNA, 768 bp, Artificial Sequence
  Antigen binding construct or fragment thereof
atggagaccgacaccctgctgctctgggtcctcctgctgtgggtgcctggcagcacaggacaggtgcaactggtgcagagcggcgccgaggtcaagaaacctggcgccaccgtgaagatcagctgcaaggtgtccggcttcaacatcaaggacacctacatccactgggtccaacaagcccccggaaagggcctggaatggatgggccggattgaccccgccaacgacaacaccctctatgccagcaagttccagggcagggtcaccatcaccgccgacaccagcaccgacaccgcctacatggagctgagcagcctgcggagcgaagacaccgccgtgtactactgcgccaggggctacggctactacgtcttcgaccattggggacagggcaccctcgtgacagtgtccagcggaggaggatccggcggaggaggagatatccagatgacccagagcccttccagcctgtccgcttccgtgggagatcgggtgaccatcacatgcaggacctccaggtccatctcccagtacctggcctggtaccaacagaagcccggcaaggtgcccaagctgctgatctacagcggcagcacactgcaatccggcgtcccttcccggttttccggatccggatccggcaccgacttcaccctgaccatcagctccctgcaacccgaggacgtggccacctactactgtcagcagcacaacgagaaccccctcacctttggcggcggaaccaaggtcgagatcaagggcggctgc

SEQ ID NO: 26 - DNA, 321 bp, Murine
gatgtccagataaaccagtctccatcttttcttgctgcgtctcctggagaaaccattactataaattgcaggacaagtaggagtattagtcaatatttagcctggtatcaagagaaacctgggaaaactaataagcttcttatctactctggatccactctgcaatctggaattccatcaaggttcagtggcagtggatctggtacagatttcactctcaccatcagtggcctggagcctgaagattttgcaatgtattactgtcaacagcataatgaaaacccgctcacgttcggtgctgggaccaagctggagctgaag

SEQ ID NO: 27 - protein, 107 aa, Murine; identical to SEQ ID NO: 7

SEQ ID NO: 28 - DNA, 321 bp, Artificial Sequence
  Antigen binding construct or fragment thereof
gacgtccagataacccagtctccatcctccctgtctgcatctgtaggagacagagtcaccatcacttgcaggacaagtaggagtattagtcaatatttagcctggtatcagcagaaaccagggaaagttcctaagctcctgatctattctggatccactctgcaatctggagtcccatctcggttcagtggcagtggatctgggacagatttcactctcaccatcagcagcctgcagcctgaagatgttgcaacttattactgtcaacagcataatgaaaacccgctcacgttcggcggagggaccaaggtggagatcaaa

SEQ ID NO: 29 - protein, 107 aa, Artificial Sequence; identical to SEQ ID NO: 9
  Antigen binding construct or fragment thereof

SEQ ID NO: 30 - DNA, 354 bp, Murine
gaggtccagctgcagcagtctggggcagagcttgtgaagccaggggcctcagtcaagttgtcctgcacagcttctggcttcaacattaaagacacctatatacacttcgtgaggcagaggcctgaacagggcctggagtggattggaaggattgatcctgcgaatgataatactttatatgcctcaaagttccagggcaaggccactataacagcagacacatcatccaacacagcctacatgcacctctgcagcctgacatctggggacactgccgtctattactgtggtagaggttatggttactacgtatttgaccactggggccaaggcaccactctcacagtctcctca

SEQ ID NO: 31 - protein, 118 aa, Murine; identical to SEQ ID NO: 1

SEQ ID NO: 32 - DNA, 354 bp, Artificial Sequence
  Antigen binding construct or fragment thereof
gaagtgcagctggtggaaagcggcggcggcctggtgcagccgggcggcagcctgcgcctgagctgcgcggcgagcggctttaacattaaagatacctatattcattttgtgcgccaggcgccgggcaaaggcctggaatggattggccgcattgatccggcgaacgataacaccctgtatgcgagcaaatttcagggcaaagcgaccattagcgcggataccagcaaaaacaccgcgtatctgcagatgaacagcctgcgcgcgggagataccgcggtgtattattgcggccgcggctatggctattatgtgtttgatcattggggccagggcaccctggtgaccgtgagcagc

SEQ ID NO: 33 - protein, 118 aa, Artificial Sequence
  Antigen binding construct or fragment thereof
EVQLVESGGGLVQPGGSLRLSCAASGFNIKDTYIHFVRQAPGKGLEWIGRIDPANDNTLYASKFQGKATISADTSKNTAYLQMNSLRAGDTAVYYCGRGYGYYVFDHWGQGTLVTVSS

SEQ ID NO: 34 - DNA, 354 bp, Artificial Sequence
  Antigen binding construct or fragment thereof
gaagtgcagctggtggaaagcggcggcggcctggtgcagccgggcggcagcctgcgcctgagctgcgcggcgagcggctttaacattaaagatacctatattcattttgtgcgccaggcgccgggcaaaggcctggaatggattggccgcattgatccggcgaacgataacaccctgtatgcgagcaaatttcagggcaaagcgaccattagcgcggataccagcaaaaacaccgcgtatctgcagatgaacagcctgcgcgcggaagataccgcggtgtattattgcggccgcggctatggctattatgtgtttgatcattggggccagggcaccctggtgaccgtgagcagc

SEQ ID NO: 35 - protein, 118 aa, Artificial Sequence
  Antigen binding construct or fragment thereof
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSS

SEQ ID NO: 36 - DNA, 354 bp, Artificial Sequence
  Antigen binding construct or fragment thereof
caggtgcagctggtgcagagcggcgcggaagtgaaaaaaccgggcgcgaccgtgaaaattagctgcaaagtgagcggctttaacattaaagatacctatattcattttgtgcagcaggcgccgggcaaaggcctggaatggattggccgcattgatccggcgaacgataacaccctgtatgcgagcaaatttcagggcaaagcgaccattaccgcggataccagcaccgataccgcgtatatggaactgagcagcctgcgcagcggagataccgcggtgtattattgcggccgcggctatggctattatgtgtttgatcattggggccagggcaccctggtgaccgtgagcagc

SEQ ID NO: 37 - protein, 118 aa, Artificial Sequence
  Antigen binding construct or fragment thereof
QVQLVQSGAEVKKPGATVKISCKVSGFNIKDTYIHFVQQAPGKGLEWIGRIDPANDNTLYASKFQGKATITADTSTDTAYMELSSLRSGDTAVYYCGRGYGYYVFDHWGQGTLVTVSS

SEQ ID NO: 38 - DNA, 354 bp, Artificial Sequence
  Antigen binding construct or fragment thereof
caggtgcagctggtgcagagcggcgcggaagtgaaaaaaccgggcgcgaccgtgaaaattagctgcaaagtgagcggctttaacattaaagatacctatattcattttgtgcagcaggcgccgggcaaaggcctggaatggattggccgcattgatccggcgaacgataacaccctgtatgcgagcaaatttcagggcaaagcgaccattaccgcggataccagcaccgataccgcgtatatggaactgagcagcctgcgcagcgaagataccgcggtgtattattgcggccgcggctatggctattatgtgtttgatcattggggccagggcaccctggtgaccgtgagcagc

SEQ ID NO: 39 - protein, 117 aa, Artificial Sequence
  Antigen binding construct or fragment thereof
QVQLVQSGAEVKKPGATVKISCKVSGFNIKDTYIHFVQQAPGKGLEWIGRIDPANDNTLYASKFQGKATITADTSTDTAYMELSSLRSEDTAVYYCGRGYGYYVFDHWGQGTLVTVS

SEQ ID NO: 40 - protein, 18 aa, Artificial Sequence
  Linker sequence
GSTSGGGSGGGSGGGGSS

SEQ ID NO: 41 - protein, 118 aa, Artificial Sequence; identical to SEQ ID NO: 35
  IAB22M variable heavy sequence

SEQ ID NO: 42 - protein, 22 aa, Artificial Sequence
  Human IgG2 EH1
ERKCCVECPPCPGGGSSGGGSG

SEQ ID NO: 43 - protein, 9 aa, Homo sapiens
CCVECPPCP

SEQ ID NO: 44 - protein, 10 aa, Homo sapiens
GGGSSGGGSG

SEQ ID NO: 45 - protein, 107 aa, Artificial Sequence
  Human IgG2 CH3
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - protein, 22 aa, Artificial Sequence
  Human IgG2 EH2
ERKSCVECPPCPGGGSSGGGSG

SEQ ID NO: 47 - protein, 9 aa, Homo sapiens
SCVECPPCP

SEQ ID NO: 48 - protein, 24 aa, Artificial Sequence
  Human IgG1 EH1
EPKSCDKTHTCPPCGGGSSGGGSG

SEQ ID NO: 49 - protein, 10 aa, Artificial Sequence
  Upper hinge
EPKSCDKTHT

SEQ ID NO: 50 - protein, 107 aa, Artificial Sequence
  Human IgG1 CH3 (G1m1 allotype)
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 107 aa, Artificial Sequence
  Human IgG1 CH3 (nG1m1 allotype)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 19 aa, Artificial Sequence
  Human IgG2 NH1
ERKCCVECPPCPAPPVAGP

SEQ ID NO: 53 - protein, 7 aa, Homo sapiens
APPVAGP

SEQ ID NO: 54 - protein, 19 aa, Artificial Sequence
  Human IgG2 NH2
ERKSCVECPPCPAPPVAGP

SEQ ID NO: 55 - protein, 27 aa, Artificial Sequence
  Human IgG1 EH3
EPKSSDKTHTCPPCPPCGGGSSGGGSG

SEQ ID NO: 56 - protein, 10 aa, Artificial Sequence
  Upper hinge
EPKSSDKTHT

SEQ ID NO: 57 - protein, 7 aa, Homo sapiens
CPPCPPC

SEQ ID NO: 58 - protein, 30 aa, Artificial Sequence
  Human IgG1 EH5
EPKSSDKTHTCPPCPPCPPCGGGSSGGGSG

SEQ ID NO: 59 - protein, 10 aa, Homo sapiens
CPPCPPCPPC

SEQ ID NO: 60 - protein, 29 aa, Artificial Sequence
  IgG3/IgG1 EH6
ELKTPLGDTTHTCVECPPCGGGSSGGGSG

SEQ ID NO: 61 - protein, 12 aa, Artificial Sequence
  Upper hinge
ELKTPLGDTTHT

SEQ ID NO: 62 - protein, 7 aa, Homo sapiens
CVECPPC

SEQ ID NO: 63 - protein, 29 aa, Artificial Sequence
  IgG3/IgG1 EH7
ELKTPLGDTTHTCPPCPPCGGGSSGGGSG

SEQ ID NO: 64 - protein, 32 aa, Artificial Sequence
  IgG3/IgG1 EH8
ELKTPLGDTTHTCPPCPPCPPCGGGSSGGGSG

SEQ ID NO: 65 - protein, 24 aa, Artificial Sequence
  Human IgG1 EH2
EPKSSDKTHTCPPCGGGSSGGGSG

SEQ ID NO: 66 - protein, 396 aa, Artificial Sequence
  IAB22M gamma 1 EH3(Minibody 1)
METDTLLLWVLLLWVPGSTGDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - DNA, 1191 bp, Artificial Sequence
  IAB22M gamma 1 EH3(M1)
atggagaccgataccctgctgctgtgggtgctgctgctgtgggtgcccggctccacaggcgatgtgcagatcacacagagccctagcagcctgagcgccagcgtgggagatagagtcaccatcacatgcaggaccagcagaagcattagccagtacctggcctggtaccagcaaaagcccggcaaggtgcccaagctgctgatctacagcggctccaccctgcagagcggcgtgcccagcagattctccggctccggaagcggcacagactttaccctgaccatctcctccctgcagcccgaggatgtcgccacctactactgccagcagcacaacgaaaaccccctgacatttggcggcggcaccaaggtggagatcaagggcagcaccagcggtggaggaagtggaggtggaagtggaggaggcggaagcagcgaggtgcagctggtggagagtggtggaggactggtgcagcccggaggcagcctgagactgagctgtgctgcctccggattcaatatcaaggacacctacatccacttcgtgagacaggcccccggcaagggactggagtggattggaaggatcgaccccgccaacgacaacaccctgtacgccagcaaattccagggcaaggccacaatcagcgccgacaccagcaagaacaccgcctatctgcagatgaactccctgagagccgaggacacagccgtgtactactgcggcaggggctacggctattacgtgttcgaccactggggccagggcaccctggtgacagtgagcagcgaacccaagagctccgacaagacccacacctgtcccccttgccctccttgtggcggaggaagctccggaggcggaagcggaggacagcctagggagccccaggtgtataccctccccccctccagggaagagatgaccaagaaccaggtgagcctgacctgcctcgtgaagggcttttatccctccgatatcgccgtggagtgggagagcaacggccagcctgagaacaattacaagaccaccccccctgtgctggactccgatggcagcttcttcctgtattccaagctgaccgtcgacaagtccaggtggcaacagggcaacgtcttcagctgcagcgtgatgcacgaggccctgcacaatcactacacccagaagtccctctccctgagccccggctga

SEQ ID NO: 68 - DNA, 1200 bp, Artificial Sequence
  IAB22M gamma 1 EH5(M1)
atggagacagacaccctcctcctgtgggtgctgctgctgtgggtgcccggatccaccggagacgtgcagatcacacagagccccagctccctgtccgctagcgtgggcgacagagtgaccatcacctgcaggaccagcaggagcatctcccagtacctcgcttggtaccagcagaagcctggcaaggtgcccaagctgctgatttacagcggatccaccctgcagagcggcgtgcctagcaggtttagcggcagcggatccggaacagacttcaccctgaccatcagcagcctgcagcctgaagatgtggccacctactactgtcagcagcacaacgaaaaccccctcaccttcggcggcggcacaaaggtggaaatcaagggcagcacctccggaggaggcagcggcggaggcagcggaggcggcggctccagcgaagtgcagctggtcgagagcggaggcggactggtgcaacccggaggaagcctgaggctgagctgtgccgccagcggcttcaacatcaaggacacatacattcactttgtgaggcaggctcctggaaagggcctggagtggatcggcagaatcgaccccgctaacgacaacaccctgtacgccagcaagttccagggcaaggccaccatctccgccgacacaagcaagaataccgcctacctgcagatgaactccctgagggccgaggataccgccgtgtactactgcggcaggggctatggctactacgtgtttgaccactggggccagggcacactggtgacagtgagctccgagcccaagagctccgacaagacacacacctgccctccttgccccccttgtcctccctgtggaggaggaagcagcggaggaggaagcggcggacagcccagagagcctcaagtgtataccctgcccccctccagggaagagatgaccaagaaccaggtgagcctgacatgcctggtcaaaggcttctaccccagcgatattgctgtggagtgggagagcaacggccagcccgagaacaactacaagaccacaccccccgtcctggatagcgatggcagcttcttcctgtacagcaagctgaccgtggacaagtccaggtggcagcagggcaacgtcttctcctgcagcgtgatgcacgaggctctgcataaccactacacacagaagtccctcagcctgagccctggatga

SEQ ID NO: 69 - protein, 399 aa, Artificial Sequence
  IAB22M gamma 1 EH5(M1)
METDTLLLWVLLLWVPGSTGDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSEPKSSDKTHTCPPCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 398 aa, Artificial Sequence
  IAB22M gamma 1 EH7(Minibody 1)
METDTLLLWVLLLWVPGSTGDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSELKTPLGDTTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - DNA, 1197 bp, Artificial Sequence
  IAB22M gamma 1 EH7(Minibody 1)
atggagacagacaccctcctcctgtgggtgctgctgctgtgggtgcccggatccaccggagacgtgcagatcacacagagccccagctccctgtccgctagcgtgggcgacagagtgaccatcacctgcaggaccagcaggagcatctcccagtacctcgcttggtaccagcagaagcctggcaaggtgcccaagctgctgatttacagcggatccaccctgcagagcggcgtgcctagcaggtttagcggcagcggatccggaacagacttcaccctgaccatcagcagcctgcagcctgaagatgtggccacctactactgtcagcagcacaacgaaaaccccctcaccttcggcggcggcacaaaggtggaaatcaagggcagcacctccggaggaggcagcggcggaggcagcggaggcggcggctccagcgaagtgcagctggtcgagagcggaggcggactggtgcaacccggaggaagcctgaggctgagctgtgccgccagcggcttcaacatcaaggacacatacattcactttgtgaggcaggctcctggaaagggcctggagtggatcggcagaatcgaccccgctaacgacaacaccctgtacgccagcaagttccagggcaaggccaccatctccgccgacacaagcaagaataccgcctacctgcagatgaactccctgagggccgaggataccgccgtgtactactgcggcaggggctatggctactacgtgtttgaccactggggccagggcacactggtgacagtgagctccgagctgaagacacctctgggcgacacaacacacacctgccccccttgtcctccctgtggaggaggaagcagcggaggaggaagcggcggacagcccagagagcctcaagtgtataccctgcccccctccagggaagagatgaccaagaaccaggtgagcctgacatgcctggtcaaaggcttctaccccagcgatattgctgtggagtgggagagcaacggccagcccgagaacaactacaagaccacaccccccgtcctggatagcgatggcagcttcttcctgtacagcaagctgaccgtggacaagtccaggtggcagcagggcaacgtcttctcctgcagcgtgatgcacgaggctctgcataaccactacacacagaagtccctcagcctgagccctggatga

SEQ ID NO: 72 - protein, 401 aa, Artificial Sequence
  IAB22M gamma 1 EH8(Minibody 1)
METDTLLLWVLLLWVPGSTGDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSELKTPLGDTTHTCPPCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - DNA, 1206 bp, Artificial Sequence
  IAB22M gamma 1 EH8(Minibody 1)
atggagacagacaccctcctcctgtgggtgctgctgctgtgggtgcccggatccaccggagacgtgcagatcacacagagccccagctccctgtccgctagcgtgggcgacagagtgaccatcacctgcaggaccagcaggagcatctcccagtacctcgcttggtaccagcagaagcctggcaaggtgcccaagctgctgatttacagcggatccaccctgcagagcggcgtgcctagcaggtttagcggcagcggatccggaacagacttcaccctgaccatcagcagcctgcagcctgaagatgtggccacctactactgtcagcagcacaacgaaaaccccctcaccttcggcggcggcacaaaggtggaaatcaagggcagcacctccggaggaggcagcggcggaggcagcggaggcggcggctccagcgaagtgcagctggtcgagagcggaggcggactggtgcaacccggaggaagcctgaggctgagctgtgccgccagcggcttcaacatcaaggacacatacattcactttgtgaggcaggctcctggaaagggcctggagtggatcggcagaatcgaccccgctaacgacaacaccctgtacgccagcaagttccagggcaaggccaccatctccgccgacacaagcaagaataccgcctacctgcagatgaactccctgagggccgaggataccgccgtgtactactgcggcaggggctatggctactacgtgtttgaccactggggccagggcacactggtgacagtgagctccgagctgaagacacctctgggcgacacaacacacacctgccctccttgccccccttgtcctccctgtggaggaggaagcagcggaggaggaagcggcggacagcccagagagcctcaagtgtataccctgcccccctccagggaagagatgaccaagaaccaggtgagcctgacatgcctggtcaaaggcttctaccccagcgatattgctgtggagtgggagagcaacggccagcccgagaacaactacaagaccacaccccccgtcctggatagcgatggcagcttcttcctgtacagcaagctgaccgtggacaagtccaggtggcagcagggcaacgtcttctcctgcagcgtgatgcacgaggctctgcataaccactacacacagaagtccctcagcctgagccctggatga

SEQ ID NO: 74 - protein, 391 aa, Artificial Sequence
  IAB22M gamma 2 EH2(Minibody 1)
METDTLLLWVLLLWVPGSTGDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSERKSCVECPPCPGGGSSGGGSGGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 75 - DNA, 1236 bp, Artificial Sequence
  IAB22M gamma 2 EH2(Minibody 1)
atggaaaccgacacactgctgctgtgggtgctgctgctgtgggtccctggctccaccggagacgtccagatcacacagagccccagcagcctgtccgccagcgtgggagacagggtgaccgacgtccagatcacacagagccccagcagcctgtccgccagcgtgggagacagggtgaccgacgtccagatcacacagagccccagcagcctgtccgccagcgtgggagacagggtgaccggcaaggtccccaaactgctgatctacagcggctccaccctgcagtccggcgtgcctagcaggttctccggcagcggatccggcaccgacttcaccctgaccatcagctccctgcagcctgaggacgtggctacctactactgccaacagcacaacgagaaccccctgacctttggaggcggcaccaaggtggaaatcaagggcagcaccagcggcggaggaagcggaggaggatccggaggaggcggaagctccgaggtgcagctggtggaaagcggcggcggactggtgcagcctggaggaagcctcagactgagctgtgccgccagcggattcaacatcaaagacacctacattcatttcgtgagacaggcccccggcaagggcctcgaatggatcggaaggatcgaccccgctaacgacaataccctgtacgcctccaagttccagggaaaggccaccatctccgccgatacctccaagaacaccgcctacctccagatgaactccctgagggccgaagataccgccgtctactactgtggcaggggctacggctactatgtgttcgatcactggggccaaggaaccctggtgaccgtgagcagcgaaaggaagagctgcgtggagtgtcctccttgtcccggcggcggctccagcggcggaggctccggcggccagcctagagaacctcaggtgtacaccctccccccctccagagaggagatgaccaagaaccaggtgtccctgacctgcctggtgaaaggcttctatcccagcgacatcgccgtggaatgggagtccaacggccagcccgagaacaactacaagaccacccctcccatgctggattccgacggcagctttttcctgtacagcaagctcaccgtggacaagagcagatggcagcagggcaacgtgttcagctgcagcgtgatgcacgaggctctgcataaccactacacccagaagagcctgtccctgtcccccggatga

SEQ ID NO: 76 - protein, 391 aa, Artificial Sequence
  IAB22M gamma 2 EH2(Minibody 1)(VH-K67R)
METDTLLLWVLLLWVPGSTGDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGRATISADTSKNTAYLQMNSLRAEDTAVYYCGRGYGYYVFDHWGQGTLVTVSSERKSCVECPPCPGGGSSGGGSGGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 77 - DNA, 1176 bp, Artificial Sequence
  IAB22M gamma 2 EH2(Minibody 1)
atggagaccgacaccctcctcctgtgggtgctgctgctgtgggtgcctggaagcaccggcgatgtgcagatcacccagagccctagcagcctgtccgcttccgtgggcgacagggtgaccatcacctgtaggacctccaggagcatctcccagtacctggcctggtaccagcagaagcccggcaaggtgcccaaactgctcatctactccggcagcacactccagagcggcgtccctagcagattcagcggaagcggcagcggcaccgacttcaccctgaccatcagctccctgcagcccgaggacgtggccacctattactgtcagcagcacaacgaaaaccccctgaccttcggcggcggcacaaaagtggagatcaagggcagcaccagcggaggcggatccggcggcggcagcggcggcggaggatccagcgaagtgcagctggtcgaaagcggaggcggactggtgcagcctggaggaagcctgagactcagctgcgccgcctccggattcaacatcaaggacacctacatccacttcgtgaggcaggctcccggcaaaggcctcgagtggattggaaggattgaccccgccaacgacaacaccctgtacgccagcaagttccaaggaagggccaccatctccgccgacaccagcaagaataccgcctacctgcagatgaactccctgagggctgaggacaccgccgtgtactactgcggcagaggctacggctactacgtgttcgaccactggggacagggcacactggtgacagtgagcagcgagaggaaaagctgcgtggagtgccccccctgccctggcggcggcagctccggcggaggaagcggaggacaacccagggagccccaggtgtacacactcccccctagcagggaagagatgaccaagaaccaggtgtccctgacctgcctcgtgaagggattctaccccagcgacattgccgtcgagtgggagagcaacggccagcctgagaacaactacaagacaaccccccctatgctcgatagcgatggctccttcttcctgtactccaagctcaccgtcgacaagagcaggtggcagcagggcaacgtcttctcctgtagcgtgatgcacgaggctctgcacaaccactacacccagaagagcctgagcctgagccccggctga

SEQ ID NO: 78 - protein, 118 aa, Artificial Sequence
  IAB22M variable heavy (VH-K67R) sequence
EVQLVESGGGLVQPGGSLRLSCAASGFNIKDTYIHFVRQAPGKGLEWIGRIDPANDNTLYASKFQGRATISADTSKNTAYLQMNSLRAEDTAVYYCGRGYGYYVFDHWGQGTLVTVSS

SEQ ID NO: 79 - protein, 376 aa, Artificial Sequence
  IAB22 (CD8)
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSEVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 81 - protein, 235 aa, Homo sapiens; identical to SEQ ID NO: 80

SEQ ID NO: 82 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPVTLGLLVAGVLVLLVSLGVAMHLCCRRRRARLRFMKQFYK

SEQ ID NO: 83 - protein, 56 aa, Artificial Sequence
  MAX16H5
EVQLQQSGTVLARPGASVQMSCKASGYSFANYWMHWVKQRPGQGLQWIGALYPGNV

SEQ ID NO: 84 - protein, 56 aa, Artificial Sequence
  IAB41
QVQLVQSGAEVKKPGASVKVSCKASGYSFANYWMHWVRQAPGQRLEWIGALYPGNV

SEQ ID NO: 85 - protein, 58 aa, Artificial Sequence
  MAX16H5
QIVLTQSPAIMSASPGEKVAMTCSARSSVSYLYWYQQKPGSSPRLLIYDTSNLASGVP

SEQ ID NO: 86 - protein, 58 aa, Artificial Sequence
  IAB41
EIVLTQSPATLSLSPGERATLSCSARSSVSYLYWYQQKPGQAPRLLIYDTSNLASGIP

SEQ ID NO: 87 - protein, 20 aa, Artificial Sequence
  signal peptide
METDTLLLWVLLLWVPGSTG

SEQ ID NO: 88 - protein, 377 aa, Artificial Sequence
  minibody
EIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKGSTSGGGSGGGSGGGGSSQVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 380 aa, Artificial Sequence
  minibody
EIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKGSTSGGGSGGGSGGGGSSQVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSEPKSSDKTHTCPPCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 377 aa, Artificial Sequence
  minibody
QVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSGSTSGGGSGGGSGGGGSSEIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKEPKSSDKTHTCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 380 aa, Artificial Sequence
  minibody
QVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSGSTSGGGSGGGSGGGGSSEIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKEPKSSDKTHTCPPCPPCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 234 aa, Artificial Sequence
  Cys-Diabody
EIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKSGGGGQVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSGGC

SEQ ID NO: 93 - protein, 237 aa, Artificial Sequence
  cys-diabody
EIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKSGGGGQVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSGGCPPC

SEQ ID NO: 94 - protein, 240 aa, Artificial Sequence
  cys-diabody
EIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKSGGGGQVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSGGCPPCPPC

SEQ ID NO: 95 - protein, 243 aa, Artificial Sequence
  cys-diabody
EIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKSGGGGQVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSGGCPPCPPCPPC

SEQ ID NO: 96 - protein, 234 aa, Artificial Sequence
  cys-diabody
QVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSSGGGGEIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKGGC

SEQ ID NO: 97 - protein, 237 aa, Artificial Sequence
  cys-diabody
QVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSSGGGGEIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKGGCPPC

SEQ ID NO: 98 - protein, 240 aa, Artificial Sequence
  cys-diabody
QVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSSGGGGEIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKGGCPPCPPC

SEQ ID NO: 99 - protein, 243 aa, Artificial Sequence
  cys-diabody
QVQLVQSGAEVKKPGASVKVSCKASGYSFANYWMHWVRQAPGQRLEWIGALYPGNVDTTYNQKFQGRVTITRDTSASTAYMELSSLRSEDTAVYYCTRMGTTLEAPLDYWGQGTLVTVSSSGGGGEIVLTQSPATLSLSPGERATLSCSARSSVSYLYWYQQKPGQAPRLLIYDTSNLASGIPDRFSGSGSGTDFTLTISRLEPEDAAVYYCQQWSDYPLTFGGGTKLEIKGGCPPCPPCPPC

SEQ ID NO: 100 - protein, 433 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 101 - protein, 106 aa, Mus musculus [sequence omitted]

SEQ ID NO: 102 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTKVEIK

SEQ ID NO: 103 - protein, 106 aa, Artificial Sequence
  Antigen binding construct or subpart thereof
EIVLTQSPATLSLSPGERATLSCSASSSVSYMNWYQQKPGQAPRLLIYDTSKLASGVPAHFRGSGSGTDYTLTISSLEPEDFAVYYCQQWSSNPFTFGQGTKVEIK

SEQ ID NO: 104 - protein, 119 aa, Mus musculus [sequence omitted]

SEQ ID NO: 105 - protein, 115 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDEYFQHWGQGTLVTVSS

SEQ ID NO: 106 - protein, 119 aa, Artificial Sequence
  Antigen binding construct or subpart thereof
QVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSS

SEQ ID NO: 107 - protein, 394 aa, Artificial Sequence
  Antigen binding construct or subpart thereof
METDTLLLWVLLLWVPGSTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - DNA, 1185 bp, Artificial Sequence
  Antigen binding construct or subpart thereof
atggaaaccgacactctgctgctgtgggtcctgctgctgtgggtgcccggatcaactggacagatcgtgctgactcagtctcccgctatcatgtctgcctcacctggcgaaaaagtgacaatgacctgttccgcctcttcttccgtgtcttacatgaattggtaccagcagaaatctgggactagtcctaaacggtggatctacgatactagcaaactggcttctggcgtgcctgctcatttccgtggttctggctctggaacctcttactctctgaccatctctggcatggaggccgaggatgccgccacctactactgccagcagtggagttcaaaccctttcacattcggctccggcacaaaactggagatcaacggctctactagtggtggaggatctggtggtggatctggagggggcggatcatctcaggtccagctgcagcagtctggtgctgaactggcacgtcctggtgcctccgtgaaaatgtcttgtaaggcctctggttacacctttacccggtacactatgcattgggtcaaacagcgccctgggcagggactggaatggattggctacatcaacccttctcgtggctacacaaactacaatcagaaattcaaggacaaggccaccctgacaaccgacaaatcttcttcaaccgcctacatgcagctgtcatccctgacctctgaggatagtgctgtgtactactgtgctcggtactacgacgatcactactgtctggactactggggacagggaacaacactgactgtgtcctccgaacccaaatcctgtgacaaaacccacacctgtccaccttgtggcggtggatcatctggcggagggagtggagggcagcctagggagcctcaggtctacacactgccaccttctcgggacgaactgacaaaaaaccaggtgtccctgacatgtctggtgaagggcttctacccttccgatatcgctgtggagtgggagtcaaatggccagcccgaaaacaactacaaaaccaccccacctgtgctggattccgatggctctttcttcctgtactctaaactgaccgtggataagagtcgatggcagcagggaaacgtgttctcttgctccgtgatgcacgaggccctgcataatcattacacccagaaatcactgtctctgtcacccggcaaatga

SEQ ID NO: 109 - protein, 394 aa, Artificial Sequence
  Antigen binding construct or subpart thereof
METDTLLLWVLLLWVPGSTGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGSTSGGGSGGGSGGGGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 1185 bp, Artificial Sequence
  Antigen binding construct or subpart thereof
atggaaaccgacactctgctgctgtgggtcctgctgctgtgggtgcccggatcaactggtgacatccagatgacacagtctccctcttctctgtccgcctctgtgggcgatcgagtgacaatcacctgtagcgcttcatcctccgtgtcttacatgaattggtaccagcagacccctggcaaagctcctaaacgatggatctacgacacctccaaactggcttccggcgtgccttcacgattttctggttctggttctgggaccgactacacctttaccatctcatcactgcagcctgaggatatcgccacatactactgtcagcagtggtctagcaaccctttcacattcgggcagggcacaaaactgcagatcaccggctcaacctctggcggtggctctggcggcggtagtggtggtggtggttctagtcaggtccagctggtccagtctggtggaggagtggtccagcccgggagatcactgaggctgtcctgtaaggctagtggctacacttttacacggtacaccatgcattgggtgaggcaggcacctgggaaaggcctggaatggatcggatacatcaaccctagtaggggatacacaaactacaatcagaaagtcaaggaccggttcacaatctctagggacaactctaaaaacaccgcttttctgcagatggactcactgaggcctgaggacactggagtgtacttttgtgctcggtactacgatgatcattactgcctggattactggggacaggggacacctgtcactgtctcttccgaacccaaatcttgtgacaaaacccacacatgccctccatgtggtggcggatcctctggtggcggttctggggggcagcctagggaacctcaggtgtacacactgccaccttctcgtgacgaactgaccaaaaaccaggtgtcactgacctgtctggtcaagggcttttacccttccgacattgctgtggagtgggagtcaaatggccagcctgaaaacaactacaaaaccacaccccccgtcctggattccgatggctctttcttcctgtactctaaactgaccgtcgacaaatctcgatggcagcagggaaacgtgttctcttgttccgtcatgcacgaggctctgcacaatcactacacacagaaatcactgagcctgagccctggaaaatga

SEQ ID NO: 111 - protein, 394 aa, Artificial Sequence; identical to SEQ ID NO: 109
  Antigen binding construct or subpart thereof

SEQ ID NO: 112 - DNA, 1185 bp, Artificial Sequence; identical to SEQ ID NO: 110
  Antigen binding construct or subpart thereof

SEQ ID NO: 113 - protein, 253 aa, Artificial Sequence
  Antigen binding construct or subpart thereof
METDTLLLWVLLLWVPGSTGEIVLTQSPATLSLSPGERATLSCSASSSVSYMNWYQQKPGQAPRLLIYDTSKLASGVPAHFRGSGSGTDFTLTISSLEPEDFAVYYCQQWSSNPFTFGQGTKVEIKSGGGGQVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSSGGC

SEQ ID NO: 114 - DNA, 759 bp, Artificial Sequence
  Antigen binding construct or subpart thereof
atggaaaccgacactctgctgctgtgggtcctgctgctgtgggtgcccggatcaactggagaaatcgtgctgactcagtcccctgctacactgtctctgtcacctggcgaacgagcaacactgtcctgttctgcctcttcttctgtctcatacatgaactggtaccagcagaaacctggacaggctcctagactgctgatctacgacacctctaaactggcatctggcgtgcccgctcattttcgtggctctggatctggaaccgactttaccctgaccatctcttccctggaacctgaggattttgccgtgtactactgccagcagtggtctagtaaccctttcacttttggccagggcactaaagtggagatcaaatccggtggtggcggacaggtccagctggtccagagtggagctgaggtgaaaaaacccggcgcttccgtcaaagtctcctgtaaggctagcggatacacattcacacgctacaccatgcattgggtccggcaggctcccggacagggcctggaatggatgggatacatcaacccttctcggggctacacaaactacaaccagaaattcaaggatcgagtgaccatgacaaccgacactagcatctctaccgcctacatggaactgagccggctgagatccgatgataccgctgtctactactgtgctcggtactacgatgatcattactgcctggattactgggggcagggcacactggtgactgtgagttccggaggatgt

SEQ ID NO: 115 - protein, 435 aa, Artificial Sequence
  Antigen binding construct or subpart thereof
METDTLLLWVLLLWVPGSTGMDWVWTLLFLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSMDFQVQIFSFLLISASVIISRGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - DNA, 1308 bp, Artificial Sequence
  Antigen binding construct or subpart thereof
atggaaaccgacaccctgctgctgtgggtgctgctgctctgggtcccaggctccaccggtatggattgggtgtggaccttgctattcctgttgtcagtaactgcaggtgtccactcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggctccacatccggcggaggctctggcggtggatctggcggaggcggctcatccatggattttcaagtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaacgagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaagtga

SEQ ID NO: 117 - protein, 253 aa, Artificial Sequence
  Antigen binding construct or subpart thereof
METDTLLLWVLLLWVPGSTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINSGGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGC

SEQ ID NO: 118 - DNA, 762 bp, Artificial Sequence
  Antigen binding construct or subpart thereof
atggaaaccgacaccctgctgctgtgggtcctgctcctctgggtgccaggctctaccggccaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaactctggtggaggcgggcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggcggatgctga

SEQ ID NO: 119 - protein, 253 aa, Artificial Sequence
  Antigen binding construct or subpart thereof
METDTLLLWVLLLWVPGSTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGC

SEQ ID NO: 120 - DNA, 759 bp, Artificial Sequence
  Antigen binding construct or subpart thereof
atggaaaccgacaccctgctgctgtgggtgctgctgctctgggtcccaggctccaccggtcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaagtggtggaggaggccaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaacggcggctgc

SEQ ID NO: 121 - protein, 256 aa, Artificial Sequence
  Antigen binding construct or subpart thereof
METDTLLLWVLLLWVPGSTGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGSGGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGC

SEQ ID NO: 122 - DNA, 768 bp, Artificial Sequence
  Antigen binding construct or subpart thereof
atggaaaccgacaccctgctgctgtgggtgctgctgctctgggtcccaggctccaccggtcaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaacggcggagggagtggcggaggcggccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggcggctgc

SEQ ID NO: 123 - protein, 256 aa, Artificial Sequence
  Antigen binding construct or subpart thereof
METDTLLLWVLLLWVPGSTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGSGGGGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGC

SEQ ID NO: 124 - DNA, 768 bp, Artificial Sequence
  Antigen binding construct or subpart thereof
atggaaaccgacaccctgctgctgtgggtgctgctgctctgggtcccaggctccaccggtcaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggcggagggagtggcggaggcggccaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaacggcggctgc

SEQ ID NO: 125 - protein, 394 aa, Artificial Sequence
  Antigen binding construct or subpart thereof
METDTLLLWVLLLWVPGSTGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGSTSGGGSGGGSGGGGSSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - DNA, 1185 bp, Artificial Sequence
  Antigen binding construct or subpart thereof
atggaaaccgacaccctgctgctgtgggtgctgctgctctgggtcccaggctccaccggtcaggtccagctggtccagtctggtggaggagtggtccagcccgggagatcactgaggctgtcctgtaaggctagtggctacacttttacacggtacaccatgcattgggtgaggcaggcacctgggaaaggcctggaatggatcggatacatcaaccctagtaggggatacacaaactacaatcagaaagtcaaggaccggttcacaatctctagggacaactctaaaaacaccgcttttctgcagatggactcactgaggcctgaggacactggagtgtacttttgtgctcggtactacgatgatcattactgcctggattactggggacaggggacacctgtcactgtctcttccggctccacatccggcggaggctctggcggtggatctggcggaggcggctcatccgacatccagatgacacagtctccctcttctctgtccgcctctgtgggcgatcgagtgacaatcacctgtagcgcttcatcctccgtgtcttacatgaattggtaccagcagacccctggcaaagctcctaaacgatggatctacgacacctccaaactggcttccggcgtgccttcacgattttctggttctggttctgggaccgactacacctttaccatctcatcactgcagcctgaggatatcgccacatactactgtcagcagtggtctagcaaccctttcacattcgggcagggcacaaaactgcagatcaccgagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaagtga

SEQ ID NO: 127 - protein, 253 aa, Artificial Sequence
  Antigen binding construct or subpart thereof
METDTLLLWVLLLWVPGSTGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITSGGGGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGC

SEQ ID NO: 128 - DNA, 759 bp, Artificial Sequence
  Antigen binding construct or subpart thereof
atggaaaccgacaccctgctgctgtgggtgctgctgctctgggtcccaggctccaccggtgacatccagatgacacagtctccctcttctctgtccgcctctgtgggcgatcgagtgacaatcacctgtagcgcttcatcctccgtgtcttacatgaattggtaccagcagacccctggcaaagctcctaaacgatggatctacgacacctccaaactggcttccggcgtgccttcacgattttctggttctggttctgggaccgactacacctttaccatctcatcactgcagcctgaggatatcgccacatactactgtcagcagtggtctagcaaccctttcacattcgggcagggcacaaaactgcagatcaccagtggtggaggaggccaggtccagctggtccagtctggtggaggagtggtccagcccgggagatcactgaggctgtcctgtaaggctagtggctacacttttacacggtacaccatgcattgggtgaggcaggcacctgggaaaggcctggaatggatcggatacatcaaccctagtaggggatacacaaactacaatcagaaagtcaaggaccggttcacaatctctagggacaactctaaaaacaccgcttttctgcagatggactcactgaggcctgaggacactggagtgtacttttgtgctcggtactacgatgatcattactgcctggattactggggacaggggacacctgtcactgtctcttccggcggctgc

SEQ ID NO: 129 - protein, 253 aa, Artificial Sequence
  Antigen binding construct or subpart thereof
METDTLLLWVLLLWVPGSTGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSSGGGGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGC

SEQ ID NO: 130 - DNA, 759 bp, Artificial Sequence
  Antigen binding construct or subpart thereof
atggaaaccgacaccctgctgctgtgggtgctgctgctctgggtcccaggctccaccggtcaggtccagctggtccagtctggtggaggagtggtccagcccgggagatcactgaggctgtcctgtaaggctagtggctacacttttacacggtacaccatgcattgggtgaggcaggcacctgggaaaggcctggaatggatcggatacatcaaccctagtaggggatacacaaactacaatcagaaagtcaaggaccggttcacaatctctagggacaactctaaaaacaccgcttttctgcagatggactcactgaggcctgaggacactggagtgtacttttgtgctcggtactacgatgatcattactgcctggattactggggacaggggacacctgtcactgtctcttccagtggtggaggaggcgacatccagatgacacagtctccctcttctctgtccgcctctgtgggcgatcgagtgacaatcacctgtagcgcttcatcctccgtgtcttacatgaattggtaccagcagacccctggcaaagctcctaaacgatggatctacgacacctccaaactggcttccggcgtgccttcacgattttctggttctggttctgggaccgactacacctttaccatctcatcactgcagcctgaggatatcgccacatactactgtcagcagtggtctagcaaccctttcacattcgggcagggcacaaaactgcagatcaccggcggctgc

SEQ ID NO: 131 - protein, 256 aa, Artificial Sequence
  Antigen binding construct or subpart thereof
METDTLLLWVLLLWVPGSTGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGSGGGGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGC

SEQ ID NO: 132 - DNA, 768 bp, Artificial Sequence
  Antigen binding construct or subpart thereof
atggaaaccgacaccctgctgctgtgggtgctgctgctctgggtcccaggctccaccggtgacatccagatgacacagtctccctcttctctgtccgcctctgtgggcgatcgagtgacaatcacctgtagcgcttcatcctccgtgtcttacatgaattggtaccagcagacccctggcaaagctcctaaacgatggatctacgacacctccaaactggcttccggcgtgccttcacgattttctggttctggttctgggaccgactacacctttaccatctcatcactgcagcctgaggatatcgccacatactactgtcagcagtggtctagcaaccctttcacattcgggcagggcacaaaactgcagatcaccggcggagggagtggcggaggcggccaggtccagctggtccagtctggtggaggagtggtccagcccgggagatcactgaggctgtcctgtaaggctagtggctacacttttacacggtacaccatgcattgggtgaggcaggcacctgggaaaggcctggaatggatcggatacatcaaccctagtaggggatacacaaactacaatcagaaagtcaaggaccggttcacaatctctagggacaactctaaaaacaccgcttttctgcagatggactcactgaggcctgaggacactggagtgtacttttgtgctcggtactacgatgatcattactgcctggattactggggacaggggacacctgtcactgtctcttccggcggctgc

SEQ ID NO: 133 - protein, 256 aa, Artificial Sequence
  Antigen binding construct or subpart thereof
METDTLLLWVLLLWVPGSTGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGSGGGGDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGC

SEQ ID NO: 134 - DNA, 768 bp, Artificial Sequence
  Antigen binding construct or subpart thereof
atggaaaccgacaccctgctgctgtgggtgctgctgctctgggtcccaggctccaccggtcaggtccagctggtccagtctggtggaggagtggtccagcccgggagatcactgaggctgtcctgtaaggctagtggctacacttttacacggtacaccatgcattgggtgaggcaggcacctgggaaaggcctggaatggatcggatacatcaaccctagtaggggatacacaaactacaatcagaaagtcaaggaccggttcacaatctctagggacaactctaaaaacaccgcttttctgcagatggactcactgaggcctgaggacactggagtgtacttttgtgctcggtactacgatgatcattactgcctggattactggggacaggggacacctgtcactgtctcttccggcggagggagtggcggaggcggcgacatccagatgacacagtctccctcttctctgtccgcctctgtgggcgatcgagtgacaatcacctgtagcgcttcatcctccgtgtcttacatgaattggtaccagcagacccctggcaaagctcctaaacgatggatctacgacacctccaaactggcttccggcgtgccttcacgattttctggttctggttctgggaccgactacacctttaccatctcatcactgcagcctgaggatatcgccacatactactgtcagcagtggtctagcaaccctttcacattcgggcagggcacaaaactgcagatcaccggcggctgc

SEQ ID NO: 135 - protein, 394 aa, Artificial Sequence
  Antigen binding construct or subpart thereof
METDTLLLWVLLLWVPGSTGQVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSSGSTSGGGSGGGSGGGGSSEIVLTQSPATLSLSPGERATLSCSASSSVSYMNWYQQKPGQAPRLLIYDTSKLASGVPAHFRGSGSGTDFTLTISSLEPEDFAVYYCQQWSSNPFTFGQGTKVEIKEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - DNA, 1185 bp, Artificial Sequence
  Antigen binding construct or subpart thereof
atggaaaccgacaccctgctgctgtgggtgctgctgctctgggtcccaggctccaccggtcaggtccagctggtccagagtggagctgaggtgaaaaaacccggcgcttccgtcaaagtctcctgtaaggctagcggatacacctttactcgctacaccatgcattgggtccggcaggctcccggacagggcctggaatggatgggatacatcaacccttctcggggctacacaaactacaatcagaaattcaaggatcgagtgaccatgacaaccgacacttcaatctctaccgcttacatggaactgtctcggctgaggagtgacgataccgctgtctactactgtgctcggtactacgacgaccattactgcctggattactgggggcagggcacactggtgactgtgtctagcggctccacatccggcggaggctctggcggtggatctggcggaggcggctcatccgaaatcgtgctgactcagtcccctgctacactgtctctgtcacctggcgaacgagcaacactgtcctgttctgcctcttcttctgtctcatacatgaactggtaccagcagaaacctggacaggctcctagactgctgatctacgacacctctaaactggcatctggcgtgcccgctcattttcgtggctctggatctggaaccgactttaccctgaccatctcttccctggaacctgaggattttgccgtgtactactgccagcagtggtctagtaaccctttcacttttggccagggcactaaagtggagatcaaggagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaagtga

SEQ ID NO: 137 - protein, 253 aa, Artificial Sequence; identical to SEQ ID NO: 113
  Antigen binding construct or subpart thereof

SEQ ID NO: 138 - DNA, 759 bp, Artificial Sequence
  Antigen binding construct or subpart thereof
atggaaaccgacaccctgctgctgtgggtgctgctgctctgggtcccaggctccaccggtgaaatcgtgctgactcagtcccctgctacactgtctctgtcacctggcgaacgagcaacactgtcctgttctgcctcttcttctgtctcatacatgaactggtaccagcagaaacctggacaggctcctagactgctgatctacgacacctctaaactggcatctggcgtgcccgctcattttcgtggctctggatctggaaccgactttaccctgaccatctcttccctggaacctgaggattttgccgtgtactactgccagcagtggtctagtaaccctttcacttttggccagggcactaaagtggagatcaagagtggtggaggaggccaggtccagctggtccagagtggagctgaggtgaaaaaacccggcgcttccgtcaaagtctcctgtaaggctagcggatacacctttactcgctacaccatgcattgggtccggcaggctcccggacagggcctggaatggatgggatacatcaacccttctcggggctacacaaactacaatcagaaattcaaggatcgagtgaccatgacaaccgacacttcaatctctaccgcttacatggaactgtctcggctgaggagtgacgataccgctgtctactactgtgctcggtactacgacgaccattactgcctggattactgggggcagggcacactggtgactgtgtctagcggcggctgc

SEQ ID NO: 139 - protein, 253 aa, Artificial Sequence
  Antigen binding construct or subpart thereof
METDTLLLWVLLLWVPGSTGQVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSSSGGGGEIVLTQSPATLSLSPGERATLSCSASSSVSYMNWYQQKPGQAPRLLIYDTSKLASGVPAHFRGSGSGTDFTLTISSLEPEDFAVYYCQQWSSNPFTFGQGTKVEIKGGC

SEQ ID NO: 140 - DNA, 759 bp, Artificial Sequence
  Antigen binding construct or subpart thereof
atggaaaccgacaccctgctgctgtgggtgctgctgctctgggtcccaggctccaccggtcaggtccagctggtccagagtggagctgaggtgaaaaaacccggcgcttccgtcaaagtctcctgtaaggctagcggatacacctttactcgctacaccatgcattgggtccggcaggctcccggacagggcctggaatggatgggatacatcaacccttctcggggctacacaaactacaatcagaaattcaaggatcgagtgaccatgacaaccgacacttcaatctctaccgcttacatggaactgtctcggctgaggagtgacgataccgctgtctactactgtgctcggtactacgacgaccattactgcctggattactgggggcagggcacactggtgactgtgtctagcagtggtggaggaggcgaaatcgtgctgactcagtcccctgctacactgtctctgtcacctggcgaacgagcaacactgtcctgttctgcctcttcttctgtctcatacatgaactggtaccagcagaaacctggacaggctcctagactgctgatctacgacacctctaaactggcatctggcgtgcccgctcattttcgtggctctggatctggaaccgactttaccctgaccatctcttccctggaacctgaggattttgccgtgtactactgccagcagtggtctagtaaccctttcacttttggccagggcactaaagtggagatcaagggcggctgc

SEQ ID NO: 141 - protein, 236 aa, Artificial Sequence
  Antigen binding construct or subpart thereof
METDTLLLWVLLLWVPGSTGEIVLTQSPATLSLSPGERATQAPRLLIYDTSKLASGVPAHFRGSGSGTDFTLTISSLEPEDFAVYYCQQWSSNPFTFGQGTKVEIKGGGSGGGGQVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSSGGC

SEQ ID NO: 142 - DNA, 768 bp, Artificial Sequence
  Antigen binding construct or subpart thereof
atggaaaccgacaccctgctgctgtgggtgctgctgctctgggtcccaggctccaccggtgaaatcgtgctgactcagtcccctgctacactgtctctgtcacctggcgaacgagcaacactgtcctgttctgcctcttcttctgtctcatacatgaactggtaccagcagaaacctggacaggctcctagactgctgatctacgacacctctaaactggcatctggcgtgcccgctcattttcgtggctctggatctggaaccgactttaccctgaccatctcttccctggaacctgaggattttgccgtgtactactgccagcagtggtctagtaaccctttcacttttggccagggcactaaagtggagatcaagggcggagggagtggcggaggcggccaggtccagctggtccagagtggagctgaggtgaaaaaacccggcgcttccgtcaaagtctcctgtaaggctagcggatacacctttactcgctacaccatgcattgggtccggcaggctcccggacagggcctggaatggatgggatacatcaacccttctcggggctacacaaactacaatcagaaattcaaggatcgagtgaccatgacaaccgacacttcaatctctaccgcttacatggaactgtctcggctgaggagtgacgataccgctgtctactactgtgctcggtactacgacgaccattactgcctggattactgggggcagggcacactggtgactgtgtctagcggcggctgc

SEQ ID NO: 143 - protein, 256 aa, Artificial Sequence
  Antigen binding construct or subpart thereof
METDTLLLWVLLLWVPGSTGQVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSSGGGSGGGGEIVLTQSPATLSLSPGERATLSCSASSSVSYMNWYQQKPGQAPRLLIYDTSKLASGVPAHFRGSGSGTDFTLTISSLEPEDFAVYYCQQWSSNPFTFGQGTKVEIKGGC

SEQ ID NO: 144 - DNA, 768 bp, Artificial Sequence
  Antigen binding construct or subpart thereof
atggaaaccgacaccctgctgctgtgggtgctgctgctctgggtcccaggctccaccggtcaggtccagctggtccagagtggagctgaggtgaaaaaacccggcgcttccgtcaaagtctcctgtaaggctagcggatacacctttactcgctacaccatgcattgggtccggcaggctcccggacagggcctggaatggatgggatacatcaacccttctcggggctacacaaactacaatcagaaattcaaggatcgagtgaccatgacaaccgacacttcaatctctaccgcttacatggaactgtctcggctgaggagtgacgataccgctgtctactactgtgctcggtactacgacgaccattactgcctggattactgggggcagggcacactggtgactgtgtctagcggcggagggagtggcggaggcggcgaaatcgtgctgactcagtcccctgctacactgtctctgtcacctggcgaacgagcaacactgtcctgttctgcctcttcttctgtctcatacatgaactggtaccagcagaaacctggacaggctcctagactgctgatctacgacacctctaaactggcatctggcgtgcccgctcattttcgtggctctggatctggaaccgactttaccctgaccatctcttccctggaacctgaggattttgccgtgtactactgccagcagtggtctagtaaccctttcacttttggccagggcactaaagtggagatcaagggcggctgc

SEQ ID NO: 145 - protein, 389 aa, Artificial Sequence
  Antigen binding construct or subpart thereof
METDTLLLWVLLLWVPGSTGDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGSTSGGGSGGGSGGGGSSDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - DNA, 1170 bp, Artificial Sequence
  Antigen binding construct or subpart thereof
atggaaaccgacaccctgctgctgtgggtgctgctgctctgggtcccaggctccaccggtgacatccagctgactcagcccaactctgtgtctacgtctctaggaagcacagtcaagctgtcttgcacactcagctctggtaacatagaaaacaactatgtgcactggtaccagctatatgagggaagatctcccaccactatgatttatgatgatgataagagaccggatggtgtccctgacaggttctctggctccattgacaggtcttccaactcagccttcctgacaatccataatgtggcaattgaagatgaagctatctacttctgtcattcttatgttagtagttttaatgttttcggcggtggaacaaagctcactgtccttggctccacatccggcggaggctctggcggtggatctggcggaggcggctcatccgacatccagctgactcagcccaactctgtgtctacgtctctaggaagcacagtcaagctgtcttgcacactcagctctggtaacatagaaaacaactatgtgcactggtaccagctatatgagggaagatctcccaccactatgatttatgatgatgataagagaccggatggtgtccctgacaggttctctggctccattgacaggtcttccaactcagccttcctgacaatccataatgtggcaattgaagatgaagctatctacttctgtcattcttatgttagtagttttaatgttttcggcggtggaacaaagctcactgtccttgagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaagtga

SEQ ID NO: 147 - protein, 389 aa, Artificial Sequence; identical to SEQ ID NO: 145
  Antigen binding construct or subpart thereof

SEQ ID NO: 148 - DNA, 1170 bp, Artificial Sequence; identical to SEQ ID NO: 146
  Antigen binding construct or subpart thereof

SEQ ID NO: 149 - protein, 248 aa, Artificial Sequence
  Antigen binding construct or subpart thereof
METDTLLLWVLLLWVPGSTGDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLSGGGGDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGGC

SEQ ID NO: 150 - DNA, 744 bp, Artificial Sequence
  Antigen binding construct or subpart thereof
atggaaaccgacaccctgctgctgtgggtgctgctgctctgggtcccaggctccaccggtgacatccagctgactcagcccaactctgtgtctacgtctctaggaagcacagtcaagctgtcttgcacactcagctctggtaacatagaaaacaactatgtgcactggtaccagctatatgagggaagatctcccaccactatgatttatgatgatgataagagaccggatggtgtccctgacaggttctctggctccattgacaggtcttccaactcagccttcctgacaatccataatgtggcaattgaagatgaagctatctacttctgtcattcttatgttagtagttttaatgttttcggcggtggaacaaagctcactgtccttagtggtggaggaggcgacatccagctgactcagcccaactctgtgtctacgtctctaggaagcacagtcaagctgtcttgcacactcagctctggtaacatagaaaacaactatgtgcactggtaccagctatatgagggaagatctcccaccactatgatttatgatgatgataagagaccggatggtgtccctgacaggttctctggctccattgacaggtcttccaactcagccttcctgacaatccataatgtggcaattgaagatgaagctatctacttctgtcattcttatgttagtagttttaatgttttcggcggtggaacaaagctcactgtccttggcggctgc

SEQ ID NO: 151 - protein, 248 aa, Artificial Sequence; identical to SEQ ID NO: 149
  Antigen binding construct or subpart thereof

SEQ ID NO: 152 - DNA, 744 bp, Artificial Sequence; identical to SEQ ID NO: 150
  Antigen binding construct or subpart thereof

SEQ ID NO: 153 - protein, 251 aa, Artificial Sequence
  Antigen binding construct or subpart thereof
METDTLLLWVLLLWVPGSTGDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGGGSGGGGDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGGC

SEQ ID NO: 154 - DNA, 753 bp, Artificial Sequence
  Antigen binding construct or subpart thereof
atggaaaccgacaccctgctgctgtgggtgctgctgctctgggtcccaggctccaccggtgacatccagctgactcagcccaactctgtgtctacgtctctaggaagcacagtcaagctgtcttgcacactcagctctggtaacatagaaaacaactatgtgcactggtaccagctatatgagggaagatctcccaccactatgatttatgatgatgataagagaccggatggtgtccctgacaggttctctggctccattgacaggtcttccaactcagccttcctgacaatccataatgtggcaattgaagatgaagctatctacttctgtcattcttatgttagtagttttaatgttttcggcggtggaacaaagctcactgtccttggcggagggagtggcggaggcggcgacatccagctgactcagcccaactctgtgtctacgtctctaggaagcacagtcaagctgtcttgcacactcagctctggtaacatagaaaacaactatgtgcactggtaccagctatatgagggaagatctcccaccactatgatttatgatgatgataagagaccggatggtgtccctgacaggttctctggctccattgacaggtcttccaactcagccttcctgacaatccataatgtggcaattgaagatgaagctatctacttctgtcattcttatgttagtagttttaatgttttcggcggtggaacaaagctcactgtccttggcggctgc

SEQ ID NO: 155 - protein, 251 aa, Artificial Sequence; identical to SEQ ID NO: 153
  Antigen binding construct or subpart thereof

SEQ ID NO: 156 - DNA, 753 bp, Artificial Sequence; identical to SEQ ID NO: 154
  Antigen binding construct or subpart thereof

SEQ ID NO: 157 - protein, 395 aa, Artificial Sequence
  Antigen binding construct or subpart thereof
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGSTSGGGSGGGSGGGGSSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - DNA, 1188 bp, Artificial Sequence
  Antigen binding construct or subpart thereof
atggaaaccgacaccctgctgctgtgggtgctgctgctctgggtcccaggctccaccggtgaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgctgacctttggcggcggcaccaaagtggaaattaaaggctccacatccggcggaggctctggcggtggatctggcggaggcggctcatcccaggtgcagctggtggaaagcggcggcggcgtggtgcagccgggccgcagcctgcgcctgagctgcgcggcgagcggctttaaatttagcggctatggcatgcattgggtgcgccaggcgccgggcaaaggcctggaatgggtggcggtgatttggtatgatggcagcaaaaaatattatgtggatagcgtgaaaggccgctttaccattagccgcgataacagcaaaaacaccctgtatctgcagatgaacagcctgcgcgcggaagataccgcggtgtattattgcgcgcgccagatgggctattggcattttgatctgtggggccgcggcaccctggtgaccgtgagcagcgagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaagtga

SEQ ID NO: 159 - protein, 395 aa, Artificial Sequence
  Antigen binding construct or subpart thereof
METDTLLLWVLLLWVPGSTGQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGSTSGGGSGGGSGGGGSSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 1188 bp, Artificial Sequence
  Antigen binding construct or subpart thereof
atggaaaccgacaccctgctgctgtgggtgctgctgctctgggtcccaggctccaccggtcaggtgcagctggtggaaagcggcggcggcgtggtgcagccgggccgcagcctgcgcctgagctgcgcggcgagcggctttaaatttagcggctatggcatgcattgggtgcgccaggcgccgggcaaaggcctggaatgggtggcggtgatttggtatgatggcagcaaaaaatattatgtggatagcgtgaaaggccgctttaccattagccgcgataacagcaaaaacaccctgtatctgcagatgaacagcctgcgcgcggaagataccgcggtgtattattgcgcgcgccagatgggctattggcattttgatctgtggggccgcggcaccctggtgaccgtgagcagcggctccacatccggcggaggctctggcggtggatctggcggaggcggctcatccgaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgctgacctttggcggcggcaccaaagtggaaattaaagagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaagtga

SEQ ID NO: 161 - protein, 254 aa, Artificial Sequence
  Antigen binding construct or subpart thereof
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKSGGGGQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGC

SEQ ID NO: 162 - DNA, 762 bp, Artificial Sequence
  Antigen binding construct or subpart thereof
atggaaaccgacaccctgctgctgtgggtgctgctgctctgggtcccaggctccaccggtgaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgctgacctttggcggcggcaccaaagtggaaattaaaagtggtggaggaggccaggtgcagctggtggaaagcggcggcggcgtggtgcagccgggccgcagcctgcgcctgagctgcgcggcgagcggctttaaatttagcggctatggcatgcattgggtgcgccaggcgccgggcaaaggcctggaatgggtggcggtgatttggtatgatggcagcaaaaaatattatgtggatagcgtgaaaggccgctttaccattagccgcgataacagcaaaaacaccctgtatctgcagatgaacagcctgcgcgcggaagataccgcggtgtattattgcgcgcgccagatgggctattggcattttgatctgtggggccgcggcaccctggtgaccgtgagcagcggcggctgc

SEQ ID NO: 163 - protein, 254 aa, Artificial Sequence
  Antigen binding construct or subpart thereof
METDTLLLWVLLLWVPGSTGQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSSGGGGEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGC

SEQ ID NO: 164 - DNA, 762 bp, Artificial Sequence
  Antigen binding construct or subpart thereof
atggaaaccgacaccctgctgctgtgggtgctgctgctctgggtcccaggctccaccggtcaggtgcagctggtggaaagcggcggcggcgtggtgcagccgggccgcagcctgcgcctgagctgcgcggcgagcggctttaaatttagcggctatggcatgcattgggtgcgccaggcgccgggcaaaggcctggaatgggtggcggtgatttggtatgatggcagcaaaaaatattatgtggatagcgtgaaaggccgctttaccattagccgcgataacagcaaaaacaccctgtatctgcagatgaacagcctgcgcgcggaagataccgcggtgtattattgcgcgcgccagatgggctattggcattttgatctgtggggccgcggcaccctggtgaccgtgagcagcagtggtggaggaggcgaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgctgacctttggcggcggcaccaaagtggaaattaaaggcggctgc

SEQ ID NO: 165 - protein, 257 aa, Artificial Sequence
  Antigen binding construct or subpart thereof
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGSGGGGQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGC

SEQ ID NO: 166 - DNA, 771 bp, Artificial Sequence
  Antigen binding construct or subpart thereof
atggaaaccgacaccctgctgctgtgggtgctgctgctctgggtcccaggctccaccggtgaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgctgacctttggcggcggcaccaaagtggaaattaaaggcggagggagtggcggaggcggccaggtgcagctggtggaaagcggcggcggcgtggtgcagccgggccgcagcctgcgcctgagctgcgcggcgagcggctttaaatttagcggctatggcatgcattgggtgcgccaggcgccgggcaaaggcctggaatgggtggcggtgatttggtatgatggcagcaaaaaatattatgtggatagcgtgaaaggccgctttaccattagccgcgataacagcaaaaacaccctgtatctgcagatgaacagcctgcgcgcggaagataccgcggtgtattattgcgcgcgccagatgggctattggcattttgatctgtggggccgcggcaccctggtgaccgtgagcagcggcggctgc

SEQ ID NO: 167 - protein, 257 aa, Artificial Sequence
  Antigen binding construct or subpart thereof
METDTLLLWVLLLWVPGSTGQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGSGGGGEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGC

SEQ ID NO: 168 - DNA, 771 bp, Artificial Sequence
  Antigen binding construct or subpart thereof
atggaaaccgacaccctgctgctgtgggtgctgctgctctgggtcccaggctccaccggtcaggtgcagctggtggaaagcggcggcggcgtggtgcagccgggccgcagcctgcgcctgagctgcgcggcgagcggctttaaatttagcggctatggcatgcattgggtgcgccaggcgccgggcaaaggcctggaatgggtggcggtgatttggtatgatggcagcaaaaaatattatgtggatagcgtgaaaggccgctttaccattagccgcgataacagcaaaaacaccctgtatctgcagatgaacagcctgcgcgcggaagataccgcggtgtattattgcgcgcgccagatgggctattggcattttgatctgtggggccgcggcaccctggtgaccgtgagcagcggcggagggagtggcggaggcggcgaaattgtgctgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgctgacctttggcggcggcaccaaagtggaaattaaaggcggctgc

SEQ ID NO: 169 - DNA, 318 bp, Artificial Sequence
  Antigen binding construct or subpart thereof
caaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaac

SEQ ID NO: 170 - DNA, 318 bp, Homo sapiens
gacatccagatgacccagtctccttcttctctgtctgcttctgtcggagacagagtcacaatcacatgttctgcttctagctctgtctcttacatgaactggtaccagcagacacctggaaaggctcctaagcggtggatctacgacacatctaagctcgcttctggagtcccttctagattctctggttctggctctggaacagactacacattcacaatctcttctctccaacctgaggacatcgctacatactactgccaacagtggtctagcaatcctttcacattcggacagggaacaaagctgcagatcaca

SEQ ID NO: 171 - protein, 106 aa, Homo sapiens [sequence omitted]

SEQ ID NO: 172 - DNA, 318 bp, Artificial Sequence
  Antigen binding construct or subpart thereof
gaaattgtgttgacacagtctccagccaccctgtctttgtctccaggggaaagagccaccctctcctgcagtgccagctcaagtgtaagttacatgaactggtaccaacagaaacctggccaggctcccaggctcctcatctatgacacatccaaactggcttctggagtccctgctcacttcaggggcagtgggtctgggacagacttcactctcaccatcagcagcctagagcctgaagattttgcagtttattactgtcagcagtggagtagtaacccattcacgttcggccaagggaccaaggtggaaatcaaa

SEQ ID NO: 173 - protein, 106 aa, Artificial Sequence
  Antigen binding construct or subpart thereof
EIVLTQSPATLSLSPGERATLSCSASSSVSYMNWYQQKPGQAPRLLIYDTSKLASGVPAHFRGSGSGTDFTLTISSLEPEDFAVYYCQQWSSNPFTFGQGTKVEIK

SEQ ID NO: 174 - DNA, 357 bp, Mus musculus
caggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctca

SEQ ID NO: 175 - DNA, 357 bp, Artificial Sequence
  Antigen binding construct or subpart thereof
caggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaaatgcaactgagcagcctgacatctgaggactctgcagtctattactgtgcaagatattattcagaagttcaaggacaaggccacattgactacagacaaatcctccagcacagcctacgatgatcattactcacttgactactggggccaaggcaccactctcacagtctcctca

SEQ ID NO: 176 - protein, 119 aa, Artificial Sequence
  Antigen binding construct or subpart thereof
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 177 - DNA, 357 bp, Homo sapiens
caggttcagctggtgcagtctggaggaggagtcgtccagcctggaaggtccctgagactgtcttgtaaggcttctggatacaccttcactagatacacaatgcactgggtcagacaggctcctggaaagggactcgagtggattggatacattaatcctagcagaggttatactaactacaatcagactgcagatggactcactcagacctgaggataccggagtctatttttgtgctagatattacaggtgaaggacagattcacaatttctagagacaattctaagaatacagccttcgatgaccactactgtctggactactggggccaaggtaccccggtcaccgtgagctca

SEQ ID NO: 178 - protein, 119 aa, Homo sapiens [sequence omitted]

SEQ ID NO: 179 - DNA, 357 bp, Artificial Sequence
  Antigen binding construct or subpart thereof
caggttcagctggtgcagtctggaggaggagtcgtccagcctggaaggtccctgagactgtcttgtaaggcttctggatacaccttcactagatacacaatgcactgggtcagacaggctcctggaaagggactcgagtggattggatacattaatcctagcagaggttatactaactacaatcagaaggtgaaggacagattcacaatttctagagacaattctaagaatacagccttcctgcagatggactcactcagacctgaggataccggagtctatttttgtgctagatattacgatgaccactactcactggactactggggccaaggtaccccggtcaccgtgagctca

SEQ ID NO: 180 - protein, 119 aa, Artificial Sequence
  Antigen binding construct or subpart thereof
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 181 - DNA, 357 bp, Artificial Sequence
  Antigen binding construct or subpart thereof
caggtgcagctggtgcagtctggggctgaggtgaagaagcctggggcctcagtgaaggtctcctgcaaggcttctggatacaccttcaccaggtacacgatgcactgggtgcgacaggcccctggacaagggcttgagtggatgggatacattaatcctagccgtggttatactaattacaatcagaagttcaaggacagggtcaccatgaccacagacacgtccatcagcacagcctacatggagctgagcaggctgagatctgacgacacggccgtgtattactgtgcgagatattatgatgatcattactgccttgactactggggccagggcaccctggtcaccgtctcctca

SEQ ID NO: 182 - protein, 119 aa, Artificial Sequence; identical to SEQ ID NO: 106
  Antigen binding construct or subpart thereof

SEQ ID NO: 183 - DNA, 357 bp, Artificial Sequence
  Antigen binding construct or subpart thereof
caggtgcagctggtgcagtctggggctgaggtgaagaagcctggggcctcagtgaaggtctcctgcaaggcttctggatacaccttcaccaggtacacgatgcactgggtgcgacaggcccctggacaagggcttgagtggatgggatacattaatcctagccgtggttatactaattacaatcagaagttcaaggacagggtcaccatgaccacagacacgtccatcagcacagcctacatggagctgagcaggctgagatctgacgacacggccgtgtattactgtgcgagatattatgatgatcattactcacttgactactggggccagggcaccctggtcaccgtctcctca

SEQ ID NO: 184 - protein, 119 aa, Artificial Sequence
  Antigen binding construct or subpart thereof
QVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYSLDYWGQGTLVTVSS

SEQ ID NO: 185 - protein, 22 aa, Artificial Sequence
  Antigen binding construct or subpart thereof
MQSGTHWRVLGLCLLSVGVWGQ

SEQ ID NO: 186 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 187 - protein, 23 aa, Artificial Sequence
  Human IgG1 NH1
EPKSCDKTHTCPPCPAPELLGGP

SEQ ID NO: 188 - protein, 24 aa, Artificial Sequence
  Human IgG1 EH1
EPKSCDKTHTCPPCGGGSSGGGSG

SEQ ID NO: 189 - protein, 23 aa, Homo sapiens
EPKSSDKTHTCPPCPAPELLGGP

SEQ ID NO: 190 - protein, 24 aa, Artificial Sequence
  Human IgG1 EH2
EPKSSDKTHTCPPCGGGSSGGGSG

SEQ ID NO: 191 - protein, 25 aa, Artificial Sequence
  Human IgG1 NH3
EPKSSDKTHTCPPCPPCAPELLGGP

SEQ ID NO: 192 - protein, 27 aa, Artificial Sequence
  Human IgG1 EH3
EPKSSDKTHTCPPCPPCGGGSSGGGSG

SEQ ID NO: 193 - protein, 28 aa, Artificial Sequence
  Human IgG1 NH4
EPKSSDKTHTCPPCVECPPCAPELLGGP

SEQ ID NO: 194 - protein, 30 aa, Artificial Sequence
  Human IgG1 EH4
EPKSSDKTHTCPPCVECPPCGGGSSGGGSG

SEQ ID NO: 195 - protein, 28 aa, Artificial Sequence
  Human IgG1 NH5
EPKSSDKTHTCPPCPPCPPCAPELLGGP

SEQ ID NO: 196 - protein, 30 aa, Artificial Sequence; identical to SEQ ID NO: 58
  Human IgG1 EH5

SEQ ID NO: 197 - protein, 19 aa, Artificial Sequence
  Human IgG2 NH1
ERKCCVECPPCPAPPVAGP

SEQ ID NO: 198 - protein, 22 aa, Artificial Sequence
  Human IgG2 EH1
ERKCCVECPPCPGGGSSGGGSG

SEQ ID NO: 199 - protein, 19 aa, Artificial Sequence
  Human IgG2 NH2
ERKSCVECPPCPAPPVAGP

SEQ ID NO: 200 - protein, 22 aa, Artificial Sequence
  Human IgG2 EH2
ERKSCVECPPCPGGGSSGGGSG

SEQ ID NO: 201 - protein, 29 aa, Artificial Sequence
  IgG3/IgG1 EH6
ELKTPLGDTTHTCVECPPCGGGSSGGGSG

SEQ ID NO: 202 - protein, 29 aa, Artificial Sequence
  IgG3/IgG1 EH7
ELKTPLGDTTHTCPPCPPCGGGSSGGGSG

SEQ ID NO: 203 - protein, 32 aa, Artificial Sequence; identical to SEQ ID NO: 64
  IgG3/IgG1 EH8

SEQ ID NO: 204 - protein, 20 aa, Artificial Sequence
  IgG4 NH
ESKYGPPCPPCPAPEFLGGP

SEQ ID NO: 205 - protein, 22 aa, Artificial Sequence
  IgG4 EH
ESKYGPPCPPCPGGGSSGGGSG

SEQ ID NO: 206 - protein, 10 aa, Artificial Sequence
  Upper hinge
EPKSCDKTHT

SEQ ID NO: 207 - protein, 10 aa, Artificial Sequence
  Upper hinge
EPKSSDKTHT

SEQ ID NO: 208 - protein, 3 aa, Artificial Sequence
  Upper hinge
ERK

SEQ ID NO: 209 - protein, 12 aa, Artificial Sequence
  Upper hinge
ELKTPLGDTTHT

SEQ ID NO: 210 - protein, 7 aa, Artificial Sequence
  Upper hinge
ESKYGPP

SEQ ID NO: 211 - protein, 4 aa, Homo sapiens
CPPC

SEQ ID NO: 212 - protein, 5 aa, Homo sapiens
CPPCP

SEQ ID NO: 213 - protein, 7 aa, Homo sapiens
CPPCPPC

SEQ ID NO: 214 - protein, 10 aa, Homo sapiens
CPPCVECPPC

SEQ ID NO: 215 - protein, 10 aa, Homo sapiens
CPPCPPCPPC

SEQ ID NO: 216 - protein, 9 aa, Homo sapiens
CCVECPPCP

SEQ ID NO: 217 - protein, 9 aa, Homo sapiens
SCVECPPCP

SEQ ID NO: 218 - protein, 7 aa, Homo sapiens
CVECPPC

SEQ ID NO: 219 - protein, 10 aa, Homo sapiens
GGGSSGGGSG

SEQ ID NO: 220 - protein, 7 aa, Homo sapiens
APPVAGP

SEQ ID NO: 221 - protein, 8 aa, Homo sapiens
APEFLGGP

SEQ ID NO: 222 - protein, 10 aa, Artificial Sequence
  HCDR3
YYDDHYCLDY

SEQ ID NO: 223 - protein, 10 aa, Artificial Sequence
  HCDR3
YYDDHYSLDY

SEQ ID NO: 224 - protein, 10 aa, Artificial Sequence
  HCDR3
  VARIANT 7: Xaa = C or S
YYDDHYXLDY

SEQ ID NO: 225 - protein, 5 aa, Artificial Sequence
  Synthetic Peptide
HGRGH

SEQ ID NO: 226 - protein, 5 aa, Artificial Sequence
  Synthetic Peptide
  VARIANT (1)...(5): D-amino acid
WPLRF

SEQ ID NO: 227 - protein, 9 aa, Artificial Sequence
  Synthetic Peptide
AAEGLDTQR

SEQ ID NO: 228 - protein, 9 aa, Artificial Sequence
  Synthetic Peptide
SQFRVSPLD

SEQ ID NO: 229 - protein, 5 aa, Artificial Sequence
  Synthetic Peptide
AKYRG

SEQ ID NO: 230 - protein, 10 aa, Artificial Sequence
  Synthetic Peptide
FLLYLSQNKP

SEQ ID NO: 231 - protein, 5 aa, Artificial Sequence
  Synthetic Peptide
PWTHG

SEQ ID NO: 232 - protein, 5 aa, Artificial Sequence
  Synthetic Peptide
HGSYG

SEQ ID NO: 233 - protein, 5 aa, Artificial Sequence
  Synthetic Peptide
KRLGA

SEQ ID NO: 234 - protein, 5 aa, Artificial Sequence
  Synthetic Peptide
  VARIANT (1)...(5): D-amino acid
HALLW

SEQ ID NO: 235 - protein, 5 aa, Artificial Sequence
  Synthetic Peptide
  VARIANT (1)...(2): D-amino acid
  VARIANT (4)...(5): D-amino acid
LRGYW

SEQ ID NO: 236 - protein, 5 aa, Artificial Sequence
  Synthetic Peptide
  VARIANT (1)...(5): D-amino acid
VASHF

SEQ ID NO: 237 - protein, 5 aa, Artificial Sequence
  Synthetic Peptide
  VARIANT (1)...(1): D-amino acid
  VARIANT (3)...(5): D-amino acid
NGNVH

SEQ ID NO: 238 - protein, 5 aa, Artificial Sequence
  Synthetic Peptide
  VARIANT (1)...(5): D-amino acid
RWFNV

SEQ ID NO: 239 - protein, 5 aa, Artificial Sequence
  Synthetic Peptide
  VARIANT (1)...(5): D-amino acid
HAVWH

SEQ ID NO: 240 - protein, 5 aa, Artificial Sequence
  Synthetic Peptide
  VARIANT (1)...(5): D-amino acid
WVPLW

SEQ ID NO: 241 - protein, 5 aa, Artificial Sequence
  Synthetic Peptide
  VARIANT (1)...(5): D-amino acid
FFRLY

SEQ ID NO: 242 - protein, 5 aa, Artificial Sequence
  Synthetic Peptide
  VARIANT (1)...(3): D-amino acid
  VARIANT (5)...(5): D-amino acid
WYYGF

SEQ ID NO: 243 - protein, 5 aa, Artificial Sequence
  Synthetic Peptide
AGDSW

SEQ ID NO: 244 - protein, 5 aa, Artificial Sequence
  Synthetic Peptide
HVRHG

SEQ ID NO: 245 - protein, 5 aa, Artificial Sequence
  Synthetic Peptide
  VARIANT (2)...(5): D-amino acid
GHTWP

SEQ ID NO: 246 - protein, 5 aa, Artificial Sequence
  Synthetic Peptide
  VARIANT (1)...(1): D-amino acid
  VARIANT (3)...(3): D-amino acid
  VARIANT (5)...(5): D-amino acid
HGRGH

SEQ ID NO: 247 - protein, 5 aa, Artificial Sequence
  Synthetic Peptide
THPTT

SEQ ID NO: 248 - protein, 5 aa, Artificial Sequence
  Synthetic Peptide
FAGYH

SEQ ID NO: 249 - protein, 5 aa, Artificial Sequence
  Synthetic Peptide
WTEHG

SEQ ID NO: 250 - protein, 5 aa, Artificial Sequence
  Synthetic Peptide
TNDFD

SEQ ID NO: 251 - protein, 5 aa, Artificial Sequence
  Synthetic Peptide
LFPFD

SEQ ID NO: 252 - protein, 5 aa, Artificial Sequence
  Synthetic Peptide
  VARIANT (1)...(4): D-amino acid
SLRFG

SEQ ID NO: 253 - protein, 5 aa, Artificial Sequence
  Synthetic Peptide
  VARIANT (1)...(3): D-amino acid
  VARIANT (5)...(5): D-amino acid
YFRGS

SEQ ID NO: 254 - protein, 5 aa, Artificial Sequence
  Synthetic Peptide
  VARIANT (1)...(4): D-amino acid
WNWVG

SEQ ID NO: 255 - protein, 5 aa, Artificial Sequence
  Synthetic Peptide
  VARIANT (1)...(4): D-amino acid
VAWLG

SEQ ID NO: 256 - protein, 5 aa, Artificial Sequence
  Synthetic Peptide
  VARIANT (1)...(4): D-amino acid
FHVHG

SEQ ID NO: 257 - protein, 5 aa, Artificial Sequence
  Synthetic Peptide
  VARIANT (1)...(5): D-amino acid
WVSNV

SEQ ID NO: 258 - protein, 5 aa, Artificial Sequence
  Synthetic Peptide
  VARIANT (1)...(5): D-amino acid
WSVNV

SEQ ID NO: 259 - protein, 5 aa, Artificial Sequence
  Synthetic Peptide
  VARIANT (1)...(4): Leu Asn Ser His Gly
LNSHG

SEQ ID NO: 260 - protein, 5 aa, Artificial Sequence
  Synthetic Peptide
  VARIANT (1)...(1): D-amino acid
  VARIANT (4)...(5): D-amino acid
LNSHG

SEQ ID NO: 261 - protein, 5 aa, Artificial Sequence
  Synthetic Peptide
  VARIANT (1)...(4): D-amino acid
NSVHG

SEQ ID NO: 262 - protein, 5 aa, Artificial Sequence
  Synthetic Peptide
  VARIANT (1)...(4): D-amino acid
TTVHG

SEQ ID NO: 263 - protein, 5 aa, Artificial Sequence
  Synthetic Peptide
  VARIANT (1)...(3): D-amino acid
  VARIANT (5)...(5): D-amino acid
FDVGH

SEQ ID NO: 264 - protein, 5 aa, Artificial Sequence
  Synthetic Peptide
  VARIANT (1)...(2): D-amino acid
  VARIANT (4)...(5): D-amino acid
RHGWK

SEQ ID NO: 265 - protein, 5 aa, Artificial Sequence
  Synthetic Peptide
  VARIANT (1)...(1): D-amino acid
  VARIANT (3)...(3): D-amino acid
  VARIANT (5)...(5): D-amino acid
HGRGH